(19) United States
(12) Patent Application Publication
(10) Pub. No.: US 2024/0398767 A1
(43) Pub. Date: Dec. 5, 2024

(54) INHIBITORS OF THE MTDH-SND1 PROTEIN COMPLEX FOR CANCER THERAPY

(71) Applicant: The Trustees of Princeton University, Princeton, NJ (US)

(72) Inventors: Minhong Shen, Kendall Park, NJ (US); Hahn Kim, Princeton, NJ (US); Yibin Kang, Princeton, NJ (US)

(21) Appl. No.: 18/693,401

(22) PCT Filed: Sep. 21, 2022
(86) PCT No.: PCT/US2022/076803

§ 371 Date: Mar. 19, 2024

Related U.S. Application Data
(60) Provisional application No. 63/262,474, filed on Oct. 13, 2021.
(60) Provisional application No. 63/246,518, filed on Sep. 21, 2021.

(51) Int. Cl. A61K 31/437; A61K 31/444; A61K 31/4985; A61K 31/5025; A61K 31/519; A61K 39/395; A61K 45/06; A61P 35/00; C07D 471/04; C07D 487/04

(52) U.S. Cl. CPC A61K 31/437; A61K 31/444; A61K 31/4985; A61K 31/5025; A61K 31/519; A61K 39/3955; A61K 45/06; A61P 35/00; C07D 471/04; C07D 487/04

(57) ABSTRACT

Provided herein are methods of using small molecule inhibitors of the MTDH-SND1 protein-protein interaction, such as a compound of the following structural formula: (I) or a pharmaceutically acceptable salt thereof, wherein values for the variables (e.g., X¹, X², X³, X⁴, X⁵, X⁶, X⁷, R³, R⁴, m) are as described herein. The compounds described herein can be used, for example, to treat cancer as, for example, by inhibiting metastasis of a cancer, sensitizing a cancer to treatment with an additional therapy, and/or promoting T-cell activation and/or infiltration in response to a cancer.

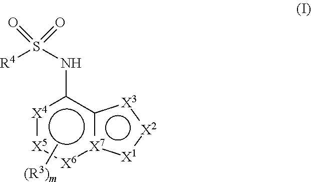

### RELATED APPLICATIONS

This application claims the benefit of U.S. Provisional Application No. 63/262,474, filed Oct. 13, 2021, and U.S. Provisional Application No. 63/246,518, filed Sep. 21, 2021. The entire teachings of the above applications are incorporated herein by reference.

### GOVERNMENT SUPPORT

This invention was made with government support under Grant No. R01CA134519 awarded by the National Institutes of Health and Grant No. W81XWH-16-1-0419 awarded by the U.S. Department of Defense, Army Medical Research & Materiel Command. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
File name: 53911033002.xml; created Sep. 9, 2022, 34,623 Bytes in size.

### BACKGROUND

U.S. Pat. No. 9,932,587 describes methods of identifying and treating poor prognosis cancers by administering an agent that inhibits an activity of metadherin. U.S. Pat. No. 10,357,539 describes use of peptides that block metadherin-Staphylococcal nuclease domain-containing 1 (MTDH-SND1) interaction to treat cancer.

There remains a need for small molecule inhibitors of the MTDH-SND1 protein-protein interaction.

### SUMMARY OF THE INVENTION

Provided herein are small molecule inhibitors of the MTDH-SND1 protein-protein interaction, and methods of using the inhibitors, for example, to treat cancer as, for example, by inhibiting metastasis of a cancer, sensitizing a cancer to treatment with an additional therapy, and/or promoting T-cell activation and/or infiltration in response to a cancer.

One embodiment provides a compound of the following structural formula:

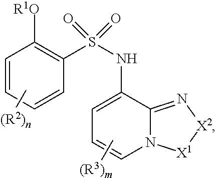

or a pharmaceutically acceptable salt thereof, wherein values for the variables (e.g., X¹, X², R¹, R², R³, n, m) are as described herein.

Another embodiment provides a compound of the following structural formula:

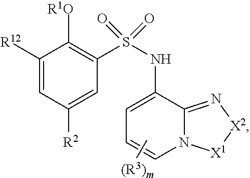

or a pharmaceutically acceptable salt thereof, wherein values for the variables (e.g., X¹, X², R¹, R², R³, R¹², m) are as described herein.

Another embodiment provides a composition (e.g., pharmaceutical composition) comprising a compound of the disclosure and one or more pharmaceutically acceptable carriers.

Yet other embodiments provide a method of inhibiting an interaction between MTDH and SND1; stabilizing or increasing the level or expression of transporter associated with antigen processing (TAP); inhibiting degradation of Tap; and/or promoting tumor antigen presentation in a cell, comprising contacting the cell with a compound of the disclosure, such as a compound of one of the following structural formulas:

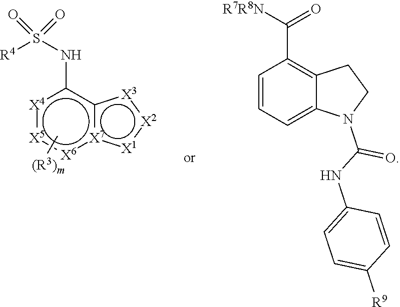

or a pharmaceutically acceptable salt thereof, wherein values for the variables (e.g., X¹, X², X³, X⁴, X⁵, X⁶, X⁷, R³, R⁴, R⁷, R⁸, R⁹, m) are as described herein.

Other embodiments provide methods of treating a cancer; inhibiting metastasis of a cancer; sensitizing a cancer to treatment with radiation therapy, chemotherapy, and/or immune therapy; and/or promoting T-cell activation and/or infiltration in response to a cancer in a subject in need thereof, comprising administering to the subject an effective amount of a compound of the disclosure.

Other embodiments provide a compound of the disclosure for use in any of the methods described herein (e.g., inhibiting an interaction between MTDH and SND1, treating a cancer). Yet other embodiments provide a composition (e.g., pharmaceutical composition) comprising a compound of the disclosure for use in any of the methods described herein (e.g., inhibiting an interaction between MTDH and SND1, treating a cancer). Other embodiments provide use of a compound of the disclosure for the manufacture of a medicament for use as described herein (e.g., inhibiting an interaction between MTDH and SND1, treating a cancer).

Other embodiments provide a nucleic acid molecule comprising the nucleotide sequence of SEQ ID NO:1 or SEQ ID NO:3, or a nucleotide sequence having at least 75%, at least 85%, at least 90% or at least 95% identity to the nucleotide sequence of SEQ ID NO:1 or SEQ ID NO:3, respectively.

Other embodiments provide a protein comprising the amino acid sequence of SEQ ID NO:2 or SEQ ID NO:4, or an amino acid sequence having at least 75%, at least 85%, at least 90% or at least 95% identity to the amino acid sequence of SEQ ID NO:2 or SEQ ID NO: 4, respectively.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIGS. 1A-1H show induced Mtdh knockout suppresses breast cancer progression and metastasis.

FIG. 1A, Schematic diagram of Mtdh^{floxed/floxed} (Mtdh^{fl/fl}) mice (top). Genotyping result of Mtdh wild type (Mtdh^{+/+}, 371 bp), conditional Mtdh knockout heterozygous (Mtdh^{fl/+}, 371 bp and 522 bp), and conditional Mtdh knockout homozygous (Mtdh^{fl/fl}, 522 bp) (bottom left). Western blotting of MTDH in splenocytes from indicated strains cultured with multiplicity of infection (MOI, 100) adenovirus expressing Cre for 0, 3, or 5 days (bottom right). F, forward primer and R, reverse primer for genotyping.

FIG. 1B, Schematic diagram of generation of Mtdh inducible knockout mice (Top). Cre expression is induced by tamoxifen (Tmx) in FVB.UBC-Cre^{ERT+/-} strain.

FIG. 1C, FVB.UBC-Cre^{ERT+/-};Mtdh^{fl/fl} strain was bred with FVB.MMTV-PyMT strain to generate breast cancer mouse model with Mtdh inducible knockout. Mice with matched tumor sizes were treated with Tmx or vehicle for 5 consecutive days via i.p. Tumors were measured weekly and lung metastasis was evaluated at endpoint.

FIG. 1D, FVB.PyMT;UBC-Cre^{ERT+/-}; Mtdh^{fl/fl} mice with tumors established were split into two groups with matched tumor sizes for vehicle (n=18 mice) or Tmx (n=30 mice) treatment, respectively. Tumor burden before treatment was showed. Data represent mean SEM. Significance determined by two tailed Student's t-test.

FIG. 1E, Tumor progression curves are shown after treatment starts. Each primary tumor was measured, and sizes were added as tumor burden in each mouse after treatment. Vehicle, n=18; Tmx, n=30. Data represent mean±SEM. Significance determined by Two-Way Repeated Measures ANOVA test.

FIG. 1F, Tumor burden-based survival was plotted. 500 mm³ was used as cutoff for moribund condition as defined in the IACUC protocol. p value by two-sided Log-rank test. Vehicle, n=18; Tmx, n=30.

FIG. 1G, Lungs were collected, fixed and subjected to hematoxylin and eosin (H&E) staining (left). Metastatic nodules were counted (right). The metastatic nodules of the representative lungs were highlighted with red and blue respectively (right). Vehicle, lung=18; Tmx, lung=30. Size bar, 5 mm. Data represent mean±SEM. Significance determined by two tailed Student's t-test.

FIG. 1H, MTDH expression in tumors from the mice treated with vehicle or Tmx was evaluated with western blot.

FIGS. 2A-2I show MTDH-SND1 interaction is essential for breast cancer progression and metastasis.

FIG. 2A, Primary tumors from FVB.PyMT;UBC-Cre^{ERT+/-};Mtdh^{fl/fl} mice were cultured to generate cell line (left). The cell line treated with 4-OHT was harvested for western blotting (right). 4-OHT, (Z)-4-Hydroxytamoxifen.

FIG. 2B, Tumorsphere assay was performed with FVB.PyMT; UBC-Cre^{ERT+/-};Mtdh^{fl/fl} cells. Sphere number and size were determined and normalized to vehicle group. Data represent mean±SEM. n=3 independent experiments. Significance determined by two tailed Student's t-test.

FIG. 2C, 10 k of FVB.PyMT; UBC-Cre^{ERT+/-};Mtdh^{fl/fl} cells were orthotopically inoculated into FVB females. Two weeks after injection, mice were treated with or without Tmx. Tumors were measured before treatment. Vehicle, n=12, Tmx, n=8. Data represent mean±SEM. Significance determined by two tailed Student's t-test.

FIG. 2D, 10 k of FVB.PyMT; UBC-Cre^{ERT+/-};Mtdh^{fl/fl} cells were orthotopically inoculated into FVB females. Two weeks after injection, mice were treated with or without Tmx. Tumors were measured after treatment. Vehicle, n=12, Tmx, n=8. Data represent mean±SEM. Significance determined by Two-Way Repeated Measures ANOVA test.

FIG. 2E, 11 weeks after injection, lungs were collected, and metastatic nodules were counted. The metastatic nodules of the representative lungs were highlighted with red and blue respectively. Size bar, 5 mm. Vehicle, lung=12; Tmx, lung=8. Data represent mean±SEM. Significance determined by two tailed Student's t-test.

FIG. 2F, PyMT;UBC-Cre^{ERT+/-};Mtdh^{fl/fl} cells expressing GFP (vector), wild type MTDH (MTDH-WT), or SND1 interaction deficient MTDH (MTDH-13D) were treated with 4-OHT followed by western blotting.

FIG. 2G, Sphere number and size in 4-OHT treatment groups were determined and normalized to vehicle controls of the same cell line. Data represent mean±SEM. n=3 independent experiments. Significance determined by one-way ANOVA analysis with Sidak's test for multiple comparisons.

FIG. 2H, 50,000 (50 k) of the indicated cells were orthotopically injected into FVB female mice. One week after injection, the mice were treated with or without Tmx. 6 weeks later, tumor size and weight were measured. n=6 mice per group. Data represent mean±SEM. Significance determined by two tailed Student's t-test.

FIG. 2I, Lungs were fixed, and the metastatic nodules were quantified. The metastatic nodules of the representative lungs were highlighted with red. n=6 lungs per group. Size bar, 5 mm. Data represent mean±SEM. Significance determined by two tailed Student's t-test (n.s. p>0.05, ****p<0.0001).

FIGS. 3A-3F show identification of small chemical inhibitors that block MTDH-SND1 interaction.

FIG. 3A, Schematic diagram of the small molecule screening platform Split & Linked-luciferase (Split-luc, Linked-luc) assay.

FIG. 3B, Schematic diagram of the small molecule screening platform FRET assay.

FIG. 3C, Workflow of the screening.

FIG. 3D, Structure of the three positive candidates.

FIG. 3E, Split-luciferase assay was performed with multiple doses of indicated compounds or MTDH wild type peptide (Pep-WT). Data represent mean±SEM. Luciferase inhibitory efficiency was calculated, and curves were fit. IC50s (μM) are shown following each compound/peptide in parentheses.

FIG. 3F, SCP28 cells grown confluent in each 10-cm dish were lysed with 1 ml IP lysis buffer. 500 μM of the compounds were added into each 1 ml of the samples and IP lysis buffer with 2 μg of anti-MTDH antibody. Western blot was then performed to detect SND1 that binds to MTDH.

FIGS. 4A-4G show C26-A2 and A6 suppress tumor formation in vitro.

FIG. 4A, SCP28 cells that stably express split- or linked-luciferase components were treated with multiple doses of C26-A2 or C26-A6. 30 minutes (min) after treatment, culture media was removed and the luciferase activity in the cells was measured. Data represent mean±SEM. n=3 independent experiments.

FIG. 4B, The same cells as in FIG. 4A were treated with 100 μM of the compounds for indicated days, and were harvested to measure the luciferase activity. Data represent mean±SEM. n=3 independent experiments.

FIG. 4C, PyMT;UBC-Cre^{ERT+/-};Mtdh^{fl/fl} cells without 5 days of 0.02 μg/ml 4-OHT pre-treatment was employed for tumorsphere assay. 50 k per well of cells were seed and treated with indicated compounds the next day. 5 days after treatment, sphere number and size were assessed and normalized to vehicle control group. Data represent mean±SEM. n=3 independent experiments. Significance determined by one-way ANOVA analysis with Sidak's test for multiple comparisons.

FIG. 4D, PyMT;UBC-Cre^{ERT+/-};Mtdh^{fl/fl} cells with 5 days of 0.02 μg/ml 4-OHT pre-treatment was employed for tumorsphere assay. 50 k per well of cells were seed and treated with indicated compounds the next day. 5 days after treatment, sphere number and size were assessed and normalized to vehicle control group. Data represent mean±SEM. n=3 independent experiments. Significance determined by one-way ANOVA analysis with Sidak's test for multiple comparisons.

FIG. 4E, PyMT;UBC-Cre^{ERT+/-};Mtdh^{fl/fl} cells with or without SND1 knockdown, or without 5 days of 0.02 μg/ml 4-OHT pre-treatment were subjected to the tumorsphere assay and then treated with 200 μM of C26-A6 similar to FIG. 4C. Data represent mean SEM. n=3 independent experiments. Significance determined by two tailed Student's t-test.

FIG. 4F, PyMT;UBC-Cre^{ERT+/-};Mtdh^{fl/fl} cells with or without SND1 knockdown, or with 5 days of 0.02 μg/ml 4-OHT pre-treatment were subjected to the tumorsphere assay and then treated with 200 μM of C26-A6 similar to FIG. 4C. Data represent mean±SEM. n=3 independent experiments. Significance determined by two tailed Student's t-test.

FIG. 4G, The expression of SND1 and MTDH was validated by western blot analysis.

FIGS. 5A-5M show MTDH-SND1 complex disruption suppresses breast cancer progression and metastasis.

FIG. 5A, Schematic diagram of the treatments in FVB female mice.

FIG. 5B, Tumor size was determined. Vehicle, n=10 mice, C26-A6, n=12 mice. Significance determined by Two-Way Repeated Measures ANOVA test.

FIG. 5C, Tumor mass was determined. Vehicle, n=10 mice, C26-A6, n=12 mice. Size bar, 2 cm.

FIG. 5D, H&E staining was performed with lungs, and metastatic nodules were counted. The metastatic nodules of the representative lungs were highlighted with red and blue respectively. Vehicle, n=10 lungs; C26-A6, n=12 lungs. Size bar, 5 mm.

FIG. 5E, Gene set enrichment analysis plot showing the enrichment of Tmx treatment-upregulated (left), -downregulated (middle), or SND1-upregulated (right) gene signatures. p and q values were determined by Kolmogorov-Smirnov statistic with GSEA v3.0.

FIG. 5F, PyMT;UBC-Cre^{ERT+/-};Mtdh^{fl/fl} tumor cells with 4-OHT pre-treated were assessed by western blot.

FIG. 5G, 10 k of the cells of FIG. 5F were inoculated into FVB female mice and treated with or without C26-A6 after primary tumors reached to approximately 2 mm in diameter. 6 weeks after treatment, tumor size was determined. Vehicle, n=10 mice; C26-A6, n=10 mice.

FIG. 5H, 10 k of the cells of FIG. 5F were inoculated into FVB female mice and treated with or without C26-A6 after primary tumors reached to approximately 2 mm in diameter. 6 weeks after treatment, spontaneous lung metastasis was determined. The lung metastatic areas of the representative lungs were highlighted with red. Vehicle, n=10 mice; C26-A6, n=10 mice. Size bar, 5 mm.

FIG. 5I, PyMT tumor cells with endogenous SND1 stably knockdown was confirmed by western blot.

FIG. 5J, 10 k of the cells of FIG. 5I were injected into FVB females and treated similarly as in FIGS. 5G, 5H. Tumor size was assessed. Vehicle, n=10 mice; C26-A6, n=10 mice.

FIG. 5K, 10 k of the cells of FIG. 5I were injected into FVB females and treated similarly as in FIGS. 5G, 5H. Lung metastasis was assessed. The lung metastatic areas of the representative lungs were highlighted with red. Vehicle, n=10 mice; C26-A6, n=10 mice. Size bar, 5 mm.

FIG. 5L, 2 k PyMT tumor cells were injected into FVB females via tail-vein. 3 days after injection, the mice were treated with vehicle or C26-A6.

FIG. 5M, 2 k PyMT tumor cells were injected into FVB females via tail-vein. 3 days after injection, the mice were treated with vehicle or C26-A6. 5 weeks later, lung metastatic nodules were counted. n=6 mice per group. Data represent mean±SEM.

Significance determined by two tailed Student's t-test (FIGS. 5C, 5D, 5J, 5H, 5K, 5M). AU: arbitrary units.

FIGS. 6A-6J show MTDH-SND1-targeting and chemotherapy synergistically suppress breast cancer progression and metastasis.

FIG. 6A, PyMT;UBC-Cre^{ERT+/-};Mtdh^{fl/fl} mice treatment scheme.

FIG. 6B, Primary tumors were quantified. n=5 mice per group.

FIG. 6C, Spontaneous lung metastatic nodules were quantified. The metastatic nodules of the representative lungs were highlighted with red. n=5 mice per group. Size bar, 5 mm.

FIG. 6D, Kaplan-Meier plots of overall survival (OS), relapse-free survival (RFS), and lung metastasis-free survival (LMFS) of TNBC patients.

FIG. 6E, NSG female mice were injected with 10 k of SCP28 cells. One week after injection the mice were treated with C26-A6 or paclitaxel (Pac) alone or in combination for 5 weeks. Primary tumor size was measured. n=6 mice per group.

FIG. 6F, Lungs from FIG. 6E were harvested and BLI signal was measured to determine spontaneous lung metastasis. Data represent mean±SEM. n=6 lungs.

FIG. 6G, Balb/C females were injected with 1000 4T1 cells via tail-vein. 3 days after injection, the mice received similar treatment as in FIG. 6E for 5 weeks. Lungs were fixed, and the metastatic nodules were counted. The metastatic nodules of the representative lungs were highlighted with red. Vehicle, n=11 mice; n=12 mice for other groups. Size bar, 5 mm.

FIG. 6H, Survival rate of the mice in experiment of FIG. 6G was plotted.

FIG. 6I, SCP28 primary tumors were removed from NSG female mice when they reached approximately 5 mm in diameter. The mice were treated with C26-A6 and Pac alone or in combination. Lungs were collected to count metastatic nodules at endpoint. The metastatic nodules of the representative lungs were highlighted with red. n=12 mice per group. Size bar, 5 mm. Data represent mean±SEM.

FIG. 6J, SCP28 primary tumors were removed in NSG female mice when they reached to approximately 5 mm in diameter. The mice were treated with C26-A6 and Pac alone or in combination. Survival rate in each group was analyzed. n=12 mice per group. Size bar, 5 mm. Data represent mean±SEM.

Significance determined by one-way ANOVA analysis with Sidak's test for multiple comparisons (FIGS. 6B, 6C, 6E, 6F, 6G, 6I) and two-sided Log-rank test (FIGS. 6D, 6H, 6J).

FIGS. 7A-7H show C26-A6 enhances chemotherapy response in metastatic breast cancer model without additional toxicity.

For FIGS. 7A-7D, 2 k 4TO7 cells were injected into Balb/C females. 2 weeks after the injections, the mice were randomized based on lung metastasis that indicated by BLI, and were divided into four groups followed by vehicle, paclitaxel (Pac), and C26-A6 treatment alone or in combination. For Pac, the mice were treated with 20 mg/kg of Pac twice per week for the first two week and then once per week after that, For C26-A6, the mice were treated with 15 mg/kg of C26-A6 5 days per week. n=6 mice per group. Data represent mean±SEM.

FIG. 7A, Representative mice right before the treatment (week 2) and at week 10 are shown.

FIG. 7B, The BLI signal was quantified at week 2. Significance determined by one-way ANOVA analysis with Dunnett's test for multiple comparisons.

FIG. 7C, The metastasis progression of each individual is shown.

FIG. 7D, Survival rate in each group was analyzed. Significance determined by two-sided Log-rank test.

FIG. 7E, Serum from mice in FIG. 7A were collected for ALT and AST activity measurement following the standard protocol (Sigma). Data represent mean±SEM. AST, n=8 replicates from 6 mice; ALT, n=9 replicates from 6 mice. Significance determined by one-way ANOVA analysis with Sidak's test for multiple comparisons.

FIG. 7F, Blood samples were drawn from the heart of mice in FIG. 7A, and blood cell counts were performed with the Sysemx XN-3000 Hematology System (Sysmex America, Inc.). Data represent mean±SEM. n=6 mice per group. Significance determined by one-way ANOVA analysis with Sidak's test for multiple comparisons.

FIG. 7G, Small intestine samples were obtained from mice in FIG. 7A. H&E and Alcian blue staining was performed on processed, sliced samples. Scale bar: 200 μm.

FIG. 7H, Quantification of Alcian blue staining results from FIG. 7G. Data represent mean±SEM. n=12 fields from 6 mice in each group. Significance determined by one-way ANOVA analysis with Sidak's test for multiple comparisons.

FIGS. 8A-8N show Mtdh acute knockout inhibits breast cancer progression and metastasis. C3 model: Vehicle, n=9; Tmx, n=9 (FIGS. 8C-8F). WNT model: Vehicle, n=9; Tmx, n=12 (FIGS. 8I-8L).

FIG. 8A, Treatment response of each individual mouse in FIG. 1E.

FIG. 8B, More representative lungs for FIG. 1G. Size bars, 5 mm.

FIG. 8C, Tumor burden of FVB.C3;UBC-Cre^{ERT+/-};Mtdh^{fl/fl} mice before treatment.

FIG. 8D, Tumor burdens were showed as in groups in C3 tumor model after treatment.

FIG. 8E, Tumor burdens were showed as in individuals in C3 tumor model after treatment.

FIG. 8F, Tumor burden-based survival was plotted. 500 mm³ was used as cutoff based on the moribund criteria set in the IACUC protocol. p value by Log-rank test.

FIG. 8G, MTDH expression in tumors from C3 mice that were treated with vehicle or Tmx was evaluated with western blot.

FIG. 8H, Lungs from C3 mice were fixed. H&E staining was performed and metastatic incidence was quantified. C3: Vehicle, n=9 lungs; Tmx, n=9 lung; WNT: Vehicle, n=9 lungs; Tmx, n=12 lung. Size bar, 5 mm.

FIG. 8I, Tumor burden of FVB.WNT;UBC-Cre^{ERT+/-};Mtdh^{fl/fl} mice before treatment.

FIG. 8J, Tumor burdens were showed as in groups in WNT tumor models after treatment.

FIG. 8K, Tumor burdens were showed as in individuals in WNT tumor models after treatment.

FIG. 8L, Tumor burden-based survival was plotted. 500 mm³ was used as cutoff based on the moribund criteria set in the IACUC protocol. p value by Log-rank test.

FIG. 8M, MTDH expression in tumors from WNT mice that were treated with vehicle or Tmx was evaluated with western blot.

FIG. 8N, Lungs from WNT mice were fixed. H&E staining was performed and nodules were quantified. The metastatic nodules of the representative lungs were highlighted with red and blue respectively. C3: Vehicle, n=9 lungs; Tmx, n=9 lung; WNT: Vehicle, n=9 lungs; Tmx, n=12 lung. Size bar, 5 mm.

Data represent mean±SEM. Significance determined by two tailed Student's t-test (FIGS. 8C, 8H, 8I, 8N), two-sided Log-rank test (FIGS. 8F, 8L), Two-Way Repeated Measures ANOVA test (FIGS. 8D, 8J).

FIGS. 9A-9H show tamoxifen by itself does not affect tumorsphere formation.

FIG. 9A, Primary tumors from PyMT, C3, or WNT mice with vehicle or Tmx treatment were stained with Ki67 or cleaved caspase 3 (Casp-3). Images were acquired at non-necrotic/apoptotic areas that were close to tumor border. Size bar, 50 μm.

FIG. 9B, Positive cells in FIG. 9A were quantified. Data represent mean±SEM. Significance determined by two tailed Student's t-test.

FIG. 9C, PyMT; UBC-Cre^{ERT+/-};Mtdh^{fl/fl} cells that were pretreated with 0.02 μg/ml of 4-OHT for 5 days were recovered for another 2 weeks. 25 k cells were then seeded in each well of the 24-well low attachment plate. One day after seeding, cells were treated with vehicle or 0.02 μg/ml of 4-OHT. 10 days after treatment, sphere number and size were measured and normalized to vehicle control group. Data represent mean±SEM. n=3 independent experiments. Significance determined by two tailed Student's t-test.

FIG. 9D, Representative images for tumorspheres in FIG. 2B and FIG. 9C are shown. Size bar, 200 μm.

FIG. 9E, Tumors from FIG. 2D were dissected. Size bar, 2 cm.

FIG. 9F, Tumor mass of tumors from FIG. 2D was measured. Data represent mean±SEM. Significance determined by two tailed Student's t-test.

FIG. 9G, H&E-stained sections of tumors in FIG. 9E are complemented by high-magnification images. Size bar, 5 mm.

FIG. 9H, Cell lines in FIG. 2F were pretreated with 0.02 μg/ml of 4-OHT for 5 days and then recovered for another 2 weeks. The cells were employed for tumorsphere assay with 25 k cells per well. Similar treatment as in FIG. 9C was performed and number and size of the spheres in 4-OHT treatment groups were measured and normalized to vehicle controls of the same cell line. Data represent mean±SEM. n=3 independent experiments. Significance determined by two tailed Student's t-test.

FIGS. 10A-10E show screening of small chemical compounds that disrupt MTDH/SND1 interaction.

FIG. 10A, 293T cells that expressed wild type luciferase or indicated split-luciferase components were lysed and subjected to luciferase assay. Data represent mean SEM. n=3 independent experiments.

FIG. 10B, 293T cells that were transfected with CLuc-MTDH-HA and Myc-SND1-NLuc plasmids were lysed for Co-IP assay 3 days later.

FIG. 10C, 293T cells that express split or linked luciferase components were lysed for luciferase assay. 50 μM of wild type (WT) or SND1 interaction-deficient (MT) MTDH peptides were added into the luciferase assay system. Luciferase activity was measured and normalized to control sample. Data represent mean±SEM. n=3 independent experiments. Significance determined by one-way ANOVA analysis with Dunnett's test for multiple comparisons.

FIG. 10D, 0.5 μM of CFP-MTDH and 2 μM of TC-SND1 that labeled with 2.4 μM of FIAsH-EDT₂ labeling reagent was used to performed FRET assay in 50 μL system. Indicated concentration of wild type (WT) or mutant (MT) MTDH peptides were added and FRET efficiency was calculated. Data represent mean±SEM. n=3 independent experiments. Significance determined by one-way ANOVA analysis with Sidak's test for multiple comparisons.

FIG. 10E, Schematic diagram of Co-IP based confirmation of MTDH-SND1 inhibitory compounds (left). SCP28 cells were lysed for IP assay.2 μg of MTDH antibody together with 500 μM of MTDH wild type (Pep-WT) or mutant (Pep-MT) peptides were added into each 1 ml of samples. Red star indicates wild type MTDH peptide competing off SND1 that binds to MTDH.

FIGS. 11A-11E show C26-A2 and A6 inhibits tumorsphere formation in vitro.

FIG. 11A, Caco-2 cells were employed to test cell permeability of C26-A2 and A6. 5 μM of compounds were dosed on both apical side (A-to-B) and basolateral side (B-to-A). Samples were taken from the donor and receiver chambers at 120 min after treatment. All samples were assayed by LC-MS/MS using electrospray ionization. The apparent permeability (Pₐₚₚ) and percent recovery were calculated.

For FIGS. 11B-11E, 50 k per well of cells were seeded and treated with indicated compounds the next day. 5 days after treatment, sphere number and size were assessed and normalized to vehicle control group. Data represent mean±SEM. n=3 independent experiments. Significance determined by one-way ANOVA analysis with Dunnett's test for multiple comparisons.

FIG. 11B, C3; UBC-Cre^{ERT+/-};Mtdh^{fl/fl} tumor cells (FIGS. 11D, 11E) without 5 days of 0.02 μg/ml 4-OHT pre-treatment were subjected to the tumorsphere assay.

FIG. 11C, C3; UBC-Cre^{ERT+/-};Mtdh^{fl/fl} tumor cells with 5 days of 0.02 μg/ml 4-OHT pre-treatment were subjected to the tumorsphere assay.

FIG. 11D, Wnt;UBC-Cre^{ERT+/-};Mtdh^{fl/fl} tumor cells without 5 days of 0.02 μg/ml 4-OHT pre-treatment were subjected to the tumorsphere assay.

FIG. 11E, Wnt;UBC-Cre^{ERT+/-};Mtdh^{fl/fl} tumor cells with 5 days of 0.02 μg/ml 4-OHT pre-treatment were subjected to the tumorsphere assay.

FIGS. 12A-12H show C26-A6 treatment blocks MTDH/SND1 interaction in vivo with limited toxicity.

FIG. 12A, NSG female mice were inoculated with 10 k of SCP28 cells that express split-luciferase components by MFP injection. Two weeks after injection, the mice were treated with 0.25 mg/mouse or 0.5 mg/mouse of C26-A6 via tail-vein injection. 30 min after the treatment, luciferase activity at primary tumors was measured.

FIG. 12B, Luciferase activity at primary tumors from mice in FIG. 12A. Data represent mean±SEM. n=3 mice. Significance determined by one-way ANOVA analysis with Dunnett's test for multiple comparisons.

FIG. 12C, H&E-stained sections of FIG. 5D are complemented by high-magnification images. Size bar, 5 mm.

FIG. 12D, Body weight of the mice in experiment from FIG. 5B was measured. Vehicle, n=10 mice; C26-A6, n=12 mice.

FIG. 12E, Serum from mice in experiment in FIG. 5B were collected for ALT and AST activity measurement following the standard protocol (Sigma). Three FVB females treated with 200 μl of 8% CCl4 in corn oil for 2 days served as positive control. Data represent mean±SEM. n=5 mice per group. Significance determined by one-way ANOVA analysis with Dunnett's test for multiple comparisons.

FIG. 12F, Blood samples were drawn from the heart of mice in experiment FIG. 5B, and blood cell counts were performed with the Sysemx XN-3000 Hematology System (Sysmex America, Inc.) Data represent mean±SEM. Vehicle, n=6 mice; C26-A6, n=5 mice. Significance determined by two tailed Student's t-test.

FIG. 12G, Small intestine samples were obtained from mice in experiment from FIG. 5B. H&E and Alcian blue staining was performed on processed, sliced samples. Scale bar: 200 μm.

FIG. 12H, Quantification of Alcian blue staining results from (FIG. 12G). Data represent mean±SEM. n=12 fields from 5 mice in each group. Significance determined by two tailed Student's t-test.

FIGS. 13A-13L show C26-A6 inhibits breast cancer progression and metastasis.

FIG. 13A, NGS female mice injected with 2 k SCP28 cells orthotopically were subjected to vehicle or C26-A6 treatment after two weeks. Primary tumor volumes were measured.

FIG. 13B, 8 weeks after treatment, tumor mass was assessed. Vehicle, n=10 mice; C26-A6, n=10 mice. Size bar, 2 cm.

FIG. 13C, 8 weeks after treatment, lung metastasis was assessed. Vehicle, n=10 mice; C26-A6, n=10 mice. Size bar, 5 mm.

FIG. 13D, Primary tumors from experiment in FIG. 5B were stained with Ki67 and Cleaved-Caspase 3 (Casp-3) antibodies. Size bar, 100 μm.

FIG. 13E, Positive cells were quantified. Data represent mean±SEM. n=6 mice.

FIG. 13F, Fresh HCI-001 PDX tumors were implanted into the mammary glands of female NSG mice. One day after implantation, the mice were treated with vehicle or C26-A6. Primary tumors were monitored.

FIG. 13G, Primary tumors from FIG. 13F were weighed. Representative tumors are shown. Size bar, 2 cm. n=12 tumors per group.

FIG. 13H, Primary tumors from FIG. 13F were stained with Ki67 and cleaved-Caspase 3 (Casp-3) antibodies. Size bar, 200 μm.

FIG. 13I, Positive cells from FIG. 13H were quantified. n=5 tumors per group.

FIG. 13J, Heatmap representation of next-generation sequencing data displaying the expression of genes in tumors that treated with vehicle (Ctrl), 60 mg/kg of Tmx for 5 consecutive days, or 15 mg/kg of C26-A6 5 days per week. Color key indicates log 2 values. n=4 mice per group.

FIG. 13K, Ingenuity pathway analysis shows the top five molecular and cellular functions of C26-A6 treatment-downregulated genes (n=620, fold change >2, p<0.05). p values were automatically determined by QIAGEN Ingenuity Pathway Analysis (QIAGEN IPA).

FIG. 13L, Effects of C26-A6 treatment-downregulated genes in cell death and survival functions. p values were automatically determined by QIAGEN Ingenuity Pathway Analysis (QIAGEN IPA).

Data represent mean±SEM. Significance determined by Two-Way Repeated Measures ANOVA test (FIGS. 13A, 13F) and two tailed Student's t-test (FIGS. 13B, 13C, 13E, 13G, 13I).

FIGS. 14A-14I show C26-A6 induces cell cycle arrest and reduces cell viability.

FIG. 14A, Spheres were treated with vehicle or indicated concentrations of C26-A6 for 1 week. The viability of the spheres was then quantified by MTT assay.

For FIGS. 14B-14I, similar sphere assay as in FIG. 14A was performed. n≥3 independent experiments. Data represent mean±SEM and significance determined by two tailed Student's t-test for all panels in FIGS. 14B-14I.

FIG. 14B, The apoptosis was determined.

FIG. 14C, The live cells were quantified.

FIG. 14D, The cell cycle status was determined.

FIG. 14E, The percentage of the cells in each cell cycle phase was quantified.

FIG. 14F, The apoptosis was determined.

FIG. 14G, The live cells were quantified.

FIG. 14H, The cell cycle status was determined.

FIG. 14I, The percentage of the cells in each cell cycle phase were quantified.

FIGS. 15A-15H show pathways that are altered upon C26-A6 treatment.

FIG. 15A, Gene set enrichment analysis plot showing the top four gene signatures in ranked list of genes.

FIG. 15B, Leading edge analysis was performed with the four gene signature and the heatmap of top candidate genes was shown. Color key indicates log 2 values.

FIG. 15C, Sphere assay was performed and treated with vehicle and C26-A6 as in FIG. 14A. The spheres were collected for western blot to analyze the expression of the candidates.

FIG. 15D, Primary tumors from experiment in FIG. 13A were stained with indicated antibodies. Size bars, 50 μm.

FIG. 15E, Positive cells from FIG. 15D were quantified. n=5 tumors per group. Data represent mean±SEM and significance determined by two tailed Student's t-test.

FIG. 15F, Mammary epithelial cell (MEC) spheres were treated with vehicle or C26-A6 for one week. The spheres were then harvested for RNA-sequencing and followed by gene set enrichment analysis.

FIG. 15G, The normalized enrichment scores of the indicated signatures in C26-A6 treated MECs and tumors from FIG. 15F are shown.

FIG. 15H, MEC spheres from FIG. 15F were collected for western blot analysis with indicated antibodies.

FIGS. 16A-16B show representative H&E-stained sections.

FIG. 16A, More representative H&E-stained lung sections that are complemented by high-magnification images for FIG. 5H are shown. Size bars, 5 mm.

FIG. 16B, More representative H&E-stained lung sections that are complemented by high-magnification images for FIG. 5K are shown. Size bars, 5 mm.

FIGS. 17A-17L show C26-A6 inhibits metastatic breast cancer progression.

FIG. 17A, Indicated cells were injected into NSG females orthotopically and followed by vehicle or C26-A6 treatment after 2 weeks. Tumor volumes were measured 8 weeks after injection. Vehicle, n=6 mice; C26-A6, n=6 mice.

FIG. 17B, Spontaneous lung metastasis of the mice in FIG. 17A were assessed by BLI (right). Size bar, 5 mm. Vehicle, n=5 lungs; C26-A6, n=6 lungs.

FIG. 17C, The SND1 and MTDH expression of the cells used in FIG. 17A was evaluated.

FIG. 17D, Tail-vein injection lung metastasis was determined by BLI right before (Week 0) or after (5 weeks) vehicle or C26-A6 treatment. Vehicle, n=11 lungs; C26-A6, n=12 lungs.

FIG. 17E, Lung metastatic nodules were quantified. The metastatic nodules of the representative lungs were highlighted with red and blue respectively. Size bar, 5 mm. Vehicle, n=11 lungs; C26-A6, n=12 lungs.

FIG. 17F, SUM159-M1a cells were injected into NSG females orthotopically. Two weeks after injection, the mice were treated with vehicle or C26-A6. Five weeks later, primary tumors were measured. n=10 mice per group.

FIG. 17G, SUM159-M1a cells were injected into NSG females orthotopically. Two weeks after injection, the mice were treated with vehicle or C26-A6. Five weeks later, spontaneous lung metastasis was measured. n=10 mice per group.

FIG. 17H, Tail-vein injection lung metastasis was determined by BLI right before (Week 0) or after (5 weeks) vehicle or C26-A6 treatment. n=12 mice per group.

FIG. 17I, The metastatic nodules of the representative lungs were highlighted with red and blue respectively. n=12 mice per group. Size bar, 5 mm.

FIG. 17J, 4T1 cells were injected into Balb/C females orthotopically. 1 week after injection, the mice were treated with vehicle or C26-A6. 5 weeks after the treatment, primary tumors were measured. n=10 mice per group.

FIG. 17K, 4T1 cells were injected into Balb/C females orthotopically. 1 week after injection, the mice were treated with vehicle or C26-A6. 5 weeks after the treatment, spontaneous lung metastasis was measured. n=10 mice per group.

FIG. 17L, 4T1 cells were injected into Balb/C females intravenously. 5 weeks after vehicle or C26-A6 treatment, lung metastatic nodules were counted. The metastatic nodules of the representative lungs were highlighted with red and blue respectively. Vehicle, n=5 mice; C26-A6, n=6 mice. Size bar, 5 mm.

Data represent mean±SEM and significance determined by two tailed Student's t-test for all panels of FIGS. 17A-17L.

FIGS. 18A-18I show MTDH promotes metastatic breast cancer by enhancing immune evasion.

FIG. 18A, Three WT/PyMT (Mtdh^{+/+}) and KO/PyMT (Mtdh^{-/-}) cell lines were injected into FVB female mice via tail vein. Five weeks after injection, lungs were fixed, and numbers of metastatic nodules were quantified. n=6 mice per group.

FIG. 18B, Representative lungs from FIG. 18A are shown.

FIG. 18C, KO/PyMT cell lines that were rescued with vector or wild type MTDH were employed for tail vein injection. Five weeks after injection, lung metastatic nodules were counted. n=6 female FVB mice per group.

FIG. 18D, WT/PyMT cells with/without endogenous Mtdh knockdown were injected in to female FVB mice intravenously. Numbers of lung nodules and representative lungs were shown. n=6 mice per group.

FIG. 18E, PyMT; UBC-Cre^{ERT+/-};Mtdh^{fl/fl} tumors with or without MTDH acute knockout (KO) were collected for RNA sequencing and GSEA. p and q values automatically determined by GSEA 3.0. Tmx, Tamoxifen. n=4 mice per group.

FIG. 18F, FVB female mice were injected with CD4 and CD8 neutralizing antibodies or isotype control (IgG) for 3 days. Peripheral blood was collected to examine the depletion of CD4 and CD8. % of CD4 and CD8 in CD45⁺ populations are shown. n=6 mice.

FIG. 18G, FVB females were inoculated with WT/PyMT or KO/PyMT cells via tail vein. The mice were treated with anti-CD4 or CD8 neutralization antibodies or IgG. Five weeks after injection, numbers of lung metastasis nodules were counted. n=6 mice per group.

FIG. 18H, FVB females inoculated with KO/PyMT cells via tail vein were treated with anti-CD8 antibody or IgG. The survival of mice was plotted. KO+IgG, n=11 mice, KO+α-CD8, n=12 mice.

FIG. 18I, WT/PyMT and KO/PyMT cells were inoculated into FVB females via mammary fat pad injections. The mice were treated with anti-CD8 neutralizing antibody or IgG. Eight weeks after injection, metastatic nodules were quantified. n=5 mice per group.

In all lung images in FIGS. 18A, 18I, size bar, 5 mm. Data represent mean±SEM. Significance determined by two tailed Student's t-test (FIGS. 18A, 18C, 18D, 18I), one-way ANOVA analysis with Sidak's test for multiple comparisons (FIG. 18G), or Log-rank test (FIG. 18H).

FIGS. 19A-19F show MTDH inhibits T cell activation.

FIG. 19A, Schematic diagram of in vitro tumor-immune cell co-culture assay. Ovalbumin (OVA) expressing Py8119 tumor cells were seeded into plates. One day after the seeding, splenocytes or CD8⁺ T cells isolated from OT-I mice were co-cultured with tumor cells at 10:1 ratio. 24 hr after co-culture, the cells or culture media were collected for the following analysis.

FIG. 19B, Indicated tumor cells were co-cultured with CD8⁺ T cells. 24 hours after co-culture, culture media were collected for cytotoxicity analysis. Data represent mean SEM. n=3 independent experiments. Significance determined by one-way ANOVA analysis with Sidak's test for multiple comparisons.

FIG. 19C, The tumor cells from FIG. 19B were harvested for western blot to examine with apoptotic markers PARP and cleaved-caspase 3 (CC-3).

FIG. 19D, OT-I splenocytes co-cultured with indicated tumor cells for 24 hr were harvested for flow cytometry analysis. The expression of activation marker CD137 in CD8⁺ T cells was determined.

FIG. 19E, OT-I splenocytes co-cultured with indicated tumor cells for 24 hr were harvested for flow cytometry analysis. The expression of activation marker IFN-γ in CD8⁺ T cells was determined.

FIG. 19F, The culture media from FIG. 19D and FIG. 19E were collected for ELISA assay to examine the concentration of IFN-γ. MFI, Mean Fluorescence Intensity.

Data represent mean±SEM. n=3 independent experiments. Significance determined by one-way ANOVA analysis with Sidak's test for multiple comparisons.

FIGS. 20A-20E show MTDH destabilizes Tap1/2.

FIG. 20A, Py8119-OVA cells with (shMTDH) or without (shCtrl) Mtdh knockdown were co-cultured with splenocytes from OT-I mice. At 0 hr or 24 hr after the co-culture, tumor cells were collected, RNA was extracted, and qRT-PCR was performed to test the indicated genes. Data represent mean±SEM. n=3 independent experiments. Significance determined by two tailed Student's t-test.

FIG. 20B, Tumor cells in FIG. 20A were also harvested for western blot.

FIG. 20C, Indicated tumor cells were co-cultured with OT-I splenocytes for 24 hr, and then treated with 10 μg/ml of actinomycin D. The tumor cells were harvested at indicated time points after the treatment. RNA was extracted and qRT-PCR was performed to test Tap1/2 levels. Data represent mean±SEM. n=3 independent experiments. Significance determined by two tailed Student's t-test.

FIG. 20D, Py8119-OVA cells co-cultured with OT-1 splenocytes for 24 hr were collected for RNA-binding protein immunoprecipitation (RIP) assay. MTDH protein was pull-down, the binding RNA was extracted, and Tap1/2 was amplified with PCR.

FIG. 20E, Indicated tumor cells were co-cultured with OT-I splenocytes for 0 or 24 hr. The tumor cells were then collected and subjected to flow cytometry to determine the surface presentation of Ovalbumin (H-2K^{b}-SIINFEKL). MFI, Mean Fluorescence Intensity. Data represent mean±SEM. n=3 independent experiments. Significance determined by two tailed Student's t-test.

FIGS. 21A-H show MTDH forms complex with SND1 to inhibit tumor antigen presentation and T cell activation.

FIG. 21A, Py8119-OVA cells were co-cultured with OT-1 splenocytes for 24 hr, and then collected for RIP assay. SND1 pull-down was confirmed by immunoblotting (IB). The binding RNA was extracted, and Tap1/2 was amplified with PCR.

FIG. 21B, Py8119-OVA cells with endogenous Mtdh KD (shMTDH) and indicated rescues (shMTDH+WT, +W391D, or +W398D) were co-cultured with OT-1 splenocytes for 24 hr, and then collected for RIP assay. MTDH protein pull-down was confirmed by immunoblotting (IB). The binding RNA was extracted, and Tap1/2 was amplified with PCR. WT, wildtype MTDH; W391D and W398D, SND1 interaction-deficient mutants MTDH-W391D and MTDH-W398D.

FIG. 21C, Indicated Py8119-OVA cells co-cultured with OT-1 splenocytes for 24 hr were harvested for RNA extraction. Levels of Tap1/2 were determined by qRT-PCR.

FIG. 21D, Indicated Py8119-OVA tumor cells co-cultured for 24 hr were treated with 10 μg/ml of actinomycin D. 8 hr after treatment, RNA levels of Tap1/2 were determined by qRT-PCR.

FIG. 21E, Indicated Py8119-OVA tumor cells after co-culture were collected to test the OVA (H-2K^{b}-SIINFEKL) presentation on tumor cells.

FIG. 21F, Indicated OT-1 splenocytes after co-culture were collected to test the CD137 expression in splenocytes.

FIG. 21G, Indicated OT-1 splenocytes after co-culture were collected to test the IFN-γ expression in splenocytes.

FIG. 21H, Media from FIG. 21E was employed for ELSA to test IFN-γ concentration and cytotoxicity assay. AU, arbitrary units. In all panels data represent mean SEM. n=3 independent experiments. Significance determined by one-way ANOVA analysis with Dunnett's test for multiple comparisons.

FIGS. 22A-22G show MTDH-SND1 blocking activates T cells by enhancing antigen presentation in tumors.

FIG. 22A, The correlation of the gene sets that significantly enriched (FDR<0.01) in Mtdh acute loss and C26-A6 treated tumors. Tmx, Tamoxifen.

FIG. 22B, Gene set enrichment analysis showing the enrichment of interferon signatures in C26-A6 treated PyMT tumors as compared to control PyMT tumors. p and q values automatically determined by GSEA 3.0.

FIG. 22C, Py8119-OVA cells co-cultured with OT-I splenocytes were treated with 200 μM of C26-A6 or same amount of vehicle. The binding between MTDH and Tap1/2 in tumor cells were determined by RIP assay.

FIG. 22D, Tap1/2 RNAs that bind to MTDH in (c) were quantified and normalized to the pulled down MTDH levels.

FIG. 22E, Tap1/2 levels in Py8119-OVA cells with/without 200 μM of C26-A6 treatment during co-culture were examined by qRT-PCR.

FIG. 22F, OVA (H-2K^{b}-SIINFEKL) presentation in Py8119-OVA cells with/without 200 μM of C26-A6 treatment in co-culture were determined by flow cytometry. MFI, Mean Fluorescence Intensity; AU, arbitrary units.

FIG. 22G, Media in (E) was collected for IFN-γ ELISA and cytotoxicity assay. AU, arbitrary units.

In panels FIGS. 22C-22G, data represent mean±SEM. n=3 independent experiments. Significance determined by two tailed Student's t-test.

FIGS. 23A-23F show MTDH-SND1 disruption and anti-PD-1 treatment synergistically inhibits metastatic breast cancer progression.

FIG. 23A, OT-I splenocytes co-cultured with indicated Py8119-OVA cells were harvested after 24 hr. The expression of PD-1 in CD8⁺ T cells was examined by flow cytometry. % of CD8⁺PD-1⁺ cells in live cell populations are shown. shCtrl, Py8119-OVA without MTDH KD; shMTDH, Py8119-OVA with MTDH KD; shMTDH+WT, Py8119-OVA with MTDH KD and rescued with wildtype MTDH; shMTDH+W391D, Py8119-OVA with MTDH KD and rescued with SND1 interaction-deficient mutant MTDH-W391D; shMTDH+W398D, Py8119-OVA with MTDH KD and rescued with SND1 interaction-deficient mutant MTDH-W398D. n=3 independent experiments. Significance determined by one-way ANOVA analysis with Dunnett's test for multiple comparisons.

FIG. 23B, Cells in FIG. 23A were gated on the CD8⁺ T cell population and the mean fluorescence intensity (MFI) of PD-1 expression was measured. AU, arbitrary units. n=3 independent experiments. Significance determined by one-way ANOVA analysis with Sidak's test for multiple comparisons.

FIG. 23C, PyMT;UBC-Cre^{ERT+/-};Mtdh^{fl/fl} females with tumors established were treated with Tmx and anti-PD-1 alone or in combination as illustrated in Extended Data FIG. 9c. Primary tumors were measured weekly during the treatments. n=6 mice per group. Significance determined by Two-Way Repeated Measures ANOVA test.

FIG. 23D, Lungs were collected and fixed at endpoint. Metastatic nodules were counted. The % of metastatic tumor area in lungs was also quantified based on H&E staining. Size bars, 5 mm. n=6 lungs per group. Significance determined by one-way ANOVA analysis with Sidak's test for multiple comparisons.

FIG. 23E, Primary tumors and lungs from FIG. 23C were fixed for CD8 IHC staining. Infiltrated CD8⁺ T cells were quantified. n=3 lungs per group. Significance determined by one-way ANOVA analysis with Sidak's test for multiple comparisons.

FIG. 23F, Primary tumors and lungs from mice as treated in FIG. 23C were dissociated for flow cytometry analysis. Activated T cells were quantified using CD69 as a marker. Percent of CD8⁺CD69⁺ cells in live cell populations are shown. n=4 lungs per group.

Significance determined by one-way ANOVA analysis with Sidak's test for multiple comparisons. Data represent mean±SEM in all panels.

FIGS. 24A-H show C26-A6 treatment synergizes with anti-PD-1 therapy for metastatic breast cancer.

For FIGS. 24A, 24B, FVB-PyMT females with primary tumors established were divided into four groups and treated with vehicle, anti-PD-1, or C26-A6 alone or in combination. Anti-PD-1, 200 μg/mouse i.p. injection, twice per week for the first week and then once per week after that; C26-A6, 15 mg/kg i.v. injection, 5 days per week. Data represent mean±SEM. Significance determined by one-way ANOVA analysis with Sidak's test for multiple comparisons.

FIG. 24A, Six weeks after treatment, primary tumor burdens were quantified. n=10 mice per group.

FIG. 24B, Six weeks after treatment, lung metastatic burdens were quantified. n=5 lungs per group. Size bar, 5 mm.

FIG. 24C, CD8⁺ T cell infiltration of the sample in FIG. 23A was determined by flow cytometry. Percentage of CD8⁺ cells in CD45⁺ populations are shown. Data represent mean±SEM. n=5 tumors/lungs per group. Significance determined by one-way ANOVA analysis with Sidak's test for multiple comparisons.

FIG. 24D, IFN-γ expression in CD8⁺ T cells of the sample in FIG. 23A was determined by flow cytometry. Percentage of CD8⁺IFN-γ⁺ cells in CD45⁺ populations are shown. Data represent mean±SEM. n=5 tumors/lungs per group. Significance determined by one-way ANOVA analysis with Sidak's test for multiple comparisons.

FIG. 24E, FVB females were injected with 100 k of luciferase stably expressed PyMT cells. Three weeks after the injection, lung metastases were established and the mice were randomized into two groups.

FIG. 24F, BLI signals in the groups from FIG. 24E were determined before the treatment. Data represent mean±SEM. Significance determined by two tailed Student's t-test.

FIG. 24G, The mice from FIG. 24E were treated with vehicle or C26-A6+anti-PD-1, and the metastasis was monitored by BLI.

FIG. 24H, Kaplan-Meier survival curves of the two groups from FIG. 24G. n=6 mice per group. Significance determined by two-sided Log-rank test.

FIGS. 25A-25B show MTDH expression negatively correlates with CD8⁺ T cell infiltration and PD-1 expression in TNBC patients.

FIG. 25A, IHC staining shows negative correlation between MTDH expression and CD8⁺ T cell infiltration or PD-1 expression in primary tumors from TNBC patients. n=286 patients. Representative images were shown. Scale bar, 100 μm.

FIG. 25B, Kaplan-Meier plot of relapse free survival (RFS) and distant metastasis free survival (DMFS) of TNBC patients stratified by MTDH protein levels and infiltrated CD8⁺ T cells. p-value by two-sided Log-rank test.

FIGS. 26A-26H show MTDH depletion reshapes immune cell populations in tumors.

FIG. 26A, KO/PyMT cells were rescued with vector or wild type MTDH. The expression of MTDH was validated by western blot.

FIG. 26B, Western blot analysis of endogenous MTDH knockdown in WT/PyMT cells.

FIG. 26C, Western blot analysis of endogenous MTDH knockdown in E0771 cells with stable luciferase expression.

FIG. 26D, 1×10⁶ of E0771 cells with (shMTDH-1, -2) or without (shCtrl) MTDH knockdown were injected into female C57/BL6 mice via tail-vein. 6 weeks after injection, lungs were collected (left) and metastatic nodules were counted (right). n=6 mice per group. Size bar, 5 mm. Data represent mean±SEM. Significance determined by one-way ANOVA analysis with Dunnett's test for multiple comparisons.

FIG. 26E, PyMT; UBC-Cre^{ERT+/-};Mtdh^{fl/fl} mice with tumors established were treated with vehicle or tamoxifen (Tmx) for five consecutive days. One week after the treatment, tumors were collected, and RNA was extracted for RNA sequencing. Gene sets that are significantly enriched in ranked gene list of Tmx treated versus control cells. n=4 mice per group.

FIG. 26F, Tumors from PyMT; UBC-Cre^{ERT+/-};Mtdh^{fl/fl} mice treated with vehicle or Tmx were collected for immunohistochemistry (IHC) staining with indicated antibodies. Size bar, 50 μm.

FIG. 26G, The numbers of positive cells per field were quantified. Data represent mean±SEM. n=8 fields from four mice in each group. Significance determined by two tailed Student's t-test.

FIG. 26H, PyMT; UBC-Cre^{ERT+/-};Mtdh^{fl/fl} tumorspheres were treated with vehicle (Ctrl) or 4-hydroxytamoxifen (4-OHT) (MTDH-KO). The spheres were then collected for RNA sequencing. Gene set enrichment analysis demonstrates the enrichment of the indicated gene sets. p and q values automatically determined by GSEA 3.0. The enrichment scores of the indicated signatures from tumorspheres in vitro or tumor samples in vivo (FIG. 1-2e) were presented (right).

FIGS. 27A-27D show CD8⁺ T cells depletion partially restores MTDH knockdown induced metastatic inhibition.

FIG. 27A, FVB females were treated with 125 μg/mouse of anti-CD8 antibody or isotype control for 3 days. Peripheral blood was collected for flow cytometry analysis at indicated days after treatment (top). % of CD8⁺ cells in CD45⁺ populations are shown. Anti-CD8 antibody treatment scheme that used for the in vivo experiments in this study (bottom).

FIG. 27B, 1×10⁶ E0771-shCtrl or shMTDH-1 (shMTDH hereafter) cells were injected into C57/BL6 females intravenously. The mice were subjected to isotype control or anti-CD4, anti-CD8 neutralizing antibodies treatment as in FIG. 27A. 6 weeks after injection, the mice were euthanized, lungs were collected and fixed with Bouin's solution. Representative lungs are shown and lung metastatic nodules were counted. n=6 mice per group. Size bar, 5 mm. Data represent mean±SEM. Significance determined by one-way ANOVA analysis with Sidak's's test for multiple comparisons.

FIG. 27C, Kaplan-Meier survival curve of C57/BL6 female mice injected with 1×10⁶ E0771-shMTDH that were treated with IgG or anti-CD8 neutralizing antibodies. n=6 mice per group. Significance determined by Log-rank test.

FIG. 27D, 5×10⁵-shCtrl or shMTDH E0771 cells were injected into the mammary fat pad of female C57BL6 mice, and the mice were treated with IgG or anti-CD8 neutralizing antibody as in FIG. 27A. 6 weeks after injection, lungs were harvested and bioluminescent imaging (BLI) was performed to measure lung metastasis. Representative lungs (left), quantitative BLI signals (middle), and fold change of BLI signal between IgG and anti-CD8 groups (right) are shown. n=16 mice per group. Data represent mean±SEM. Significance determined by two tailed Student's t-test.

FIGS. 28A-28I show characterization of in vitro tumor-immune cell co-culture system.

FIG. 28A, MHC-I (H-2K^{d}/H-2D^{d}) presentation in parental Py8119 cells were analyzed by flow cytometry. Isotype IgG served as negative control.

FIG. 28B, Stable expression of Ovalbumin (OVA) in the resulted Py8119-OVA cells were confirmed with western blot.

FIG. 28C, Surface presentation of OVA (H-2K^{b}-SIINFEKL) in parental Py8119 and Py8119-OVA cells with or without OT-1 splenocytes co-culture were analyzed with flow cytometry.

FIG. 28D, Splenocytes isolated from OT-I mice were treated with PBS or 2 μg/ml of Ovalbumin peptide (OVAp257) for 2 hr. The cells were washed with PBS and plated in fresh media for another 24 hr followed by flow cytometry analysis. % of CD137⁺ or IFN-γ⁺ cells in live populations are shown.

FIG. 28E, Splenocytes on the CD8 population were gated and the mean fluorescence intensity (MFI) of CD137 and IFN-γ were measured. n=3 independent experiments.

FIG. 28F, The indicated cells with endogenous Mtdh knockdown and vector (Vec) or wild type MTDH rescue were confirmed with western blot.

FIG. 28G, The tumorigenesis ability of indicate cell lines were evaluated. Indicated cells were inoculated into the mammary fat pad of C57BL/6 female mice. Ten weeks after injection, lung metastatic nodules were counted. shCtrl, Py8119-shCtrl; KD, Py8119-shMTDH; shCtrl-OVA, Py8119-OVA-shCtrl; KD-OVA, Py8119-OVA-shMTDH; KD-OVA-Vec, Py8119-OVA-shMTDH rescued with vector; KD-OVA-MTDH, Py8119-OVA-shMTDH rescued with wild type MTDH. n=5 mice per group.

FIG. 28H, Py8119-OVA cells with/without endogenous Mtdh knockdown or with/without wild type MTDH rescued were employed for mammary fat pad injections. The injected OT-I female mice were treated with/without anti-CD8 neutralization antibody or IgG. Six weeks after treatment, lung metastasis was determined. n=9 mice per group.

FIG. 28I, Splenocytes co-cultured with indicated Py8119-OVA tumor cells (same as in FIG. 19B) for 24 hr were harvested for flow cytometry analysis. The expression of Granzyme B in CD8⁺ T cells were examined.

n=3 independent experiments. Data represent mean±SEM. Significance determined by two tailed Student's t-test (FIGS. 28E, 28G), or one-way ANOVA analysis with Sidak's's test for multiple comparisons (FIGS. 28H, 28I).

FIGS. 29A-29C show Mtdh acute loss enhances tumor antigen presentation.

FIG. 29A, PyMT; UBC-Cre^{ERT+/-};Mtdh^{fl/fl} mice with tumors were treated with vehicle or 60 mg/kg of tamoxifen (Tmx) for five consecutive days. One week after the treatment, tumors were collected, and RNA was extracted for RNA sequencing. Ingenuity pathway analysis shows the top five molecular and cellular functions of Mtdh acute loss up-regulated genes. p values automatically generated by QIAGEN Ingenuity Pathway Analysis (QIAGEN IPA).

FIG. 29B, RNA samples from FIG. 29A were subjected to qRT-PCR test of indicated genes. Data represent mean±SEM. n=3 independent experiments. Significance determined by two tailed Student's t-test.

FIG. 29C, Py8119-OVA-shCtrl or shMTDH tumor cells were co-cultured with OT-I splenocytes for 0 or 24 hr. Tumor cells were then collected and subjected to flow cytometry to determine the surface presentation of MHC-I. MFI, mean fluorescence intensity. Data represent mean±SEM. n=3 independent experiments. Significance determined by two tailed Student's t-test.

FIGS. 30A-30H show SND1 binds to Tap1/2 and promotes their degradation.

FIG. 30A, Endogenous SND1 knockdown in Py8119-OVA cells was confirmed with western blot.

FIG. 30B, Tap1/2 mRNA levels in the indicated Py8119-OVA cells that were co-cultured with OT-I splenocytes for 0 or 24 hr was examined by qRT-PCR. n=3 independent experiments.

FIG. 30C, Indicated Py8119-OVA tumor cells co-cultured for 24 hr were treated with 10 μg/ml of actinomycin D for another 8 hr. RNA levels of Tap1/2 in tumor cells were determined by qRT-PCR. n=3 independent experiments.

FIG. 30D, Py8119-OVA cells with SND1 knockdown were subjected to RIP assay after 24 hr co-culture. The interaction between SND1 and Tap1/2 was determined by PCR.

FIG. 30E, Tap1/2 RNAs that bind to MTDH were quantified and normalized to the pulled down MTDH levels. n=3 independent experiments.

FIG. 30F, Py8119-OVA cells with MTDH knockdown were subjected to RIP assay after 24 hr co-culture. The interaction between MTDH and Tap1/2 was determined by PCR.

FIG. 30G, Tap1/2 RNAs that bind to SND1 were quantified and normalized to the pulled down SND1 levels. n=3 independent experiments.

FIG. 30H, Electrophoretic mobility gel shift assay was performed with in vitro transcribed TAP1/2 mRNA incubated with PBS, recombinant MTDH and SND1 alone or in combination.

Data represent mean±SEM. Significance determined by one-way ANOVA analysis with Dunnett's test for multiple comparisons (FIGS. 30B, 30C, 30E), or two tailed Student's t-test (FIG. 30G).

FIGS. 31A-31F show SND1 inhibits antigen presentation and T cell activation.

FIG. 31A, Py8119-OVA tumor cells after co-culture were collected to test OVA (H-2K^{b}-SIINFEKL) presentation.

FIG. 31B, Py8119-OVA tumor cells after co-culture were collected to test MHC-I presentation.

FIG. 31C, OT-I splenocytes after co-culture were collected to test CD137 expression.

FIG. 31D, OT-I splenocytes after co-culture were collected to test IFN-γ expression.

FIG. 31E, Media from FIG. 31A was employed for ELSA to test IFN-γ concentration and cytotoxicity assay.

FIG. 31F, PyMT tumor cells with MTDH KD or indicated rescues were injected into FVB females intravenously. Five weeks after injection, lung metastatic nodules were counted, and representative lungs were shown. n=6 lungs per group. Data represent mean SEM.

In panels from FIGS. 31A-31E, n=3 independent experiments. Significance determined by one-way ANOVA analysis with Dunnett's test for multiple comparisons (FIGS. 31A-31E), or one-way ANOVA analysis with Sidak's test for multiple comparisons (FIG. 31F).

FIGS. 32A-32F show C26-A6 treatment elevates immune responses in tumors.

FIG. 32A, The correlation between all the gene sets that alters by Mtdh acute loss and C26-A6 treatment.

FIG. 32B, Py8119-OVA cells co-cultured with OT-I splenocytes were treated with 200 μM of C26-A6 or same amount of vehicle. The binding between SND1 and Tap1/2 in tumor cells were determined by RIP assay.

FIG. 32C, Tap1/2 RNAs that bind to SND1 were quantified and normalized to the pulled down SND1 levels.

FIG. 32D, MHC-I presentation in Py8119-OVA cells with/without 200 μM of C26-A6 treatment in co-culture were determined by flow cytometry. MFI, mean fluorescence intensity; AU, arbitrary units.

FIGS. 32E, The expression of CD137 in splenocytes co-cultured with Py8119-OVA was determined by flow cytometry upon 200 μM of C26-A6 or vehicle treatment.

FIGS. 32F, The expression of IFN-γ in splenocytes co-cultured with Py8119-OVA were determined by flow cytometry upon 200 μM of C26-A6 or vehicle treatment.

In panels from FIGS. 32B-32F, data represent mean±SEM. n=3 independent experiments. Significance determined by two tailed Student's t-test.

FIGS. 33A-33I show MTDH-SND1 complex promotes immune evasion through Tap1/2.

FIG. 33A, Western blot analyzing to confirm the knock down of Tap1/2 in E0771 cells (E0771-OVA) stably expressing luciferase and OVA after lentiviral transduction of respective shRNAs.

For FIGS. 33B-33D, E0771-OVA cells with (shTap1/2) or without (shCtrl) Tap1/2 knockdown were co-cultured with OT-I splenocytes with the ratio of 1:10 (tumor cells:splenocytes). Data represent mean±SEM. n=3 independent experiments. Significance determined by one-way ANOVA analysis with Sidak's test for multiple comparisons.

FIG. 33B, 24 hr after co-culture, tumor cells were collected for examining OVA (H-2K^{b}-SIINFEKL) expression.

FIG. 33C, 24 hr after co-culture, tumor cells were collected for examining MHC-I expression.

FIG. 33D, 24 hr after co-culture, splenocytes were collected for examining CD137 expression.

FIG. 33E, The same co-culture experiment as in FIG. 33B were performed. The live tumor cells were indicated by luciferase signal. The percentage of live cells were determined as normalized to the non-co-culture control. Data represent mean±SEM. n=5 independent experiments. Significance determined by one-way ANOVA analysis with Sidak's test for multiple comparisons.

FIG. 33F, PresentER-Vector or PresentER-OVA (H-2K^{b}-SIINFEKL) system was stably expressed in Py8119 cells. The surface presentation of OVA (H-2K^{b}-SIINFEKL) was validated by flow cytometry analysis.

FIG. 33G, Western blot analysis confirming the knock down of MTDH in Py8119 cells stably expressing luciferase and PresentER-OVA (Py8119-PresentER-OVA) after lentiviral transduction of respective shRNAs.

FIG. 33H, Py8119-PresentER-OVA cells with (shMTDH) or without (shCtrl) MTDH knockdown were co-cultured with OT-I splenocytes with the ratio of 1:10 (tumor cells:splenocytes). 24 hr after co-culture, Tumor cells were collected for examining OVA (H-2K^{b}-SIINFEKL). Data represent mean±SEM. n=3 independent experiments. Significance determined by two tailed Student's t-test.

FIG. 33I, The same co-culture experiment as in FIG. 33H was performed. The live tumor cells were indicated by luciferase signal. The percentage of live cells were determined as normalized to the non-co-culture control. Data represent mean±SEM. n=3 independent experiments. Significance determined by two tailed Student's t-test.

FIGS. 34A-34D show MTDH-SND1 disruption and anti-PD-1 treatment synergistically enhance anti-tumor immune response.

FIG. 34A, OT-I splenocytes were co-cultured with Py8119-OVA cells with or without 200 μM of C26-A6 treatment for 24 hr. The expression of PD-1 in CD8⁺ T cells was examined by flow cytometry. % of CD8⁺PD-1⁺ in live populations are shown. Data represent mean±SEM. n=3 independent experiments. Significance determined by two tailed Student's t-test.

FIG. 34B, Cells in FIG. 34A were gated on the CD8⁺ T cell population and the mean fluorescence intensity (MFI) of PD-1 expression was measured. AU, arbitrary units. Data represent mean±SEM. n=3 independent experiments. Significance determined by two tailed Student's t-test.

FIG. 34C, Schematic diagram of treatment. PyMT; UBC-Cre^{ERT+/-};Mtdh^{fl/fl} females with tumors established were treated with Tmx and anti-PD-1 alone or in combination. Tmx, Tamoxifen, 60 mg/kg i.p. for 5 consecutive days; anti-PD-1, 200 μg/mouse i.p. injection, twice per week for the first week and then once per week after that.

FIG. 34D, Primary tumors and lungs from experiment in FIG. 6-2c were fixed for CD8 IHC staining. Scale bar, 100 μm.

FIGS. 35A-35C show C26-A6 combined with anti-PD-1 treatment reshapes the tumor immune microenvironment. For FIGS. 35A-35C, 100 k PyMT tumor cells were orthotopically injected into the mammary glands of FVB females. The mice were randomized and divided into three groups when primary tumors were established, followed by vehicle, C26-A6, or C26-A6+anti-PD-1 treatment. Six weeks after treatment, primary tumors and lung with metastatic lesions were collected for flow analysis with indicated antibodies. Anti-PD-1, 200 μg/mouse i.p. injection, twice per week for the first week and then once per week after that; C26-A6, 15 mg/kg i.v. injection, 5 days per week. n=6 mice per group. Data represent mean±SEM. Significance determined by one-way ANOVA analysis with Dunnett's test for multiple comparisons.

FIG. 35A, Percentages of CD11b⁺F4/80⁺, Ly6G^{low}Ly6C^{high}, Ly6G^{high}Ly6C^{low}, CD3-NK1.1⁺ in CD45⁺ population and percentage of CD4⁺FOXP3⁺ in CD3⁺ population are shown.

FIG. 35B, Percentages of GITR⁺LAG-3⁺ in CD8⁺ population are shown.

FIG. 35C, Negative correlation between MTDH expression and CD8⁺ T cell infiltration or PD-1 expression in TNBC patients. Representative IHC images are shown in FIG. 25A. p-value by two-sided chi square test tests.

FIG. 36A, taken together with FIGS. 36B and 36C, show the gating strategy used in Example 2.

FIG. 36B, taken together with FIGS. 36A and 36C, show the gating strategy used in Example 2.

FIG. 36C, taken together with FIGS. 36A and 36B, show the gating strategy used in Example 2.

FIG. 37 is a graph of inhibitory efficiency of the analogs in the cell free split luciferase assay described in Example 1.

FIG. 38A is a graph of inhibitory efficiency of the indicated compounds in the cell free split luciferase assay described in Example 1.

FIG. 38B is a graph of inhibitory efficiency of the indicated compounds in the cell free split luciferase assay described in Example 1.

FIG. 38C is a graph of inhibitory efficiency of the indicated compounds in the cell free split luciferase assay described in Example 1.

### DETAILED DESCRIPTION

A description of example embodiments follows.

### Definitions

As used herein and in the appended claims, the singular forms “a,” “an,” and “the” include plural referents unless the context clearly dictates otherwise. Thus, for example, reference to “a pharmaceutically acceptable carrier” includes a plurality of such carriers, each of which may be the same or different.

Similarly, “comprise,” “comprises,” “comprising,” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMIDRAW®, version 17.0.0.206, PerkinElmer Informatics, Inc.).

“Alkyl” refers to a saturated, aliphatic, branched or straight-chain, monovalent, hydrocarbon radical having the specified number of carbon atoms. Thus, “(C₁-C₆)alkyl” means a radical having from 1-6 carbon atoms in a linear or branched arrangement. Examples of alkyl groups include methyl, ethyl, n-propyl, isopropyl, n-butyl, isobutyl, sec-butyl, t-butyl, n-pentyl, isopentyl, neopentyl, 2-methylpentyl, n-hexyl, and the like.

“Alkoxy” refers to an alkyl radical attached through an oxygen linking atom, wherein alkyl is as described herein. Examples of alkoxy include, but are not limited to, methoxy, ethoxy, propoxy, isopropoxy, and the like.

“Amino” refers to —NH₂.

“Alkylamino” refers to —N(H)(alkyl), wherein alkyl is as described herein. Examples of alkylamino include methylamino, ethylamino, propylamino, isopropylamino, and the like.

“Dialkylamino” refers to —N(alkyl)₂, wherein the alkyl groups are the same or different, and alkyl is as described herein. Examples of alkylamino include dimethylamino, ethylmethylamino, diethylamino, dipropylamino, isopropylethylamino, and the like.

“Aryl” refers to a monocyclic or polycyclic (e.g., bicyclic, tricyclic), carbocyclic, aromatic ring system having the specified number of ring atoms, and includes aromatic rings fused to non-aromatic rings, as long as one of the fused rings is an aromatic hydrocarbon. Thus, “(C₆-C₁₅)aryl” means an aromatic ring system having from 6-15 ring atoms. Examples of aryl include phenyl and naphthyl.

“Carboxy” refers to —COOH.

“Cyano” refers to —C≡N.

“Cycloalkyl” refers to a saturated, aliphatic, monovalent, monocyclic or polycyclic, hydrocarbon ring radical having the specified number of ring atoms. Thus, “(C₃-C₆)cycloalkyl” means a ring radical having from 3-6 ring carbons. Typically, cycloalkyl is monocyclic. Cycloalkyl includes, but is not limited to, cyclopropyl, cyclobutyl, cyclopentyl, cyclohexyl, and the like. In some embodiments, cycloalkyl is (C₃-C₁₅)cycloalkyl. In some embodiments, cycloalkyl is (C₃-C₁₂) cycloalkyl. In some embodiments, cycloalkyl is (C₃-C₈)cycloalkyl. In some embodiments, cycloalkyl is (C₃-C₆)cycloalkyl.

“Halogen” and “halo” are used interchangeably herein and each refers to fluorine, chlorine, bromine, or iodine. In some embodiments, halogen is fluoro, bromo or chloro. In some embodiments, halogen is fluoro or chloro. In some embodiments, halogen is fluoro or bromo.

“Haloalkyl” refers to an alkyl radical wherein at least one hydrogen of the alkyl radical is replaced with a halo, and alkyl is as described herein. Haloalkyl includes mono, poly, and perhaloalkyl groups, wherein each halogen is independently selected from fluorine, chlorine, bromine and iodine (e.g., fluorine, chlorine and bromine), and alkyl is as described herein. In one aspect, haloalkyl is perhaloalkyl (e.g., perfluoroalkyl). Haloalkyl includes, but is not limited to, fluoromethyl, difluoromethyl, trifluoromethyl and pentafluoroethyl.

“Haloalkoxy” refers to a haloalkyl radical attached through an oxygen linking atom, wherein haloalkyl is as described herein. Haloalkoxy includes trifluoromethoxy.

“Heteroaryl” refers to a monocyclic or polycyclic (e.g., bicyclic, tricyclic), aromatic, hydrocarbon ring system having the specified number of ring atoms, wherein at least one carbon atom in the ring system has been replaced with a heteroatom selected from N, S and O. “Heteroaryl” includes heteroaromatic rings fused to non-aromatic rings, as long as one of the fused rings is a heteroaromatic hydrocarbon. Thus, “(C₅-C₁₅)heteroaryl” means a heterocyclic aromatic ring system having from 5-15 ring atoms consisting of carbon, nitrogen, sulfur and oxygen. A heteroaryl can contain 1, 2, 3 or 4 (e.g., 1 or 2) heteroatoms independently selected from N, S and O. In one embodiment, heteroaryl has 5 or 6 ring atoms (e.g., five ring atoms). Monocyclic heteroaryls include, but are not limited to, furan, oxazole, thiophene, triazole, triazene, thiadiazole, oxadiazole, imidazole, isothiazole, isoxazole, pyrazole, pyridazine, pyridine, pyrazine, pyrimidine, pyrrole, tetrazole and thiazole. Bicyclic heteroaryls include, but are not limited to, indolizine, indole, isoindole, indazole, benzimidazole, benzofuran, benzothiazole, purine, quinoline, isoquinoline, cinnoline, phthalazine, quinazoline, quinoxaline, naphthyridine and pteridine. In some embodiments, heteroaryl is (C₅-C₆)heteroaryl.

“Heterocyclyl” or “heterocycloalkyl” refers to a saturated, aliphatic, monocyclic or polycyclic (e.g., bicyclic, tricyclic), monovalent, hydrocarbon ring system having the specified number of ring atoms, wherein at least one carbon atom in the ring system has been replaced with a heteroatom selected from N, S and O. Thus, “(C₃-C₆)heterocyclyl” means a heterocyclic ring system having from 3-6 ring atoms. A heterocyclyl can be monocyclic, fused bicyclic, bridged bicyclic or polycyclic, but is typically monocyclic. A heterocyclyl can contain 1, 2, 3 or 4 (e.g., 1) heteroatoms independently selected from N, S and O. When one heteroatom is S, it can be optionally mono- or di-oxygenated (i.e., —S(O)— or —S(O)₂). Examples of monocyclic heterocyclyls include, but are not limited to, aziridine, azetidine, pyrrolidine, piperidine, piperazine, azepane, tetrahydrofuran, tetrahydropyran, morpholine, thiomorpholine, dioxide, oxirane. In some embodiments, heterocycloalkyl is (C₃-Cis)heterocycloalkyl. In some embodiments, heterocycloalkyl is (C₃-C₁₂)heterocycloalkyl. In some embodiments, heterocycloalkyl is (C₃-C₈)heterocycloalkyl. In some embodiments, heterocycloalkyl is (C₃-C₆)heterocycloalkyl.

“Hydroxy” refers to —OH.

It is understood that substituents on the compounds of the invention can be selected by one of ordinary skill in the art to provide compounds that are chemically stable and that can be readily synthesized by techniques known in the art, as well as those methods set forth below. The term “stable,” as used herein, refers to compounds that are not substantially altered when subjected to conditions to allow for their production, detection and, in certain embodiments, recovery, purification and use for one or more of the purposes disclosed herein.

Combinations of substituents envisioned by this invention are preferably those that result in the formation of stable or chemically feasible compounds.

A designated group is unsubstituted, unless otherwise indicated, e.g., by provision of a variable that denotes allowable substituents for a designated group. For example, R³ in Structural Formula I denotes optional allowable substituents for the ring system to which R³ is attached. When the term “substituted” precedes a designated group, it means that one or more hydrogens of the designated group are replaced with a suitable substituent. Unless otherwise indicated, an “optionally substituted” group or “substituted or unsubstituted” group can have a suitable substituent at each substitutable position of the group and, when more than one position in any given structure may be substituted with more than one substituent selected from a specified group, the substituent can be the same or different at every position. Alternatively, an “optionally substituted” group or “substituted or unsubstituted” group can be unsubstituted. An “optionally substituted” group is, in some embodiments, substituted with 0-5 (e.g., 0-3, 0, 1, 2, 3, 4, 5) substituents. In some embodiments, an “optionally substituted” group is unsubstituted.

Suitable substituents for a substituted or optionally substituted group include, but are not limited to, for example, halogen, hydroxyl, carbonyl (such as carboxyl, alkoxycarbonyl, formyl, or acyl), thiocarbonyl (such as thioester, thioacetate, or thioformate), alkyl, alkoxy, alkylthio, acyloxy, phosphoryl, phosphate, phosphonate, amino, amido, amidino, imino, cyano, nitro, azido, sulfhydryl, alkylthio, sulfate, sulfonate, sulfamoyl, sulfonamido, sulfonyl, cycloalkyl, heterocyclyl, aryl or heteroaryl. It will be understood by those skilled in the art that substituents can themselves be substituted, if appropriate and where indicated. For instance, substituent(s) of a substituted alkyl may include substituted and unsubstituted forms of hydroxyl, amino, imino, amido, phosphoryl (including phosphonate and phosphinate), sulfonyl (including sulfate, sulfonamido, sulfamoyl and sulfonate) and carbonyls (including ketones, aldehydes, carboxylates, and esters), and the like.

In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R¹⁰⁰, (C₃-C₁₀)cycloalkyl, (C₃-C₁₀)heterocyclyl, (C₆-C₁₀)aryl or (C₅-C₁₀)heteroaryl, wherein R¹⁰⁰ is hydroxy, (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, or —C(O)R¹⁰⁰, wherein R¹⁰⁰ is hydroxy, (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, or —C(O)R¹⁰⁰, wherein R¹⁰⁰ is hydroxy, (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, carboxy, cyano, (C₁-C₆)alkyl, (C₁-C₆)haloalkyl, (C₁-C₆)alkoxy, or (C₁-C₆)haloalkoxy.

In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R¹⁰⁰, (C₃-C₁₀)cycloalkyl, (C₃-C₁₀)heterocyclyl, (C₆-C₁₀)aryl or (C₅-C₁₀)heteroaryl, wherein R¹⁰⁰ is (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, or —C(O)R¹⁰⁰, wherein R¹⁰⁰ is (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, (C₁-C₆)haloalkoxy, or —C(O)R¹⁰⁰, wherein R¹⁰⁰ is (C₁-C₆)alkoxy, amino, (C₁-C₆)alkylamino or (C₁-C₆)dialkylamino. In some embodiments, a substituted group or optionally substituted group is substituted or optionally substituted, respectively, with one or more (e.g., one, two, three, four or five) substituents independently selected from halo, hydroxy, cyano, (C₁-C₆)alkyl, (C₁-C₆)haloalkyl, (C₁-C₆)alkoxy, or (C₁-C₆)haloalkoxy.

As used herein, the term “pharmaceutically acceptable” refers to species which are, within the scope of sound medical judgment, suitable for use in contact with the tissues of mammals without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like. Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as various “solvates” or “hydrates.” A “hydrate” is a compound that exists in a composition with one or more water molecules. The composition can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention. In all provided structures, any hydrogen atom can also be independently selected from deuterium (²H), tritium (³H) and/or fluorine (F). Such compounds are useful, for example, as analytical tools, as probes in biological assays, or as therapeutic agents in accordance with the present invention.

Compounds disclosed herein may exist as stereoisomers. For example, compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers. Unless otherwise indicated, all possible isomers and mixtures thereof, including optical isomers, rotamers, tautomers and cis- and trans-isomers, are intended to be encompassed by the present disclosure.

When a disclosed compound is depicted by structure without indicating the stereochemistry, and the compound has one chiral center, it is to be understood that the structure encompasses one enantiomer or diastereomer of the compound separated or substantially separated from the corresponding optical isomer(s), a racemic mixture of the compound and mixtures enriched in one enantiomer or diastereomer relative to its corresponding optical isomer(s). When a disclosed compound is depicted by a structure indicating stereochemistry, and the compound has more than one chiral center, the stereochemistry indicates relative stereochemistry, rather than the absolute configuration of the substituents around the one or more chiral carbon atoms. “R” and “S” are used to indicate the absolute configuration of substituents around one or more chiral carbon atoms.

“Enantiomers” are pairs of stereoisomers that are non-superimposable mirror images of one another, most commonly because they contain an asymmetrically substituted carbon atom that acts as a chiral center.

“Diastereomers” are stereoisomers that are not related as mirror images, most commonly because they contain two or more asymmetrically substituted carbon atoms.

“Racemate” or “racemic mixture,” as used herein, refer to a mixture containing equimolar quantities of two enantiomers of a compound. Such mixtures exhibit no optical activity (i.e., they do not rotate a plane of polarized light).

Percent enantiomeric excess (ee) is defined as the absolute difference between the mole fraction of each enantiomer multiplied by 100% and can be represented by the following equation:

$$ee = \vert \frac{R - S}{R + S} \vert \times 100 \% ,$$

where R and S represent the respective fractions of each enantiomer in a mixture, such that R+S=1. An enantiomer may be present in an ee of at least or about 50%, about 60%, about 70%, about 80%, about 90%, about 95%, about 96%, about 97%, about 98%, about 99% or about 99.9%.

Percent diastereomeric excess (de) is defined as the absolute difference between the mole fraction of each diastereomer multiplied by 100% and can be represented by the following equation:

$$de = \vert \frac{D 1 - ( D 2 + D 3 + D 4 … )}{D 1 + ( D 2 + D 3 + D 4 … )} \vert \times 100 \% ,$$

where D1 and (D2+D3+D1+(D2+D3+D4 . . . ) represent the respective fractions of each diastereomer in a mixture, such that D1+(D2+D3+D4 . . . )=1. A diastereomer may be present in a de of at least or about 50%, about 60%, about 70%, about 80%, about 90%, about 95%, about 96%, about 97%, about 98%, about 99% or about 99.9%.

Methods of obtaining an optical isomer separated or substantially separated from the corresponding optical isomer(s) are known in the art. For example, an optical isomer can be purified from a racemic mixture by well-known chiral separation techniques, such as, but not limited to, normal- and reverse-phase chromatography, and crystallization. An optical isomer can also be prepared by the use of chiral intermediates or catalysts in synthesis. In some cases, compounds having at least some degree of enantiomeric enrichment can be obtained by physical processes, such as selective crystallization of salts or complexes formed with chiral adjuvants.

As used herein, the term “compound of the disclosure” refers to a compound of any structural formula depicted herein (e.g., a compound of structural formula I or a subformula thereof, a compound of Table A, B or C), as well as isomers, such as stereoisomers (including diastereoisomers, enantiomers and racemates) and tautomers thereof, isotopologues thereof, and inherently formed moieties (e.g., polymorphs and/or solvates, such as hydrates) thereof. When a moiety is present that is capable of forming a salt, then salts are included as well, in particular, pharmaceutically acceptable salts.

“Pharmaceutically acceptable carrier” refers to a carrier or excipient that does not destroy the pharmacological activity of the agent with which it is formulated and is, within the scope of sound medical judgment, suitable for use in contact with the tissues of mammals without undue toxicity, irritation, allergic response and the like, and is commensurate with a reasonable benefit/risk ratio. Pharmaceutically acceptable carriers include any and all clinically useful solvents, dispersion media, coatings, antibacterial and antifungal agents, isotonic and absorption delaying agents and the like. Non-limiting examples of pharmaceutically acceptable carriers include excipients such as adjuvants, binders, fillers, diluents, disintegrants, emulsifying agents, wetting agents, lubricants, glidants, sweetening agents, flavoring agents, and coloring agents. Suitable pharmaceutically acceptable carriers are described in Remington's Pharmaceutical Sciences, 19th Ed. (Mack Publishing Co., Easton, 1995). The choice of a pharmaceutically acceptable carrier often depends upon the intended route of administration of the agent(s) with which it is formulated.

As used herein, “inhibitor of the metadherin (MTDH)-Staphylococcal nuclease domain containing 1 (SND1) protein-protein interaction,” “MTDH-SND1 inhibitor” and similar terms refer to an agent (e.g., a compound of the disclosure) that inhibits the interaction of MTDH and SND1. The crystal structure of MTDH-SND1 complex has been resolved, and revealed a unique interface between the two N-terminal SN domains of SND1 and a peptide motif of MTDH. The surface contour of SND1 revealed two deep pockets that specifically interact with the MTDH residues. In particular, the bulky and hydrophobic side chains of W394 and W401 of MTDH were found to bind deeply into the two hydrophobic binding pockets of SND1. Point mutations of these two evolutionarily conserved tryptophan residues in MTDH, which blocked the interaction with SND1, also completely eliminated the tumor-supportive function of MTDH. Without wishing to be bound by any particular theory, it is believed that the MTDH-SND1 inhibitors described herein may exert their inhibitory effect at the site of MTDH and/or SND1 where MTDH and SND1 bind to one another, e.g., at residues 393-403 of MTDH.

“Treating,” as used herein, refers to taking steps to deliver a therapy to a subject, such as a mammal, in need thereof (e.g., as by administering to a mammal one or more therapeutic agents). “Treating” includes inhibiting the disease or condition (e.g., as by slowing or stopping its progression or causing regression of the disease or condition), and relieving the symptoms resulting from the disease or condition.

“Administering,” as used herein, refers to taking steps to deliver an agent to a subject, such as a mammal, in need thereof (e.g., as by administering to a mammal one or more therapeutic agents). Administering can be performed, for example, once, a plurality of times, and/or over one or more extended periods. Administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, “subject” encompasses mammals. Examples of mammals include, but are not limited to, humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In various embodiments, the subject is human.

### Compounds

A first embodiment is a compound of the following structural formula:

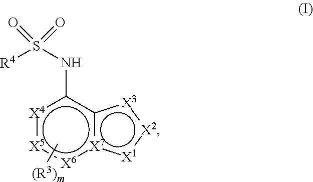

or a pharmaceutically acceptable salt thereof, wherein:
X¹—X²—X³ is N—C(R²⁰)—N, C(R¹⁰)—N—N, C(R¹⁰)—C(R²⁰)—N, N—C(R²⁰)—O, O—C(R²⁰)—N, C(R¹⁰)—N—O, N—C(R²⁰)—S, S—C(R²⁰)—N, C(R¹⁰)—C(R²⁰)—O, N(R¹¹)—C(R²⁰)—N or N(H)—C(O)—O;
   R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
   R¹¹ is H, (C₁-C₆)alkyl, (C₁-C₆)haloalkyl, (C₃-C₁₀)cycloalkyl, (C₃-C₁₀)heterocyclyl, (C₆-C₁₀)aryl or (C₅-C₁₀)heteroaryl;
   R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
X⁴, X⁵ and X⁶ are each independently C(H) or N;
X⁷ is C or N;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
   each R³⁰ is independently hydroxy, (C₁-C₆)alkoxy, amino, (C₁-C₆)alkylamino or (C₁-C₆)dialkylamino;
R⁴ is (C₃-C₁₀)cycloalkyl, (C₃-C₁₀)heterocyclyl, (C₆-C₁₀)aryl or (C₅-C₁₀)heteroaryl optionally substituted with one or more R⁴⁰;
   R⁴⁰, for each occurrence, is independently halo, cyano, (C₁-C₆)alkyl, (C₁-C₆)haloalkyl, (C₁-C₆)alkoxy(C₁-C₆)alkoxy, carboxy(C₁-C₆)alkoxy, HO—N(H)C(O)—(C₁-C₆)alkoxy, HOS(O)₂—(C₁-C₆)alkoxy, H₂NS(O)₂—(C₁-C₆)alkoxy, P(O)(OH)₂—(C₁-C₆)alkoxy, P(O)(OH)(H)—(C₁-C₆)alkoxy, (HO)₂B—(C₁-C₆)alkoxy, tetrazole-(C₁-C₆)alkoxy, thiazolidinedione-(C₁-C₆)alkoxy, oxazolidinedione-(C₁-C₆)alkoxy, isothiazole-(C₁-C₆)alkoxy, isoxazole-(C₁-C₆)alkoxy, oxooxadiazole-(C₁-C₆)alkoxy, oxothiadiazole-(C₁-C₆)alkoxy, thioxooxadiazole-(C₁-C₆)alkoxy, (C₁-C₆)alkoxy, (C₁-C₆)haloalkoxy, amino, (C₁-C₆)alkylamino, (C₁-C₆)dialkylamino, —C(O)R⁴¹, (C₃-C₁₀)cycloalkyl, (C₃-C₁₀)heterocyclyl, (C₆-C₁₀)aryl or (C₅-C₁₀)heteroaryl; or
   two R⁴⁰ on adjacent atoms of R⁴, taken together with the atoms to which they are attached, form a 5- or 6-membered cycle optionally substituted with one or more R⁴²;
   R⁴¹ is (C₁-C₆)alkoxy, amino, (C₁-C₆)alkylamino or (C₁-C₆)dialkylamino;
   R⁴², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

In a first aspect of the first embodiment, X¹—X²—X³ is C(R¹⁰)—N—N, N—C(R²⁰)—N, C(R¹⁰)—C(R²⁰)—N, C(R¹⁰)—C(R²⁰)—O or N(R¹¹)—C(R²⁰)—N. Values for the remaining variables are as described in the first embodiment.

In a second aspect of the first embodiment, R¹⁰ is H, (C₁-C₆)alkyl or (C₃-C₁₀)cycloalkyl. Values for the remaining variables are as described in the first embodiment, or first aspect thereof.

In a third aspect of the first embodiment, R¹⁰ is H, methyl, ethyl, cyclopropyl or cyclobutyl. Values for the remaining variables are as described in the first embodiment, or first or second aspect thereof.

In a fourth aspect of the first embodiment, R¹¹ is H, (C₁-C₆)alkyl or (C₃-C₁₀)cycloalkyl. Values for the remaining variables are as described in the first embodiment, or first through third aspects thereof.

In a fifth aspect of the first embodiment, R¹¹ is H, methyl, ethyl, cyclopropyl or cyclobutyl. Values for the remaining variables are as described in the first embodiment, or first through fourth aspects thereof.

In a sixth aspect of the first embodiment, R²⁰ is H, (C₁-C₆)alkyl or (C₃-C₁₀)cycloalkyl. Values for the remaining variables are as described in the first embodiment, or first through fifth aspects thereof.

In a seventh aspect of the first embodiment, R²⁰ is H, methyl, ethyl, cyclopropyl or cyclobutyl. Values for the remaining variables are as described in the first embodiment, or first through sixth aspects thereof.

In an eighth aspect of the first embodiment, X⁴, X⁵ and X⁶ are each C(H); and X⁷ is N. Values for the remaining variables are as described in the first embodiment, or first through seventh aspects thereof.

In a ninth aspect of the first embodiment, one of X⁴, X⁵ and X⁶ is N, and the other two are each C(H); and X⁷ is C. Values for the remaining variables are as described in the first embodiment, or first through eighth aspects thereof.

In a tenth aspect of the first embodiment, each R³ is independently hydroxy, halo, cyano, (C₁-C₆)alkyl, (C₁-C₆)haloalkyl, (C₁-C₆)alkoxy or (C₁-C₆)haloalkoxy. Values for the remaining variables are as described in the first embodiment, or first through ninth aspects thereof.

In an eleventh aspect of the first embodiment, R⁴ is optionally substituted (C₆-C₁₀)aryl or (C₅-C₁₀)heteroaryl. Values for the remaining variables are as described in the first embodiment, or first through tenth aspects thereof.

In a twelfth aspect of the first embodiment R⁴ is optionally substituted phenyl or pyridinyl. Values for the remaining variables are as described in the first embodiment, or first through eleventh aspects thereof.

In a thirteenth aspect of the first embodiment, R⁴⁰, for each occurrence, is independently halo, (C₁-C₆)alkyl or (C₁-C₆)alkoxy. Values for the remaining variables are as described in the first embodiment, or first through twelfth aspects thereof.

In a fourteenth aspect of the first embodiment, R⁴⁰, for each occurrence, is independently fluoro, chloro, methyl or methoxy. Values for the remaining variables are as described in the first embodiment, or first through thirteenth aspects thereof.

In a fifteenth aspect of the first embodiment, m is 0. Values for the remaining variables are as described in the first embodiment, or first through fourteenth aspects thereof.

In a sixteenth aspect of the first embodiment, X¹—X²—X³ is N—C(R²⁰)—N or C(R¹⁰)—N—N. Values for the remaining variables are as described in the first embodiment, or first through sixteenth aspects thereof.

In a seventeenth aspect of the first embodiment, R¹⁰ is H, (C₁-C₃)alkyl or (C₃-C₆)cycloalkyl. Values for the remaining variables are as described in the first embodiment, or first through sixteenth aspects thereof.

In an eighteenth aspect of the first embodiment, R¹⁰ is H, methyl, ethyl, n-propyl, iso-propyl, cyclopropyl, cyclobutyl, cyclopentyl, cyclohexyl, phenyl, carboxyphenyl, cyano, or carboxy. Values for the remaining variables are as described in the first embodiment, or first through seventeenth aspects thereof.

In a nineteenth aspect of the first embodiment, R²⁰ is H, (C₁-C₃)alkyl or (C₃-C₆)cycloalkyl. Values for the remaining variables are as described in the first embodiment, or first through eighteenth aspects thereof.

In a twentieth aspect of the first embodiment, R¹⁰ is H, methyl, ethyl, n-propyl, iso-propyl, cyclopropyl, cyclobutyl, cyclopentyl, cyclohexyl, phenyl, carboxyphenyl, cyano, or carboxy. Values for the remaining variables are as described in the first embodiment, or first through nineteenth aspects thereof.

In a twenty-first aspect of the first embodiment, each R³ is independently halo. Values for the remaining variables are as described in the first embodiment, or first through twentieth aspects thereof.

In a twenty-second aspect of the first embodiment, R⁴⁰, for each occurrence, is independently halo, (C₁-C₆)alkyl, (C₁-C₆)alkoxy, (C₁-C₆)alkoxy(C₁-C₆)alkoxy or carboxy(C₁-C₆)alkoxy, or two R⁴⁰ on adjacent atoms of R⁴, taken together with the atoms to which they are attached, form a 5- or 6-membered cycle optionally substituted with one or more R⁴². Values for the remaining variables are as described in the first embodiment, or first through twenty-first aspects thereof.

In a twenty-third aspect of the first embodiment, R⁴⁰, for each occurrence, is independently fluoro, chloro, methyl, trifluoromethyl, difluoromethyl, fluoromethyl, methoxy, methoxymethoxy or —OCH₂CO₂H, or two R⁴⁰ on adjacent atoms of R⁴ are —N(H)C(O)O— or —CH₂CH₂O—. Values for the remaining variables are as described in the first embodiment, or first through twenty-second aspects thereof.

In a twenty-fourth aspect of the first embodiment:
X¹—X²—X³ is C(R¹⁰)—N—N, N—C(R²⁰)—N, C(R¹⁰)—C(R²⁰)—N, N—C(R²⁰)—O, O—C(R²⁰)—N, C(R¹⁰)—N—O, N—C(R²⁰)—S, S—C(R²⁰)—N, C(R¹⁰)—C(R²⁰)—O or N(R¹¹)—C(R²⁰)—N;
   R¹⁰ is H, OH, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl or (C₅-C₁₀)heteroaryl;
   R¹¹ is H, (C₁-C₆)alkyl, (C₁-C₆)haloalkyl, (C₃-C₁₀)cycloalkyl, (C₃-C₁₀)heterocyclyl, (C₆-C₁₀)aryl or (C₅-C₁₀)heteroaryl;
   R²⁰ is H, OH, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl or (C₅-C₁₀)heteroaryl;
X⁴, X⁵ and X⁶ are each independently C(H) or N;
X⁷ is C or N;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
   each R³⁰ is independently (C₁-C₆)alkoxy, amino, (C₁-C₆)alkylamino or (C₁-C₆)dialkylamino;
R⁴ is (C₃-C₁₀)cycloalkyl, (C₃-C₁₀)heterocyclyl, (C₆-C₁₀)aryl or (C₅-C₁₀)heteroaryl optionally substituted with one or more R⁴⁰;
   R⁴⁰, for each occurrence, is independently halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R⁴¹, (C₃-C₁₀)cycloalkyl, (C₃-C₁₀)heterocyclyl, (C₆-C₁₀)aryl or (C₅-C₁₀)heteroaryl;
   R⁴¹ is (C₁-C₆)alkoxy, amino, (C₁-C₆)alkylamino or (C₁-C₆)dialkylamino; and
m is 0, 1, 2 or 3. Alternative values for the variables are as described in the first embodiment, or first through twenty-third aspects thereof.

A second embodiment is a compound of the following structural formula:

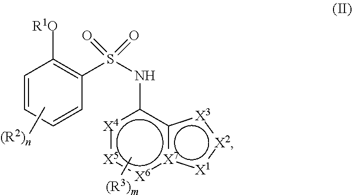

or a pharmaceutically acceptable salt thereof, wherein:
R¹ is (C₁-C₆)alkyl;
each R² is independently halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R²¹, (C₃-C₁₀)cycloalkyl, (C₃-C₁₀)heterocyclyl, (C₆-C₁₀)aryl or (C₅-C₁₀)heteroaryl;
   R²¹ is (C₁-C₆)alkoxy, amino, (C₁-C₆)alkylamino or (C₁-C₆)dialkylamino; and
n is 0, 1 or 2. Values for the remaining variables (e.g., X¹, X², X³, X⁴, X⁵, X⁶, X⁷, R³, m) are as described in the first embodiment, or any aspect thereof.

In a first aspect of the second embodiment, R¹ is methyl. Values for the remaining variables are as described in the first embodiment, or any aspect thereof, or the second embodiment.

In a second aspect of the second embodiment, each R² is independently halo, (C₁-C₆)alkyl or (C₁-C₆)alkoxy. Values for the remaining variables are as described in the first embodiment, or any aspect thereof, or the second embodiment, or first aspect thereof.

In a third aspect of the second embodiment, each R² is independently fluoro, chloro, methyl or methoxy. Values for the remaining variables are as described in the first embodiment, or any aspect thereof, or the second embodiment, or first or second aspect thereof.

In a fourth aspect of the second embodiment, n is 0 or 1. Values for the remaining variables are as described in the first embodiment, or any aspect thereof, or the second embodiment, or first through third aspects thereof.

A third embodiment is a compound of the following structural formula:

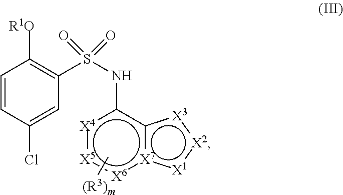

or a pharmaceutically acceptable salt thereof. Values for the variables (e.g., X¹, X², X³, X⁴, X⁵, X⁶, X⁷, R¹, R³, m) are as described in the first, second, fourth or fifth embodiment, or any aspect thereof.

A fourth embodiment is a compound represented by the following structural formula:

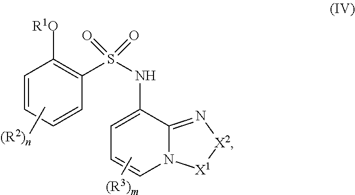

or a pharmaceutically acceptable salt thereof, wherein:
X¹ is N or C(R¹⁰);
   R¹⁰ is H, OH, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl or (C₅-C₁₀)heteroaryl;
X² is N or C(R²⁰);
   R²⁰ is H, OH, halo, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl or (C₅-C₁₀)heteroaryl;
R¹ is (C₁-C₆)alkyl;
each R² is independently halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R²¹, (C₃-C₁₀)cycloalkyl, (C₃-C₁₀)heterocyclyl, (C₆-C₁₀)aryl or (C₅-C₁₀)heteroaryl;
   R²¹ is (C₁-C₆)alkoxy, amino, (C₁-C₆)alkylamino or (C₁-C₆)dialkylamino;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
   each R³⁰ is independently (C₁-C₆)alkoxy, amino, (C₁-C₆)alkylamino or (C₁-C₆)dialkylamino;
n is 0, 1 or 2; and
m is 0, 1, 2 or 3.

In a first aspect of the fourth embodiment, X¹ is N and X² is C(R²⁰). Values for the remaining variables are as described in the fourth embodiment.

In a second aspect of the fourth embodiment, X¹ is C(R¹⁰) and X² is N. Values for the remaining variables are as described in the fourth embodiment, or first aspect thereof.

In a third aspect of the fourth embodiment, X¹ is C(R¹⁰) and X² is C(R²⁰). Values for the remaining variables are as described in the fourth embodiment, or first or second aspect thereof.

In a fourth aspect of the fourth embodiment, R¹⁰ is H, (C₁-C₆)alkyl or (C₃-C₁₀)cycloalkyl. Values for the remaining variables are as described in the fourth embodiment, or first through third aspects thereof.

In a fifth aspect of the fourth embodiment, R¹⁰ is H, methyl, ethyl, cyclopropyl or cyclobutyl. Values for the remaining variables are as described in the fourth embodiment, or first through fourth aspects thereof.

In a sixth aspect of the fourth embodiment, R²⁰ is H, (C₁-C₆)alkyl or (C₃-C₁₀)cycloalkyl. Values for the remaining variables are as described in the fourth embodiment, or first through fifth aspects thereof.

In a seventh aspect of the fourth embodiment, R²⁰ is H, methyl, ethyl, cyclopropyl or cyclobutyl. Values for the remaining variables are as described in the fourth embodiment, or first through sixth aspects thereof.

In an eighth aspect of the fourth embodiment, R¹ is methyl. Values for the remaining variables are as described in the fourth embodiment, or first through seventh aspects thereof.

In a ninth aspect of the fourth embodiment, each R² is independently halo, cyano, (C₁-C₆)alkyl, (C₁-C₆)haloalkyl, (C₁-C₆)alkoxy or (C₁-C₆)haloalkoxy. Values for the remaining variables are as described in the fourth embodiment, or first through eighth aspects thereof.

In a tenth aspect of the fourth embodiment, each R³ is independently hydroxy, halo, cyano, (C₁-C₆)alkyl, (C₁-C₆)haloalkyl, (C₁-C₆)alkoxy or (C₁-C₆)haloalkoxy. Values for the remaining variables are as described in the fourth embodiment, or first through ninth aspects thereof.

In an eleventh aspect of the fourth embodiment, n is 0 or 1. Values for the remaining variables are as described in the fourth embodiment, or first through tenth aspects thereof.

In a twelfth aspect of the fourth embodiment, m is 0. Values for the remaining variables are as described in the fourth embodiment, or first through eleventh aspects thereof.

In a thirteenth aspect of the fourth embodiment, the compound is not

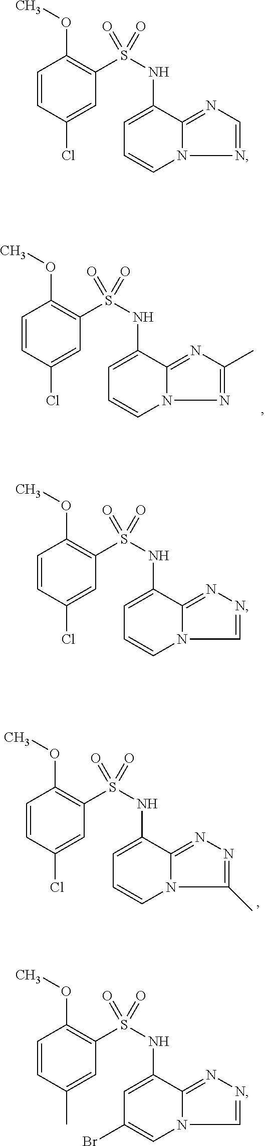 [SMILES: COc1ccc(Cl)cc1S(=O)(=O)Nc1cccn2ncnc12 ; COc1ccc(Cl)cc1S(=O)(=O)Nc1cccn2nc(C)nc12 ; COc1ccc(Cl)cc1S(=O)(=O)Nc1cccn2cnnc12 ; COc1ccc(Cl)cc1S(=O)(=O)Nc1cccn2c(C)nnc12 ; COc1ccc(C)cc1S(=O)(=O)Nc1cc(Br)cn2cnnc12]

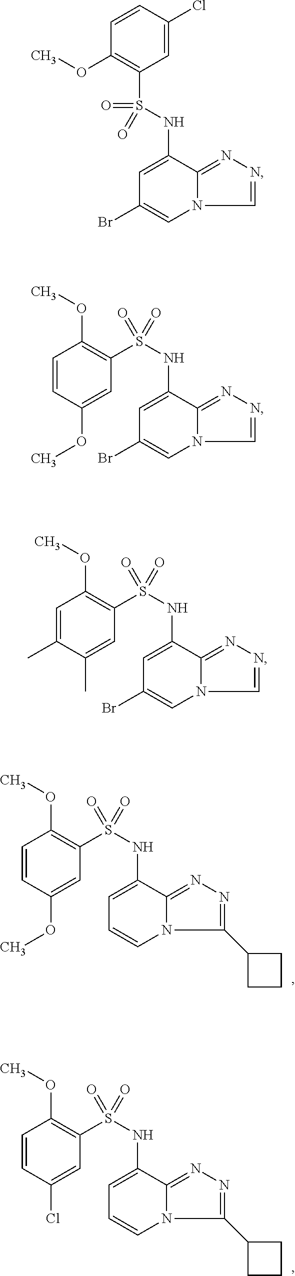 [SMILES: COc1ccc(Cl)cc1S(=O)(=O)Nc1cc(Br)cn2cnnc12 ; COc1ccc(OC)c(S(=O)(=O)Nc2cc(Br)cn3cnnc23)c1 ; COc1cc(C)c(C)cc1S(=O)(=O)Nc1cc(Br)cn2cnnc12 ; COc1ccc(OC)c(S(=O)(=O)Nc2cccn3c(C4CCC4)nnc23)c1 ; COc1ccc(Cl)cc1S(=O)(=O)Nc1cccn2c(C3CCC3)nnc12]

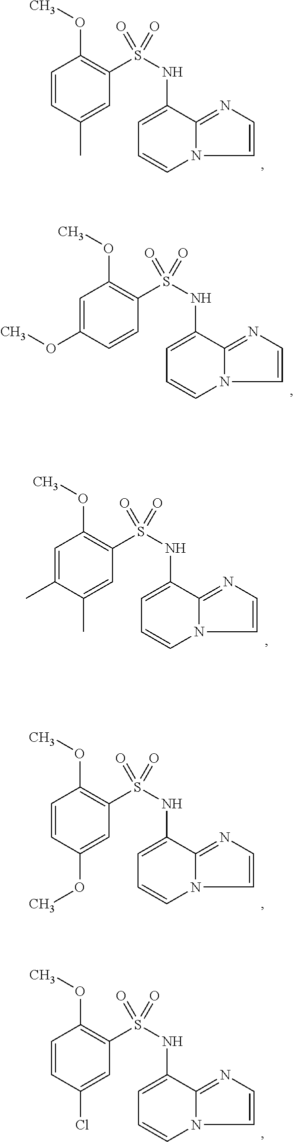 [SMILES: COc1ccc(C)cc1S(=O)(=O)Nc1cccn2ccnc12 ; COc1ccc(S(=O)(=O)Nc2cccn3ccnc23)c(OC)c1 ; COc1cc(C)c(C)cc1S(=O)(=O)Nc1cccn2ccnc12 ; COc1ccc(OC)c(S(=O)(=O)Nc2cccn3ccnc23)c1 ; COc1ccc(Cl)cc1S(=O)(=O)Nc1cccn2ccnc12]

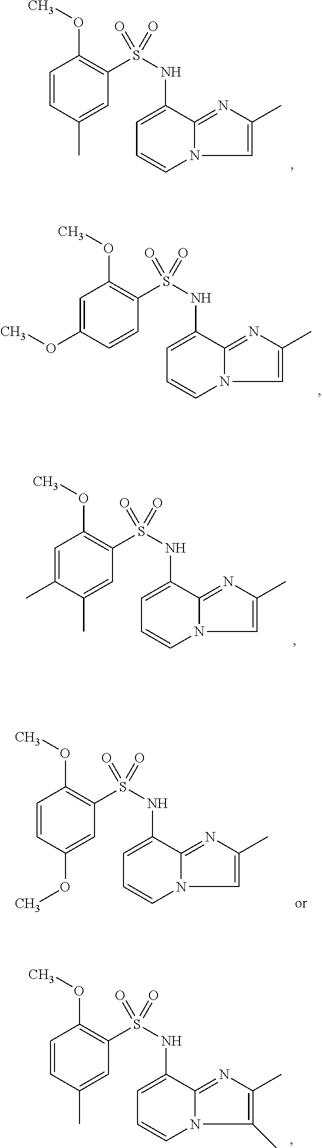

or a pharmaceutically acceptable salt thereof. Values for the variables are as described in the fourth embodiment, or first through twelfth aspects thereof.

In a fourteenth aspect of the fourth embodiment, each R² is independently fluoro, bromo, cyano, (C₂-C₆)alkyl, (C₁-C₆)haloalkyl, (C₂-C₆)alkoxy or (C₁-C₆)haloalkoxy. Values for the remaining variables are as described in the fourth embodiment, or first through thirteenth aspects thereof.

A fifth embodiment is a compound of the following structural formula:

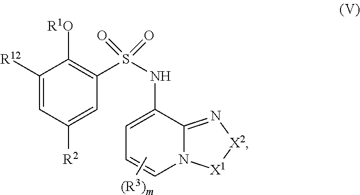

or a pharmaceutically acceptable salt thereof, wherein:
X¹ is N and X² is C(R²⁰), or X₁ is C(R¹⁰) and X² is N;
   R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
   R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹ is (C₁-C₆)alkyl, (C₁-C₆)alkoxy(C₁-C₆)alkyl, carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl;
R² is halo, (C₁-C₆)alkyl, (C₁-C₆)haloalkyl, (C₁-C₆)alkoxy or (C₁-C₆)haloalkoxy;
each R³ is independently halo;
R¹² is hydrogen or R¹ and R¹², taken together with their intervening atoms, form a 5- or 6-membered cycle optionally substituted with one or more R²²;
   R²², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

In a first aspect of the fifth embodiment, R¹ is not methyl. Values for the remaining variables and other values for R¹ are as described in the first through fourth embodiments, or any aspect thereof, or the fifth embodiment.

In a second aspect of the fifth embodiment, the compound is not

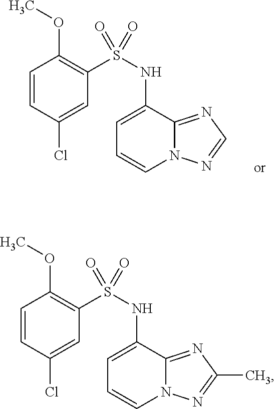 [SMILES: COc1ccc(Cl)cc1S(=O)(=O)Nc1cccn2ncnc12 ; COc1ccc(Cl)cc1S(=O)(=O)Nc1cccn2nc(C)nc12]

or a pharmaceutically acceptable salt thereof. Values for the variables are as described in the first through fourth embodiments, or any aspect thereof, or the fifth embodiment, or first aspect thereof.

In a third aspect of the fifth embodiment, the compound is not

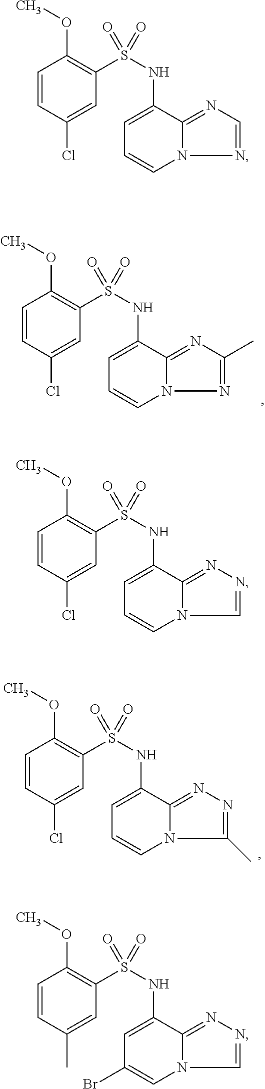 [SMILES: COc1ccc(Cl)cc1S(=O)(=O)Nc1cccn2ncnc12 ; COc1ccc(Cl)cc1S(=O)(=O)Nc1cccn2nc(C)nc12 ; COc1ccc(Cl)cc1S(=O)(=O)Nc1cccn2cnnc12 ; COc1ccc(Cl)cc1S(=O)(=O)Nc1cccn2c(C)nnc12 ; COc1ccc(C)cc1S(=O)(=O)Nc1cc(Br)cn2cnnc12]

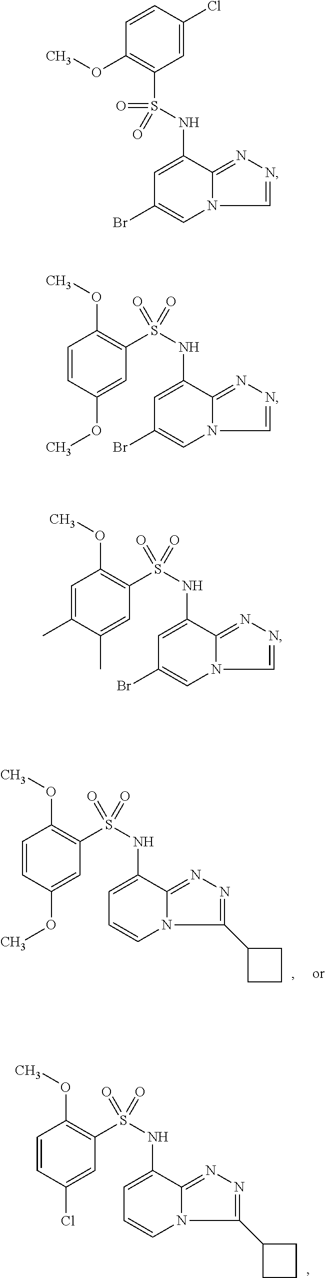 [SMILES: COc1ccc(Cl)cc1S(=O)(=O)Nc1cc(Br)cn2cnnc12 ; COc1ccc(OC)c(S(=O)(=O)Nc2cc(Br)cn3cnnc23)c1 ; COc1cc(C)c(C)cc1S(=O)(=O)Nc1cc(Br)cn2cnnc12 ; COc1ccc(OC)c(S(=O)(=O)Nc2cccn3c(C4CCC4)nnc23)c1 ; COc1ccc(Cl)cc1S(=O)(=O)Nc1cccn2c(C3CCC3)nnc12]

or a pharmaceutically acceptable salt thereof. Values for the variables are as described in the first through fourth embodiments, or any aspect thereof, or the fifth embodiment, or first aspect thereof.

In a fourth aspect of the fifth embodiment, X¹ is N and X² is C(R²⁰). Values for the remaining variables are as described in the first through fourth embodiments, or any aspect thereof, or the fifth embodiment, or first through third aspects thereof.

In a fifth aspect of the fifth embodiment, X¹ is C(R¹⁰) and X² is N. Values for the remaining variables are as described in the first through fourth embodiments, or any aspect thereof, or the fifth embodiment, or first through fourth aspects thereof.

In a sixth aspect of the fifth embodiment, R¹ is (C₁-C₆)alkyl or (C₁-C₆)alkoxy(C₁-C₆)alkyl and R¹² is hydrogen, or R¹ and R¹², taken together with their intervening atoms, form a 5- or 6-membered cycle optionally substituted with one or more R²². Values for the remaining variables are as described in the first through fourth embodiments, or any aspect thereof, or the fifth embodiment, or first through fifth aspects thereof.

In a seventh aspect of the fifth embodiment, R¹ is carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl. Values for the remaining variables are as described in the first through fourth embodiments, or any aspect thereof, or the fifth embodiment, or first through sixth aspects thereof.

In an eighth aspect of the fifth embodiment, R¹ is methyl, methoxymethyl or —CH₂CO₂H and R¹² is hydrogen, or R¹ and R¹², taken together, are —N(H)C(O)— or —CH₂CH₂—. Values for the remaining variables are as described in the first through fourth embodiments, or any aspect thereof, or the fifth embodiment, or first through seventh aspects thereof.

In a ninth aspect of the fifth embodiment, R² is chloro, fluoro, methyl, trifluoromethyl, difluoromethyl or fluoromethyl. Values for the remaining variables are as described in the first through fourth embodiments, or any aspect thereof, or the fifth embodiment, or first through eighth aspects thereof.

In a tenth aspect of the fifth embodiment, R² is chloro. Values for the remaining variables are as described the first through fourth embodiments, or any aspect thereof, or in the fifth embodiment, or first through ninth aspects thereof.

In an eleventh aspect of the fifth embodiment, R¹² is hydrogen. Values for the remaining variables are as described in the first through fourth embodiments, or any aspect thereof, or the fifth embodiment, or first through tenth aspects thereof.

A sixth embodiment is a compound of the following structural formula:

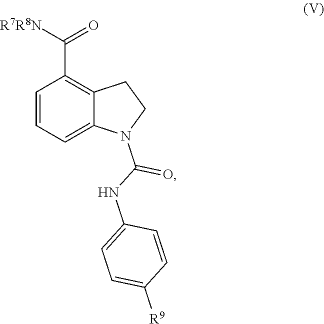

or a pharmaceutically acceptable salt thereof, wherein:
R⁷ is H or (C₁-C₆)alkyl;
R⁸ is H, (C₁-C₆)alkyl, or (C₃-C₁₀)cycloalkyl or (C₃-C₁₀)heterocyclyl optionally substituted with one or more R⁸⁰;
   R⁸⁰, for each occurrence, is independently halo, cyano, (C₁-C₆)alkyl, (C₁-C₆)haloalkyl, (C₃-C₁₀)cycloalkyl, (C₁-C₆)alkoxy or (C₁-C₆)haloalkoxy; and
R⁹ is H, halo, cyano, (C₁-C₆)alkyl, (C₁-C₆)haloalkyl, (C₁-C₆)alkoxy or (C₁-C₆)haloalkoxy.

In a first aspect of the sixth embodiment, R⁷ is H or methyl. Values for the remaining variables are as described in the sixth embodiment.

In a second aspect of the sixth embodiment, R⁸ is (C₁-C₆)alkyl, or (C₃-C₁₀)cycloalkyl or (C₃-C₁₀)heterocyclyl optionally substituted with one or more R⁸⁰. Values for the remaining variables are as described in the sixth embodiment, or first aspect thereof.

In a third aspect of the sixth embodiment, R⁸ is methyl, pentyl or piperidinyl (e.g., piperidin-4-yl) optionally substituted with one or more R⁸⁰. Values for the remaining variables are as described in the sixth embodiment, or first or second aspect thereof.

In a fourth aspect of the sixth embodiment, R⁸⁰, for each occurrence, is independently methyl or cyclopropyl. Values for the remaining variables are as described in the sixth embodiment, or first through third aspects thereof.

In a fifth aspect of the sixth embodiment, R⁹ is H, (C₁-C₆)alkyl, or (C₁-C₆)alkoxy. Values for the remaining variables are as described in the sixth embodiment, or first through fourth aspects thereof.

In a sixth aspect of the sixth embodiment, R⁹ is H, methyl or methoxy. Values for the remaining variables are as described in the sixth embodiment, or first through fifth aspects thereof.

In a seventh aspect of the sixth embodiment, R⁸⁰, for each occurrence, is independently halo, cyano, (C₁-C₆)alkyl, (C₁-C₆)haloalkyl, (C₁-C₆)alkoxy or (C₁-C₆)haloalkoxy. Values for the remaining variable are as described in the sixth embodiment, of first through sixth aspects thereof.

Examples of compounds of the disclosure, including various of the formulas and subformulas described herein, are listed in Tables A, B and C. One embodiment is a compound having a compound structure in Table A, or a pharmaceutically acceptable salt thereof. Another embodiment is a compound having a compound structure in Table B, or a pharmaceutically acceptable salt thereof. Yet another embodiment is a compound having a compound structure in Table C, or a pharmaceutically acceptable salt thereof.

TABLE A

| Analog No. | CAS No. | Compound Structure |
|---|---|---|
| C-26 | 1358210-94-2 | 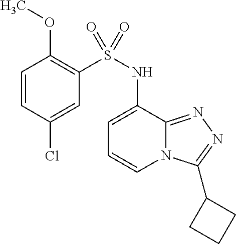 [SMILES: COc1ccc(Cl)cc1S(=O)(=O)Nc1cccn2c(C3CCC3)nnc12] |
| C-26-A1 | 1341006-82-3 | 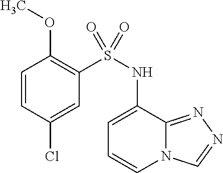 [SMILES: COc1ccc(Cl)cc1S(=O)(=O)Nc1cccn2cnnc12] |
| C-26-A2 | 1340834-16-3 | 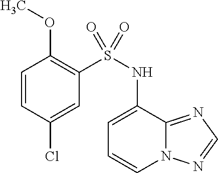 [SMILES: COc1ccc(Cl)cc1S(=O)(=O)Nc1cccn2ncnc12] |
| C-26-A3 | 1340749-12-3 | 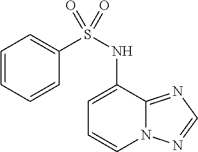 [SMILES: O=S(=O)(Nc1cccn2ncnc12)c1ccccc1] |
| C-26-A4 | 1358660-99-7 | 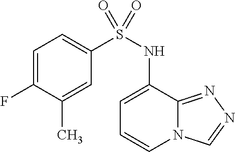 [SMILES: Cc1cc(S(=O)(=O)Nc2cccn3cnnc23)ccc1F] |
| C-26-A5 | 1341018-97-0 | 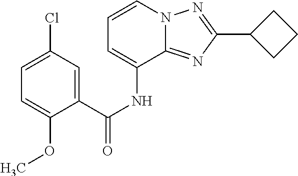 [SMILES: COc1ccc(Cl)cc1C(=O)Nc1cccn2nc(C3CCC3)nc12] |
| C-26-A6 | 1341030-00-9 | 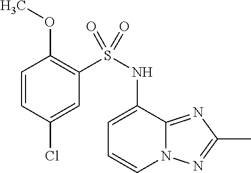 |
| C-26-A7 | 332388-62-2 | 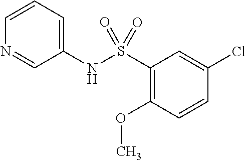 [SMILES: COc1ccc(Cl)cc1S(=O)(=O)Nc1cccnc1] |
| C-26-A8 | 1024346-65-3 | 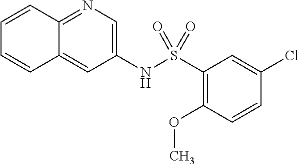 [SMILES: COc1ccc(Cl)cc1S(=O)(=O)Nc1cnc2ccccc2c1] |
| C-26-A9 | 313402-96-9 | 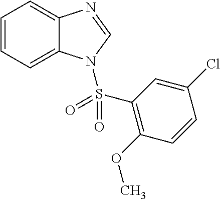 [SMILES: COc1ccc(Cl)cc1S(=O)(=O)n1cnc2ccccc21] |
| C-26-A10 | 697240-20-3 | 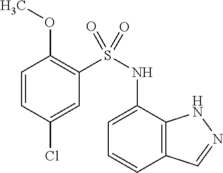 [SMILES: COc1ccc(Cl)cc1S(=O)(=O)Nc1cccc2cn[nH]c12] |
| C-26-A11 | 1040659-54-8 | 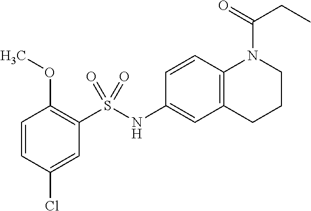 [SMILES: CCC(=O)N1CCCc2cc(NS(=O)(=O)c3cc(Cl)ccc3OC)ccc21] |
| C-26-A12 | 1115393-77-5 | 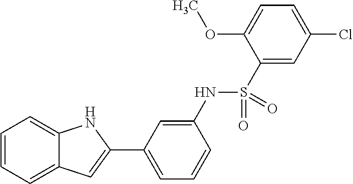 [SMILES: COc1ccc(Cl)cc1S(=O)(=O)Nc1cccc(-c2cc3ccccc3[nH]2)c1] |
| C-26-A13 | 904828-48-4 | 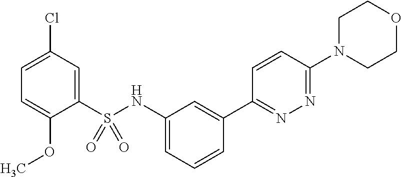 [SMILES: COc1ccc(Cl)cc1S(=O)(=O)Nc1cccc(-c2ccc(N3CCOCC3)nn2)c1] |
| C-26-A14 | 1340873-88-2 | 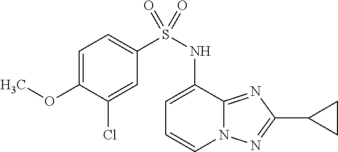 [SMILES: COc1ccc(S(=O)(=O)Nc2cccn3nc(C4CC4)nc23)cc1Cl] |
| C-26-A15 | 1111999-14-4 | 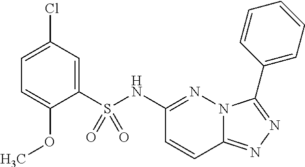 [SMILES: COc1ccc(Cl)cc1S(=O)(=O)Nc1ccc2nnc(-c3ccccc3)n2n1] |
| C-26-A16 | 1111959-63-7 | 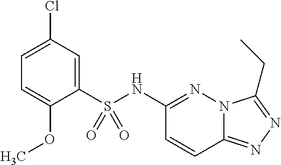 [SMILES: CCc1nnc2ccc(NS(=O)(=O)c3cc(Cl)ccc3OC)nn12] |
| C-26-A17 | 941906-40-7 | 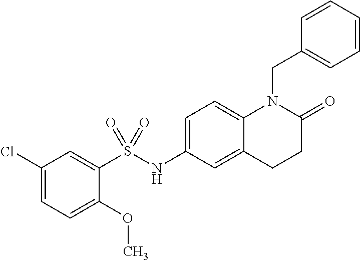 [SMILES: COc1ccc(Cl)cc1S(=O)(=O)Nc1ccc2c(c1)CCC(=O)N2Cc1ccccc1] |

TABLE B

| Analog No. | CAS No. | Compound Structure |
|---|---|---|
| C-32 | 1340976-05-7 | 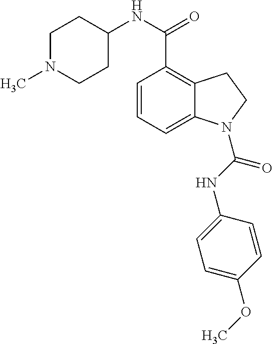 [SMILES: COc1ccc(NC(=O)N2CCc3c(C(=O)NC4CCN(C)CC4)cccc32)cc1] |
| C-32-A1 | 1340740-37-5 | 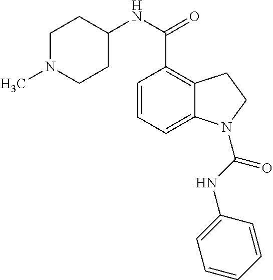 [SMILES: CN1CCC(NC(=O)c2cccc3c2CCN3C(=O)Nc2ccccc2)CC1] |
| C-32-A2 | 1340768-86-6 | 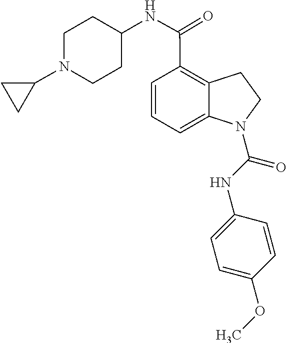 [SMILES: COc1ccc(NC(=O)N2CCc3c(C(=O)NC4CCN(C5CC5)CC4)cccc32)cc1] |
| C-32-A3 | 1340855-70-0 | 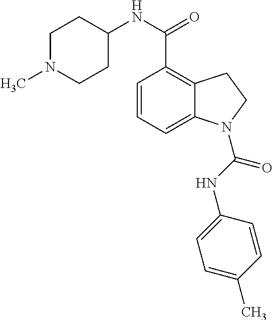 [SMILES: Cc1ccc(NC(=O)N2CCc3c(C(=O)NC4CCN(C)CC4)cccc32)cc1] |
| C-32-A4 | 1340897-71-3 | 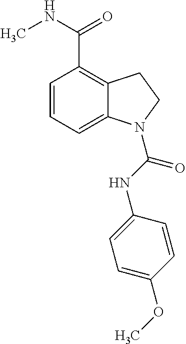 [SMILES: CNC(=O)c1cccc2c1CCN2C(=O)Nc1ccc(OC)cc1] |
| C-32-A5 | 1340740-46-6 | 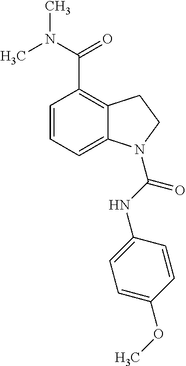 [SMILES: COc1ccc(NC(=O)N2CCc3c(C(=O)N(C)C)cccc32)cc1] |
| C-32-A6 | 1340687-88-8 | 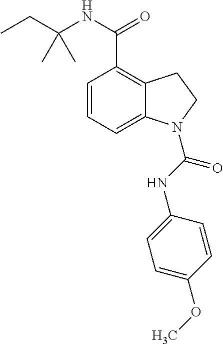 [SMILES: CCC(C)(C)NC(=O)c1cccc2c1CCN2C(=O)Nc1ccc(OC)cc1] |
| C-32-A7 | 932973-29-0 | 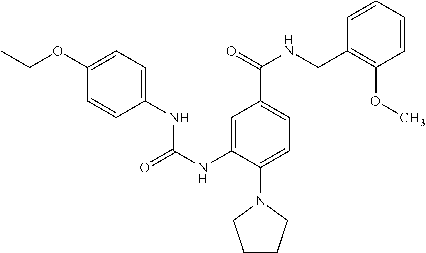 [SMILES: CCOc1ccc(NC(=O)Nc2cc(C(=O)NCc3ccccc3OC)ccc2N2CCCC2)cc1] |

TABLE C

| Analog No. | |
|---|---|
| (HKYK) | Compound Structure |
| 0001 | 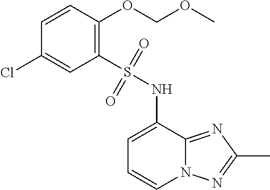 [SMILES: COCOc1ccc(Cl)cc1S(=O)(=O)Nc1cccn2nc(C)nc12] |
| 0002 | 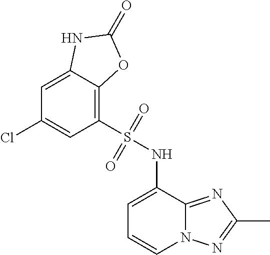 [SMILES: Cc1nc2c(NS(=O)(=O)c3cc(Cl)cc4[nH]c(=O)oc34)cccn2n1] |
| 0003 | 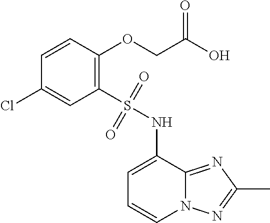 [SMILES: Cc1nc2c(NS(=O)(=O)c3cc(Cl)ccc3OCC(=O)O)cccn2n1] |
| 0004 | 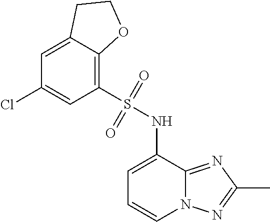 [SMILES: Cc1nc2c(NS(=O)(=O)c3cc(Cl)cc4c3OCC4)cccn2n1] |
| 0005 | 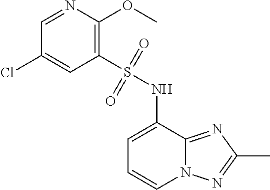 [SMILES: COc1ncc(Cl)cc1S(=O)(=O)Nc1cccn2nc(C)nc12] |
| 0006 | 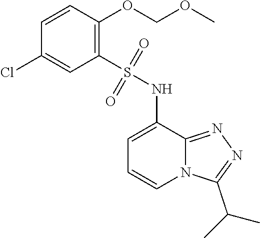 [SMILES: COCOc1ccc(Cl)cc1S(=O)(=O)Nc1cccn2c(C(C)C)nnc12] |
| 0007 | 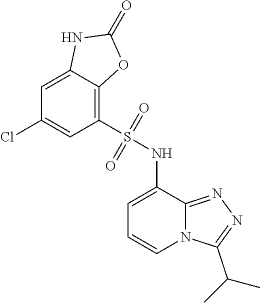 [SMILES: CC(C)c1nnc2c(NS(=O)(=O)c3cc(Cl)cc4[nH]c(=O)oc34)cccn12] |
| 0008 | 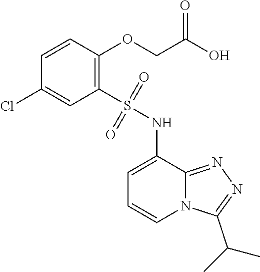 [SMILES: CC(C)c1nnc2c(NS(=O)(=O)c3cc(Cl)ccc3OCC(=O)O)cccn12] |
| 0009 | 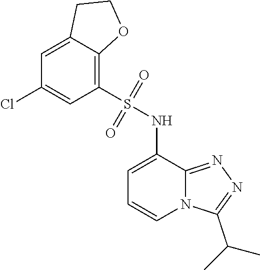 [SMILES: CC(C)c1nnc2c(NS(=O)(=O)c3cc(Cl)cc4c3OCC4)cccn12] |
| 0010 | 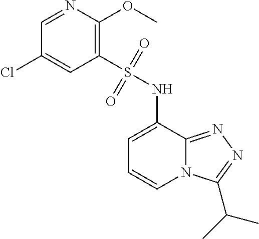 [SMILES: COc1ncc(Cl)cc1S(=O)(=O)Nc1cccn2c(C(C)C)nnc12] |
| 0011 | 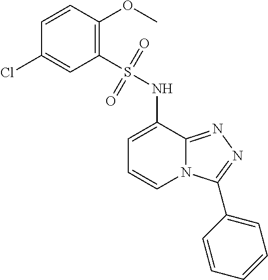 [SMILES: COc1ccc(Cl)cc1S(=O)(=O)Nc1cccn2c(-c3ccccc3)nnc12] |
| 0012 | 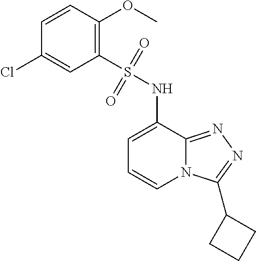 [SMILES: COc1ccc(Cl)cc1S(=O)(=O)Nc1cccn2c(C3CCC3)nnc12] |
| 0013 | 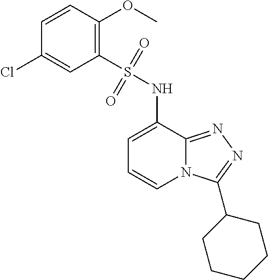 [SMILES: COc1ccc(Cl)cc1S(=O)(=O)Nc1cccn2c(C3CCCCC3)nnc12] |
| 0014 | 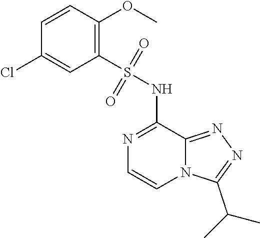 [SMILES: COc1ccc(Cl)cc1S(=O)(=O)Nc1nccn2c(C(C)C)nnc12] |
| 0015 | 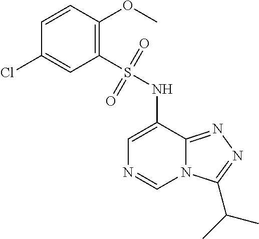 [SMILES: COc1ccc(Cl)cc1S(=O)(=O)Nc1cncn2c(C(C)C)nnc12] |
| 0016 | 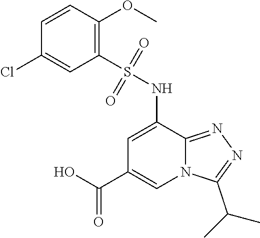 [SMILES: COc1ccc(Cl)cc1S(=O)(=O)Nc1cc(C(=O)O)cn2c(C(C)C)nnc12] |
| 0018 | 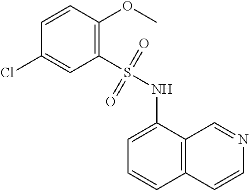 [SMILES: COc1ccc(Cl)cc1S(=O)(=O)Nc1cccc2ccncc12] |
| 0019 | 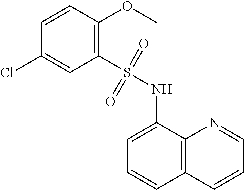 [SMILES: COc1ccc(Cl)cc1S(=O)(=O)Nc1cccc2cccnc12] |
| 0021 | 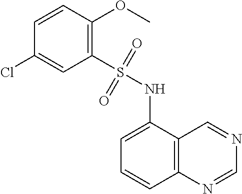 [SMILES: COc1ccc(Cl)cc1S(=O)(=O)Nc1cccc2ncncc12] |
| 0023 | 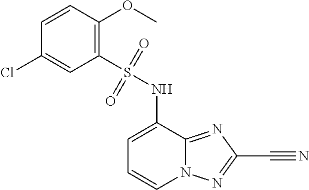 [SMILES: COc1ccc(Cl)cc1S(=O)(=O)Nc1cccn2nc(C#N)nc12] |
| 0024 | 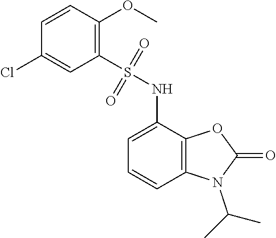 [SMILES: COc1ccc(Cl)cc1S(=O)(=O)Nc1cccc2c1oc(=O)n2C(C)C] |
| 0025 | 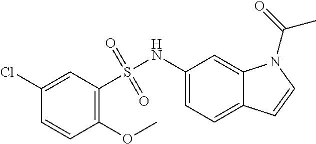 [SMILES: COc1ccc(Cl)cc1S(=O)(=O)Nc1ccc2ccn(C(C)=O)c2c1] |
| 0026 | 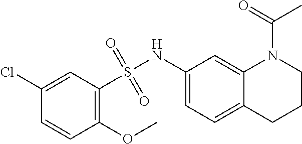 [SMILES: COc1ccc(Cl)cc1S(=O)(=O)Nc1ccc2c(c1)N(C(C)=O)CCC2] |
| 0027 | 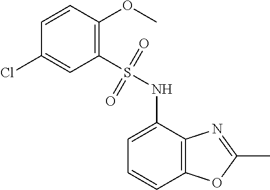 [SMILES: COc1ccc(Cl)cc1S(=O)(=O)Nc1cccc2oc(C)nc12] |
| 0028 | 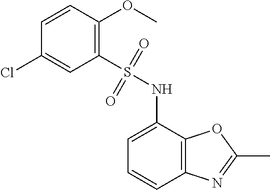 [SMILES: COc1ccc(Cl)cc1S(=O)(=O)Nc1cccc2nc(C)oc12] |
| 0029 | 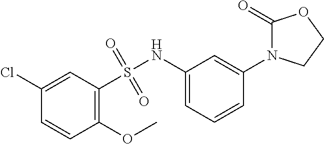 [SMILES: COc1ccc(Cl)cc1S(=O)(=O)Nc1cccc(N2CCOC2=O)c1] |
| 0030 | 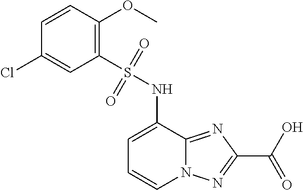 [SMILES: COc1ccc(Cl)cc1S(=O)(=O)Nc1cccn2nc(C(=O)O)nc12] |
| 0032 | 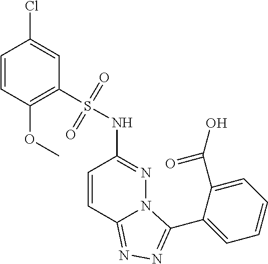 [SMILES: COc1ccc(Cl)cc1S(=O)(=O)Nc1ccc2nnc(-c3ccccc3C(=O)O)n2n1] |
| 0035 | 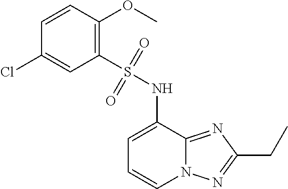 [SMILES: CCc1nc2c(NS(=O)(=O)c3cc(Cl)ccc3OC)cccn2n1] |
| 0036 | 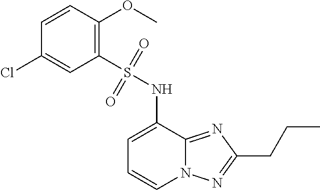 [SMILES: CCCc1nc2c(NS(=O)(=O)c3cc(Cl)ccc3OC)cccn2n1] |
| 0037 | 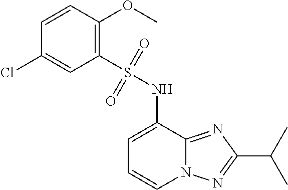 [SMILES: COc1ccc(Cl)cc1S(=O)(=O)Nc1cccn2nc(C(C)C)nc12] |

Methods of making the compounds of the present disclosure are within the abilities of a person of ordinary skill in the art. In addition, some of the compounds of the present disclosure are commercially available and/or known in the art, as indicated, in certain instances, by an associated CAS identifier.

### Compositions

It is often desirable to formulate a compound of the disclosure in a composition (e.g., pharmaceutical composition) comprising one or more pharmaceutically acceptable carriers, e.g., to administer the compound to a subject. Accordingly, some embodiments herein provide a composition (e.g., pharmaceutical composition) comprising a compound of the disclosure (e.g., a therapeutically effective amount of a compound of the disclosure) and one or more pharmaceutically acceptable carriers.

It will be appreciated that the compositions described herein are intended to encompass a composition comprising the recited ingredients, as well as any product that results, directly or indirectly, from combination, complexation or aggregation of any two or more of the ingredients, or from dissociation of one or more of the ingredients, or from other types of reactions or interactions of one or more of the ingredients. The compositions described herein can be made by admixing a compound of the disclosure and one or more pharmaceutically acceptable carriers.

The compositions described herein and, hence, the compounds of the disclosure, may be administered orally, parenterally, transocularly, intranasally, transdermally, transmucosally, by inhalation spray, vaginally, rectally, or by intracranial injection. The term “parenteral,” as used herein, includes subcutaneous, intravenous, intramuscular, and intracisternal injections, and infusion techniques. Administration by intravenous, intradermal, intramuscular, intramammary, intraperitoneal, intrathecal, retrobulbar, intrapulmonary injection and or surgical implantation at a particular site is contemplated as well. Generally, compositions for administration by any of the above methods are essentially free of pyrogens, as well as other impurities that could be harmful to the recipient. Further, compositions for administration parenterally are typically sterile. Typical modes of administration include enteral (e.g., oral) and parenteral (e.g., by subcutaneous, intramuscular, intravenous or intraperitoneal injection; or topical, transdermal, or transmucosal) administration.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release or sustained/delayed release.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants such as wetting agents, emulsifying and suspending agents, sweetening, flavoring, coloring, perfuming, and preservative agents.

Compositions suitable for buccal or sublingual administration include tablets, lozenges and pastilles, wherein the active ingredient is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

A compound of the disclosure can also be in micro-encapsulated form with one or more excipients, as noted above. In such solid dosage forms, the compound can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the active ingredient against degradation as it passes through the alimentary tract, for example, by an outer coating of the formulation on a tablet or capsule.

In another embodiment, a compound of the disclosure can be provided in an extended (or “delayed” or “sustained”) release composition. This delayed-release composition comprises the compound or pharmaceutically acceptable salt in combination with a delayed-release component. Such a composition allows targeted release of a provided agent into the lower gastrointestinal tract, for example, into the small intestine, the large intestine, the colon and/or the rectum. In certain embodiments, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH sensitive methacrylate coatings, pH sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, or other vehicles known in the art.

Compositions described herein can also be administered in the form of suppositories for rectal administration. These can be prepared by mixing a compound of the disclosure with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For other topical applications, the compositions can be formulated in a suitable ointment containing the active component suspended or dissolved in one or more carriers. Carriers for topical administration of a compound of the disclosure include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the active compound suspended or dissolved in one or more pharmaceutically acceptable carriers. Alternatively, the composition can be formulated with a suitable lotion or cream containing the active compound suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol and water and penetration enhancers.

For ophthalmic use, compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation, for example, for the treatment of asthma. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents. Without wishing to be bound by any particular theory, it is believed that local delivery of a composition described herein, as can be achieved by nasal aerosol or inhalation, for example, can reduce the risk of systemic consequences of the composition, for example, consequences for red blood cells.

Other pharmaceutically acceptable carriers, adjuvants and vehicles that can be used in the compositions described herein include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be advantageously used to enhance delivery of agents described herein.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

Compositions described herein can be administered alone or in combination with an additional therapy, e.g., an adjunct cancer therapy such as surgery, chemotherapy, radiotherapy, immune therapy, thermotherapy, and/or laser therapy. When a compound of the disclosure is administered in combination with an additional therapy, the compound of the disclosure and the additional therapy can be administered simultaneously, in a single composition. Accordingly, some embodiments herein provide compositions (e.g., pharmaceutical compositions) comprising a compound of the disclosure and an additional therapeutic agent (e.g., chemotherapeutic agent, immunotherapy).

Alternatively, a compound of the disclosure and an additional therapy can be administered in separate compositions. Accordingly, some embodiments herein provide combinations (e.g., pharmaceutical combinations) comprising a compound of the disclosure (e.g., a composition comprising a compound of the disclosure) and an additional therapeutic agent (e.g., an additional composition comprising an additional therapeutic agent).

Cytostatic and cytotoxic chemotherapeutic agents are contemplated for combination therapy, as are agents that target angiogenesis or lymphangiogenesis, and/or immune therapies, such as immune therapies targeting checkpoint pathways. Thus, in some embodiments, a composition or combination described herein further comprises a chemotherapeutic agent (e.g., a taxoid, such as paclitaxel).

Examples of chemotherapeutic agents for use in accordance with the present disclosure include, but are not limited to: alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammal1 and calicheamicin omegal1; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVIS OR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; and ABARELLX® rmRH; or a pharmaceutically acceptable salt and/or derivative of any of the above.

In some embodiments, a composition or combination described herein further comprises an immune therapy, such as an immune checkpoint inhibitor. In some embodiments, the immune checkpoint inhibitor is an antibody, or antigen binding fragment thereof, that inhibits the activity of one or more of CTLA-4, PD-L1, PD-L2, PD-1, B7-H3, B7-H4, BTLA, HVEM, TIM3, and GALS. In some embodiments, the immune checkpoint inhibitor is an antibody, or antigen binding fragment thereof, that inhibits the activity of one or more of CTLA-4, PD-L1, and PD-1 (e.g., an immune checkpoint inhibitor selected from ipilimumab, nivolumab, pembrolizumab, cemiplimab, avelumab, durvalumab or atezolizumab). In some embodiments, the immune checkpoint inhibitor is an antibody, or antigen binding fragments thereof, that inhibits the activity of PD-1 (e.g., an immune checkpoint inhibitor selected from nivolumab, pembrolizumab or cemiplimab). In some embodiments, the immune checkpoint inhibitor is an antibody, or antigen binding fragment thereof, that inhibits the activity of one or more of CTLA-4, PD-L1, PD-1, and LAG-3 (e.g., an immune checkpoint inhibitor selected from ipilimumab, nivolumab, pembrolizumab, cemiplimab, avelumab, durvalumab, atezolizumab or relatlimab).

Table 1 lists FDA-approved immunotherapies suitable for use in combination with compounds of the disclosure. Table 2 lists immunotherapies currently under investigation suitable for use in combination with compounds of the disclosure.

TABLE 1
FDA Approved Immunotherapies

| Target | Drug |
|---|---|
| CTLA4 | Ipilimumab (Yervoy) |
| PD-1 | Nivolumab (Opdivo) |
| | Pembrolizumab (Keytruda) |
| | Cemiplimab (Libtayo) |
| PD-L1 | Avelumab (Bavencio) |
| | Durvalumab (Imfinzi) |
| | Atezolizumab (Tecentriq) |
| PD-1/LAG-3 | Nivolumab/Relatlimab |
| | (OPDUALAG ™) |

TABLE 2
Immunotherapies Currently Under Investigation

| | | |
|---|---|---|
| Target | Drug | Company |

Costimulatory or agonist antibodies

| | | |
|---|---|---|
| 4-1BB (CD137) | Utomilumab | Pfizer Canada, Kirkland, QC |
| | Urelumab | Bristol-Myers Squibb, New York, |
| | | NY, U.S.A. |
| | INBRX-105 | Inhibrx, San Diego, CA, U.S.A. |
| ICOS (CD278) | GSK3359609 | GlaxoSmithKline, Mississauga, ON |
| | JTX-2011 | Jounce Therapeutics, Cambridge, MA, |
| | | U.S.A. |
| GITR (CD357) | TRX 518-001 | Leap Therapeutics, Cambridge, MA, |
| | | U.S.A. |
| | MK-4166 | Merck, Kenilworth, NJ, U.S.A. |
| | BMS-986156 | Bristol-Myers Squibb, New York, |
| | | NY, U.S.A. |
| | INCAGN01876 | Incyte Biosciences International, |
| | | Wilmington, DE, U.S.A. |
| CD70 | ARGX-110 | Argenx, Breda, Netherlands |
| | (cusatuzumab) | |
| CD27 | CDX-1127 (varlilumab) | Celldex Therapeutics, Hampton, NJ, |
| | | U.S.A. |
| OX40 (CD134) | PF-0451860 | Pfizer Canada, Kirkland, QC |
| | MEDI0562/6469/6383 | AstraZeneca Canada, Mississauga, ON |
| | GSK3174998 | GlaxoSmithKline, Mississauga, ON |
| | BMS-986178 | Bristol-Myers Squibb, New York, |
| | | NY, U.S.A. |
| CD40 | CP870893 | Pfizer Canada, Kirkland, QC |
| | APX005M | Bristol-Myers Squibb, New York, |
| | | NY, U.S.A. |

Co-inhibitory or antagonist antibodies

| | | |
|---|---|---|
| VISTA (B7-H5) | CA-170 | Curis, Lexington, MA, U.S.A. |
| CCR4 (CD194) | Mogamulizumab | Kyowa Kirin, Tokyo, Japan |
| B7-H3 (CD276) | MGD009 | Novartis Pharmaceutical, Ottawa, ON |
| | 8H9 | Y-mAbs Therapeutics, New York, |
| | | NY, U.S.A. |
| TIM-3 | TSR-022 | Tesaro, Waltham, MA, U.S.A. |
| | MBG453 | Novartis Pharmaceutical, Ottawa, ON |
| | Sym023 | Symphogen A/S, Ballerup, Denmark |
| | MEDI9447 (oleclumab) | AstraZeneca Canada, Mississauga, ON |
| LAG-3 (CD223) | BMS-986016 | Bristol-Myers Squibb, New York, |
| | (relatlimab) | NY, U.S.A. |
| | IMP321 (eftilagimod | Prima BioMed, Sydney, Australia |
| | alpha) | |
| | LAG525 | Novartis Pharmaceutical, Ottawa, ON |
| KIR (2DL1-3) | Lirilumab | Bristol-Myers Squibb, New York, |
| | | NY, U.S.A. |
| IDO-1,2 | Indoximod | NewLink Genetics, Ames, IA, U.S.A. |
| | Epacadostat | Incyte Biosciences International, |
| | | Wilmington, DE, U.S.A. |
| TIGIT | Tislelizumab | BeiGene, Beijing, P.R.C. |
| | BMS-986207 | Bristol-Myers Squibb, New York, |
| | | NY, U.S.A. |
| | MTIG7192A | Genentech, San Francisco, CA, U.S.A. |
| | AB154 | Arcus Biosciences, Hayward, CA, |
| | | U.S.A. |
| A2aR | Ciforadenant | Corvus Pharmaceuticals, Burlingame, |
| | | CA, U.S.A. |
| Transforming | M7824 | EMD Serono, Rockland, MA, U.S.A. |
| growth factor β | Galunisertib | Eli Lilly and Company, Indianapolis, |
| | | IN, U.S.A. |
| CD47 | TTI-621 | Trillium Therapeutics, Mississauga, |
| | | ON |
| CD73 | MEDI9447 (oleclumab) | AstraZeneca Canada, Mississauga, ON |

Other pathways

| | | |
|---|---|---|
| Toll-like receptors | Poly-ICIC | Ludwig Institute for Cancer Research, |
| | | New York, NY, U.S.A. |
| | MGN1703 (lefitolimod) | Mologen, Berlin, Germany |
| | SD-101 | Dynavax Technologies Corporation, |
| | | Emeryville, CA, U.S.A. |
| | DSP-0509 | Boston Biomedical, Cambridge, MA, |
| | | U.S.A. |
| | Rintatolimod | Hemispherx Biopharma, Philadelphia, |
| | | PA, U.S.A. |
| | CMP-001 | Checkmate Pharmaceuticals, |
| | | Cambridge, MA, U.S.A. |
| Interleukin 2 | NKTR-214 | Nektar Therapeutics, San Francisco, |
| receptor | | CA, U.S.A. |
| | RO6874281 | Hoffmann-La Roche, Basel, |
| | | Switzerland |
| | THOR-707 | Synthorx, La Jolla, CA, U.S.A. |
| Arginase inhibitors | CB-1158 | Incyte Corporation, Wilmington, DE, |
| | | U.S.A. |
| Oncolytic peptides | LTX-315 | Lytix Biopharma, Oslo, Norway |
| Interleukin 10 | AM0010 | Eli Lilly and Company, Indianapolis, |
| | (pegilodecakin) | IN, U.S.A. |

The disclosure also provides kits comprising a composition or combination described herein. In various embodiments, the kit contains, e.g., bottles, vials, ampoules, tubes, cartridges and/or syringes that comprise a liquid (e.g., sterile injectable) formulation or a solid (e.g., lyophilized) formulation. The kits can also contain pharmaceutically acceptable vehicles or carriers (e.g., solvents, solutions and/or buffers) for reconstituting a solid (e.g., lyophilized) formulation into a solution or suspension for administration (e.g., by injection), including without limitation reconstituting a lyophilized formulation in a syringe for injection or for diluting concentrate to a lower concentration. Furthermore, extemporaneous injection solutions and suspensions can be prepared from, e.g., sterile powder, granules, or tablets comprising a composition described herein. The kits also include, in various embodiments, dispensing devices, such as aerosol or injection dispensing devices, pen injectors, autoinjectors, needleless injectors, syringes, and/or needles. In various embodiments, the kit also or alternatively provides an oral dosage form, e.g., a tablet or capsule or other oral formulation described herein. In some embodiments, the kit also provides instructions for use (e.g., in a method described herein).

The compositions described herein can be provided in unit dosage form. As used herein, the term “unit dosage form” refers to physically discrete units suitable as unitary dosages for a subject, each unit containing a predetermined quantity of active ingredient(s) (e.g., a compound of the disclosure) optionally in association with one or more pharmaceutically acceptable carriers. The specifications for unit dosage forms depend, for example, on the particular active ingredient(s) employed, the effect to be achieved, the pharmacodynamics of the particular active ingredient(s) in the subject and the route of administration. Typically, however, the amount of active ingredient(s) in the unit dosage form is an amount sufficient to produce the desired effect, when administered according to the intended dosing schedule and route of administration.

Preferably, compositions (e.g., unit dosage forms) should be formulated so that a dosage of from about 0.01 mg/kg to about 100 mg/kg body weight/day of the compound of the disclosure can be administered to a subject receiving the composition. In some embodiments, compositions are formulated so that a dosage described herein of a compound of the disclosure can be administered to a subject receiving the composition. For example, a unit dosage form may contain from about 1 mg to about 5,000 mg, from about 10 mg to about 2,500 mg, from about 100 mg to about 1,000 mg, from about 1 mg to about 1000 mg, from about 1 mg to about 500 mg, from about 1 mg to about 250 mg, from about 1 mg to about 150 mg, from about 0.5 mg to about 100 mg, or from about 1 mg to about 50 mg of active ingredient(s). Preferably, the dosage does not cause or produces minimal adverse side effects.

Doses lower or higher than those recited above may be required. Specific dosage and treatment regimens for any particular patient will depend upon a variety of factors, for example, the activity of the specific agent employed, the age, body weight, general health status, sex, diet, time of administration, rate of excretion, drug combination, the severity and course of the disease, condition or symptoms, the patient's disposition to the disease, condition or symptoms, the judgment of the treating physician and the severity of the particular disease being treated. The amount of an agent in a composition will also depend upon the particular agent in the composition.

In some embodiments, the concentration of one or more therapeutic agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1, 0.5%, 0.4%, 0.3%, 0.2%, 0.10, or 0.010% w/w, w/v, or v/v. In some embodiments, the concentration of one or more therapeutic agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

### Therapeutic Uses

Metadherin (MTDH; also known as AEG-1, 3D3/LYRIC) was identified as a pro-metastasis gene that resides in 8q22, a frequently amplified genomic locus linked to poor relapse-free survival of breast cancer. The amino acid sequence of human metadherin can be found in Genbank Accession No. AAH45642, herein incorporated by reference. In recent years, elevated levels of MTDH have been reported in more than 20 cancer types, suggesting a potentially crucial and broad functionality of this gene in human cancer. Depending on the cancer type tested, recent studies using mainly cell culture systems have implicated MTDH in many cancer-related processes, including cellular proliferation, stress-induced cell death, invasion, chemoresistance and metastasis.

These pleiotropic tumor-promoting roles of MTDH may stem from the complex nature of this protein, as revealed by its initial identification. MTDH was originally reported as an HIV-induced gene in astrocytes, a cell-surface molecule mediating the homing of mammary tumor cells to the lung endothelium, a lysine-rich CEACAM1 co-isolated (LYRIC) protein associated with tight junctions in prostate epithelial cells, and as a novel transmembrane protein present in the different sub-cellular compartments. At the molecular level, the human MTDH is a 582-amino acid protein with no recognizable domains to indicate its biological function, except for a putative transmembrane domain and three lysine-rich nuclear localization signals.

MTDH has nevertheless been reported to interact with multiple proteins. In the nucleus, MTDH was shown to interact with PLZF, BCCIPα and NFκB subunit p65. In cytoplasm, MTDH was reported to interact with staphylococcal nuclease domain-containing protein 1 (SND1). MTDH has also been linked to multiple classical oncogenic signaling pathways such as PI3K/AKT and Wnt signaling in a cancer cell type-dependent manner. However, whether MTDH exerts its function by interacting with its binding partners, and how MTDH modulates the abovementioned oncogenic pathways remain largely unknown.

SND1 is a multifunctional protein harboring four tandem repeats of Staphylococcal nuclease (SN)-like domains at the N terminus (SN1-4), and a fusion tudor and SN domain (TSNS domain) at the C terminus. SND1 belongs to the oligonucleotide/oligosaccharide binding-fold (GB-fold) superfamily consisting of proteins that primarily participate in DNA/RNA-binding via the typical β-barrel of the GB-fold. The amino acid sequence of human SND1 can be found in Genbank Accession no. NP_055205, herein incorporated by reference. SND1 has consistently been suggested to be an essential component of the RNA-induced silencing complex (RISC) and involved in miRNA-mediated silencing. SND1 was also shown to have a nuclease activity toward hyper-edited miRNA primary transcripts. Structural and biochemical analysis of SND1 suggested that the N-terminal SN domains, particularly SN3/4, possess RNA-binding and nuclease activity, and the C-terminal TSN domain interacts with methylated Lys/Arg ligands and small nuclear ribonucleoprotein (snRNP) complexes.

SND1 is among the very few members of the GB-fold superfamily that participate in interaction with diverse proteins. It was initially identified as a cellular component that enhances the transcription of EBNA-2-activated gene, and later shown to interact with and modulate a broad spectrum of proteins involved in transcription, including oncogenic transcription factors STATS, STAT6, and c-Myb. More recently, SND1 was identified as a binding partner of MTDH in multiple types of cancer, and has been shown to be important for cancer cell survival under oncogenic or chemotherapeutic stresses.

The crystal structure of MTDH-SND1 complex has now been resolved, and revealed a unique interface between the two N-terminal SN domains of SND1 and a peptide motif of MTDH. The surface contour of SND1 revealed two deep pockets that specifically interact with the MTDH residues. In particular, the bulky and hydrophobic side chains of W394 and W401 of MTDH were found to bind deeply into the two hydrophobic binding pockets of SND1. It has now been found that compounds of the disclosure are small molecule inhibitors of the MTDH-SND1 protein-protein interaction. Without wishing to be bound by any particular theory, it is hypothesized that the compounds of the disclosure may exert their inhibitory effect by binding to MTDH and/or SND1 where MTDH and SND1 bind to one another, e.g., at residues 393-403 of MTDH.

Accordingly, provided herein is a method of inhibiting an interaction between MTDH and SND1 in a cell (e.g., a cell expressing MTDH and SND1), comprising contacting the cell with a compound of the disclosure. Also provided herein is a method of stabilizing or increasing the level or expression of transporter associated with antigen processing (TAP, e.g., TAP1 and/or TAP2) in a cell (e.g., a cell expressing TAP, such as TAP1 and/or TAP2), comprising contacting the cell with a compound of the disclosure. Also provided herein is a method of inhibiting degradation of Tap (e.g., Tap1 and/or Tap2) in a cell (e.g., a cell expressing Tap, such as Tap1 and/or Tap2), comprising contacting the cell with a compound of the disclosure. Also provided herein is a method of promoting tumor antigen presentation in a cell, comprising contacting the cell with a compound of the disclosure. In some embodiments of any of the foregoing methods, an effective amount of the compound of the disclosure is administered.

In some embodiments, any of the foregoing methods is performed in vitro. In some embodiments, any of the foregoing methods is performed ex vivo. In some embodiments, any of the foregoing methods is performed in vivo as, for example, when the cell is in a subject (e.g., a patient).

Thus, also provided herein is a method of inhibiting an interaction between MTDH and SND1 in a subject (e.g., a subject in need thereof, such as a subject having a cancer), comprising administering to the subject an effective amount of a compound of the disclosure. Also provided herein is a method of stabilizing or increasing the level or expression of TAP (e.g., TAP1 and/or TAP2) in a subject (e.g., a subject in need thereof, such as a subject having a cancer), comprising administering to the subject an effective amount of a compound of the disclosure. Also provided herein is a method of inhibiting degradation of Tap (e.g., Tap1 and/or Tap2) in a subject (e.g., a subject in need thereof, such as a subject having a cancer), comprising administering to the subject an effective amount of a compound of the disclosure. Also provided herein is a method of promoting tumor antigen presentation in a subject (e.g., a subject in need thereof, such as a subject having a cancer), comprising administering to the subject an effective amount of a compound of the disclosure.

Also provided herein is a method of treating a disease, disorder or condition mediated by the MTDH-SND1 protein-protein interaction in a subject in need thereof, comprising administering to the subject an effective amount of a compound of the disclosure. As used herein, “a disease, disorder or condition mediated by the MTDH-SND1 protein-protein interaction” refers to any disease, disorder or condition (e.g., cancer) in which the MTDH-SND1 protein-protein interaction promotes and/or sustains tumor progression and/or metastasis, and/or inhibits an immune response (e.g., to a tumor). Examples of diseases, disorders or conditions mediated by the MTDH-SND1 protein-protein interaction include those described herein, in particular, breast cancer, liver cancer, lung cancer, colorectal cancer, glioblastoma, prostate cancer, melanoma, bladder cancer, pancreatic cancer, kidney cancer and gastric cancer.

Also provided herein is a method of treating a cancer in a subject in need thereof, comprising administering to the subject an effective amount of a compound of the disclosure. In some embodiments, the cancer is a hematologic cancer. In some embodiments, the cancer is a solid tumor cancer.

Examples of cancers treatable in accordance with the methods described herein include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's tumor.

Further examples of cancers treatable according to the methods described herein include Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

In some embodiments, the cancer is breast cancer, liver cancer, lung cancer, colorectal cancer, glioblastoma, prostate cancer, melanoma, bladder cancer, pancreatic cancer, kidney cancer or gastric cancer. In some embodiments, the cancer is breast cancer, liver cancer, colon cancer, lung cancer or prostate cancer. In some embodiments, the cancer is prostate cancer. In some embodiments, the cancer is breast cancer.

Metastases of the aforementioned cancers can also be treated in accordance with the methods described herein. In some embodiments, the cancer is a metastatic cancer.

In some embodiments, the cancer is a resistant cancer (e.g., chemoresistant).

Also provided herein is a method of inhibiting metastasis in a cancer in need thereof, including any of the cancers described herein, comprising administering to the subject an effective amount of a compound of the disclosure.

Also provided herein is a method of sensitizing a cancer in a subject in need thereof to treatment with a radiation therapy, chemotherapy (e.g., a chemotherapeutic agent described herein) or immune therapy (e.g., an immunotherapy described herein), or a combination of the foregoing, comprising administering to the subject an effective amount of a compound of the disclosure.

Also provided herein is a method of promoting T-cell activation or infiltration or both in response to a cancer in a subject in need thereof, comprising administering to the subject an effective amount of a compound of the disclosure.

The compounds of the disclosure and the compositions described herein can be administered orally, parenterally, transocularly, intranasally, transdermally, transmucosally, by inhalation spray, vaginally, rectally, or by intracranial injection. Typically, administration is enteral (e.g., oral) or parenteral (e.g., by subcutaneous, intramuscular, intravenous or intraperitoneal injection; or topical, transdermal, or transmucosal). In some embodiments, administration is oral. In some embodiments, administration is by subcutaneous, intramuscular, intravenous or intraperitoneal injection.

A compound of the disclosure or a composition described herein can also be administered in combination with one or more other therapies (e.g., radiation therapy; a chemotherapy, such as a chemotherapeutic agent; an immunotherapy; or a combination of the foregoing). When administered in a combination therapy, the compound of the disclosure can be administered before, after or concurrently with the other therapy (e.g., radiation therapy, an additional agent(s)). When co-administered simultaneously (e.g., concurrently), the compound of the disclosure and other therapy can be in separate formulations or the same formulation. Alternatively, the compound of the disclosure and other therapy can be administered sequentially, as separate compositions, within an appropriate time frame as determined by a skilled clinician (e.g., a time sufficient to allow an overlap of the pharmaceutical effects of the therapies). When the compound of the disclosure and the other therapy (e.g., therapeutic agent) are administered as separate formulations or compositions, the compound of the disclosure and the other therapy can be administered by the same route of administration or by different routes of administration, including any of the routes of administration described herein.

In some embodiments, a method described herein further comprises administering to the subject an effective amount of an additional therapy (e.g., radiation therapy, chemotherapy, immunotherapy, or a combination of the foregoing). In some embodiments, a method described herein further comprises administering to the subject an effective amount of one or more additional therapeutic agents (e.g., any of the additional therapeutic agents described herein, such a chemotherapeutic agent and/or immunotherapy).

The amount of a therapeutic agent (e.g., compound of the disclosure) that is administered in accordance with the methods described herein is, of course, dependent on factors such as the age, weight, and general condition of the patient, the severity of the condition being treated, and the judgment of the prescribing physician. Suitable therapeutic amounts will be known to those skilled in the art and/or are described in the pertinent reference texts and literature.

In some embodiments, a dosage of a therapeutic agent (e.g., compound of the disclosure) is within the range of about 0.01 mg to about 1,000 mg per kg (mg/kg) of body weight per day. In some embodiments, a dose ranges from about 10 mg/kg to about 250 mg/kg, or from about 100 mg/kg to about 250 mg/kg, or from about 60 mg/kg to about 100 mg/kg or from about 50 mg/kg to about 90 mg/kg, or from about 30 mg/kg to about 80 mg/kg, or from about 20 mg/kg to about 60 mg/kg, or from about 10 mg/kg to about 50 mg/kg. In some embodiments, a dose (e.g., daily dose) may be about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 75 mg/kg, about 80 mg/kg, about 90 mg/kg, about 100 mg/kg, about 125 mg/kg, about 150 mg/kg, about 175 mg/kg, about 200 mg/kg, about 250 mg/kg, about 300 mg/kg, about 350 mg/kg, about 400 mg/kg, about 450 mg/kg, or about 500 mg/kg, or may range between any two of the foregoing values. Suitable dosages of a therapeutic agent (e.g., compound of the disclosure) also include from about 1 mg to about 5,000 mg, e.g., from about 10 mg to about 2,500 mg, from about 100 mg to about 1,000 mg, from about 1 mg to about 1000 mg, from about 1 mg to about 500 mg, from about 1 mg to about 250 mg, from about 1 mg to about 150 mg, from about 0.5 mg to about 100 mg, or from about 1 mg to about 50 mg.

The desired dose may be administered in a single dose, for example, such that the agent is administered once per day (e.g., QD), or as multiple doses administered at appropriate intervals, for example, such that the agent is administered 2, 3 or 4, or more times per day (e.g., BID, TID, QID). Typically, the compositions will be administered from about 1 to about 6 (e.g., 1, 2, 3, 4, 5 or 6) times per day or, alternatively, as an infusion (e.g., a continuous infusion). Administration may continue for at least 3 months, 6 months, 9 months, 1 year, 2 years, or more.

The treatment methods described herein optionally include monitoring the effect of the treatment (e.g., compound of the disclosure) on the tumor. For example, the size of the tumor can be determined, as can the presence of metastases. Also contemplated is measurement of the degree of metastasis, e.g., by measuring the number of metastatic modules or by measurement of ascites associated with metastasis.

### Screening Assay

A split luciferase assay was developed to assess the ability of various compounds, including compounds of the disclosure, to disrupt the MTDH-SND1 interaction. Table 3 provides DNA and protein sequences for SND1-NLuc, CLuc-MTDH and Linked-luciferase constructs used in the split luciferase assay described in Example 1. For SEQ ID NOS: 1 and 2, Myc tag sequence is indicated by underlining, SND1 sequence is indicated with no emphasis added, Link sequence is indicated by bolding, and firefly luciferase N-terminal sequence indicated by italics. For SEQ ID NOS: 3 and 4, firefly luciferase C-terminal sequence is indicated by italics, Link sequence is indicated by bolding, MTDH sequence is indicated with no emphasis added, and HA tag sequence is indicated by underlining. For SEQ ID NOS: 5 and 6, Myc tag sequence is indicated by underlining, firefly luciferase N-terminal sequence indicated by italics, Link sequence is indicated by bolding, firefly luciferase C-terminal sequence indicated by bolding and italics, and HA tag sequence indicated with no emphasis added.

TABLE 3
DNA and protein sequences for SND1-NLuc, CLuc-MTDH and Linked-luciferase
constructs used in the split luciferase assay described in Example 1.

| SEQ ID | | |
|---|---|---|
| NO | Construct Name | Sequence |
| 1 | SND1-NLuc | GAGCAGAAACTCATCTCTGAAGAGGATCTG gtccccacc |
| | DNA Sequence | gtgcagcggg gcatcatcaa |
| | | gatggtcctc tcagggtgcg ccatcattgt ccgaggtcag cctcgtggtg |
| | | ggcctcctcc |
| | | tgagcggcag atcaacctca gcaacatteg tgctggaaat cttgctcgcc |
| | | gggcagccgc |
| | | cacacaacct gatgcaaagg atacccctga tgagccctgg gcatttccag |
| | | ctcgagagtt |
| | | ccttcgaaag aagctgattg ggaaggaagt ctgtttcacg atagaaaaca |
| | | agactcccca |
| | | ggggcgagag tatggcatga tctaccttgg aaaagatacc aatggggaaa |
| | | acattgcaga |
| | | atcactggtt gcagagggct tagccacccg gagagaaggc atgagagcta |
| | | ataatcctga |
| | | gcagaaccgg ctttcagaat gtgaagaaca agcaaaggca gccaagaaag |
| | | ggatgtggag |
| | | tgaggggaac ggttcacata ctatccggga tctcaagtat accattgaaa |
| | | acccaaggca |
| | | ctttgtggac tcacaccacc agaagcctgt taatgctatc atcgagcatg |
| | | tgcgggacgg |
| | | cagtgtggtc agggccctgc tectcccaga ttactacctg gttacagtca |
| | | tgctgtcagg |
| | | catcaagtgc ccaacttttc gacgggaagc agatggcagt gaaactccag |
| | | agccttttgc |
| | | tgcagaagcc aaatttttca ctgagtegcg actgcttcag agagatgttc |
| | | agatcattct |
| | | ggagagctgc cacaaccaga acattctggg taccatcctt catccaaatg |
| | | gcaacatcac |
| | | agagctcctc ctgaaggaag gtttcgcacg ctgtgtggac tggtcgattg |
| | | cagtttacac |
| | | ccggggcgca gaaaagctga gggcggcaga gaggtttgcc aaagagcgca |
| | | ggctgagaat |
| | | atggagagac tatgtggctc ccacagctaa tttggaccaa aag |
| | | GGAGGTGGTTCA GGAGGAGGTAGT |
| | | GGTGGAGGAAGT |
| | | gaagacgccaaaaacataaagaaaggcccggcgccattctatcctctagaggatg |
| | | gaaccgctggagagcaactgcataaggctatgaagagatacgccctggttcctgga |
| | | acaattgcttttacagatgcacatatcgaggtgaacatcacgtacgcggaatacttcg |
| | | aaatgtccgttcggttggcagaagctatgaaacgatatgggctgaatacaaatcaca |
| | | gaatcgtcgtatgcagtgaaaactctcttcaattctttatgccggtgttgggcgcgttattt |
| | | atcggagttgcagttgcgcccgcgaacgacatttataatgaacgtgaattgctcaaca |
| | | gtatgaacatttcgcagcctaccgtagtgtttgtttccaaaaaggggttgcaaaaaattt |
| | | tgaacgtgcaaaaaaaattaccaataatccagaaaattattatcatggattctaaaac |
| | | ggattaccagggatttcagtcgatgtacacgttcgtcacatctcatctacctcccggtttt |
| | | aatgaatacgattttgtaccagagtcctttgatcgtgacaaaacaattgcactgataat |
| | | gaattcctctggatctactgggttacctaagggtgtggcccttccgcatagaactgcctg |
| | | cgtcagattctcgcatgccagagatcctatttttggcaatcaaatcattccggatactgc |
| | | gattttaagtgttgttccattccatcacggttttggaatgtttactacactcggatatttgat |
| | | atgtggatttcgagtcgtcttaatgtatagatttgaagaagagctgtttttacgatcccttc |
| | | aggattacaaaattcaaagtgcgttgctagtaccaaccctattttcattcttcgccaaaa |
| | | gcactctgattgacaaatacgatttatctaatttacacgaaattgcttctggggggcac |
| | | ctctttcgaaagaagtcggggaagcggttgcaaaacgcttccatcttccagggatacg |
| | | acaaggatatgggctcactgagactacatcagctattctgattacacccgaggggga |
| | | tgataaaccgggcgcggtcggtaaagtigttccattttttgaagcgaaggttgtggatct |
| | | ggataccgggaaaacgctgggcgttaatcagagaggcgaattatgtgtcagaggac |
| | | ctatgattatgtccggttatgtaaacaatccggaagcgaccaacgccttgattgacaa |
| | | ggatgga |
| 2 | SND1-NLuc | EQKLISEEDLVPTVQRGIIKMVLSGCAIIVRGQPRGGPPP |
| | Protein | ERQINLSNIRAGNLARRAAATQPDAKDTPDEPWAFPAR |
| | Sequence | EFLRKKLIGKEVCFTIENKTPQGREYGMIYLGKDINGEN |
| | | IAESLVAEGLATRREGMRANNPEQNRLSECEEQAKAAK |
| | | KGMWSEGNGSHTIRDLKYTIENPRHFVDSHHQKPVNAI |
| | | IEHVRDGSVVRALLLPDYYLVTVMLSGIKCPTFRREAD |
| | | GSETPEPFAAEAKFFTESRLLQRDVQIILESCHNQNILGTI |
| | | LHPNGNITELLLKEGFARCVDWSIAVYTRGAEKLRAAE |
| | | RFAKERRLRIWRDYVAPTANLDQK |
| | | GGGSGGGSGGGSEDAKNIKKGPAPFYPLEDGTAGEQLH |
| | | KAMKRYALVPGTIAFTDAHIEVNITYAEYFEMSVRLAE |
| | | AMKRYGLNTNHRIVVCSENSLQFFMPVLGALFIGVAVA |
| | | PANDIYNERELLNSMNISQPTVVFVSKKGLQKILNVQK |
| | | KLPIIQKHIIMDSKTDYQGFQSMYTFVTSHLPPGFNEYDF |
| | | VPESFDRDKTIALIMNSSGSTGLPKGVALPHRTACVRFS |
| | | HARDPIFGNQIIPDTAILSVVPFHHGFGMFTTLGYLICGF |
| | | RVVLMYRFEEELFLRSLQDYKIQSALLVPTLFSFFAKST |
| | | LIDKYDLSNLHEIASGGAPLSKEVGEAVAKRFHLPGIRQ |
| | | GYGLTETTSAILITPEGDDKPGAVGKVVPFFEAKVVDLD |
| | | TGKTLGVNQRGELCVRGPMIMSGYVNNPEATNALIDK |
| | | DG |
| 3 | CLuc-MTDH | atgtccggttatgtaaacaatccggaagcgaccaacgccttgattgacaaggatgga |
| | DNA Sequence | tggctacattctggagacatagcttactgggacgaagacgaacacttcttcatagttga |
| | | ccgcttgaagtctttaattaaatacaaaggatatcaggtggcccccgctgaattggaat |
| | | cgatattgttacaacaccccaacatcttcgacgcgggcgtggcaggtcttcccgacga |
| | | tgacgccggtgaacttcccgccgccgttgttgttttggagcacggaaagacgatgacg |
| | | gaaaaagagatcgtggattacgtcgccagtcaagtaacaaccgcgaaaaagttgc |
| | | gcggaggagttgtgtttgtggacgaagtaccgaaa |
| | | ggtcttaccggaaaactcgacgcaagaaaaatcagagagatcctcataaaggcca |
| | | agaagggcggaaagtccaaattg |
| | | GGAGGTGGTTCAGGAGGAGGTAGT |
| | | Tcttctgctgatcccaactctgattggaatgcaccagcagaagagtggggc |
| | | aattgggtagacgaa TAC CCA TAC GAT GTT CCA GAT TAC |
| | | GCT |
| 4 | CLuc-MTDH | MSGYVNNPEATNALIDKDGWLHSGDIAYWDEDEHFFIVDR |
| | Protein | LKSLIKYKGYQVAPAELESILLQHPNIFDAGVAGLPDDDAG |
| | Sequence | ELPAAVVVLEHGKTMTEKEIVDYVASQVTTAKKLRGGVVF |
| | | VDEVPKGLTGKLDARKIREILIKAKKGGKSKL |
| | | GGGSGGGS SSADPNSDWNAPAEEWGNWVDE |
| | | YPYDVPDYA |
| 5 | Linked- | GAGCAGAAACTCATCTCTGAAGAGGATCTG |
| | luciferase DNA | gaagacgccaaaaacataaagaaaggcccggcgccattctatcctctagaggatg |
| | Sequence | gaaccgctggagagcaactgcataaggctatgaagagatacgccctggttcctgga |
| | | acaattgcttttacagatgcacatatcgaggtgaacatcacgtacgcggaatacttcg |
| | | aaatgtccgttcggttggcagaagctatgaaacgatatgggctgaatacaaatcaca |
| | | gaatcgtcgtatgcagtgaaaactctcttcaattctttatgccggtgttgggcgcgttattt |
| | | atcggagttgcagttgcgcccgcgaacgacatttataatgaacgtgaattgctcaaca |
| | | gtatgaacatttcgcagcctaccgtagtgtttgtttccaaaaaggggttgcaaaaaattt |
| | | tgaacgtgcaaaaaaaattaccaataatccagaaaattattatcatggattctaaaac |
| | | ggattaccagggatttcagtcgatgtacacgttcgtcacatctcatctacctcccggtttt |
| | | aatgaatacgattttgtaccagagtcctttgatcgtgacaaaacaattgcactgataat |
| | | gaattcctctggatctactgggttacctaagggtgtggcccttccgcatagaactgcctg |
| | | cgtcagattctcgcatgccagagatcctatttttggcaatcaaatcattccggatactgc |
| | | gattttaagtgttgttccattccatcacggttttggaatgtttactacactcggatatttgat |
| | | atgtggatttcgagtcgtcttaatgtatagatttgaagaagagctgtttttacgatcccttc |
| | | aggattacaaaattcaaagtgcgttgctagtaccaaccctattttcattcttcgccaaaa |
| | | gcactctgattgacaaatacgatttatctaatttacacgaaattgcttctgggggcgcac |
| | | ctctttcgaaagaagtcggggaagcggttgcaaaacgcttccatcttccagggatacg |
| | | acaaggatatgggctcactgagactacatcagctattctgattacacccgaggggga |
| | | tgataaaccgggcgcggtcggtaaagttgttccattttttgaagcgaaggttgtggatct |
| | | ggataccgggaaaacgctgggcgttaatcagagaggcgaattatgtgtcagaggac |
| | | ctatgattatgtccggttatgtaaacaatccggaagcgaccaacgccttgattgacaa |
| | | ggatgga |
| | | GGAGGTGGTTCA GGAGGAGGTAGT |
| | | GGTGGAGGAAGT |
| | | atgtccggttatgtaaacaatccggaagcgaccaacgccttgattgacaaggatgga |
| | | tggctacattctggagacatagcttactgggacgaagacgaacacttcttcatagttga |
| | | ccgcttgaagtctttaattaaatacaaaggatatcaggtggcccccgctgaattggaat |
| | | cgatattgttacaacaccccaacatcttcgacgcgggcgtggcaggtcttcccgacga |
| | | tgacgccggtgaacttcccgccgccgttgttgttttggagcacggaaagacgatgacg |
| | | gaaaaagagatcgtggattacgtcgccagtcaagtaacaaccgcgaaaaagttgc |
| | | gcggaggagttgtgtttgtggacgaagtaccgaaaggtcttaccggaaaactcgacg |
| | | caagaaaaatcagagagatcctcataaaggccaagaagggcggaaagtccaaat |
| | | tg |
| | | TAC CCA TAC GAT GTT CCA GAT TAC GCT |
| 6 | Linked | EQKLISEEDL |
| | Luciferase | EDAKNIKKGPAPFYPLEDGTAGEQLHKAMKRYALVPGTIA |
| | Protein | FTDAHIEVNITYAEYFEMSVRLAEAMKRYGLNTNHRIVVCS |
| | Sequence | ENSLQFFMPVLGALFIGVAVAPANDIYNERELLNSMNISQP |
| | | TVVFVSKKGLQKILNVQKKLPIIQKIIIMDSKTDYQGFQSMY |
| | | TEVTSHLPPGFNEYDFVPESFDRDKTIALIMNSSGSTGLPK |
| | | GVALPHRTACVRFSHARDPIFGNQIIPDTAILSVVPFHHGF |
| | | GMFTTLGYLICGFRVVLMYRFEEELFERSLQDYKIQSALLV |
| | | PTLFSFFAKSTLIDKYDLSNLHEIASGGAPLSKEVGEAVAKR |
| | | FHLPGIRQGYGLTETTSAILITPEGDDKPGAVGKVVPFFEA |
| | | KVVDLDTGKTLGVNQRGELCVRGPMIMSGYVNNPEATNA |
| | | LIDKDG |
| | | GGGSGGGSGGGSMSGYVNNPEATNALIDKDGWLHSGDI |
| | | AYWDEDEHFFIVDRLKSLIKYKGYQVAPAELESILLQHPNIF |
| | | DAGVAGLPDDDAGELPAAVVVLEHGKTMTEKEIVDYVASQ |
| | | VTTAKKLRGGVVFVDEVPKGLTGKLDARKIREILIKAKKGG |
| | | KSKL YPYDVPDYA |

Accordingly, also provided herein is a nucleic acid molecule comprising, consisting essentially of or consisting of the nucleotide sequence of SEQ ID NO:1, or a nucleotide sequence having at least 75%, at least 85%, at least 90% or at least 95% identity to the sequence of SEQ ID NO:1. Also provided herein is a protein comprising, consisting essentially of or consisting of the amino acid sequence of SEQ ID NO:2, or an amino acid sequence having at least 75%, at least 85%, at least 90% or at least 95% identity to the sequence of SEQ ID NO:2. Also provided herein is a nucleic acid molecule comprising, consisting essentially of or consisting of the nucleotide sequence of SEQ ID NO:3, or a nucleotide sequence having at least 75%, at least 85%, at least 90% or at least 95% identity to the nucleotide sequence of SEQ ID NO:3. Also provided herein is a protein comprising, consisting essentially of or consisting of the amino acid sequence of SEQ ID NO:4, or an amino acid sequence having at least 75%, at least 85%, at least 90% or at least 95% identity to the amino acid sequence of SEQ ID NO:4.

Kits for performing a split luciferase assay, such as the split luciferase assay described in Example 1, are also provided. In some embodiments, the kit comprises (i) a nucleic acid molecule comprising the nucleotide sequence of SEQ ID NO:1, or a nucleotide sequence having at least 75%, at least 85%, at least 90% or at least 95% identity to the nucleotide sequence of SEQ ID NO:1, and (ii) and a nucleic acid molecule having the nucleotide sequence of SEQ ID NO:3, or a nucleotide sequence having at least 75%, at least 85%, at least 90% or at least 95% identity to the nucleotide sequence of SEQ ID NO:3. In some embodiments, the kit comprises (i) a protein comprising the amino acid sequence of SEQ ID NO:2, or an amino acid sequence having at least 75%, at least 85%, at least 90% or at least 95% identity to the amino acid sequence of SEQ ID NO:2, and (ii) a protein comprising the amino acid sequence of SEQ ID NO:4, or an amino acid sequence having at least 75%, at least 85%, at least 90% or at least 95% identity to the amino acid sequence of SEQ ID NO:4. In some embodiments, the kits further include instructions for use, for example, in a split luciferase screening assay such as that described in Example 1.

As used herein, the term “sequence identity” means that two nucleotide or amino acid sequences, when optimally aligned, such as by the programs GAP or BESTFIT using default gap weights, share at least, e.g., 70% sequence identity, or at least 80% sequence identity, or at least 85% sequence identity, or at least 90% sequence identity, or at least 95% sequence identity, or at least 98% sequence identity, or at least about 99% sequence identity or more. For sequence comparison, typically one sequence acts as a reference sequence (e.g., parent sequence) to which test sequences are compared. The sequence identity comparison can be examined throughout the entire length of a nucleotide, or within a desired fragment of a given nucleotide. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used.

One embodiment is a method of identifying a MTDH-SND1 inhibitor, comprising contacting (i) a protein comprising the amino acid sequence of SEQ ID NO:2, or an amino acid sequence having at least 75%, at least 85%, at least 90% or at least 95% identity to the amino acid sequence of SEQ ID NO:2, and (ii) a protein comprising the amino acid sequence of SEQ ID NO:4, or an amino acid sequence having at least 75%, at least 85%, at least 90% or at least 95% identity to the amino acid sequence of SEQ ID NO:4, with a test compound (e.g., a compound of the disclosure) in a medium, and detecting luciferase activity in the medium, wherein a decrease in luciferase activity compared to an appropriate control indicates the test compound is a MTDH-SND1 inhibitor.

While the disclosure has been described in conjunction with specific embodiments thereof, the foregoing description as well as the examples which follow are intended to illustrate and not limit the scope of the disclosure. Other aspects, advantages and modifications within the scope of the disclosure will be apparent to those skilled in the art.

### EXEMPLIFICATION

### Example 1

Small Molecule Inhibitors that Disrupt the MTDH-SND1 Complex Suppress Breast Cancer Progression and Metastasis

Abstract. Metastatic breast cancer is leading health burden worldwide. Previous studies have shown that metadherin (MTDH) promotes breast cancer initiation, metastasis and therapy resistance; however, the therapeutic potential of targeting MTDH remains largely unexplored. Here, genetically modified mice were used to demonstrate that genetic ablation of Mtdh inhibits breast cancer development through disrupting the interaction with Staphylococcal nuclease domain-containing 1 (SND1) which is required to sustain breast cancer progression in established tumors. Small molecule compound screening was performed to identify a class of specific inhibitors that disrupt the protein-protein interaction between MTDH-SND1, and show that compounds C26-A2 and C26-A6 suppressed tumor growth and metastasis, and enhanced chemotherapy sensitivity in preclinical models of triple-negative breast cancer. The results demonstrate a significant therapeutic potential in targeting the MTDH-SND1 complex and identify a new class of therapeutic agents for metastatic breast cancer.

Introduction. The lack of effective therapy for metastatic cancer and the frequent resistance to treatments are the two most significant hurdles for reducing the mortality of metastatic breast cancer¹. Computational analysis of gene expression profiles of breast tumor samples were previously used to identify MTDH/AEG1 as a key driver gene in poor-prognosis breast cancers^{2,3}. Functionally, MTDH is an important mediator of tumor initiation, chemoresistance and metastasis^{2,4}. Global Mtdh knockout in mice does not affect embryogenesis or postnatal development, but profoundly impairs the formation of mammary tumors⁴. Similar results were obtained from whole body genetic knockout studies of MTDH/AEG1 in the context of prostate cancer, liver, lung and colorectal cancers⁵⁻⁷. These findings suggested that Mtdh is specifically required for malignant tumors but is dispensable for normal development or homeostasis, underscoring the rationale to therapeutically target MTDH in cancer. However, the biochemical and molecular mechanisms of MTDH in breast cancer remain poorly defined. To uncover the functional partners underlying MTDH's tumor promoting role in breast cancer, MTDH immunoprecipitation followed by mass spectrum analysis were performed, and Staphylococcal nuclease domain-containing 1 (SND1) was identified as a major MTDH-interacting partner^{8,9}. SND1 has been previously characterized as a transcriptional co-activator¹⁰ or a RNA binding protein that is involved in the regulation of RNA stability, splicing, and editing^{10,11}. SND1 shares similar clinical and functional importance as MTDH in promoting metastasis and chemoresistance^{4,6,8}. Furthermore, the tumor-promoting function of MTDH is crucially dependent on the interaction with SND1⁴.

The crystal structure of MTDH-SND1 complex¹² was previously resolved, and revealed a unique interface between the two N-terminal SN domains of SND1 and a peptide motif of MTDH. The surface contour of SND1 revealed two deep pockets that specifically interact with the MTDH residues. In particular, the bulky and hydrophobic side chains of W394 and W401 of MTDH were found to bind deeply into the two hydrophobic binding pockets of SND1¹². Point mutations of these two evolutionarily conserved tryptophan residues in MTDH, which blocked the interaction with SND1, also completely eliminated the tumor-supportive function of MTDH^{4,12}. It was hypothesized that the two binding pockets could possess structural, geometrical and biochemical properties suitable for the development of small molecule inhibitors¹².

In this study, breast cancer mouse models with inducible Mtdh knockout were generated to evaluate the requirement of the MTDH-SND1 complex in the late stage breast cancer progression and metastasis. Further, a small molecule screening platform was developed to discover compounds that block MTDH-SND1 interaction and evaluate their therapeutic efficacy.

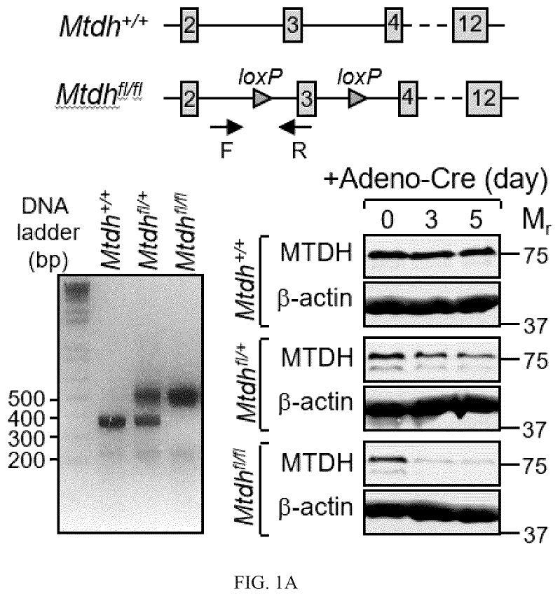

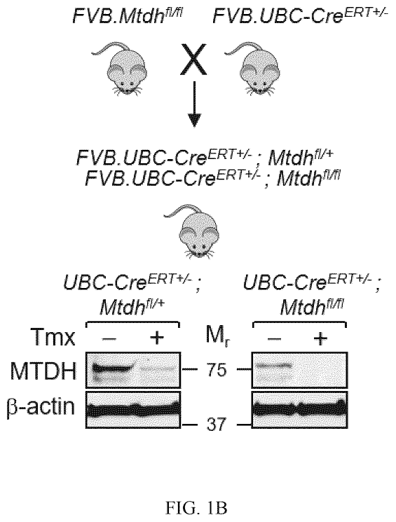

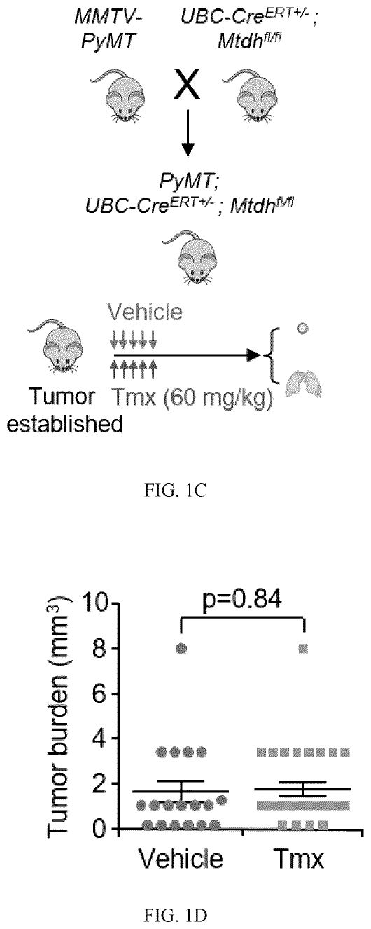

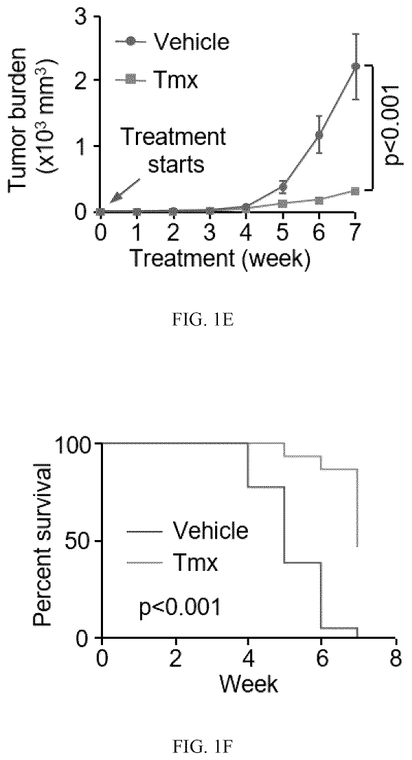

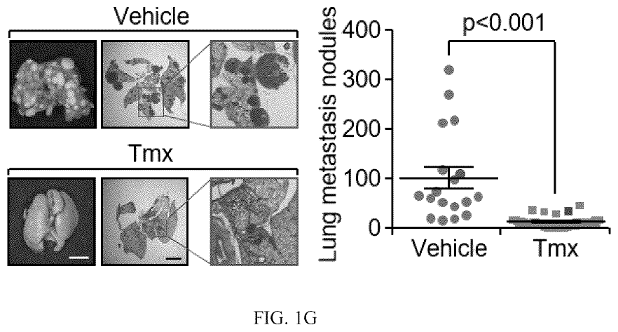

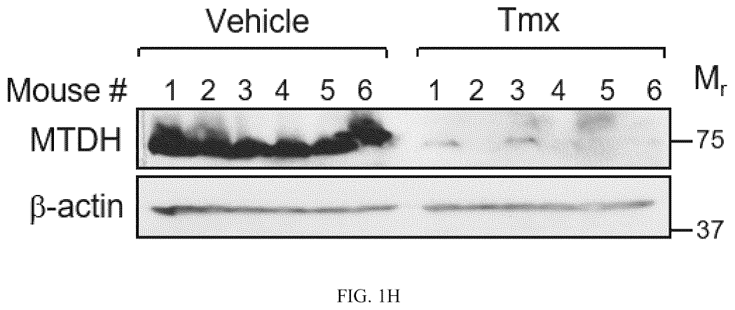

Results: Mtdh acute KO inhibits metastatic breast cancer progression. To evaluate the therapeutic potential of targeting MTDH in established tumors, Mtdh conditional knockout strain was generated. Mouse ESC cells with two loxp sites flanking exon 3 of Mtdh were injected into C57BL/6N strain to derive the Mtdh^{fl/fl} strain (FIG. 1A). The C57BL/6N.Mtdh^{fl/fl} strain was then backcrossed to FVB for more than 10 generations to change the genetic background to FVB. Splenocytes from FVB.Mtdh^{fl/fl} were isolated and infected with Cre-expressing adenovirus to validate Cre-mediated Mtdh knockout (KO) (FIG. 1A). Next, the FVB.Mtdh^{fl/fl} strain was crossed with FVB.UBC-Cre^{ERT+/-} to generate FVB.UBC-Cre^{ERT+/-};Mtdh^{fl/fl}, in which Mtdh is depleted upon 5 days of i.p. tamoxifen (Tmx) treatment at 60 mg/kg (FIG. 1). Such a dosing regimen of tamoxifen was commonly used in conditional KO of gene of interest in mouse cancer models including MMTV-PyMT and has been shown to have no direct effect on PyMT tumor growth and metastasis^{13,14}. Finally, FVB.UBC-Cre^{ERT+/-};Mtdh^{fl/fl} were crossed with the FVB.MMTV-PyMT strain, and the resulting female PyMT; UBC-Cre^{ERT+/-};Mtdh^{fl/fl} mice developed spontaneous mammary tumors with Mtdh inducible KO upon Tmx treatment (FIG. 1C).

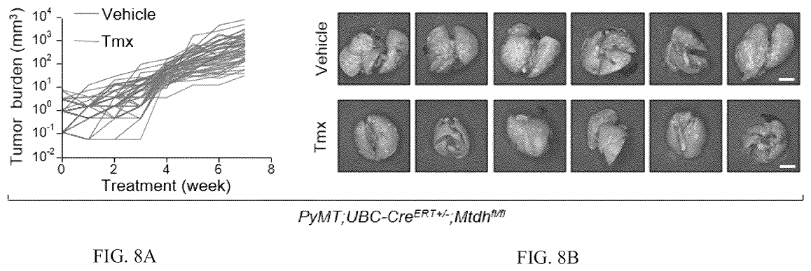

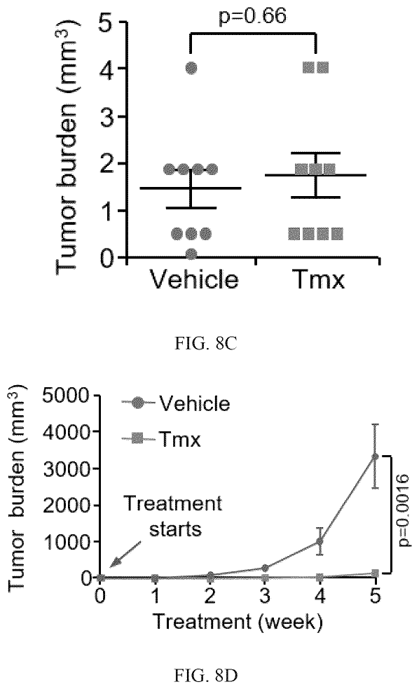

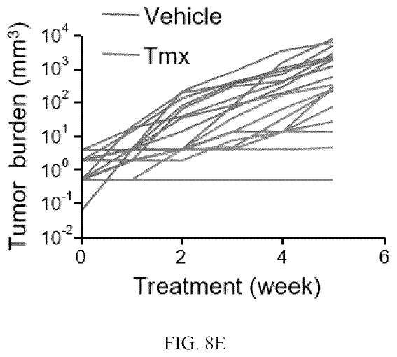

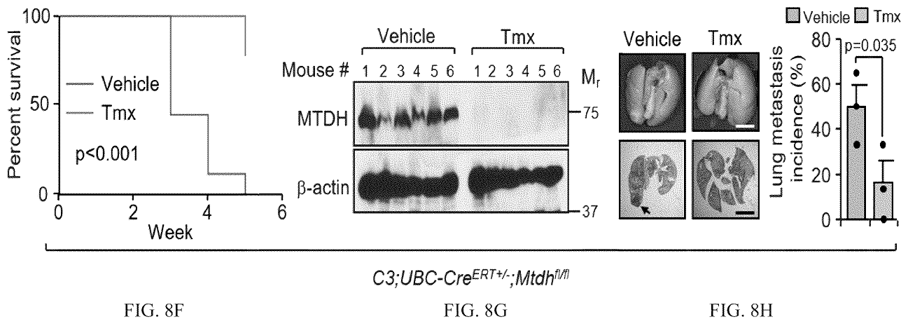

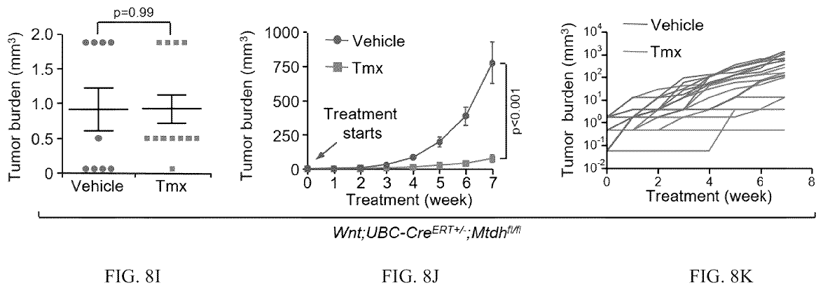

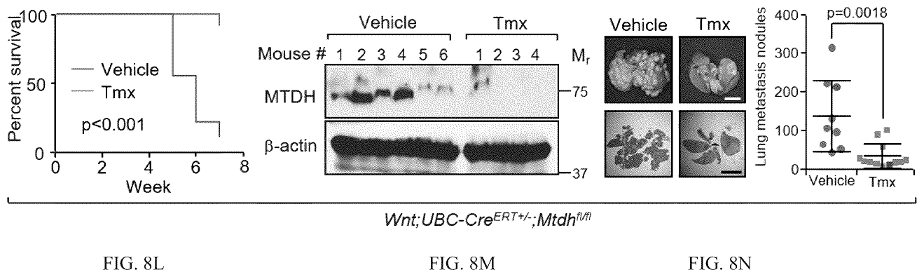

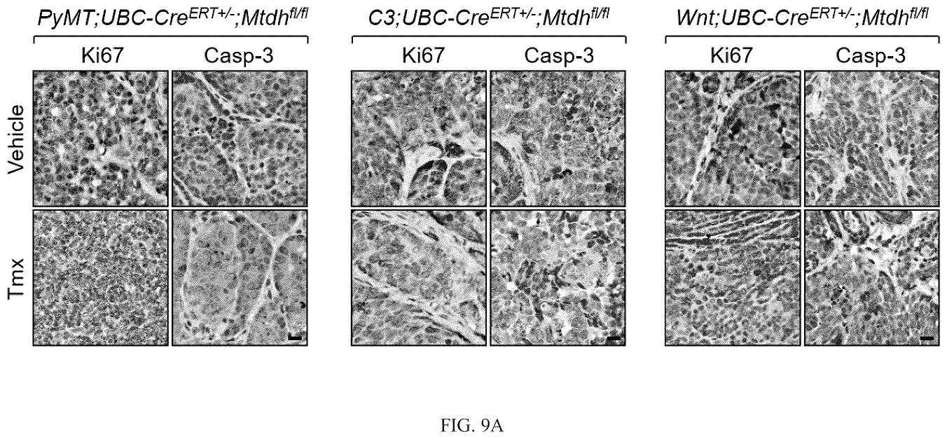

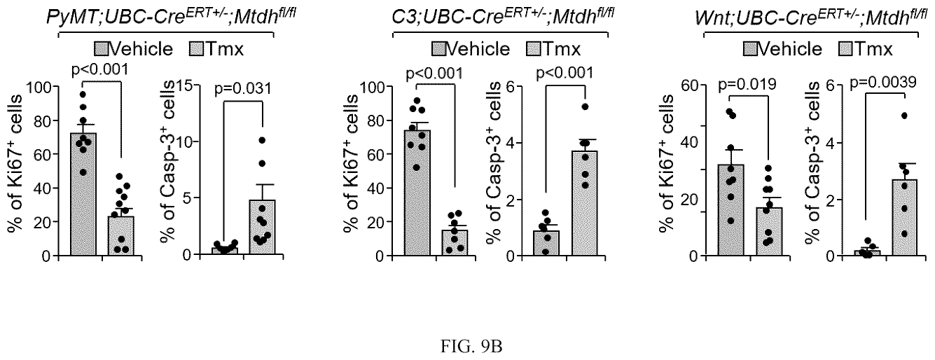

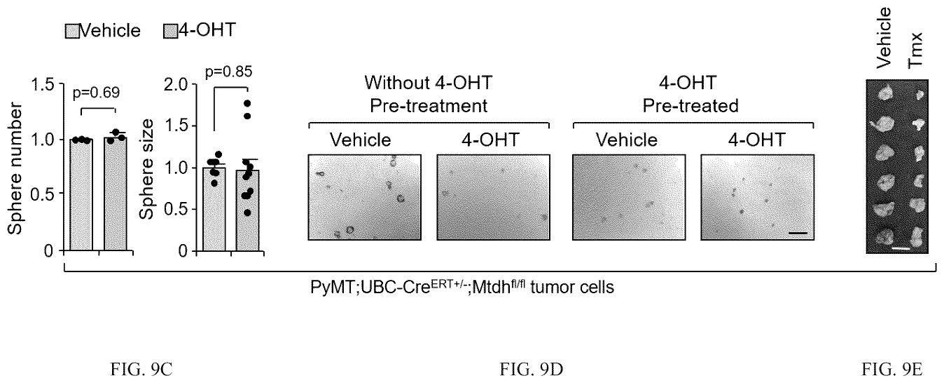

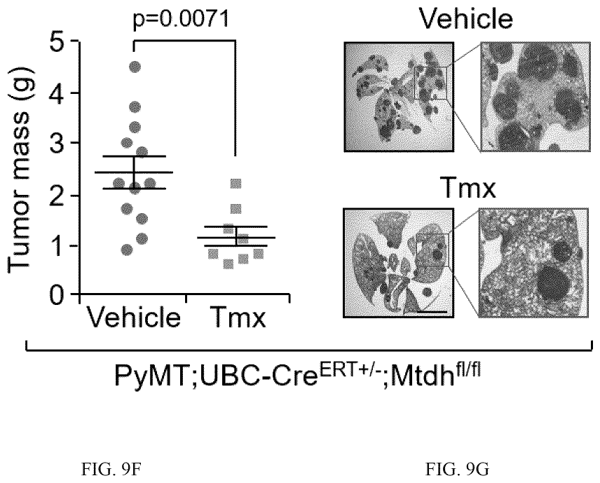

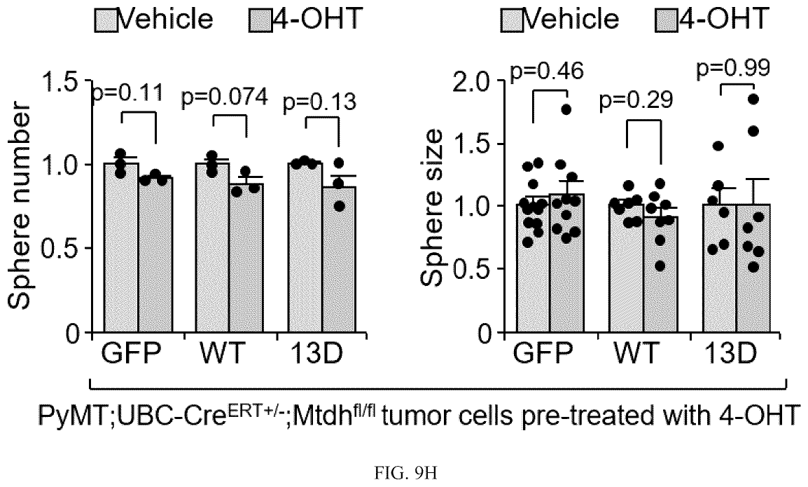

Female PyMT; UBC-Cre^{ERT+/-};Mtdh^{fl/fl} mice were separated into two groups when primary tumors were established (FIG. 1C, FIG. 1D). The mice were randomized and matched by tumor size (FIG. 1D), followed by Tmx or vehicle treatments. Tmx-induced Mtdh acute loss significantly suppressed primary tumor development, reduced spontaneous metastasis, and prolonged mouse survival (FIGS. 1E-1H, and FIGS. 8A, 8B). UBC-Cre^{ERT+/-};Mtdh^{fl/fl} with C3 and MMTV-Wnt mouse strains that develop breast tumors of basal subtype¹⁵⁻¹⁷ or diverse subtypes¹⁸⁻²⁰, respectively, were also crossed. In both models, acute Mtdh loss also suppressed tumor growth and lung metastasis (FIGS. 8C-8N). Furthermore, immunohistochemistry (IHC) staining indicated that Mtdh acute KO dramatically inhibited tumor proliferation and increased apoptosis (FIGS. 9A, 9B). Collectively, the results indicated that Mtdh acute loss in established tumors suppressed breast cancer progression and metastasis, underlying the therapeutic potential of targeting MTDH.

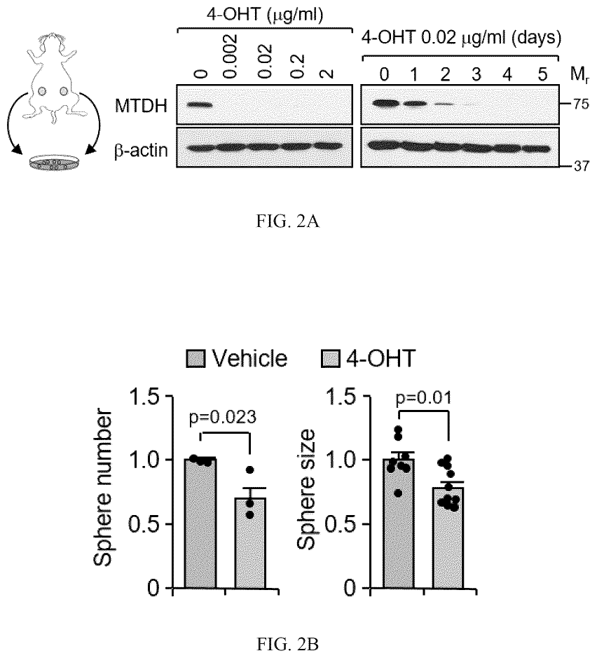

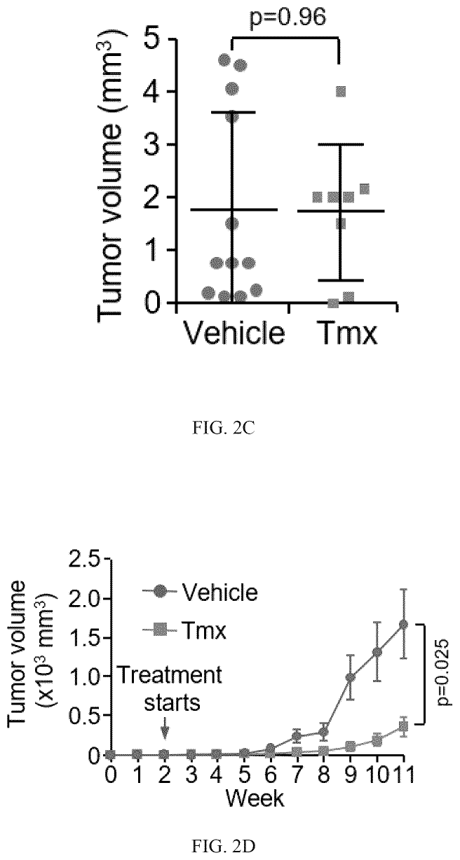

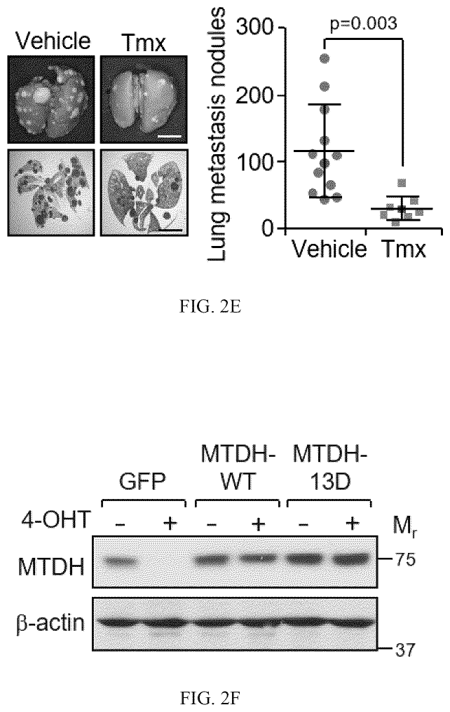

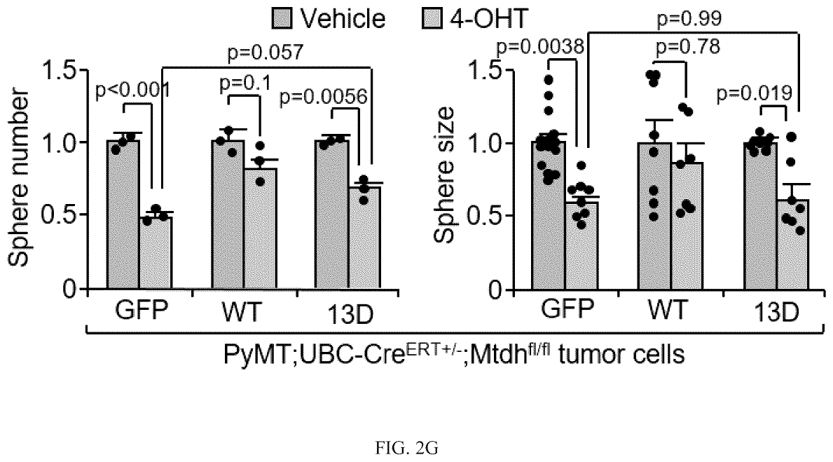

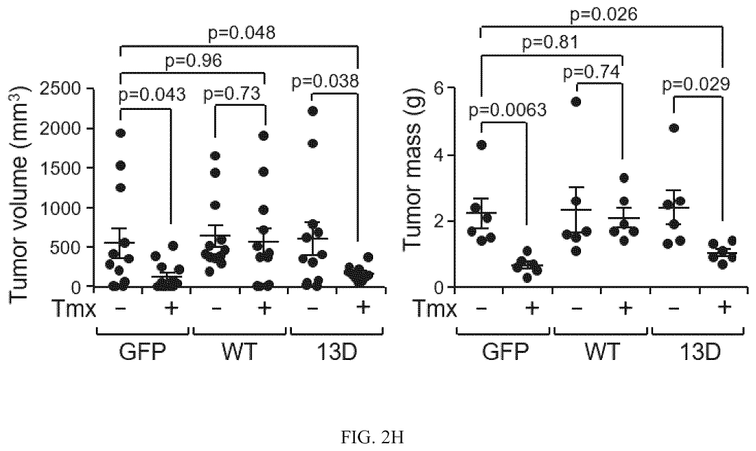

MTDH-SND1 sustains tumor progression and metastasis. It was previously found that MTDH-SND1 interaction is essential for sustaining tumor initiating cell activities during early tumorigenesis of PyMT, Wnt, Neu and carcinogen-induced mammary gland tumors and in in vitro tumorsphere formation analysis⁴. However, whether this interaction is still required for late stages of breast cancer progression is still unknown and is paramount for further clinical development of MTDH-targeting therapeutics in human patients. To address this question, a mammary tumor cell line derived from PyMT; UBC-Cre^{ERT+/-};Mtdh^{fl/fl} tumors was generated. Similar to these tumors in vivo, Mtdh can be genetically deleted with 4-OHT treatment of this cell line in cell culture (FIG. 2A). 4-OHT treatment induced MTDH KO leading to a significant decrease in tumorsphere formation (FIG. 2B and Extended Data FIG. 9C, 9D). In contrast, 4-OHT treatment no longer affected the spheres that formed by PyMT; UBC-Cre^{ERT+/-};Mtdh^{fl/fl} cells pretreated with 4-OHT (FIG. 9C 9D), suggesting that the reduction in tumorsphere upon 4-OHT treatment is due to the acute Mtdh KO rather than any inhibitory effect of 4-OHT itself.

Next, PyMT; UBC-Cre^{ERT+/-};Mtdh^{fl/fl} tumor cells were injected into FVB female mice orthotopically, and after primary tumors were established (FIG. 2-1c), the mice were treated with or without Tmx. Consistent with the results shown above, Mtdh acute KO in allograft tumors dramatically inhibited primary tumor growth and spontaneous lung metastasis (FIG. 2D, 2E and FIG. 9E-9G).

Taking advantage of the PyMT; UBC-Cre^{ERT+/-};Mtdh^{fl/fl} cell line, rescue lines with stable lentiviral expression of wild type (MTDH-WT) or SND1 interaction-deficient MTDH-W391D (MTDH-13D) were established (FIG. 2F)^{4,12}. Tumorsphere assays indicated that wild type MTDH, but not SND1 interaction deficient MTDH-13D mutant, was able to successfully rescue both tumorsphere number and size of the cell line upon acute MTDH-KO by 4-OHT treatment versus vehicle control (FIG. 2G). On the other hand, 4-OHT treatment of 4-OHT pretreated cells did not affect sphere formation, again confirming no inhibitory side effect from 4-OHT exposure per se (FIG. 9H). Next, these cell lines were injected orthotopically into FVB females followed by Tmx or vehicle treatment after tumors had been established. Consistent with the in vitro assay, wild type MTDH but not MTDH-13D restores primary tumor growth and metastasis (FIGS. 2H, 2I), validating the importance of MTDH-SND1 interaction in maintaining breast cancer progression and metastasis.

Discovery of small chemical inhibitors of MTDH-SND1. Given that MTDH-SND1 interaction is critical for breast cancer progression (FIG. 2 and Extended Data FIGS. 9C-9H) and the interaction is dependent on the two small pockets formed by SND1 that might be targeted by small chemical compounds¹², steps were taken to identify small chemical inhibitors that can disrupt MTDH-SND1 interaction by binding to these pockets. To this end, luciferase and FRET assays were established to screen a singleton small chemical library consisting of approximately 50,000 compounds with high structural diversity.

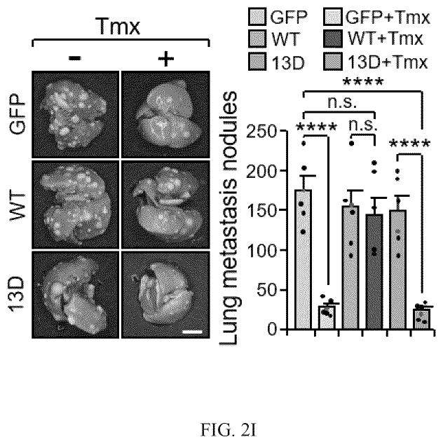

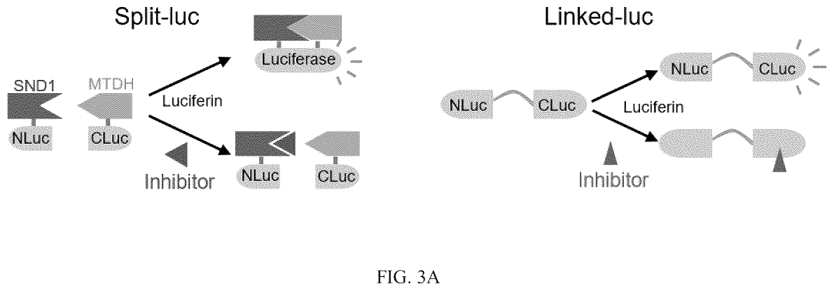

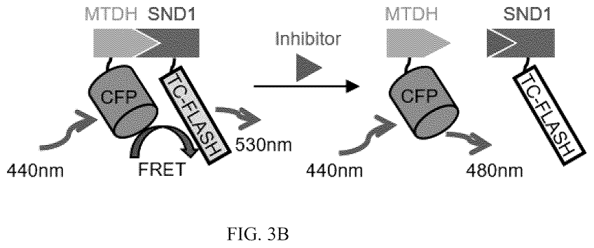

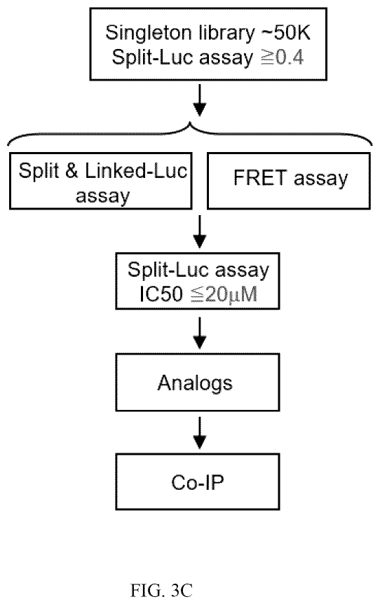

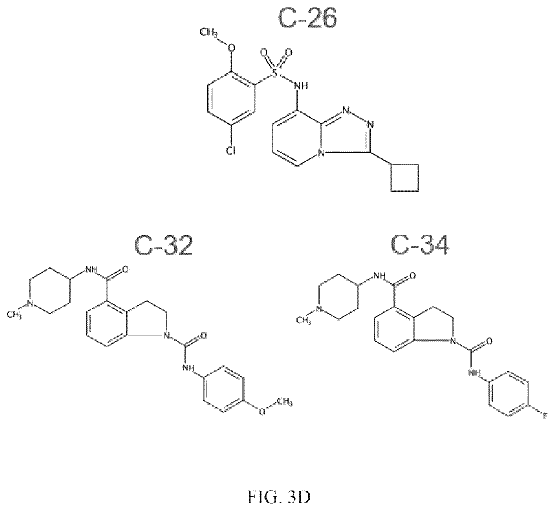

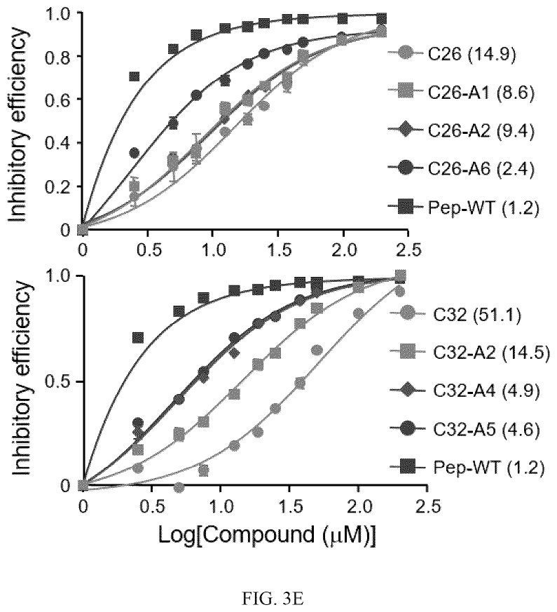

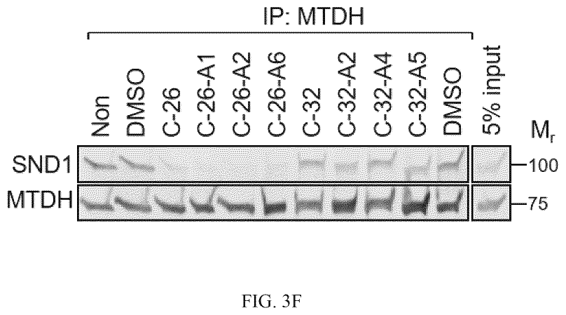

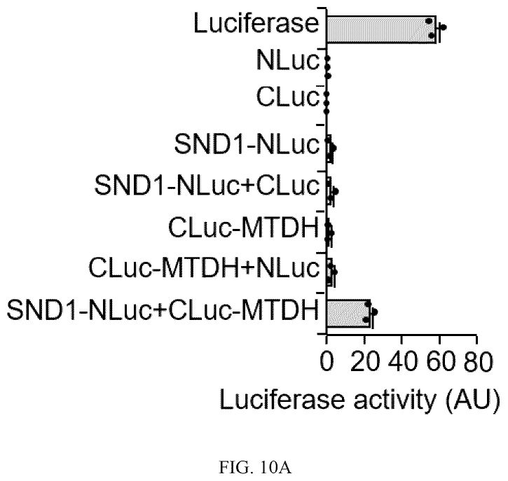

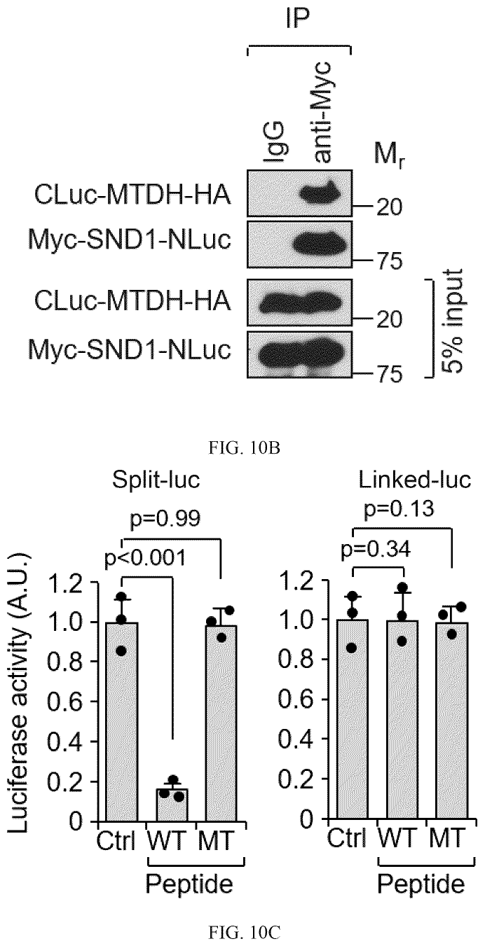

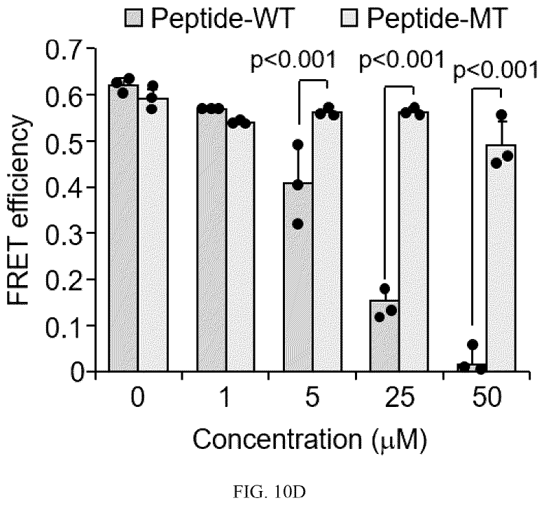

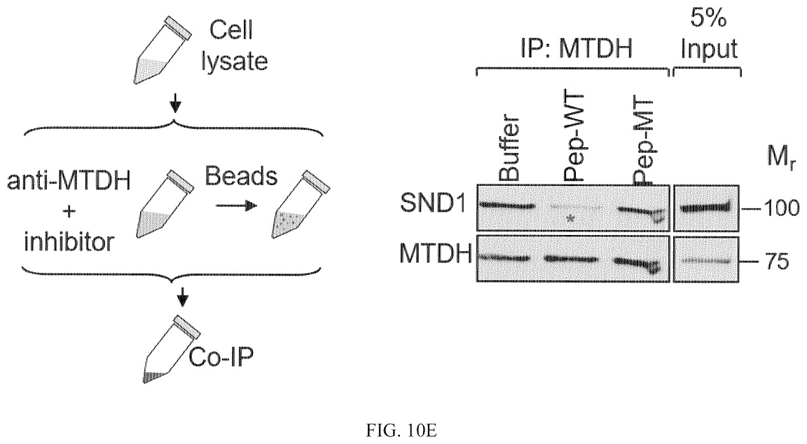

To generate a luciferase-based screening system, firefly luciferase was split into N- and C-terminals according to previous studies²¹⁻²³. Both N- and C-terminal domains alone have almost undetectable luciferase activity (FIG. 10A). Next, the two fragments were fused to SND1 and MTDH interaction domains respectively to generate SND1-NLuc and CLuc-MTDH (Split-luc) (FIG. 10A, left, and Table 3). Similarly, SND1-NLuc or CLuc-MTDH alone, or co-expression of SND1-NLuc and CLuc or CLuc-MTDH and NLuc did not produce significant luciferase signals either (FIG. 10A). However, when SND1-NLuc+CLuc-MTDH were co-expressed, N- and C-terminal domains of the luciferase fragments were brought close to each other due to the interaction between SND1 and MTDH, and substantial luciferase activity was reconstituted (FIG. 3A, left and FIG. 10A).

The interaction between SND1-NLuc and CLuc-MTDH was also validated with co-immunoprecipitation (Co-IP) experiments (FIG. 10B). In the presence of the compounds (inhibitors) that block MTDH-SND1 interaction, the reconstitution of luciferase activity was expected to be reduced (FIG. 3A). Meanwhile, considering the possibility that compounds capable of directly inhibiting the enzymatic activity of luciferase could also result in lower luciferase signal without any blocking effect on MTDH-SND1 interaction, Linked-luc that directly fuse NLuc and CLuc together was generated to be used in counter screening (FIG. 3A, right, and Table 3).

To further confirm the specificity of the system, wild type MTDH (MTDH-WT) (PNSDWNAPAEEWGNW) minimal peptide that binds to SND1 in the two hydrophobic pockets and its mutant (PNSDANAPAEEAGNW) form (MTDH-MT) with the two key tryptophans mutated to alanines and lack SND1 binding ability¹² were synthesized. MTDH-WT peptide but not the mutant significantly inhibited split-luc activity (FIG. 10C). On the other hand, linked-luc activity was not affected by either MTDH-WT or MTDH-MT peptides (FIG. 10C). These results suggested that the split-luc assay can be used as a readout to identify inhibitors that block MTDH-SND1 interaction.

Next, a FRET assay was established for secondary screening by fusing MTDH interaction domain with CFP and labeling SND1 domain with TC-FLASH²⁴. MTDH-SND1 interaction allows FRET signal to be detected, whereas the signal would be expected to be interrupted in the presence of MTDH-SND1 inhibitors (FIG. 3B). Similar to the split-luciferase assay, MTDH-WT and MTDH-MT peptides were employed to validate the specificity of the FRET assay in detecting the disruption of MTDH-SND1 interaction (FIG. 10D).

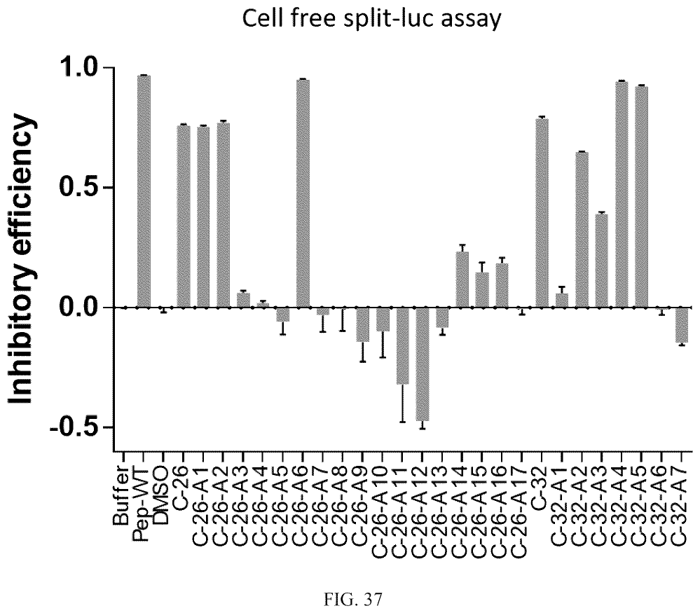

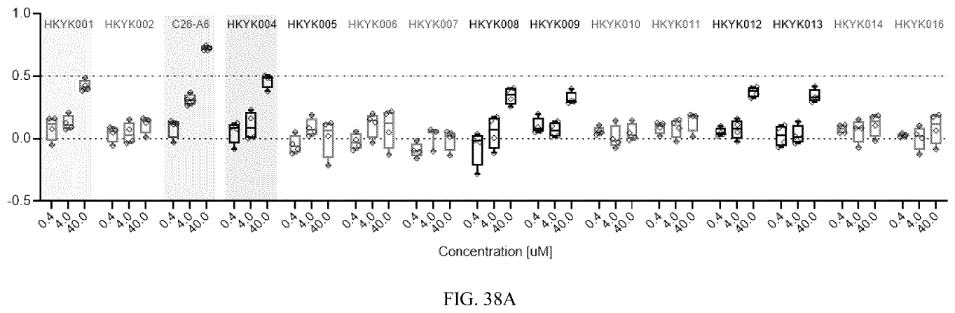

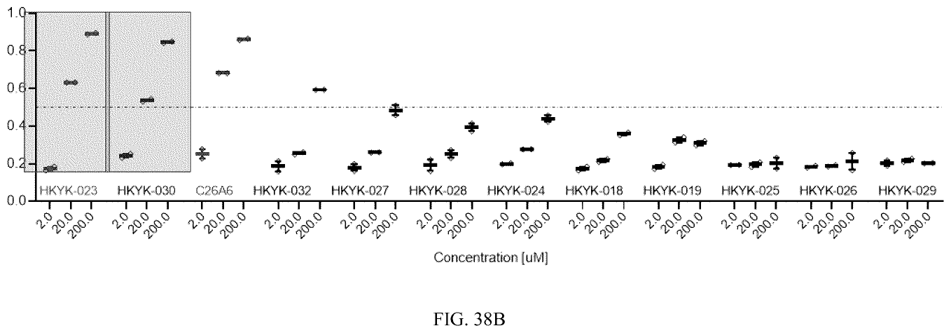

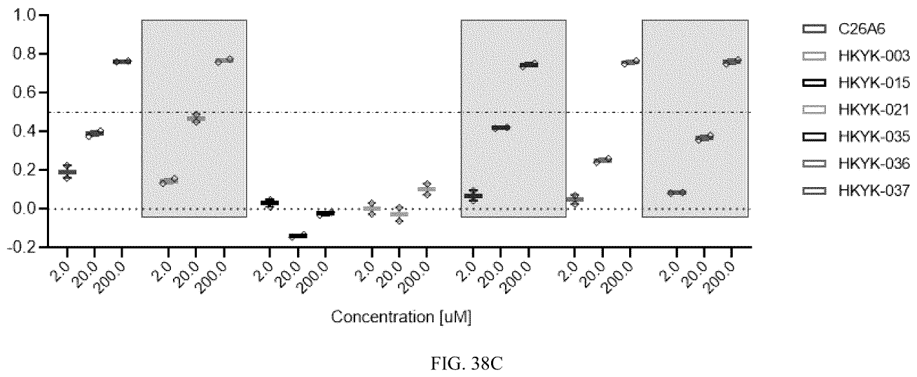

Using the screening systems described above, a 50K singleton library was first screened with the spit-luc assay with a repeat. Compounds that showed inhibitory efficiency of 0.4 or above in either one of the two rounds of screening were selected and repeated with split-luc, linked-luc and FRET assays. A set of criteria (see Methods section) was applied to narrow down the candidate list to 52 compounds. Luciferase and FRET assays were performed again for these 52 candidates and the best 12 were picked for split-luc assay in various concentrations to calculate the IC50 (FIG. 3C). Using these criteria, three compounds, C26, C32, and C34 with IC50 of less than 20 μM were selected for further study (FIG. 3-1d). Given that C32 and C34 share a similar structure (FIG. 3D), and C32 had a lower IC50, C34 was not pursued in further studies. To further understand the structure-activity relationships, focused collections of C26 and C32 analogs were strategically selected and obtained for testing with split- and linked-luc assay (FIG. 37). Candidates that showed positive results were analyzed further to generate inhibitory efficiency curves with multiple doses (FIG. 3E). Selected analogs for both C26 and C32 had better efficacy than their parent compounds (FIG. 3E).

Co-immunoprecipitation assay (Co-IP), which is a commonly used standard test to determine protein-protein interaction, was next employed to validate the candidates. Lysates of breast cancer cell line SCP28, a single cell-derived subline from the MDA-MB-231 TNBC cell line²⁵, was immunoprecipitated with anti-MTDH antibody alone or together with inhibitors (FIG. 3F and FIG. 10E). The samples were then incubated with Protein-A/G beads to pull down MTDH, followed by Western blot analysis to detect SND1. In such co-TP experiment, MTDH-WT but not MTDH-MT peptide blocks MTDH-SND1 interaction (FIG. 10E). When C26s and C32s were tested in the Co-TP assay, both series of compounds significantly blocked MTDH-SND1 interaction, with strongest inhibitory effects achieved by C26-A2 and C26-A6 (FIG. 3F). Using multiple screening and validation platforms, two classes of compounds, C26s and C32s, were obtained that inhibit MTDH-SND1 interaction.

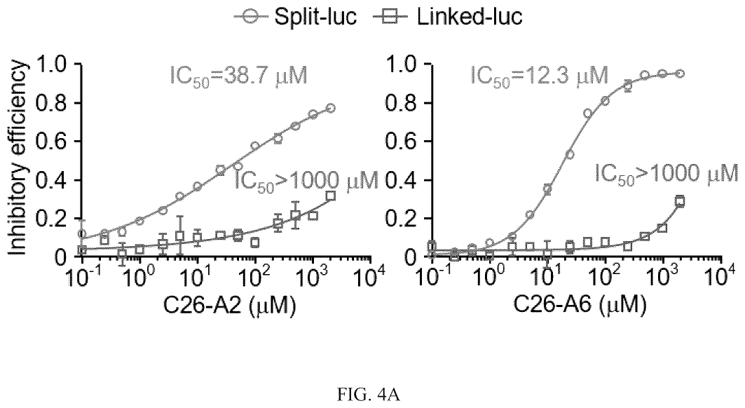

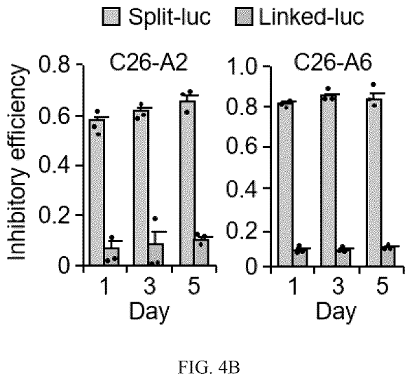

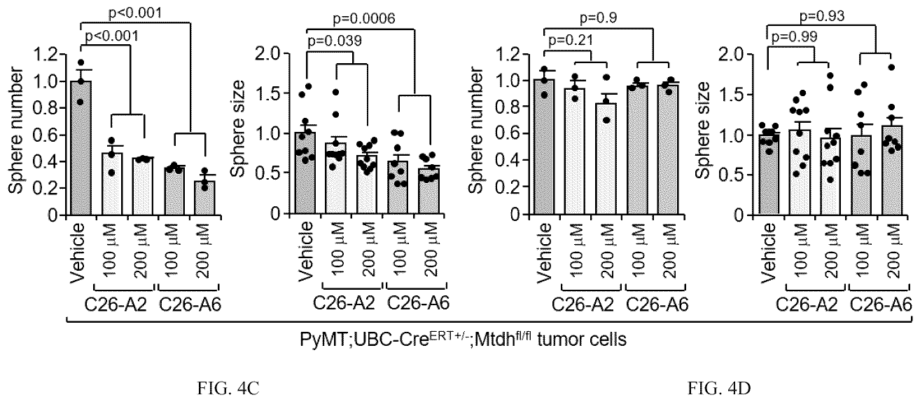

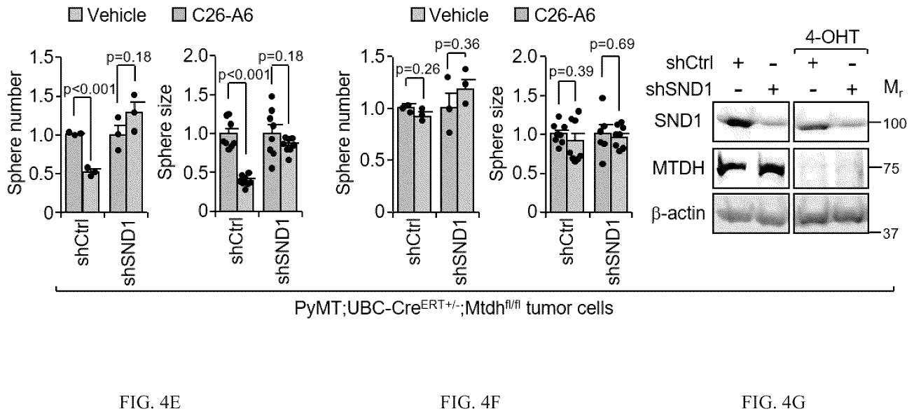

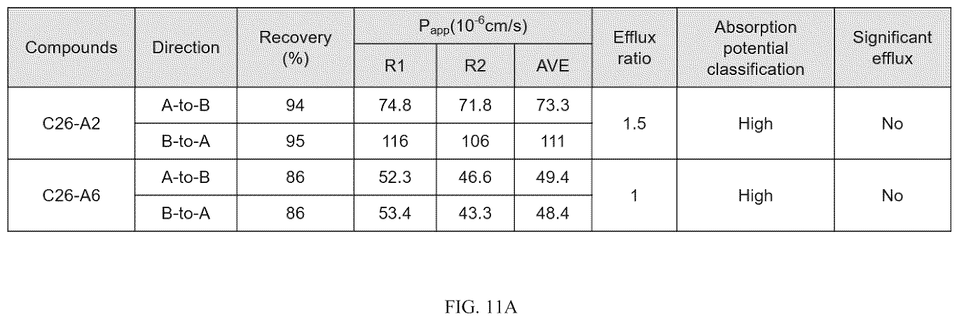

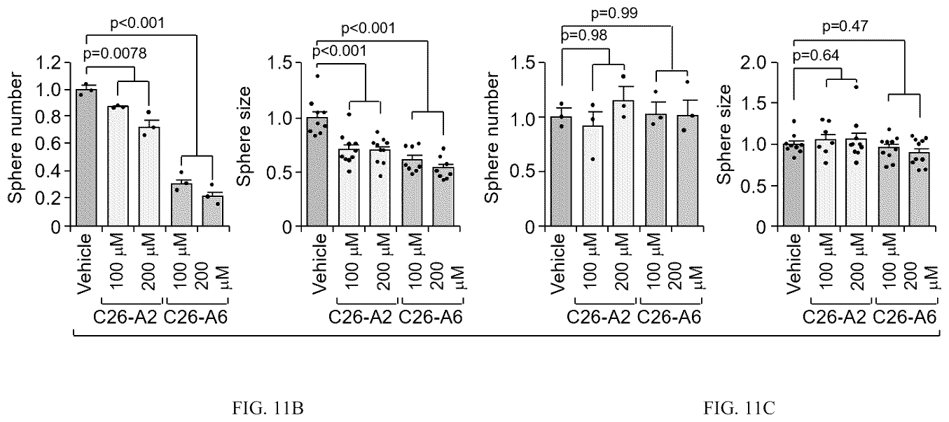

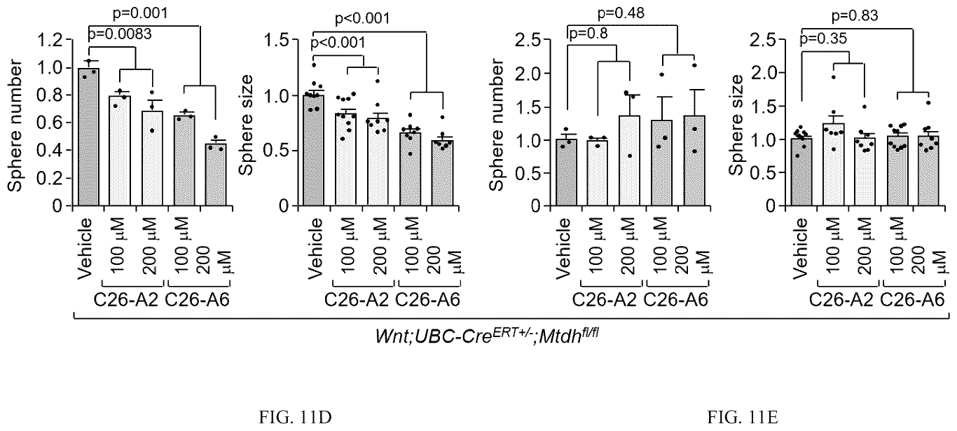

C26s inhibit breast cancer progression and metastasis. C26-A2 and A6 were picked for the functional test. To this end, it was tested whether C26-A2 and A6 could inhibit breast cancer progression. First, Caco-2 cell based permeability test²⁹ confirmed that both compounds were highly permeable (FIG. 11A). Next, SCP28 breast cancer cells that were engineered to express split- or linked-luciferase reporter were treated with C26-A2 or A6. Consistent with results from cell free system (FIG. 3E), both compounds inhibited split-luc activity in living cells in a dose dependent manner without significantly affecting linked-luc activity (FIG. 4A). Moreover, the blocking efficiency was not significantly changed 5 days after the addition of the compounds (FIG. 4B), suggesting the stability of the compounds in cells.

Next, PyMT; UBC-Cre^{ERT+/-};Mtdh^{fl/fl} tumor cells: 1) with or without Mtdh pre-depletion; 2) with or without endogenous Snd1 knockdown; or 3) in combination were employed for tumorsphere assay followed by the compound treatments. C26-A2 and A6 inhibited spheres formed by PyMT;UBC-Cre^{ERT+/-};Mtdh^{fl/fl} wild type tumor cells (FIG. 4C), however, they had no effects on the spheres with Mtdh KO or Snd1 KD (FIG. 4D-4G). The results were confirmed using C3 and Wnt;UBC-Cre^{ERT+/-};Mtdh^{fl/fl} tumorspheres (FIG. 11B-11E). Taken together, these findings confirmed that the anti-tumor effect of C26-A2 and A6 is dependent on their specific effect on blocking MTDH-SND1 interaction.

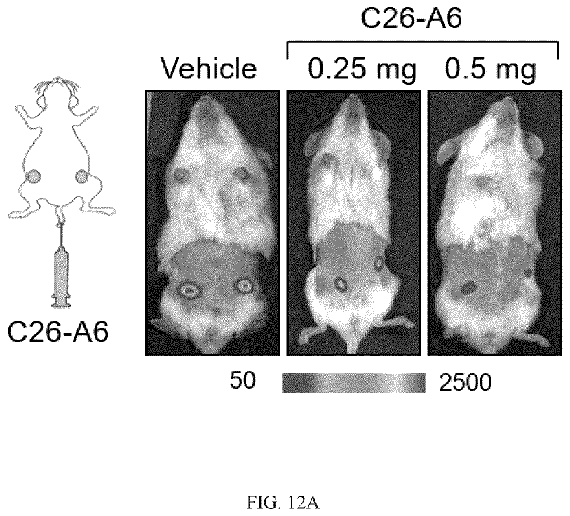

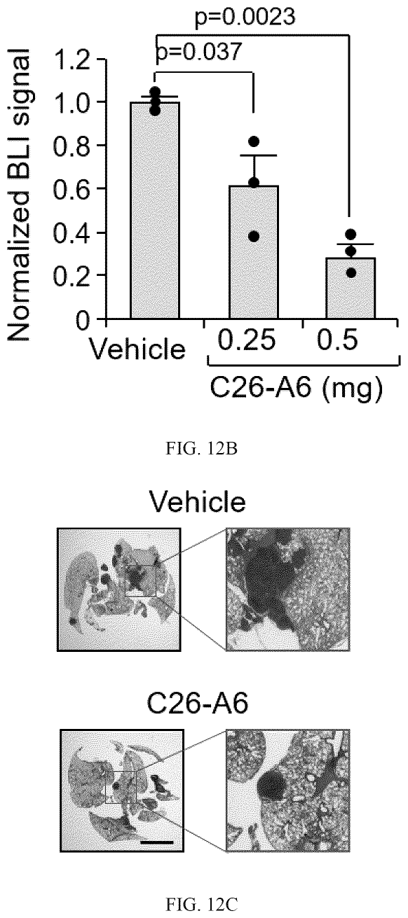

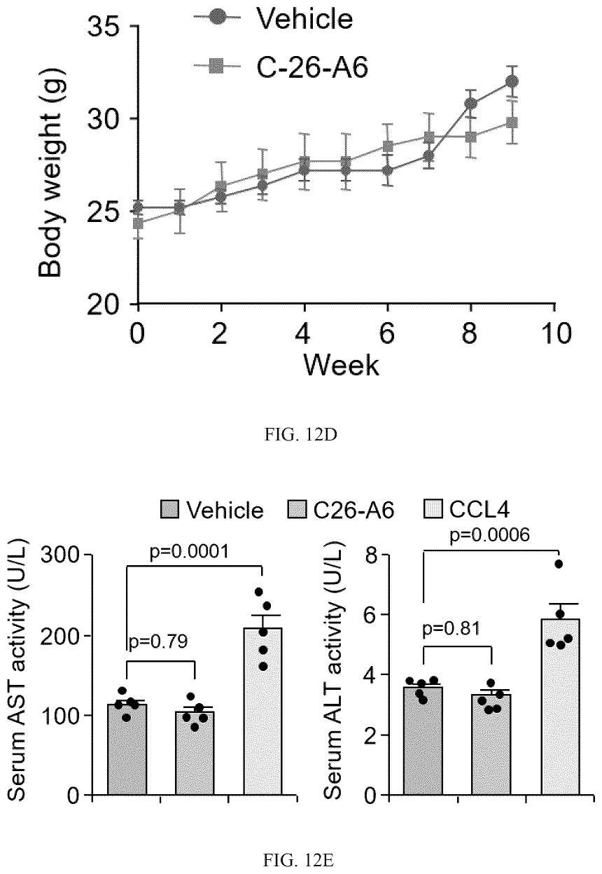

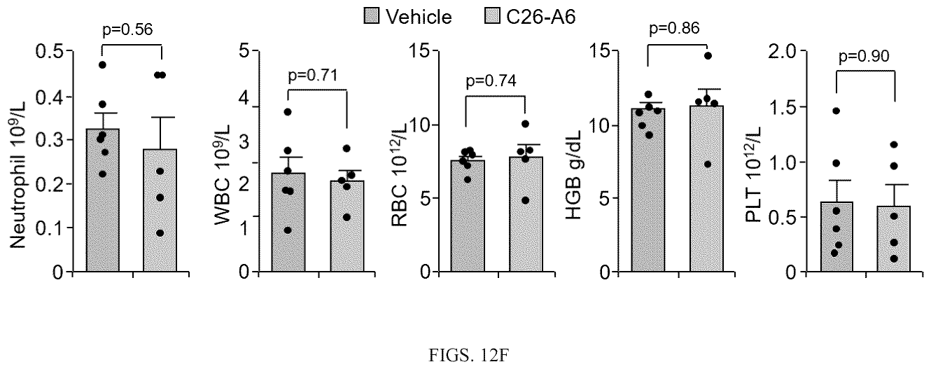

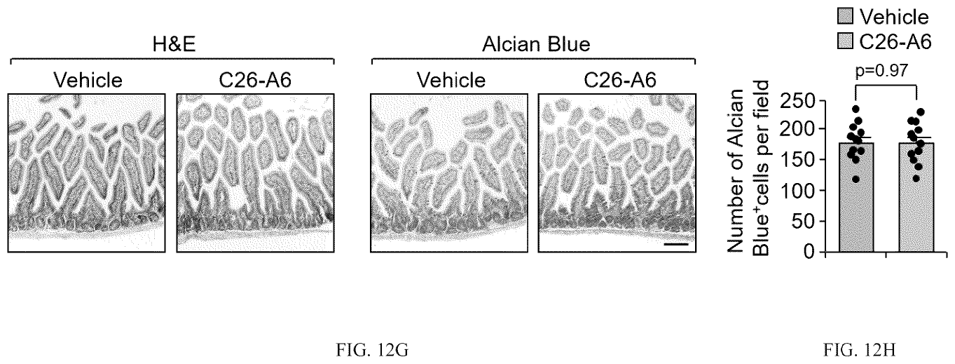

To test the in vivo blocking effect of C26-A6 on MTDH-SND1 interaction, mice with SCP28 tumors that stably express split-luciferase components were treated with vehicle, 0.25 mg or 0.5 mg of C26-A6 via tail-vein injection followed by bioluminescence imaging. The result indicated that C26-A6 blocks MTDH-SND1 interaction in vivo in a dose-dependent manner (FIGS. 12A, 12B).

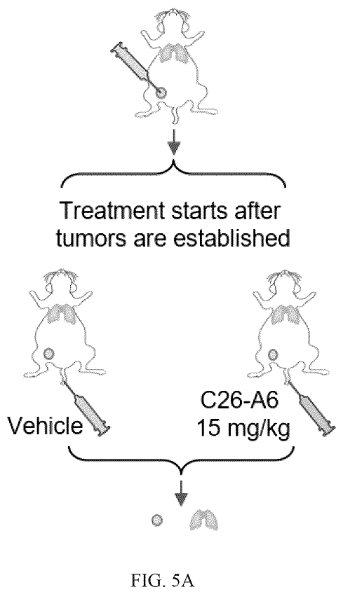

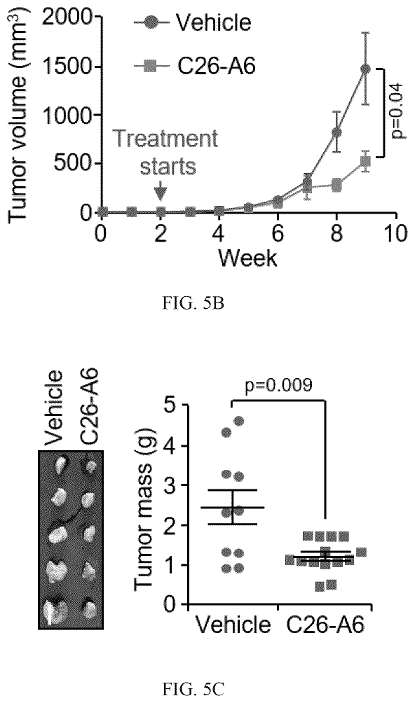

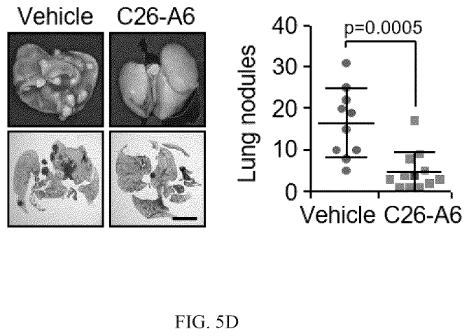

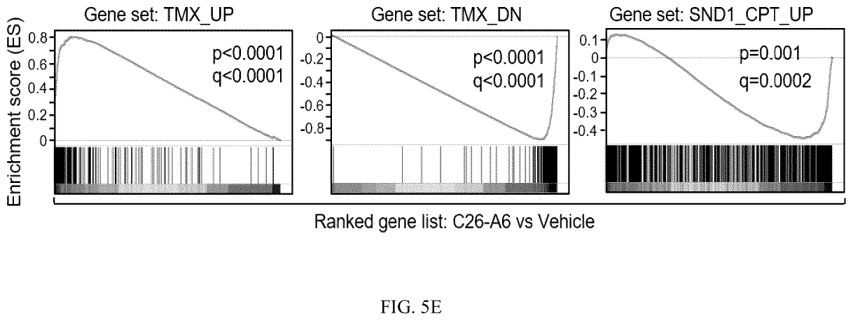

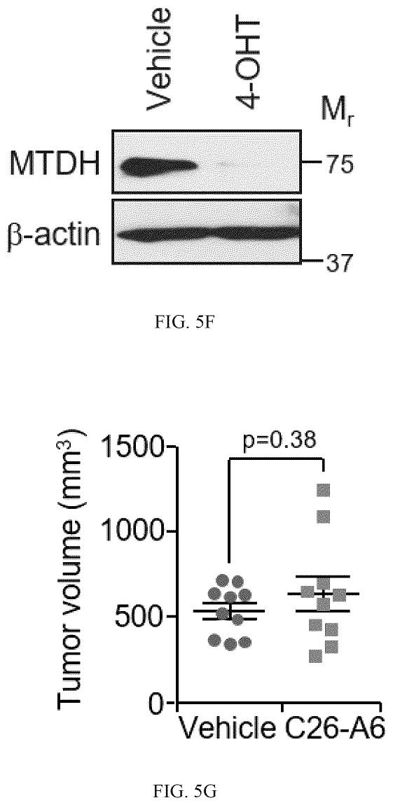

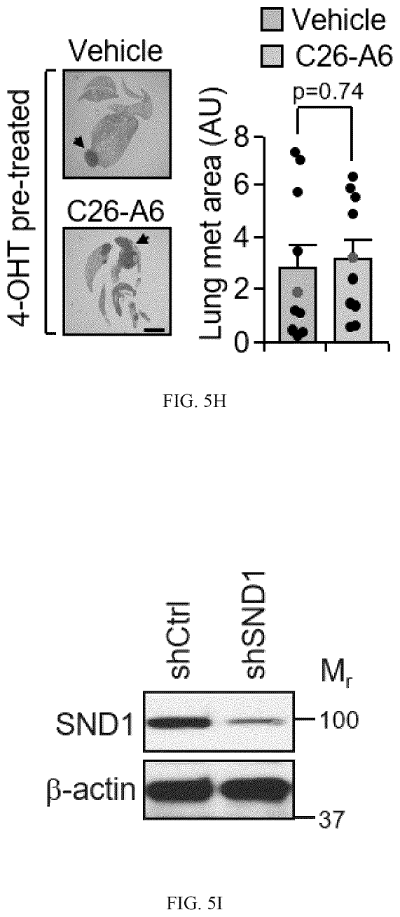

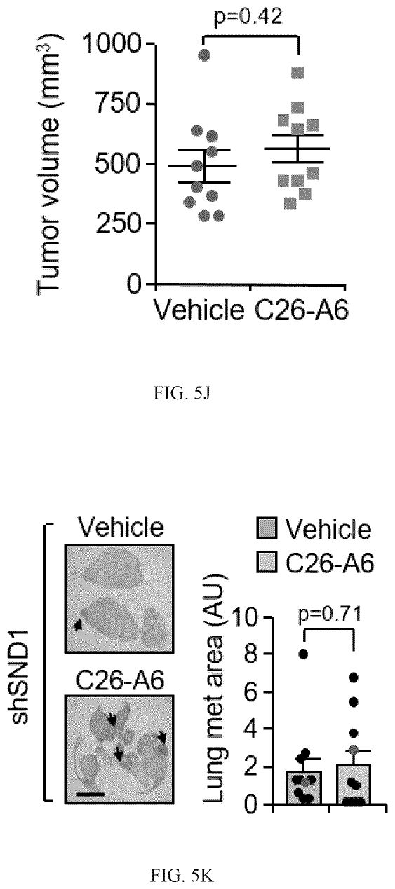

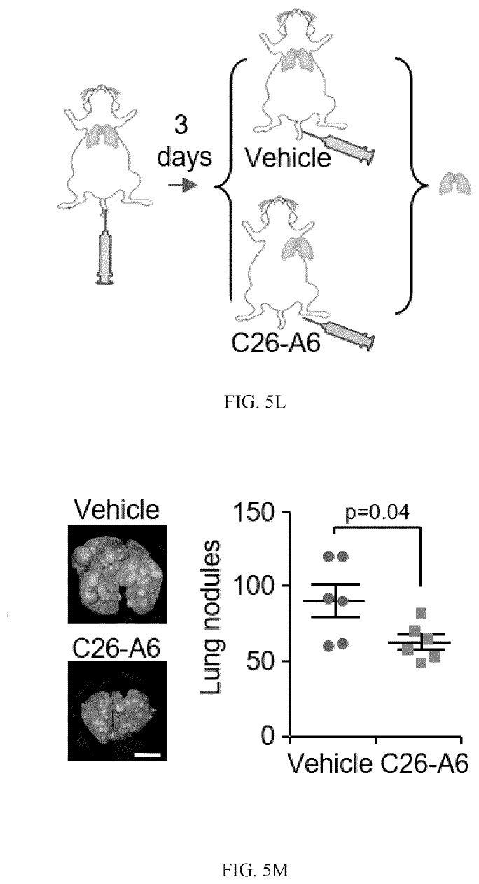

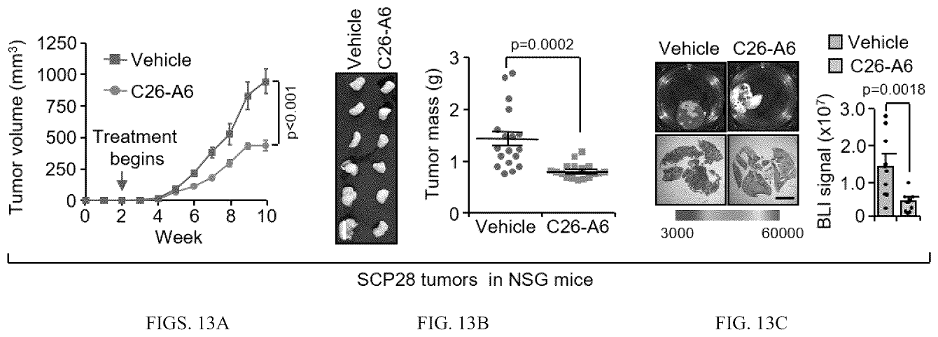

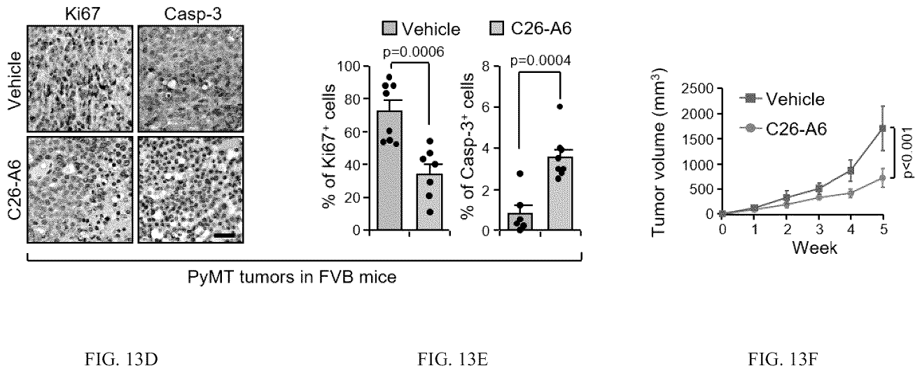

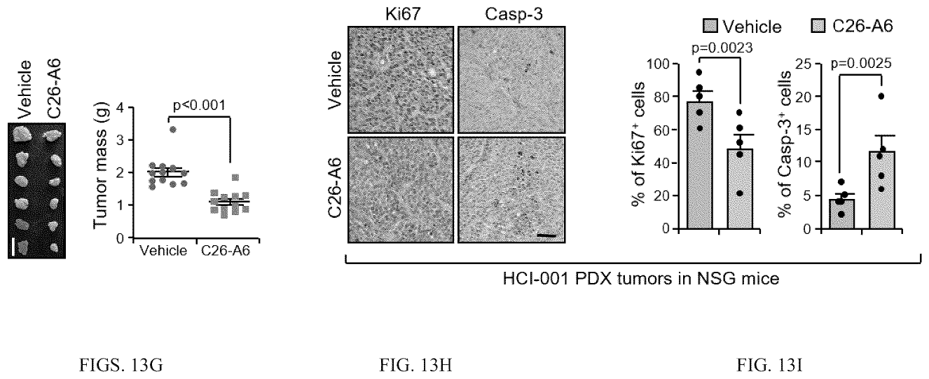

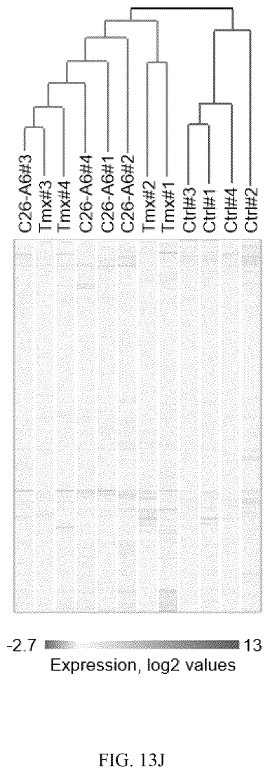

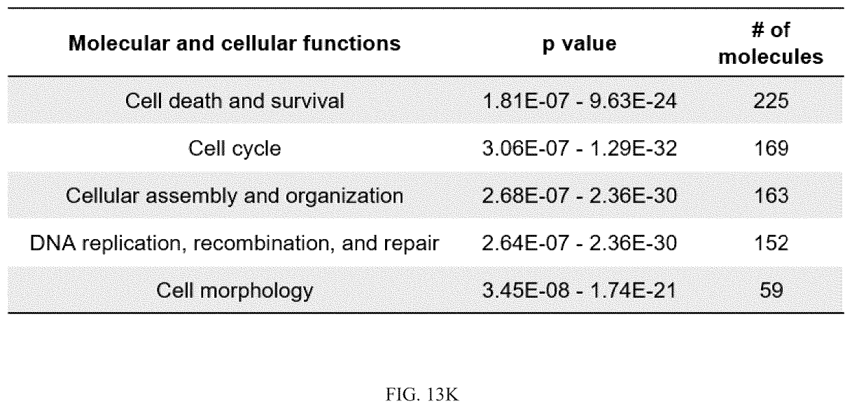

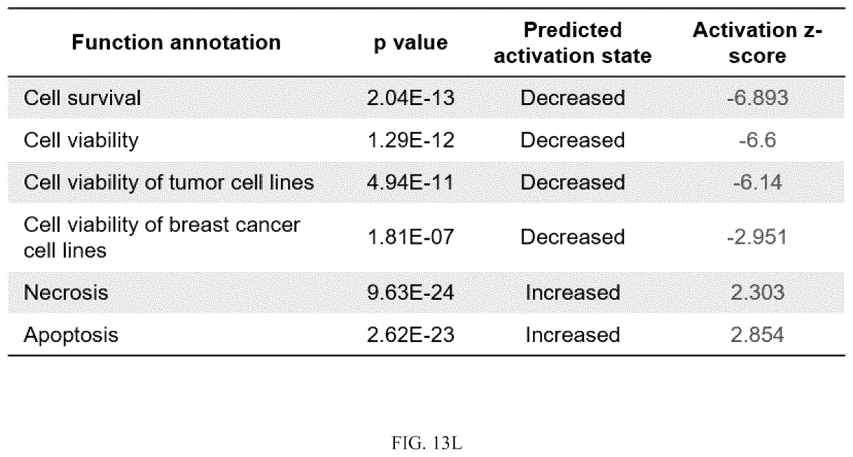

Next, established orthotopic PyMT tumors⁴ were treated with vehicle or C26-A6 (FIG. 5A). Continuous treatment of C26-A6 significantly inhibited primary tumor growth and spontaneous lung metastasis (FIGS. 5B-5D, and FIG. 12C), while having no significant hematologic, GI tract, and liver toxicity (FIGS. 12D-12H). The results were confirmed with SCP28 xenograft tumor model in NSG mice (FIGS. 13A-13C). Consistent with its tumor-suppressive effects, C26-A6 treatment reduced tumor proliferation and induced tumor apoptosis (FIGS. 13D, 13E). Similar therapeutic effects were also observed in a TNBC patient-derived xenograft (PDX, HCI-001) model³⁰ (FIG. 13F-13I).

To confirm at the molecular level that C26-A6 targets the MTDH-SND1 complex to suppress breast cancer progression, PyMT; UBC-Cre^{ERT+/-};Mtdh^{fl/fl} mice with tumors were treated with vehicle, Tmx, and C26-A6, followed by next-generation RNA sequencing (NGS). Non-supervised clustering based on global gene expression data indicated that Tmx and C26-A6 groups cluster together (FIG. 13J), suggesting that Mtdh acute KO and C26-A6 treatment had a similar effect on gene expression in tumors. Moreover, gene set enrichment analysis (GSEA) showed that Tmx treatment upregulated and downregulated genes were sharply enriched positively or negatively in C26-A6 tumors, respectively (FIG. 5E, left and middle panels), suggesting Mtdh acute KO and C26-A6 treatment regulate similar genes and pathways in breast tumors. To provide a better understanding of how C26-A6 exerts its tumor suppressive function, C26-A6 downregulated genes in comparison with vehicle were employed for Ingenuity Pathway Analysis (IPA). A significant enrichment of “Cell Death and Survival”, “Cell Cycle”, and “DNA repair” molecular and cellular functions was observed (FIG. 13K). Genes involved in cell survival and viability were decreased, whereas genes involved in apoptosis were increased (FIG. 13L) in C26-A6 treated tumors, which is similar to what was found in SND1-dependent signature during chemotherapy (SND1_CPT_UP)⁴. Furthermore, SND1_CPT_UP signature that negatively enriched in MTDH-SND1 interaction deficient tumors⁴, was also down regulated upon C26-A6 treatment (FIG. 5E, right). Taken together, these results confirm that C26-A6 targets MTDH-SND1 interactions to exert a global gene expression changes, leading to inhibition of breast cancer progression.

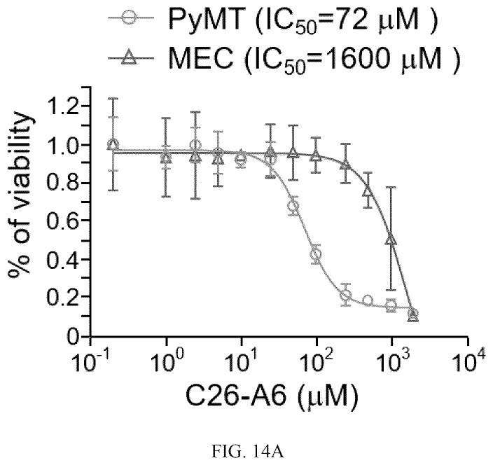

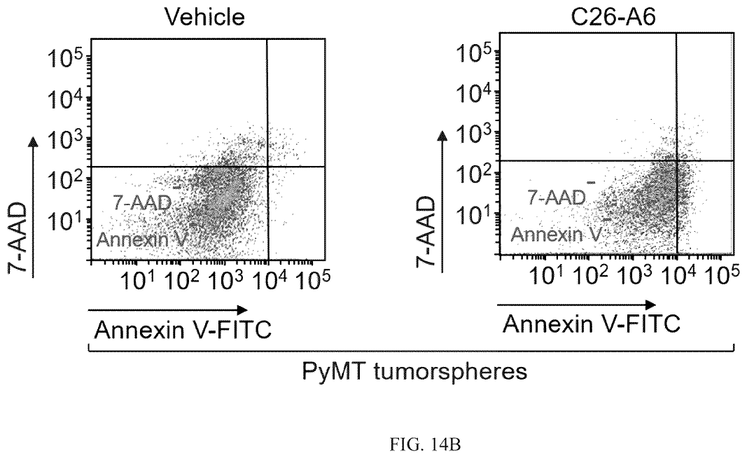

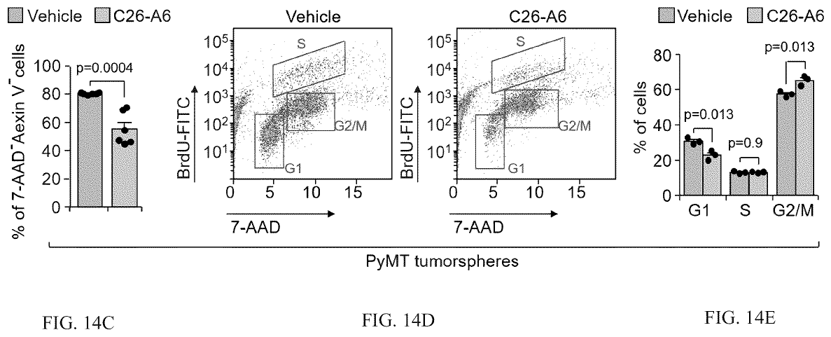

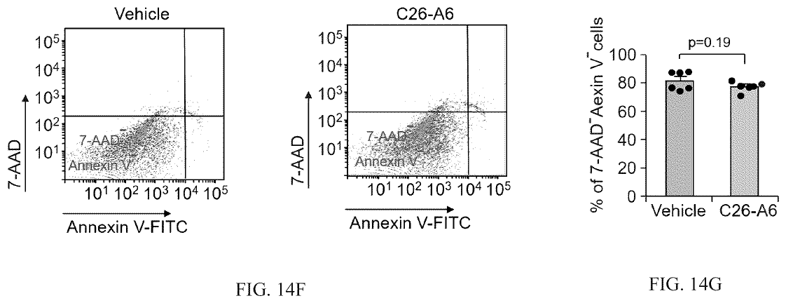

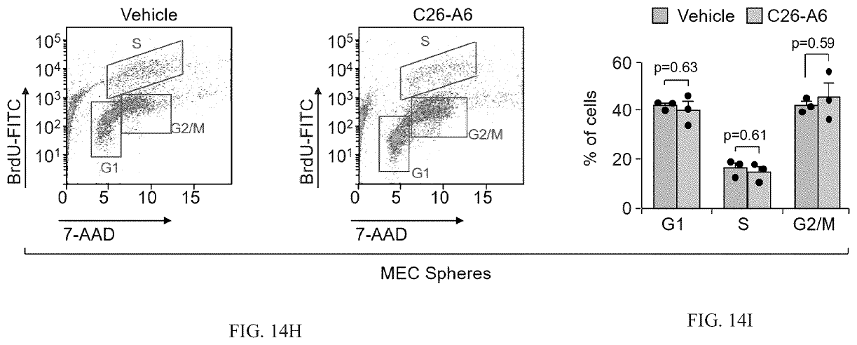

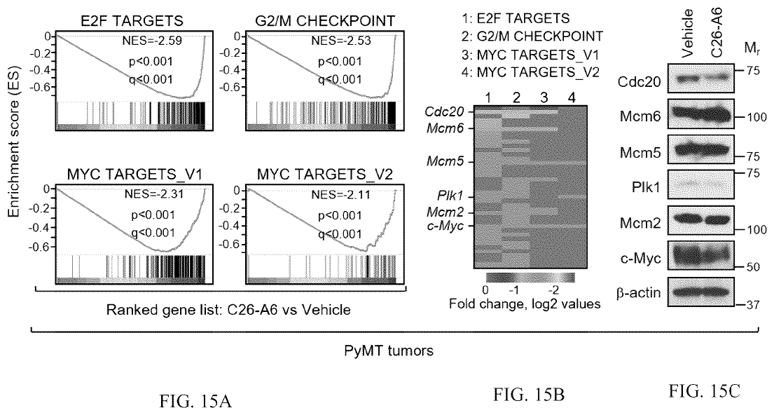

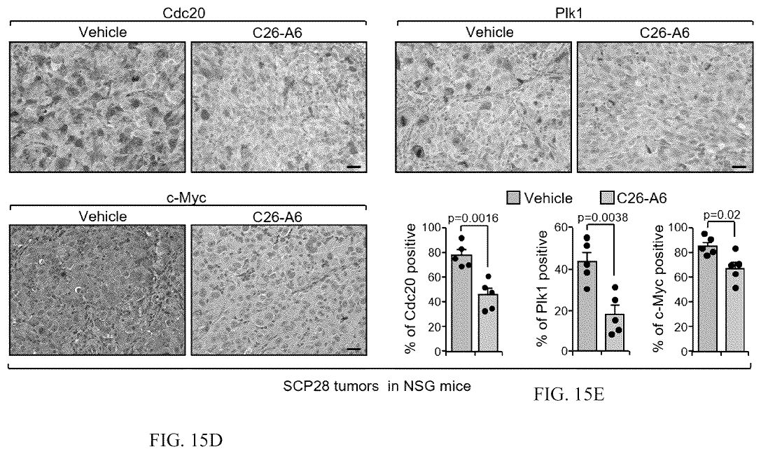

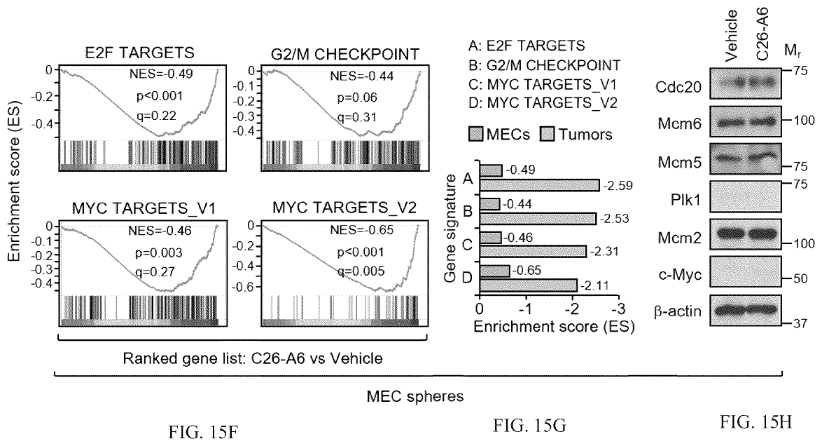

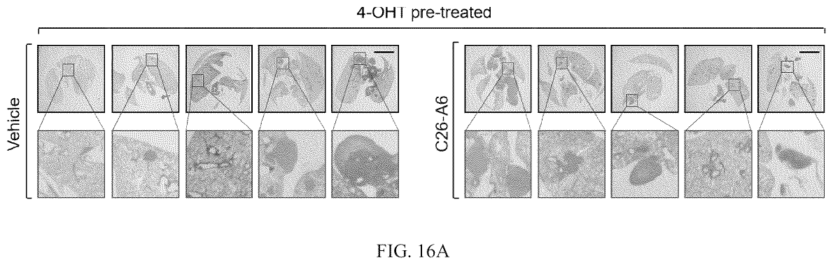

To obtain insights into the molecular mechanism underlying C26-A6 treatment-induced reduction of cell viability and increase of apoptosis, in vitro sphere assays were employed. PyMT tumorspheres are more susceptible to C26-A6 treatment than normal mammary epithelial cells (MECs) (FIG. 14A). 200 μM C26-A6 treatment significantly reduced survival and induced G2/M cell cycle arrest in PyMT tumorspheres, but not in normal MEC spheres (FIGS. 14B-14I). To further explore the downstream effectors of tumor-suppressive response to C26-A6, GSEA analysis of PyMT tumor with or without C26-A6 treatment was performed. The cell cycle and cell survival relevant signatures, E2F_TARGETS, G2M_CHECKPOINT, and MYC_TARGETS were significantly enriched in the control (vehicle) group compared to the C26-A6 treated group (FIG. 15A). Leading edge analysis of these enriched signatures results in a few downstream candidates, including Cdc20, Mcm6, Mcm5, Plk1, Mcm2 and c-Myc, that are significantly down-regulated upon C26-A6 treatment (FIG. 15B). Western blot analysis of the PyMT tumorspheres showed that, among these candidates, Cdc20, Plk1, and c-Myc were down-regulated by C26-A6 treatment (FIG. 15C), an observation that was also confirmed using SCP28 tumor samples (FIGS. 15D, 15E). Interestingly, RNA-sequencing data of the normal MEC spheres did not reveal comparable signature enrichment or gene expression changes as in tumor samples (FIGS. 15F-15H). Collectively, the data suggested Cdc20, Plk1, and c-Myc as possible downstream targets that mediate the effect of MTDH-SND1 inhibition on inducing tumor-intrinsic cell cycle arrest and apoptosis.

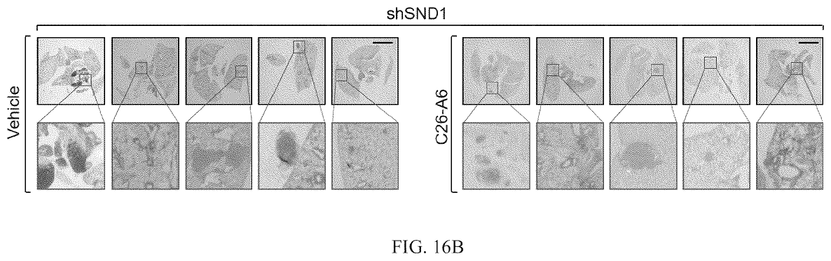

Next, to further validate that the anti-tumor effect of C26-A6 depends on its on-target effects by disrupting MTDH-SND1 complex, PyMT; UBC-Cre^{ERT+/-};Mtdh^{fl/fl} tumor cells with Mtdh depletion or with endogenous Snd1 KD were employed for the similar assay as in FIG. 5-1a. The result indicated that C26-A6 did not further inhibit tumor growth or metastasis in the models with MTDH KO or SND1 KD (FIGS. 5F-5K, and FIG. 16). Likewise, MTDH or SND1 KD abolished the C26-A6 treatment-induced primary tumor inhibition or metastasis reduction in SCP28 tumor models (Extended Data FIG. 10-1a-c).

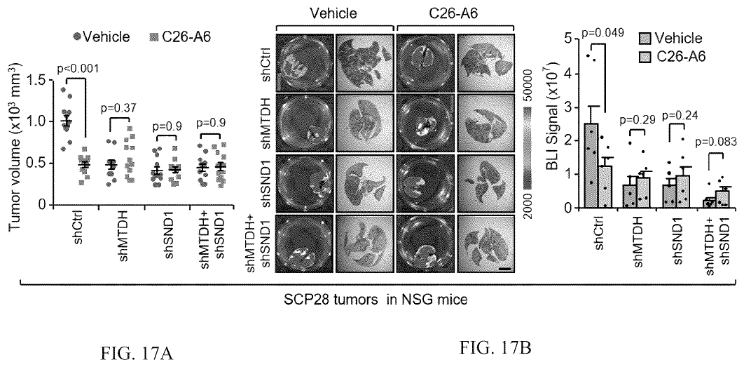

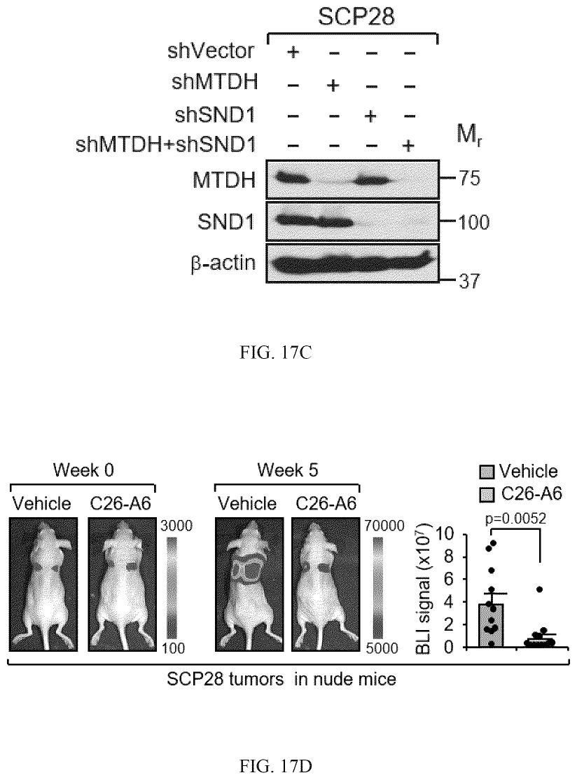

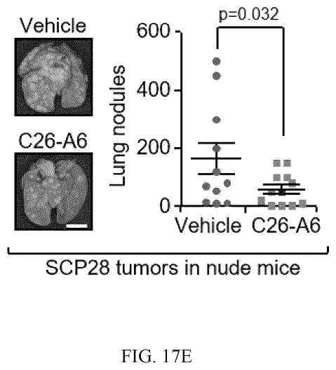

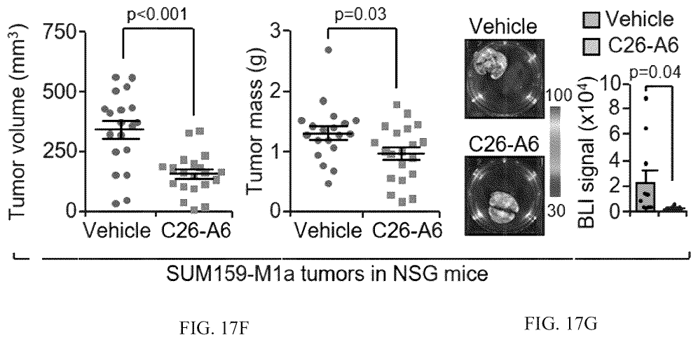

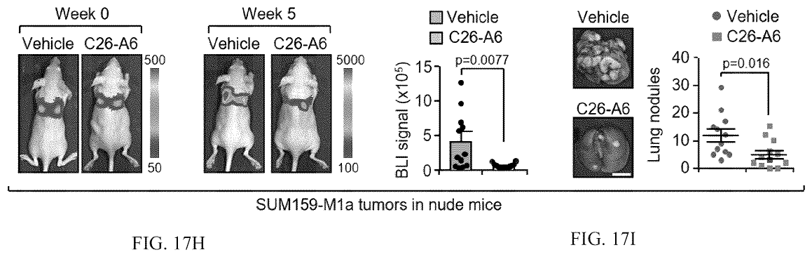

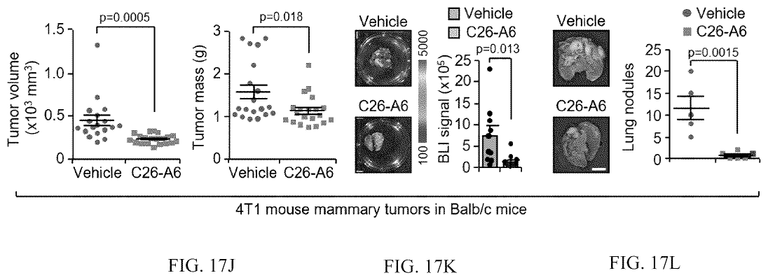

To exclude the possibility that the lung metastasis reduction upon C26-A6 treatment was due to the smaller primary tumor, tail vein injection of PyMT tumor cells was performed to form experimental lung metastasis in FVB mice. Three days later, mice were divided randomly into two groups and treated with vehicle or C26-A6 respectively (FIG. 5L). Five weeks of C26-A6 treatment significantly suppressed lung metastasis (FIG. 5M). Similarly, C26-A6 also dramatically inhibited experimental lung metastasis of SCP28 cells (FIG. 17D, 17E). The primary tumor growth and metastasis-suppressive role of C26-A6 was further validated with additional breast cancer models, including the SUM159-M1a lung-metastatic human breast cancer cell line in NSG mice^{31,32} and 4T1 mouse mammary tumor models in immunocompetent Balb/c mice (FIGS. 10F-10L). Taken together, the data revealed that C26-A6 blocks MTDH-SND1 interaction to inhibit breast cancer progression and metastasis.

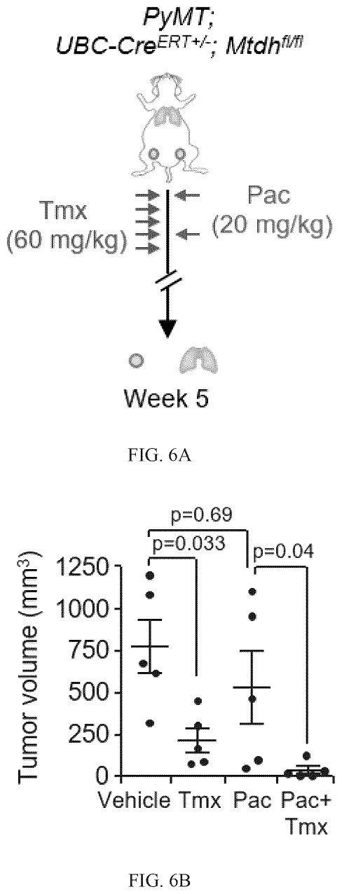

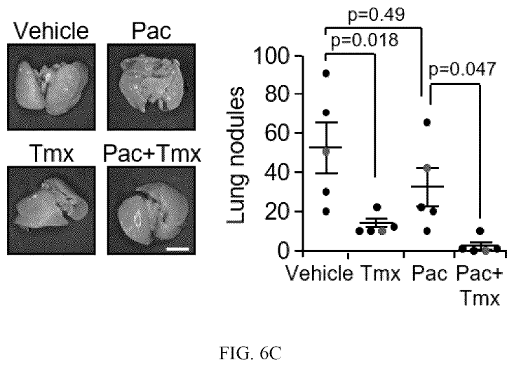

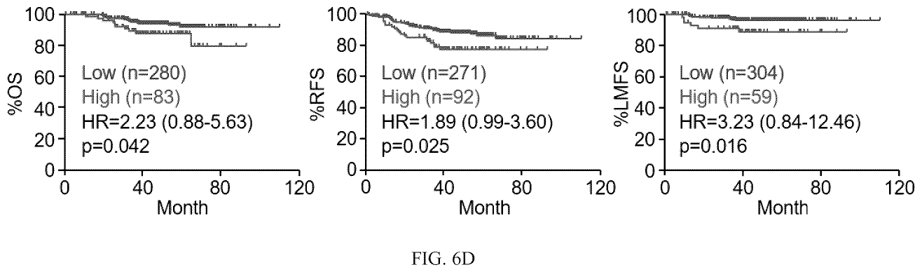

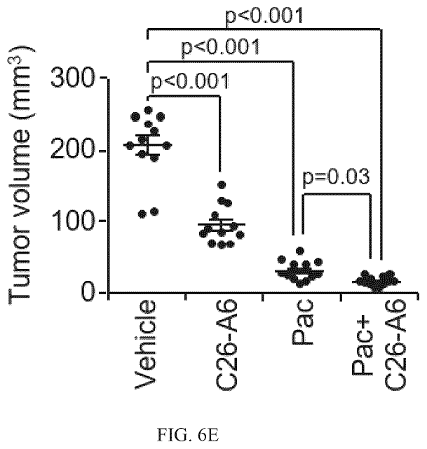

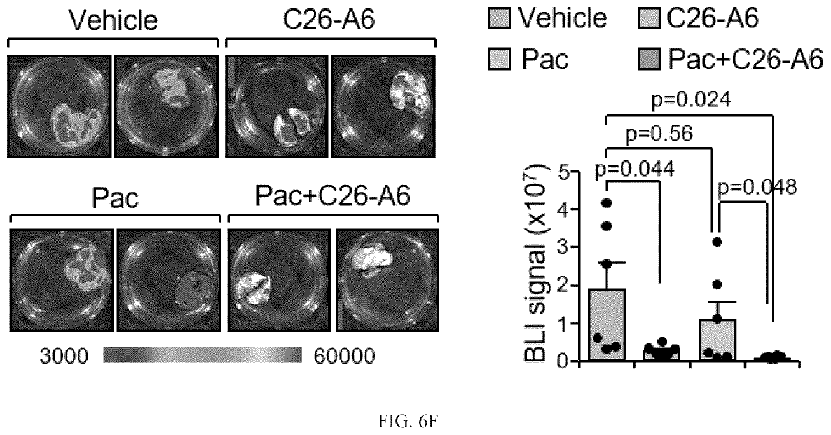

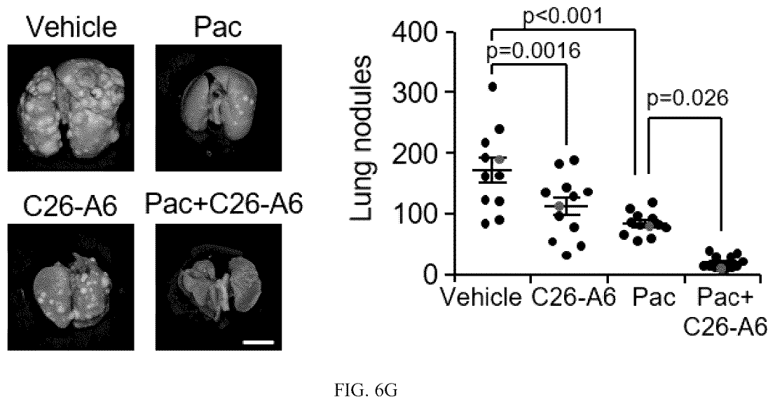

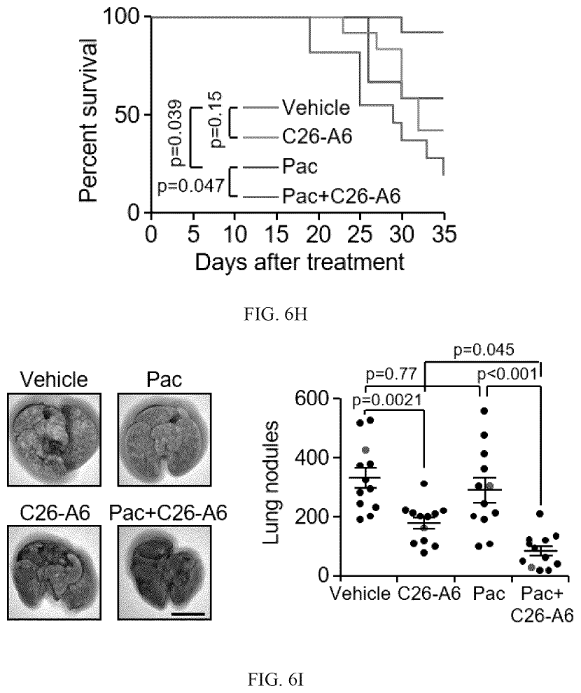

MTDH-SND1-targeting sensitizes breast cancer to chemotherapy. Given that MTDH promotes chemoresistance², it was hypothesized that MTDH-targeting could sensitize breast cancer to chemotherapy. To test this, PyMT; UBC-Cre^{ERT+/-};Mtdh^{fl/fl} mice with established tumors were treated with Tmx and paclitaxel alone or in combination (FIG. 6A). Acute loss of Mtdh by Tmx treatment significantly reduced primary tumor growth and lung metastasis (FIGS. 6B, 6C). Importantly, Tmx+paclitaxel combined treatment dramatically reduced primary tumor growth and lung metastasis to a degree that is superior to paclitaxel or Tmx treatment alone (FIGS. 6B, 6C).

Chemotherapies are commonly applied to the treatment of triple-negative breast cancer (TNBC). 363 subjects with TNBC who were treated with chemotherapy after surgeries were analyzed. Primary tumor samples were surgically removed before chemotherapy and were used to measure MTDH expression. Patients with MTDH-high expressing tumors had significantly worse overall, relapse-free, and lung metastasis-free survival (FIG. 6D) after surgery and chemotherapy. This finding indicates the possibility to target MTDH in human patients in order to sensitize the TNBC patients to chemotherapy.

Based on these findings, it was directly tested whether pharmacological inhibition of MTDH-SND1 can synergize with chemotherapy to improve treatment outcome in mouse models. Similar to experiments using genetic depletion Mtdh (FIGS. 6B, 6C), C26-A6+paclitaxel had significantly better efficacy in inhibiting SCP28 primary tumor growth and lung metastasis than C26-A6 or paclitaxel treatment alone (FIGS. 6E, 6F).

Next, the efficacy of the treatments in suppressing metastatic colonization was investigated. To this end, Balb/C mice were injected with 4T1 mouse mammary tumor cells, which mimic TNBC³³, via tail-vein and were subjected to the same treatment regime as above three days later. Consistently, C26-A6+paclitaxel significantly inhibited lung metastasis more than either treatment alone (FIG. 6G). Furthermore, mice with C26-A6+paclitaxel treatment had the best survival rate (FIG. 6H).

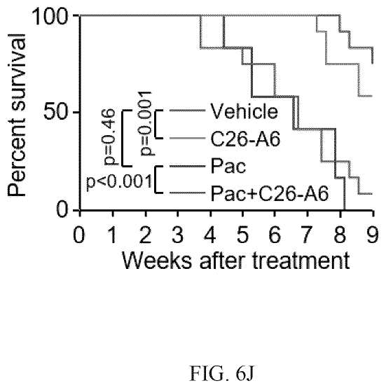

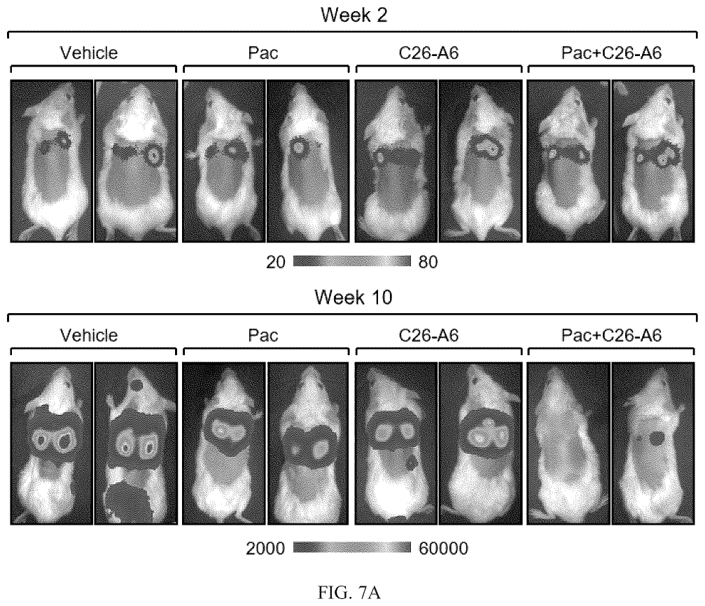

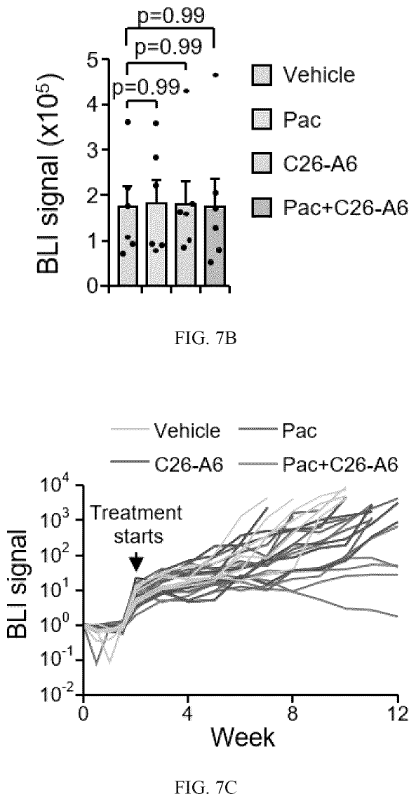

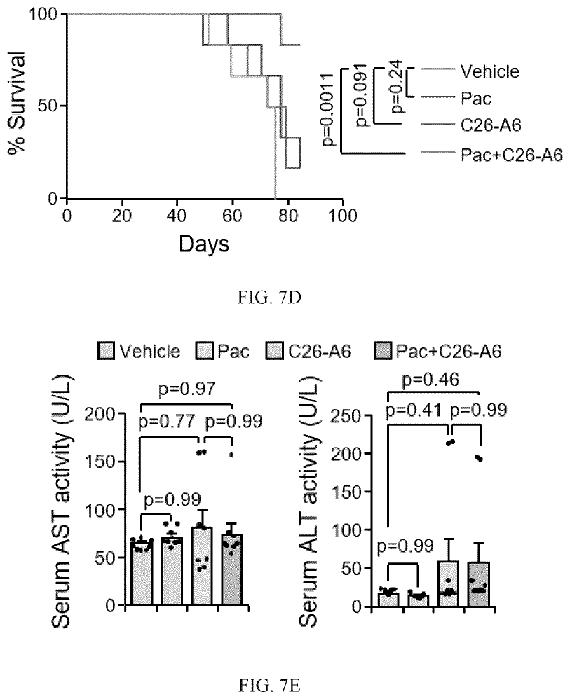

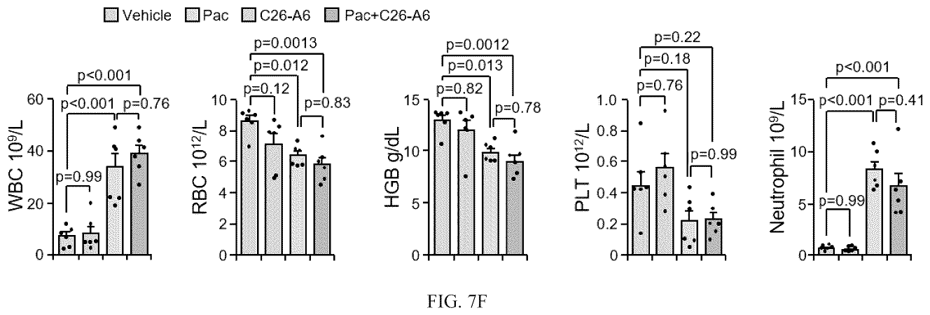

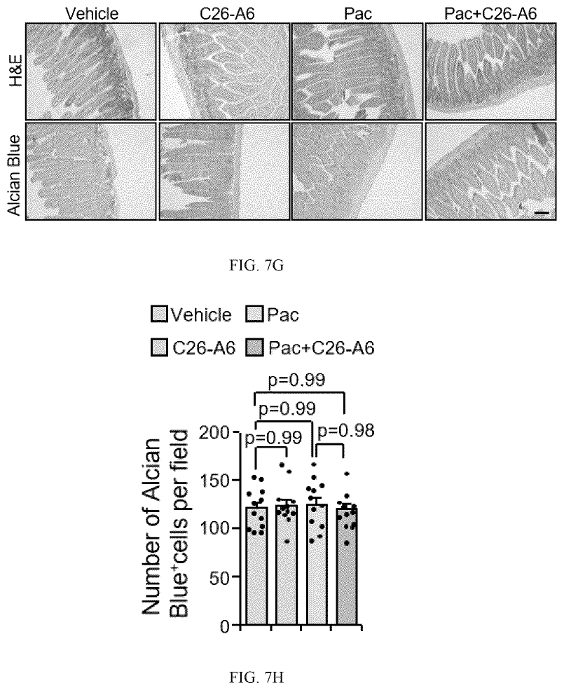

Lastly, to mimic the clinical scenario of adjuvant chemotherapy following surgical removal of the primary tumors, SCP28 mammary tumors were removed when they reached 5 mm in diameter, the mice were then randomly separated into four groups and subjected to the different treatment regimens as above. Again, C26-A6+paclitaxel treatment achieved the more effective reduction of lung metastasis and overall survival (FIGS. 61, 6J), suggesting targeting MTDH-SND1 complex together with chemotherapy could significantly improve the treatment outcome for breast cancer. To further evaluate if the treatment could result in the shrinkage of established macrometastases, tail vein injections were performed to generate 4TO7 lung metastases-bearing mice. The mice were randomized into four groups of six mice each when macrometasetases were well-established, as evidenced by robust BLI signals in the lungs (FIGS. 7A, 7B). The mice were then treated with vehicle or paclitaxel and C26-A6 alone or in combination, and the metastasis progression was monitored by BLL. Although paclitaxel or C26-A6 treatment alone slowed down metastatic growth, these monotherapies did not result in metastasis shrinkage (FIG. 7C). However, three mice in C26-A6+paclitaxel group had stabilized disease and one mouse had obvious metastasis shrinkage, leading to significantly improved survival rate (FIGS. 7C, 7D). More importantly, consistent with the results shown above (FIGS. 12D-12H), C26-A6 was well tolerated by the mice and did not further enhance the chemotherapy toxicity when combined with paclitaxel (FIGS. 7E-7H). These data suggest that C26-A6 combined with chemotherapy may have clinical benefit in metastatic breast cancer patients.

Discussion In this study, the therapeutic potential of targeting MTDH-SND1 complex using a combined genetic and pharmacological approach was evaluated. The inducible conditional Mtdh KO mouse and cell line models provided a relevant pre-clinical model to mimic MTDH-targeting in an autochthonous and immunocompetent tumor development setting and allowed more accurate assessment of its therapeutic benefit in late tumor development stages. Importantly, acute pharmacological and genetic inhibition of MTDH revealed robust and consistent global gene expression changes that were not easily discernable in previous studies using constitutive MTDH-KO mouse or cell line models. The data provided essential proof-of-concept evidence that MTDH is a suitable target for the treatment of established breast cancer and potentially other cancers. Furthermore, a series of first-in-class small chemical compounds was identified that achieved robust therapeutic effects by disrupting MTDH-SND1 interaction.

Protein-protein interactions (PPIs) are critical for all the biological processes including tumorigenesis and cancer progression^{34,35}. Despite the importance of PPIs in disease development, targeting PPI was initially considered to be impossible due to the large, flat, and featureless interaction interface³⁶. However, with recent breakthroughs in technology development, high resolution structural studies revealed that not all residues at the PPI interface were critical, but rather that small “hot spots” conferred most of the binding energy³⁷⁻³⁹, paving the path to the recent success of PPI inhibitor development⁴⁰⁻⁴⁴. Consistent with this notion, structural biology study revealed that MTDH-SND1 interaction was critically reliant on several key residues of MTDH and SND1, and that such interactions were potentially amenable to disruption by small chemical compounds¹².

Functional studies of C26s suggested potential tumor suppressive role of this structural class of compounds (FIGS. 4 to 6). Moreover, C26-A6, which is an analog of C26s, was well-tolerated in vivo with minimal toxicity (FIGS. 7 and 12). Considering the high tolerability in vivo and the compound's solubility, it was determined to use highest dose achievable (15 mg/kg) for the treatment experiments. C26-A6 treatment alone or in combination with chemotherapy dramatically suppressed breast cancer progression and metastasis (FIGS. 5 to 7), consistent with gene expression analysis showing the pro-survival and anti-apoptosis role of MTDH in cancer cells under stressful situations, such as chemotherapy.

Target specificity of the drug is critical for clinical application of novel therapeutic compounds. To this end, multiple lines of evidence show that the tumor-suppressive effect of C26-A6 is due to its on-target effects: phenotypically, tumor models with MTDH-KO or SND1-KD were developed and then treated with C26-A6. In this experimental setting, no further tumor inhibition upon C26-A6 treatment was observed (FIG. 5), suggesting that C26-A6 targets MTDH-SND1 to exert its tumor inhibition function. Moreover, RNA sequencing data together with the gene set enrichment analysis suggested that C26-A6 treatment and MTDH-KO or SND1 silencing alter the exact same set of pathways (FIG. 5 and FIG. 13). Taken together, the data suggest that the tumor- and metastasis-suppressive effects of C26-A6 treatment is due to its on-target effect on blocking the MTDH-SND1 complex and its downstream targets. Collectively, the results underscore the feasibility and therapeutic potential of targeting the MTDH-SND1 complex for the treatment of breast cancer, and establish the C26 series as promising candidates for further development as a new first-in-class cancer therapeutic agent.

Methods: Animal models. All procedures involving mice and experimental protocols were approved by the Institutional Animal Care and Use Committees (IACUC) of Princeton University. According to the approved IACUC protocol (1881-20), the primary humane endpoint of tumor burden for an individual mouse is 20 mm in any dimension or a total volume of 4000 mm³ for mice with multiple tumors. Mice were euthanized before exceeding the limit of tumor burden in this study. In the facility, mice were maintained at 20-22° C. with 14 h:10 h light:dark cycles at 40-70% relative humidity. The Mtdh^{fl/fl} ES cell lines generated by Mtdh targeting vector (CSD48311) was obtained from the KOMP Repository. The ES cells were injected into the C57BL/6 blastocysts followed by confirmation of germline transmission. Mtdh^{fl/fl} mice were crossed with FLPe mice to remove the selection marker in the vector. Genotyping (Forward primer: CCCACCCCGCTTTGACCAAATAC (SEQ ID NO:21); Reverse primer: GTGCCACCACTGCCCAGCTTC (SEQ ID NO:22)) was performed to identify positive mice before they were crossed to other strains that were indicated in each experiment. FLPe (Stock No. 003946), MMTV-PyMT (Stock No. 002374), MMTV-WNT1 (Stock No. 002934), C3 (Stock No. 013591), UBC-Cre^{ERT2} (Stock No. 007001), FVB (Stock No. 001800), Balb/C (Stock No. 000651), Athymic nude (Nude) (Stock No. 002019), and NOD Scid Gamma (NSG) (Stock No. 005557) were obtained from Jackson Laboratory. Mice in C57BL/6 background were backcrossed to FVB for at least 10 generations to change the background. For spontaneous tumor models, the mice were randomized and matched by tumor size rather than time of growth before treatment was started. For xenograft/allograft studies, 8-weeks immunocompromised NSG, Nude or immunocompetent FVB or Balb/C females were used. Injections were performed as previously described³². The mice were randomized as indicated in each specific experiment before starting the treatments. For Tamoxifen (Tmx) and Paclitaxel (Pac) treatments, mice were injected with 60 mg/kg and 20 mg/kg via i.p. respectively. For C26-A6 treatments, mice were injected with 15 mg/kg via i.v. Primary tumors were considered established when they became palpable for 2 consecutive weeks. The tumors were measured by calipers for calculation of tumor volumes (length×width²/2). For cell lines that are stably labeled with a firefly luciferase expressing vector, lung metastases were monitored by bioluminescent imaging (BLI) and images were processed with Living Image 3D Analysis (version 1.0). For PDX treatment, fresh TNBC patient-derived xenograft (PDX, HCI-001)³⁰ was chopped into ˜1-2 mm in diameter cubes and inoculated into 8-week NSG females. One day after inoculation, the mice were randomized and treated with vehicle or C26-A6.

Generation of TNBC cohort and expression determination. The generation of TNBC cohort has been detailed described in previous studies^{7,45}. As noted, this study was approved by the independent ethics committee/institutional review board of FUSCC (Shanghai Cancer Center Ethics Committee). All patients gave their written informed consent before inclusion. 386 patients were selected, and RNA sequencing was performed on 245 samples. HTA 2.0 array sequencing was performed on the other 141 samples. Detailed information of HTA data was described previously^{46,47}. Combat (“ComBat” function in R) was utilized to adjust batch effects between the RNA-seq and HTA array datasets. To calculate the prognostic efficacy ofMTDH on overall survival, relapse and lung metastasis in the TNBC patients with chemotherapy, the optimal cutoff value (“cutp” function in R) was chosen to classify the expression of MTDH into low and high expression subtypes in each prognostic calculation. Afterwards, survival analysis was performed as previously described⁷.

Cell culture. SUM159-M1a was derived from SUM159 breast cancer cell line³¹. HEK293T (CRL-3216) and 4T1 (CRL-2539) were obtained from American Type Culture Collection. SCP28, 4TO7, and H29 were obtained from Dr. Joan Massague. PyMT; UBC-Cre^{ERT+/-};Mtdh^{fl/fl}, C3; UBC-Cre^{ERT+/-};Mtdh^{fl/fl}, and Wnt; UBC-Cre^{ERT+/-};Mtdh^{fl/fl} cell lines were generated in this study. SCP28, 4T1, 4TO7, HEK293T, and the generated cell lines were grown in DMEM supplemented with 10% FBS and pen/strep. SUM159-M1a cells were culture with F12 media supplemented with 10% FBS, 10 μg/ml Insulin, 20 μg/ml EGF and pen/strep. H29 was grown in the same media supplemented with 2 μg/ml puromycin, 300 μg/ml G418 and 1 μg/ml doxycycline. All cells were regularly checked for Mycoplasma and authenticated.

Cloning, viral production and transduction of cell lines. To generate plasmids that express split- and linked-luciferase components, plasmids that express human MTDH and SND1^{2,8} were used as template. Firefly luciferase plasmid (pGL3, Promega, Cat #E1751) was also employed as template. Firefly luciferase was split into N-terminal (NLuc: 1-416aa) and C-terminal (CLuc: 398-550aa) as previously reported²¹. SND1 (16-339aa) was cloned and fused to N-terminal of NLuc with 3 repeats of GGGS as a linker. Similarly, MTDH (386-407aa) was cloned and fused to C-terminal of CLuc with 3 repeats of GGGS. SND1-NLuc and CLuc-MTDH flanked by BamH 1 and Not 1 restriction sites were inserted into pcDNA3.1 and pRVPTO (retrovrial) vectors. Human influenza hemagglutinin (HA) tag was fused to SND1-NLuc and Myc tag was fused to CLuc-MTDH. For linked-luciferase, NLuc and CLuc were linked with 3 repeats of GGGS, flanked by the same restriction sites, and inserted into the same vectors. shRNAs targeting human MTDH² and SND1⁸ was described in previous studies. shRNAs targeting mouse Snd1 was purchased from Sigma (TRCN0000295753) and validated previously^{4,12}. Mouse wild type full length MTDH and SND1 interaction deficient mutant MTDH-13D (MTDH-W391D) was reported previously⁴. All plasmids were sequenced and confirmed for accuracy. To generate SCP28 cell line that stably express split- and linked-luciferase components, retroviral vectors generated above were transfected into the H29 packaging cell line. Detailed procedure was described previously³².

Generation of tumor cell line from inducible Mtdh KO mice. PyMT;UBC-Cre^{ERT+/-};Mtdh^{fl/fl}, C3; UBC-Cre^{ERT+/-};Mtdh^{fl/fl}, and Wnt; UBC-Cre^{ERT+/-};Mtdh^{fl/fl} tumor cell lines were generated as following: FVB-PyMT/C3/Wnt;UBC-Cre^{ERT+/-};Mtdh^{fl/fl} mice that have primary tumors around 5 mm in diameter were sacrificed. The primary tumor was dissected and plated into 10 cm dished. Two days later, floating tissues were washed off with culture media and attached cells were further cultured with fresh media to become stable cell lines.

Immunoprecipitation (IP) and western blotting (WB) analysis. For IP experiment, samples were prepared as previously described³². 100 μl of the supernatant was transferred to a new tube as input, and the rest was incubated with 2 μg of IgG, anti-Myc (Santa Cruz, SC-40), or anti-MTDH (ThermoFisher, 40-6500) (as indicated in each experiment) overnight at 4° C. (small chemical compounds may be added at this step as indicated in each experiment). The rest standard IP procedures were performed as previously noted³². For WB analysis, samples were resolved with SDS-PAGE gel and immunoblotted with HA (Sigma, 11867431001), β-actin (Sigma, A1978), Cdc20 (Cell signaling, 14866S), Plk1 (Cell signaling, 4513T), c-Myc (Novus Biologicals, NB600-302), Mcm2 (Cell signaling, 3619T), Mcm5 (ProteinTech, 67049-1-Ig), and Mcm6 (ProteinTech, 13347-2-AP) antibodies with 1:1000 dilution.

Immunohistochemistry (IHC) staining. Paraffin-embedded primary tumor samples were processed as previously noted³². Slides were incubated at 4° C. overnight with Ki67 (Leica Biosystem, Ki67-MM1-L-CE-S), cleaved caspase-3 (Cell signaling, 9661S), Cdc20 (Cell signaling, 14866S), Plk1 (Cell signaling, 4513T) or c-Myc (Novus Biologicals, NB600-302) antibodies with 1:100 dilution. Following washes with PBS, slides were stained as described before³². Images were taken with Carl Zeiss Zen (version 3.0) and processed with ImageJ (bundled with Java 1.8.0_172).

NGS and GSEA. For next-generation sequencing (NGS), age matched PyMT;UBC-Cre^{ERT+/-};Mtdh^{fl/fl} female mice with similar tumor burdens were treated and the tumors were collected. For spheres, 100 k mammary epithelial cells were seeded into each well of the 6-well low attachment plates. Five days after seeding, spheres were formed and were treated with vehicle or 200 μM of C26-A6 for another one week and the spheres were harvested. Total RNA samples were prepared from the tumors or spheres using RNAeasy kit (Qiagen). The RNA-seq libraries were prepared, examined and raw reads were processed as previously described^{7,45}.

GSEA v3.0 was used for gene set enrichment analysis^{48,49}. Normalized gene expression data were pre-ranked based on the differences of expression (fold changes). SND1_CPT_UP signature was extracted from a previous study⁴.

Luciferase-based screening. Seed HEK293T cells on 3×15 cm dish at 18-24 hr before transfection with 7-8×10⁶ cells per dish targeting 70-80% confluence when start transfection. 20 μg of pCDNA3.1-SND1-NLuc, pCDNA3.1-CLuc-MTDH, or 2 μg of pCDNA3.1-NLuc-CLuc plasmids that described above were transfected to each dish. 72 hr after transfection the cells were lysed with 5 ml of luciferase lysis buffer (2 mM EDTA, 20 mM DTT, 10% glycerol, 1% Triton X-100, 25 mM Tris base, adjusted to pH7.8 with H₃PO₄) at 4° C. for 20 min. The protein lysates were centrifuged at 13,000 rpm for 10 min, and the supernatant were collected.

For small chemical compound screening, white, flat-bottom, solid 384-well plates were used. Compounds were added into each well as 0.1 μl of 10 mM DMSO solution (or same amount of DMSO, serve as control). The first and last columns were free, and MTDH wild type (PNSDWNAPAEEWGNW) or mutant (PNSDANAPAEEAGNW) peptides were added as positive and negative controls right before screening. Same amount of MTDH-CLuc and SND1-NLuc were pre-mixed at 4° C. for 30 min to generate split-luc. 5 μl of split- or linked-luc was added into each well. 15 μl of luciferase assay buffer (25 mM Glycylglycine pH 7.8, 15 mM K₃PO₄ pH 7.8, 15 mM MgSO₄, 4 mM EGTA, 2 mM ATP added just before use, 10 mM DTT added just before use and 1 mM D-Luciferin added just before use) was then added into each well to get a 20 μl reaction system with 50 μM of compounds (peptides) in each well. The plates were incubated at 4° C. for 1 hr and luciferase activity at each well was measured.

The inhibitory efficiency of each compound was calculated as following: (signal at DMSO well−signal at compound well)/signal at DMSO well. MTDH wild type or mutant peptide in each plate was served as positive and negative controls to monitor the data quality of each plate.

FRET-based screening. To perform FRET assay, purified CFP-MTDH (386-407aa) and TC-SND1 (16-339aa) proteins were reconstituted in FRET buffer (25 mM Tris-HCl pH8.0, 150 mM NaCl, 3 mM DTT, 2% DMSO). 384-well plate (Corning, black, flat bottom. Catalog number 3575) was used for this assay and the compounds/peptides were distribute into each well as above. 0.065 μl of CFP-MTDH was added to 8.9755 μl of FRET buffer, and then transferred the mixture to each well. Incubate the plate for 5 min at room temperature, avoid light. 1.86 μl of TC-SND1, 0.024 μl of FIAsH-EDT2 Labeling reagent (TC-FlAsH™ II In-Cell Tetracysteine Tag Detection Kits, Cat #T34561) together with 8.9755 μl of FRET buffer was mixed and then added to each well. The plate was measured with excitation weave length of 450 nm and emission wavelength of 495 nm and 535 nm.

The inhibitory efficiency was calculated as following: After subtracting the value of DMSO background, the emission of CFP-MTDH at 495 nm is considered as D, the emission of TC-SND1+FIAsH is A, the emission of CFP-MTDH+ TC-SND1+FIAsH mixture is DA, the efficiency is calculated as 1-(DA-A)/D. Similarly, MTDH wild type or mutant peptide in each plate was served as positive and negative controls to monitor the data quality of each plate.

Candidate selection. Singleton small molecule library was screened with split-luciferase for two rounds (R1 and R2). The candidates showed inhibitory efficiency equal or greater than 0.4 were chosen and repeated twice with split-luc, linked-luc, and FRET assay. The average of inhibitory efficiency from each assay was calculated (average inhibitory of split-luc was consider as R3). Compounds were selected if they fall into any of the following criteria: 1) The inhibitory efficiency in R1 and R2 were normalized with the linked-luc average value. The candidates still gave greater than 0.4 inhibitory efficiency after normalization in both rounds (R1/Linked-luc avg.>0.4)&(R2/Linked-luc avg.>0.4); 2) The inhibitory efficiency in R1 and R3 were normalized with the linked-luc average value. The candidates still gave greater than 0.4 inhibitory efficiency after normalization in both rounds (R1/Linked-luc avg.>0.4)&(R3/Linked-luc avg.>0.4); 3) The inhibitory efficiency in R2 and R3 were normalized with the linked-luc average value. The candidates still gave greater than 0.4 inhibitory efficiency after normalization in both rounds (R2/Linked-luc avg.>0.4)&(R3/Linked-luc avg.>0.4); 4) Only the candidates with the inhibitory efficiency between −0.2 to 0.2 in linked-luc assay were considered. The candidates were selected if they have split-luc inhibitory efficacy greater than 0.4 in both: a) R1 and R2; b) R2 and R3; c) R1 and R3; 5) The candidates showed inhibitory efficiency greater than 0.06 in both rounds of FRET assays; 6) The candidates share structure similarity with the above selected ones. 52 compounds were selected with these criteria.

The list of candidates was confirmed with split- and linked-luc assay and FRET assay again. Candidates were selected if they fall into any of the following criteria: 1) The inhibitory efficiency of split-luc is greater than 0.9; 2) The inhibitory efficiency of split-luc that normalized with linked-luc is greater than 0.2; or 3) Inhibitory efficiency in FRET assay is greater than 0.2. 12 candidates were selected after filter with these criteria.

C26s cell permeability test. The cell permeability of C26-A2 and A6 were determined with monolayer of Caco-2 cells with both the apical-to-basolateral (A-to-B) and basolateral-to-apical (B-to-A) directions by Absorption Systems LLC Detailed protocol can be found in previous studies.

Tamoxifen, C26-A6 and Paclitaxel for in vivo treatment. Tamoxifen (Tmx) (Sigma-Aldrich, T5648) was reconstituted with corn oil (Sigma-Aldrich, C8264) at 20 mg/ml. After 1 hr of shaking at 37° C. the solution is ready for use. For the treatment, indicated mice were injection with 60 mg/kg of the solution via i.p. for 5 constitutive days.

C26-A6 was synthesized by WuXi AppTec. Purity was confirmed by LC-MS/MS (>98%). The compound was reconstituted with DMSO at 50 mg/ml and was mixed with cremophor at 1:1 ratio. Right before use, The C26-A6 stock was diluted with PBS at 1:5 ratio. Mice were injected via tail-vein (T.V.). For the mice that T.V. injection was failed due to the high frequency treatment at late timepoints, i.p. injection with 2× dose was performed instead.

Paclitaxel (Sigma-Aldrich, T7402) stock was prepared at 50 mg/ml with ethanol and was diluted with cremophor with 1:1 ratio. For mouse treatment, ethanol:cremophor paclitaxel stock was diluted with PBS with 1:5 ratio by vortex right before use (no precipitates were observed).

Statistics and reproducibility. Animals were excluded only if they died or have to be euthanized according to the IACUC protocol. No statistical method was used to predetermine sample size. Data collection and analysis were not performed blinded to the conditions of the experiments. For in vivo experiments, animals were randomized and treated as indicated in each experiment. For in vitro experiments, all samples were analyzed equally with no sub-sampling; therefore, there was no requirement for randomization. Statistical analyses were indicated in figure captions. Error bars indicate means±SEM. GraphPad Prism software (version 7) was used for statistical calculations.

Data availability. All RNA sequencing data generated in this study have been deposited as a superseries at the NCBI Gene Expression Omnibus with the accession code GSE174630.

### REFERENCES

1 Nguyen, D. X., Bos, P. D. & Massague, J. Metastasis: from dissemination to organ-specific colonization. Nature reviews 9, 274-284 (2009).
2 Hu, G., Wei, Y. & Kang, Y. The multifaceted role of MTDH/AEG-1 in cancer progression. Clin Cancer Res 15, 5615-5620 (2009).
3 Wan, L. & Kang, Y. Pleiotropic roles of AEG-1/MTDH/LYRIC in breast cancer. Advances in cancer research 120, 113-134 (2013).
4 Wan, L. et al. MTDH-SND1 interaction is crucial for expansion and activity of tumor-initiating cells in diverse oncogene- and carcinogen-induced mammary tumors. Cancer cell 26, 92-105 (2014).
5 Wan, L. et al. Genetic ablation of metadherin inhibits autochthonous prostate cancer progression and metastasis. Cancer research 74, 5336-5347, doi:10.1158/0008-5472.CAN-14-1349 (2014).
6 Jariwala, N. et al. Role of the staphylococcal nuclease and tudor domain containing 1 in oncogenesis (review). International journal of oncology 46, 465-473 (2014).
7 Shen, M. et al. Therapeutic Targeting of Metadherin Suppresses Colorectal and Lung Cancer Progression and Metastasis. Cancer research 81, 1014-1025, doi:10.1158/0008-5472.CAN-20-1876 (2021).
8 Blanco, M. A. et al. Identification of staphylococcal nuclease domain-containing 1 (SND1) as a Metadherin-interacting protein with metastasis-promoting functions. The Journal of biological chemistry 286, 19982-19992 (2011).
9 Yoo, B. K. et al. Increased RNA-induced silencing complex (RISC) activity contributes to hepatocellular carcinoma. Hepatology 53, 1538-1548, doi:10.1002/hep.24216 (2011).
10 Jariwala, N. et al. Role of the staphylococcal nuclease and tudor domain containing 1 in oncogenesis (review). International journal of oncology 46, 465-473, doi:10.3892/ijo.2014.2766 (2015).
11 Van Nostrand, E. L. et al. A large-scale binding and functional map of human RNA-binding proteins. Nature 583, 711-719, doi:10.1038/s41586-020-2077-3 (2020).
12 Guo, F. et al. Structural insights into the tumor-promoting function of the MTDH-SND1 complex. Cell reports 8, 1704-1713 (2014).
13 Du, J. et al. PDK1 promotes tumor growth and metastasis in a spontaneous breast cancer model. Oncogene 35, 3314-3323, doi:10.1038/onc.2015.393 (2016).
14 Canovas, B. et al. Targeting p38alpha Increases DNA Damage, Chromosome Instability, and the Anti-tumoral Response to Taxanes in Breast Cancer Cells. Cancer cell 33, 1094-1110 e1098, doi:10.1016/j.ccell.2018.04.010 (2018).
15 Klein, A. et al. Comparison of gene expression data from human and mouse breast cancers: identification of a conserved breast tumor gene set. International journal of cancer 121, 683-688 (2007).
16 Kretschmer, C. et al. Identification of early molecular markers for breast cancer. Molecular cancer 10, 15 (2011).
17 Maroulakou, I. G., Anver, M., Garrett, L. & Green, J. E. Prostate and mammary adenocarcinoma in transgenic mice carrying a rat C3(1) simian virus 40 large tumor antigen fusion gene. Proceedings of the National Academy of Sciences of the United States of America 91, 11236-11240 (1994).
18 Cho, R. W. et al. Isolation and molecular characterization of cancer stem cells in MMTV-Wnt-1 murine breast tumors. Stem cells (Dayton, Ohio) 26, 364-371 (2008).
19 Hollern, D. P. & Andrechek, E. R. A genomic analysis of mouse models of breast cancer reveals molecular features of mouse models and relationships to human breast cancer. Breast Cancer Res 16, R59 (2014).
20 Pond, A. C. et al. Fibroblast growth factor receptor signaling dramatically accelerates tumorigenesis and enhances oncoprotein translation in the mouse mammary tumor virus-Wnt-1 mouse model of breast cancer. Cancer research 70, 4868-4879 (2010).
21 Luker, K. E. et al. Kinetics of regulated protein-protein interactions revealed with firefly luciferase complementation imaging in cells and living animals. Proceedings of the National Academy of Sciences of the United States of America 101, 12288-12293 (2004).
22 Paulmurugan, R. & Gambhir, S. S. Combinatorial library screening for developing an improved split-firefly luciferase fragment-assisted complementation system for studying protein-protein interactions. Analytical chemistry 79, 2346-2353 (2007).
23 Porter, J. R., Stains, C. I., Jester, B. W. & Ghosh, I. A general and rapid cell-free approach for the interrogation of protein-protein, protein-DNA, and protein-RNA interactions and their antagonists utilizing split-protein reporters. Journal of the American Chemical Society 130, 6488-6497 (2008).
24 Hoffmann, C. et al. A FlAsH-based FRET approach to determine G protein-coupled receptor activation in living cells. Nature methods 2, 171-176 (2005).
25 Kang, Y. et al. A multigenic program mediating breast cancer metastasis to bone. Cancer cell 3, 537-549 (2003).
26 Groftehauge, M. K., Hajizadeh, N. R., Swann, M. J. & Pohl, E. Protein-ligand interactions investigated by thermal shift assays (TSA) and dual polarization interferometry (DPI). Acta crystallographica 71, 36-44 (2015).
27 Jerabek-Willemsen, M., Wienken, C. J., Braun, D., Baaske, P. & Duhr, S. Molecular interaction studies using microscale thermophoresis. Assay and drug development technologies 9, 342-353 (2011).
28 Wienken, C. J., Baaske, P., Rothbauer, U., Braun, D. & Duhr, S. Protein-binding assays in biological liquids using microscale thermophoresis. Nature communications 1, 100 (2010).
29 van Breemen, R. B. & Li, Y. Caco-2 cell permeability assays to measure drug absorption. Expert opinion on drug metabolism & toxicology 1, 175-185 (2005).
30 DeRose, Y. S. et al. Tumor grafts derived from women with breast cancer authentically reflect tumor pathology, growth, metastasis and disease outcomes. Nat Med 17, 1514-1520, doi:10.1038/nm.2454 (2011).
31 Esposito, M. et al. Bone vascular niche E-selectin induces mesenchymal-epithelial transition and Wnt activation in cancer cells to promote bone metastasis. Nature cell biology 21, 627-639 (2019).
32 Shen, M. et al. Tinagl1 Suppresses Triple-Negative Breast Cancer Progression and Metastasis by Simultaneously Inhibiting Integrin/FAK and EGFR Signaling. Cancer cell 35, 64-80 e67 (2019).
33 Kaur, P. et al. A mouse model for triple-negative breast cancer tumor-initiating cells (TNBC-TICs) exhibits similar aggressive phenotype to the human disease. BMC Cancer 12, 120, doi:10.1186/1471-2407-12-120 (2012).
34 Arkin, M. R., Tang, Y. & Wells, J. A. Small-molecule inhibitors of protein-protein interactions: progressing toward the reality. Chemistry & biology 21, 1102-1114 (2014).
35 Lage, K. Protein-protein interactions and genetic diseases: The interactome. Biochimica et biophysica acta 1842, 1971-1980 (2015).
36 Scott, D. E., Bayly, A. R., Abell, C. & Skidmore, J. Small molecules, big targets: drug discovery faces the protein-protein interaction challenge. Nature reviews 15, 533-550 (2016).
37 Jubb, H., Higueruelo, A. P., Winter, A. & Blundell, T. L. Structural biology and drug discovery for protein-protein interactions. Trends in pharmacological sciences 33, 241-248 (2012).
38 Surade, S. & Blundell, T. L. Structural biology and drug discovery of difficult targets: the limits of ligandability. Chemistry & biology 19, 42-50 (2012).
39 Tari, L. W. The utility of structural biology in drug discovery. Methods in molecular biology (Clifton, N.J 841, 1-27 (2012).
40 Jin, L., Wang, W. & Fang, G. Targeting protein-protein interaction by small molecules. Annual review of pharmacology and toxicology 54, 435-456 (2013).
41 Nero, T. L., Morton, C. J., Holien, J. K., Wielens, J. & Parker, M. W. Oncogenic protein interfaces: small molecules, big challenges. Nat Rev Cancer 14, 248-262 (2014).
42 Vassilev, L. T. et al. In vivo activation of the p53 pathway by small-molecule antagonists of MDM2. Science (New York, N.Y 303, 844-848 (2004).
43 Zhao, J. et al. Discovery and structural characterization of a small molecule 14-3-3 protein-protein interaction inhibitor. Proceedings of the National Academy of Sciences of the United States of America 108, 16212-16216 (2011).
44 Zimmermann, G. et al. Small molecule inhibition of the KRAS-PDEdelta interaction impairs oncogenic KRAS signalling. Nature 497, 638-642 (2013).
45 Jiang, Y. Z. et al. Preoperative measurement of breast cancer overestimates tumor size compared to pathological measurement. PLoS One 9, e86676, doi:10.1371/journal.pone.0086676 (2014).
46 Jiang, Y. Z. et al. Transcriptome Analysis of Triple-Negative Breast Cancer Reveals an Integrated mRNA-lncRNA Signature with Predictive and Prognostic Value. Cancer research 76, 2105-2114 (2016).
47 Liu, Y. R. et al. Comprehensive transcriptome analysis identifies novel molecular subtypes and subtype-specific RNAs of triple-negative breast cancer. Breast Cancer Res 18, 33 (2016).
48 Subramanian, A. et al. Gene set enrichment analysis: a knowledge-based approach for interpreting genome-wide expression profiles. Proceedings of the National Academy of Sciences of the United States of America 102, 15545-15550 (2005).
49 Mootha, V. K. et al. PGC-lalpha-responsive genes involved in oxidative phosphorylation are coordinately downregulated in human diabetes. Nature genetics 34, 267-273 (2003).
50 Kozlov, A. G., Galletto, R. & Lohman, T. M. SSB-DNA binding monitored by fluorescence intensity and anisotropy. Methods in molecular biology (Chfton, N.J 922, 55-83, doi:10.1007/978-1-62703-032-8_4 (2012).
51 Okoye-Okafor, U. C. et al. New IDH1 mutant inhibitors for treatment of acute myeloid leukemia. Nat Chem Biol 11, 878-886, doi:10.1038/nchembio.1930 (2015).

The foregoing example has been published in Nat Cancer 3, 43-59 (2022), the entire content of which is incorporated herein by reference in its entirety.

### Example 2

Pharmacological Disruption of the MTDH-SND1 Complex Enhances Tumor Antigen Presentation and Synergizes with Anti-PD-1 Therapy in Metastatic Breast Cancer

Abstract. Despite the increased overall survival rates, curative options for metastatic breast cancer remain limited. It has been shown that Metadherin (MTDH) is frequently overexpressed in poor prognosis breast cancer, where it promotes metastasis and therapy resistance through its interaction with Staphylococcal nuclease domain-containing 1 (SND1). Through genetic and pharmacological targeting of the MTDH-SND1 interaction, a key role for this complex is revealed in suppressing anti-tumor T cell responses in breast cancer. The MTDH-SND1 complex reduces tumor antigen presentation and inhibits T cell infiltration and activation by binding to and destabilizing Tap1/2 mRNAs, which encode key components of the antigen presentation machinery. Following small molecule compound C26-A6 treatment to disrupt the MTDH-SND1 complex, enhanced immune surveillance and sensitivity to anti-PD-1 therapy in preclinical models of metastatic breast cancer is observed, in support of this combination therapy as a viable approach to increase immune checkpoint blockade therapy responses in metastatic breast cancer.

Introduction. Despite recent advances in immunotherapy for leukemia, melanoma, lung cancer, bladder cancer and other cancers, clinical success of immunotherapy for metastatic breast cancer has been limited so far¹⁻³. Breast tumors are not as inherently immunogenic as other solid malignancies such as melanoma. In particular, breast tumors that have metastasized may have developed multiple means to avoid immune detection and elimination. Thus, it is imperative for research efforts to focus on elucidating mechanisms to promote immune eradication of metastatic breast tumors through new immunotherapeutic approaches⁴⁻⁶

One gene that has been recently validated as a functional mediator of breast cancer initiation, metastasis and drug resistance is Metadherin (MTDH), which has been identified as a key target gene encoded in the 8q22 genomic gain frequently found in poor prognosis breast cancer⁷. As MTDH is overexpressed in >40% of human breast tumors⁷, this protein may be an ideal therapeutic target to develop new therapeutic approaches against metastatic or treatment-resistant breast cancer. Several studies have implicated MTDH in promoting tumor growth and chemoresistance through association with Staphylococcal nuclease domain-containing 1 (SND1)⁸⁻¹⁰. Example 1 describes identification of a class of small chemical inhibitors that disrupt the MTDH-SND1 complex. MTDH-SND1 inhibition by these compounds significantly reduced breast cancer progression and metastasis, and sensitized tumors to chemotherapy, supporting the therapeutic potential of this new class of inhibitors¹².

Although the critical function of MTDH in breast cancer progression and metastasis has been validated by genetic and pharmacological approaches, the underlying molecular mechanism of the pro-malignant function of MTDH-SND1 has not been fully characterized. In the present study, a previously unknown function of MTDH-SND1 in promoting immune evasion by suppressing tumor antigen presentation is revealed.

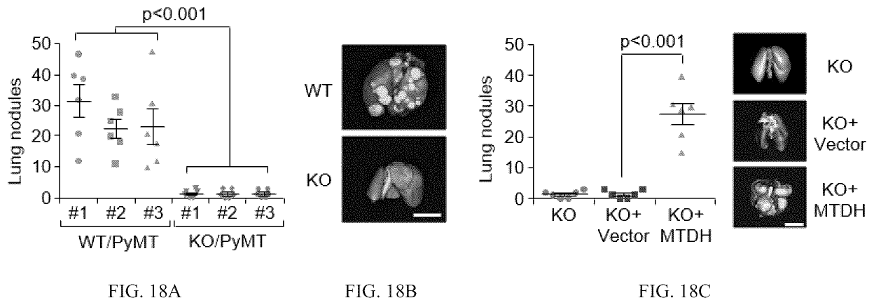

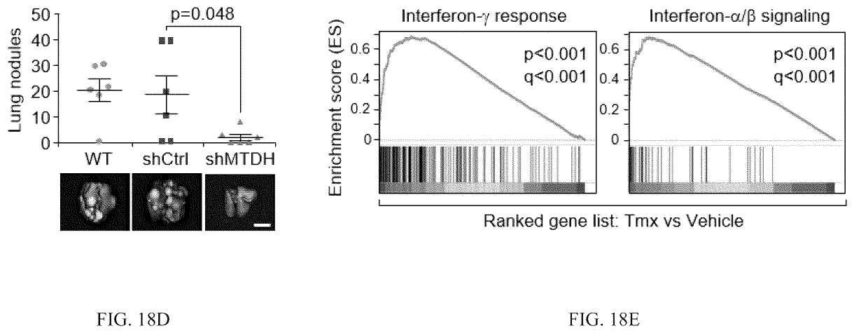

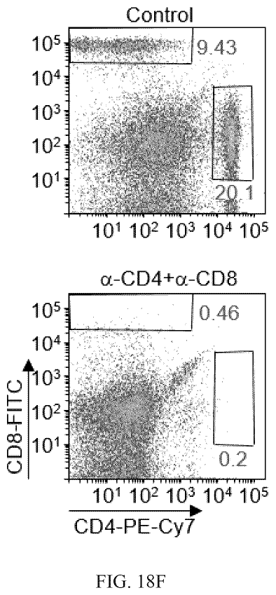

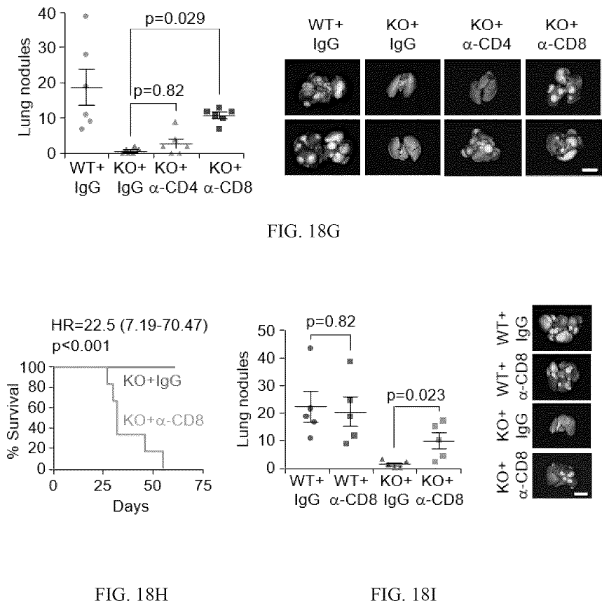

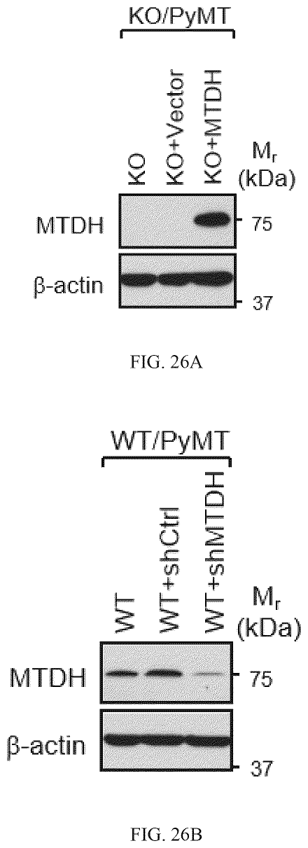

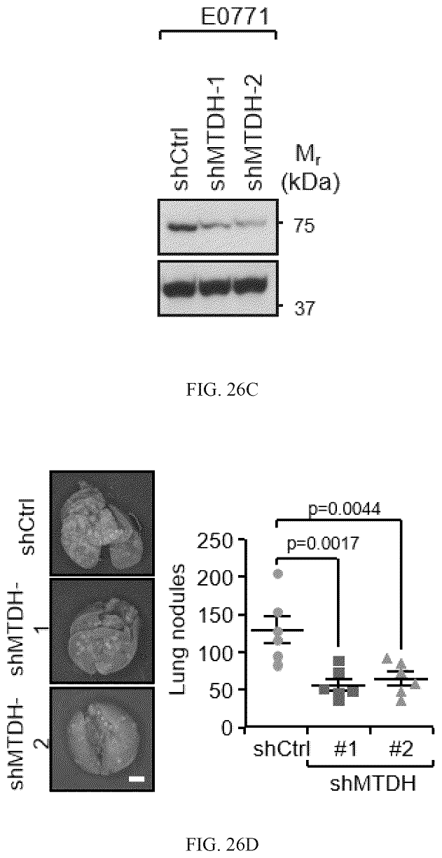

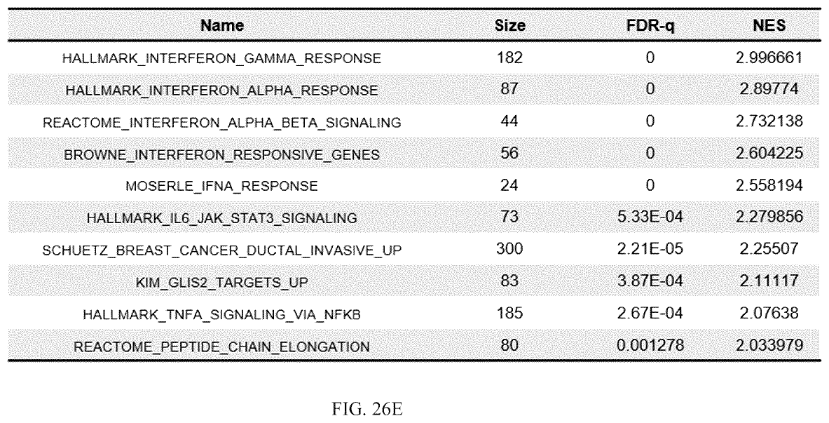

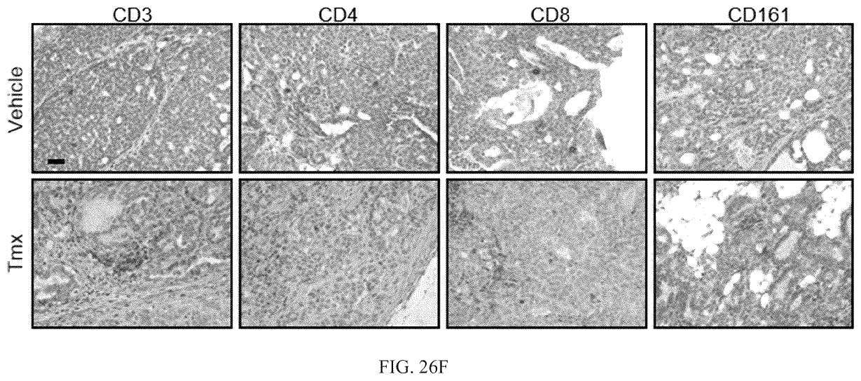

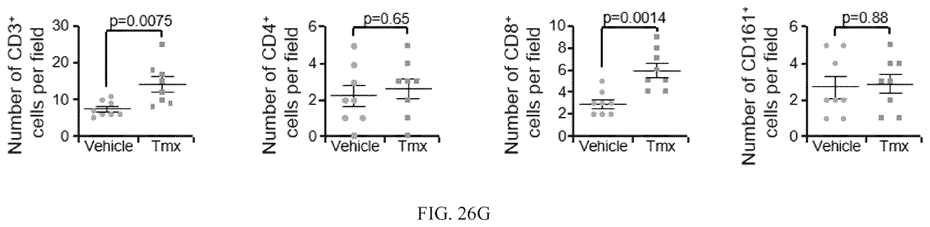

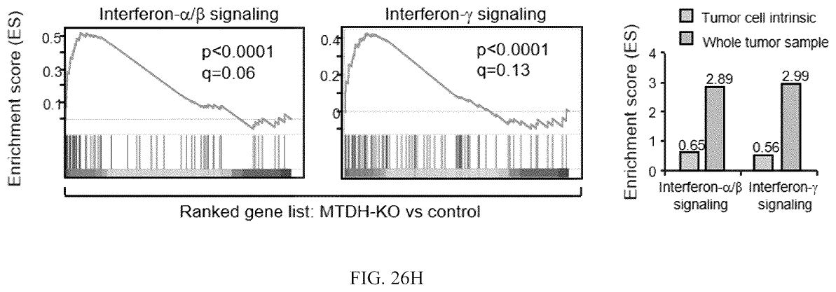

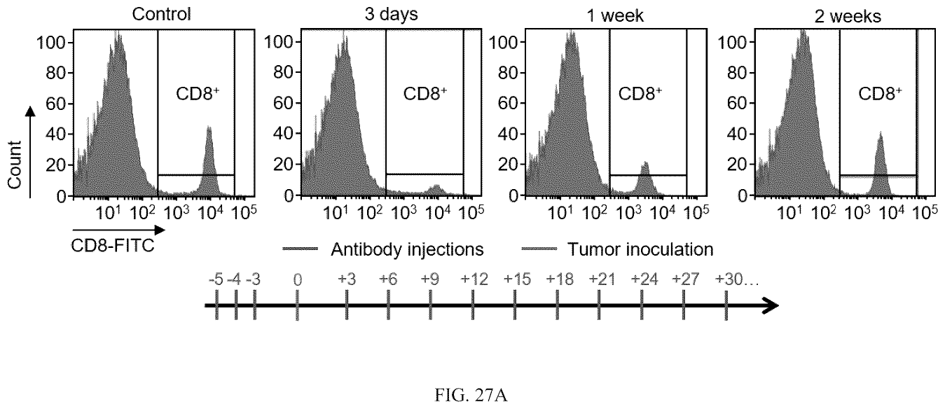

Results: MTDH promotes breast cancer immune evasion during metastasis. To explore the role of MTDH in breast cancer metastasis, MTDH knockout (FVB.Mtdh^{-/-}) mice crossed with the FVB.MMTV-PyMT tumor model was utilized as previously reported¹⁰. Several metastatic PyMT tumor cell clones were isolated from tumors spontaneously arising from PyMT;Mtdh^{+/+} (WT) or PyMT;Mtdh^{-/-} (KO) mice, and three WT/PyMT and KO/PyMT cells lines were generated. The propensity for experimental lung metastasis of these cell lines was evaluated by tail vein injections into wild type female FVB mice. MTDH-WT (WT/PyMT) tumor cells generated large numbers of metastatic lung nodules in multiple experiments with different cell lines. In contrast, MTDH-KO (KO/PyMT) tumor cell lines failed to establish significant metastases (FIGS. 18A, 18B). When MTDH expression was restored in the KO cells by lentiviral expression of the wild type Mtdh cDNA (FIG. 26A), increased numbers of metastatic lung nodules, which were comparable in number to animals injected with the WT tumor cells, were observed (FIGS. 18A, 18C). Similarly, depletion of MTDH expression by lentiviral expression of Mtdh-targeting short hairpin RNA (shRNA) in the MTDH-WT PyMT tumor cells decreases measurable metastatic lung nodules (FIGS. 26B and 26d). To further confirm this finding, mouse mammary tumor cell line E0771, which was derived from a spontaneously occurring breast tumor in the C57BL/6 background¹³ was employed in a similar assay. E0771 cells with or without MTDH knockdown (KD) (FIG. 26C) were injected into female C57BL/6 mice via tail vein. Consistently, MTDH KD significantly attenuates lung metastasis (FIG. 26D). These results demonstrate that the status of MTDH expression strongly influences the metastatic potential of breast cancer cells.

As presented in Example 1, to evaluate the therapeutic potential of targeting MTDH, a tamoxifen (Tmx)-inducible Mtdh knockout model PyMT; UBC-Cre^{ERT+/-}/−;Mtdh^{fl/fl} was generated¹². In these mice, the floxed Mtdh alleles were excised by tamoxifen-activated Cre after the mice were injected with 60 mg/kg of Tmx for 5 constitutive days. Such a dosing regimen of tamoxifen is commonly used in conditional KO of genes of interest in mouse models of breast cancer including MMTV-PyMT and been shown to have no direct effect on tumor growth and metastasis^{14,15}. Taking advantage of the strain, tumors with/without Mtdh acute knockout and after treatments with vehicle or 60 mg/kg of Tmx were collected for RNA extraction and sequencing. Gene set enrichment analysis revealed significant enrichment of interferon related signatures in the ranked listed of up-regulated genes in Mtdh acute knockout tumors versus wild type tumors (FIGS. 26E and 18E), suggesting MTDH might promote metastatic breast cancer progression by suppressing immune responses to tumors. Consistent with this notion, in MTDH KO tumors, significant higher numbers of CD3⁺ and CD8⁺ T cells infiltration were observed (FIGS. 26F, 26G), suggesting that the observed enrichment of the interferon-related signatures might be due to the increased immune cell infiltration in the local tumor microenvironment. To further test this, in vitro cultured PyMT;UBC-Cre^{ERT+/-};Mtdh^{fl/fl} tumorspheres were treated with vehicle (Ctrl) or 4-hydroxytamoxifen (4-OHT) to KO MTDH. The tumorspheres were then collected for RNA sequencing followed by gene set enrichment analysis. Although interferon related signatures showed a trend to be enriched in MTDH KO tumorspheres, the overall enrichment scores are much lower as compared to that from in vivo tumor samples (FIG. 26H), indicating that the enriched interferon-related signatures are mostly caused by changes in the tumor microenvironment. Collectively, these results suggest that MTDH KO induces a more immunogenic tumor microenvironment.

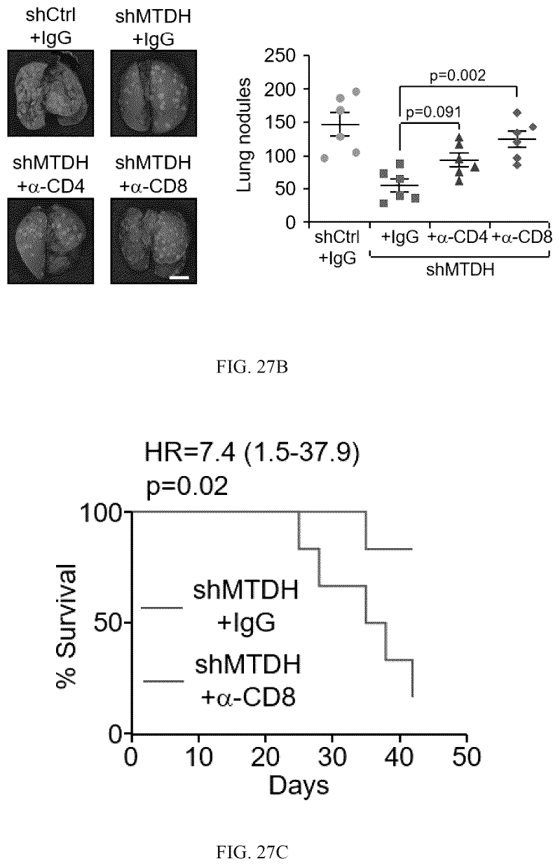

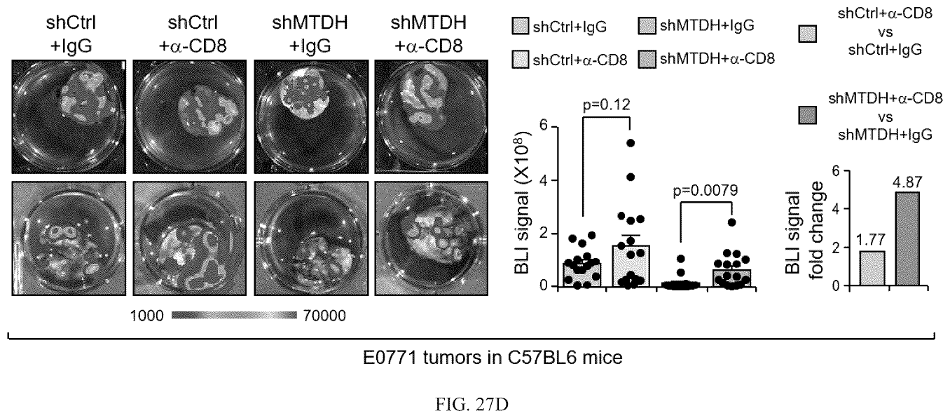

Given the importance of T lymphocytes in the tumor immune responses, the metastatic behavior of MTDH-WT and KO PyMT tumor cells in mice with T cell depletion was evaluated. To this end, anti-CD4 and anti-CD8 neutralizing antibodies were purified from hybridoma cell lines and immune-competent FVB mice were pretreated with these T-cell depleting antibodies. The antibodies ablated greater than 97% of both CD4⁺ and CD8⁺ T cells as assessed by flow cytometry compared with isotype control (FIG. 18F), and such depletion was sustained for approximately 3 days. The relevant T cell population returned to normal levels 1-2 weeks after the initial injections (FIG. 27A). Therefore, the antibody injection scheme in FIG. 27A (antibody injection every 3 days) was used to maintain T-cell depletion over the course of experimental metastasis studies. It was found that when CD4 and CD8 T cells were depleted in FVB mice, intravenous injection of MTDH-KO PyMT tumor cells generated more metastatic nodules than in wild type mice. The difference was particularly striking when CD8⁺ cytotoxic T cells were depleted (FIG. 1-2g). Importantly, when the experiment was repeated to directly compare the survival of mice intravenously injected with MTDH-KO PyMT tumor cells, depletion of CD8⁺ T cells dramatically reduced the overall survival of the mice (FIG. 18H). Similar CD4⁺ and CD8⁺ T cell depletion assays were performed with the E0771 model. Again, CD8⁺ T cell depletion restored lung metastasis of E0771-MTDH-KD cells and reduced the survival of C57BL/6 mice injected with E0771-MTDH-KD cells compared to control (FIG. 27B, 27C). To further confirm the role of MTDH in spontaneous lung metastasis, CD8⁺ T cells in FVB mice were depleted and PyMT tumor cells with or without MTDH KO were injected into the mammary fat pad to generate primary mammary gland tumors. MTDH-KO tumors developed significantly more lung metastasis when CD8⁺ T cells were depleted, whereas the depletion did not significantly alter spontaneous metastasis of MTDH-WT PyMT tumor cells (FIG. 18I). Similar observation was made using the E0771 models in C57BL/6 mice (FIG. 27D). Of note, although increased trend of metastasis in control groups upon CD8⁺ T depletion was observed, the difference did not reach statistical significance, and the increase was much lower as compared to MTDH KD groups (FIG. 27D). Overall, the results indicate that MTDH allows metastatic tumor cells to evade the attack from CD8⁺ cytotoxic T cells, and as a consequence, promotes metastatic progression of breast cancer.

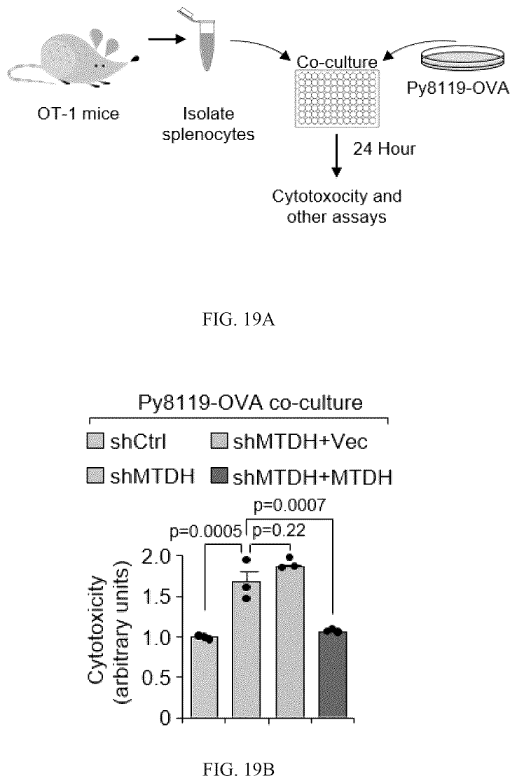

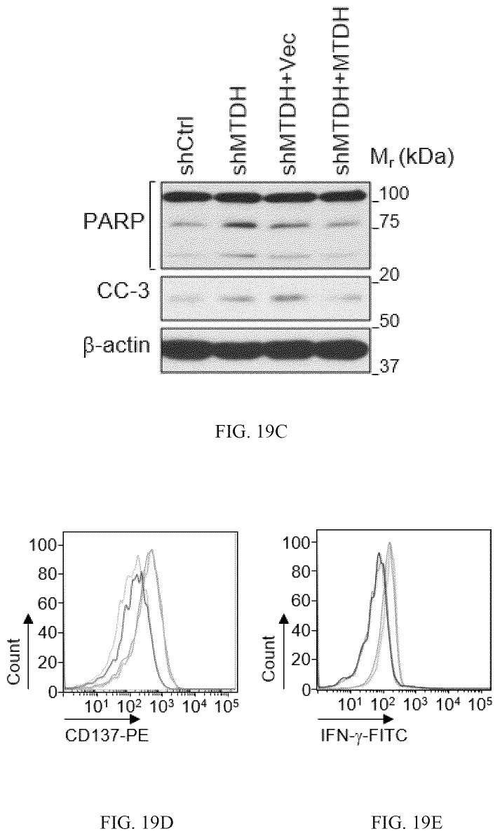

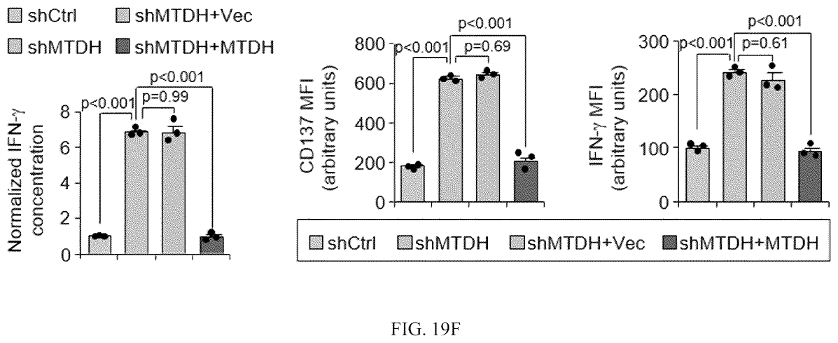

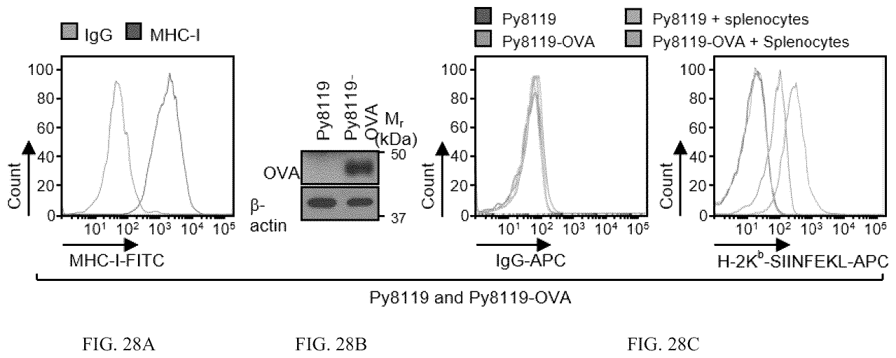

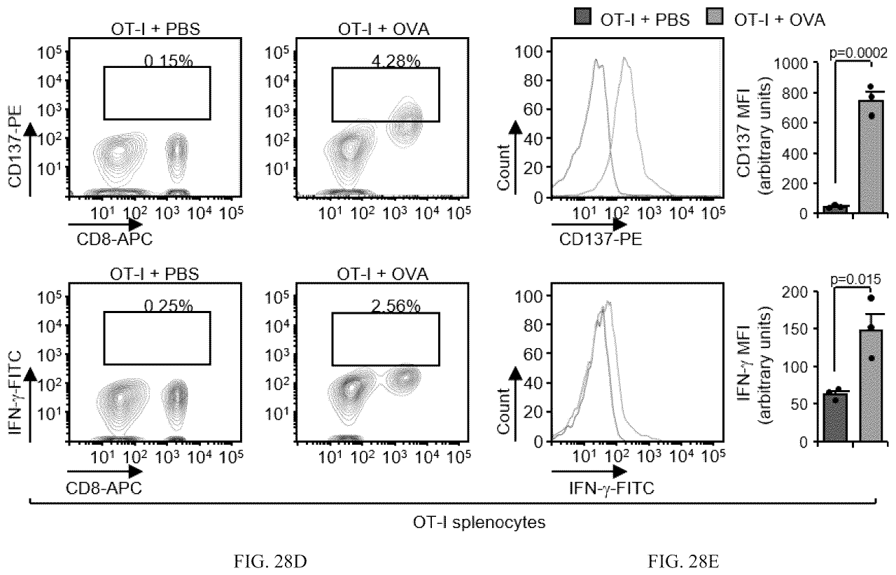

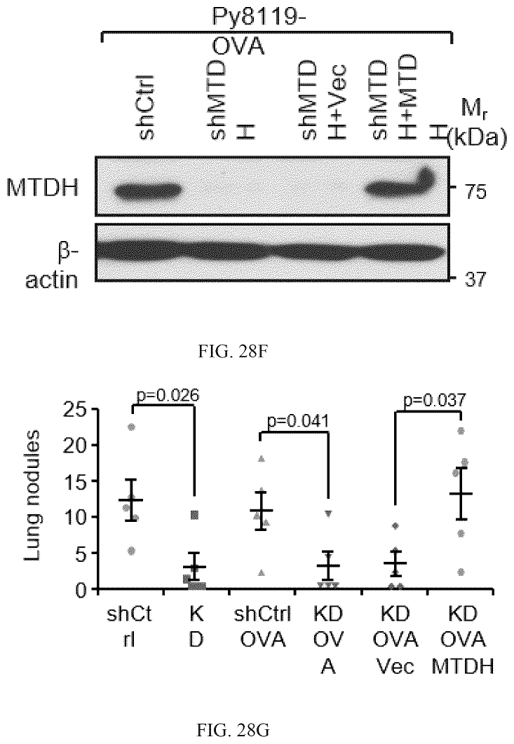

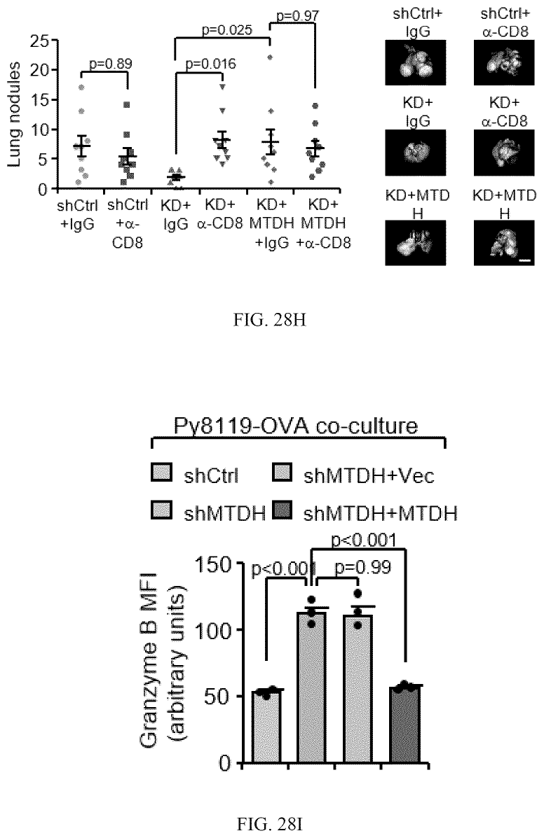

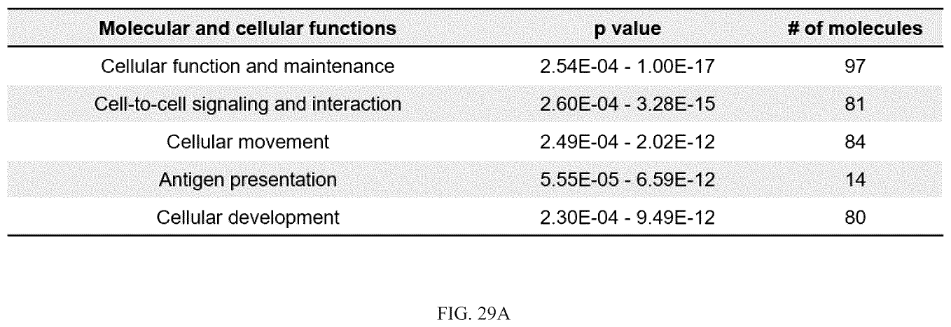

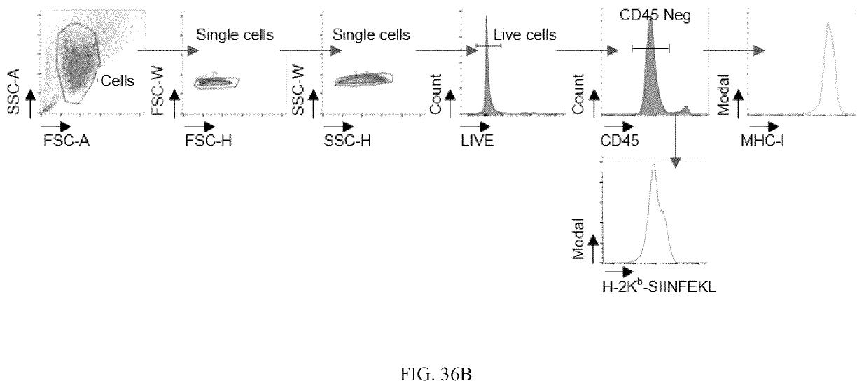

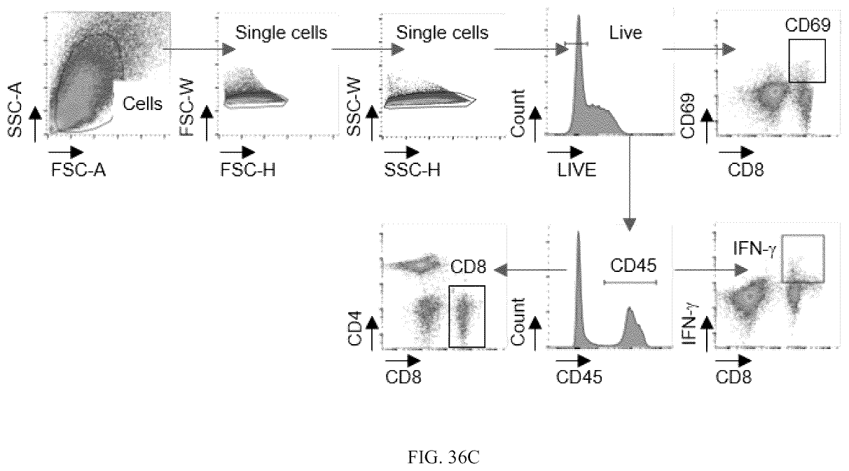

MTDH protects tumor cells from the killing of CD8⁺ T cells. To more accurately evaluate the observed MTDH-mediated immune phenotype, the widely used ovalbumin (OVA)/OT-I antigen expression system was used to elicit strong epitope-specific immune responses against MTDH-WT and KD tumor cells expressing the OVA protein. MHC class I haplotype compatible PyMT tumor cell line Py8119 that was derived from C57BL/6 mice was obtained¹⁶. Unlike the FVB PyMT cells used in the experiments described above, the Py8119 expresses the MHC class I allele H-2K^{b}, and can efficiently process and present the dominant antigen peptide (OVAp257) that is recognized by T cells from OT-I mouse^{17,18}. The MHC class I surface presentation in Py8119 cells and the OVAp257 surface presentation in OVA overexpressed Py8119 cells were validated (FIGS. 28A-28C). Moreover, the antibody recognizing OVA complexes (H-2K^{b}-SIINFEKL) has been systematically compared with its isotype control to validate the specificity (FIG. 28C). An in vitro co-culture system was established by isolating immune cells from the spleen of OT-I mouse and then co-cultured them with OVA-expressing Py8119 tumor cells to analyze in vitro T-cell mediated killing of tumor cells (FIG. 19A). To this end, the immune cells that isolated from spleens of OT-I mice were first evaluated. As expected, the splenocytes were effectively activated by OVAp257 peptide treatment (FIGS. 28D, 28E,e; gating strategy shown in FIG. 36), confirming the recognition of OVA by OT-I T cells. Next, endogenous Mtdh was knocked down and rescued with wild type MTDH in Py8119-OVA cells (FIG. 28F). Consistent with the results described above, Py8119 model reproduced the lung metastasis phenotypes that was observed in PyMT and E0771 models, and moreover, OVA expression did not alter MTDH-induced immune evasion phenotype (FIG. 28G, 28H).

Next, an in vitro co-culture assay with the Py8119-OVA cell lines and OT-I cells was performed. CD8⁺ cytotoxic T cells that were isolated from OT-I splenocytes had stronger killing effects on Py8119-OVA cells with MTDH KD, which were abolished by wild type MTDH rescue (FIG. 19B, 19C), suggesting that MTDH in tumor cells protects them from immune clearance. Consistently, CD8⁺ T cells from MTDH-KD Py8119-OVA co-culture had stronger activation as indicated by higher CD137, IFN-γ, and Granzyme B expression (FIGS. 19D, 19E and FIG. 28I). To confirm the flow cytometry data, conditioned media from indicated co-culture conditions were also collected to test the IFN-γ concentration with ELISA. Again, significant higher IFN-γ was observed in the media that co-cultured with MTDH KD cells (FIG. 19F). Collectively, tumor MTDH inhibits the activation of immune cells, and therefore, prevents the immune clearance of tumor cells.

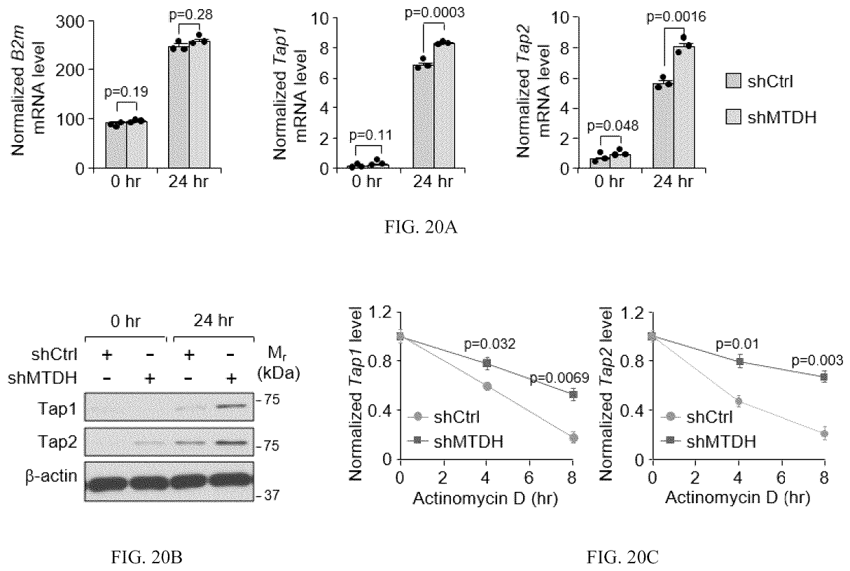

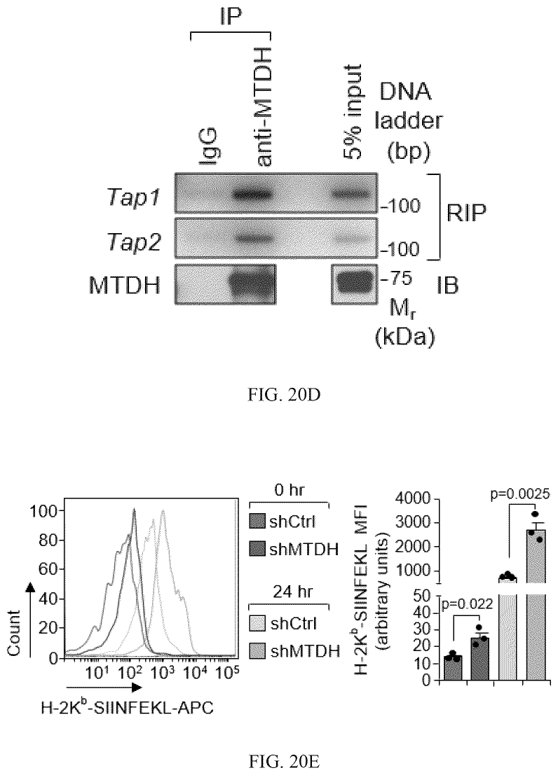

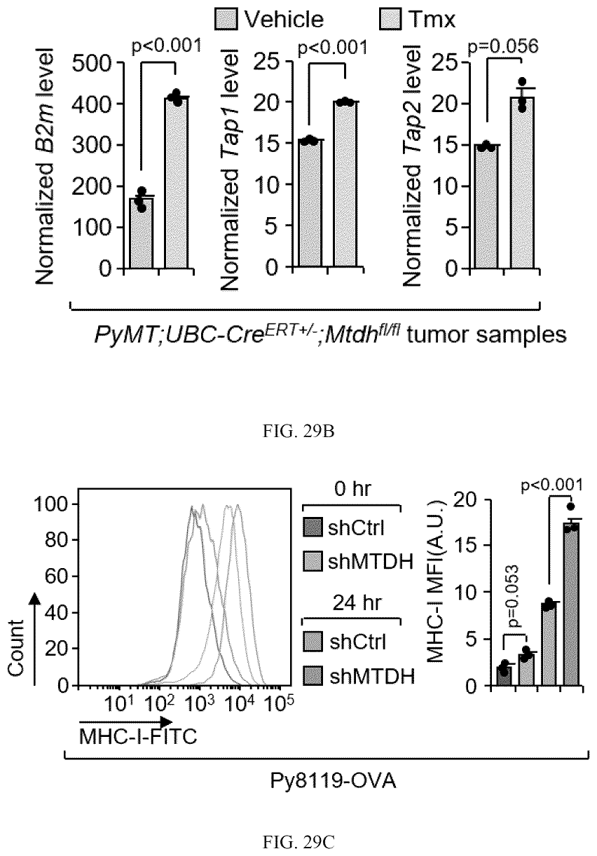

MTDH inhibits tumor antigen presentation. To uncover the mechanism underlying MTDH-mediated T cells suppression, RNA sequencing data was re-analyzed with Ingenuity Pathway Analysis by focusing on MTDH-KO up-regulated genes. Several pathways are significantly enriched in MTDH acute KO tumors (FIG. 29A), among which, the antigen presentation pathway is directly related to the phenotypes observed above. qRT-PCR analysis confirmed the notion that MTDH depletion in tumor context enhanced the mRNA expression level of several genes in the antigen presentation machinery, such as B2m, Tap1 and Tap2 (FIG. 29B)¹⁹, suggesting MTDH is involved in antigen presentation regulation. Next, the in vitro co-culture system was utilized again to investigate MTDH-mediated antigen presentation. Upon OT-I splenocytes co-culture, Tap1/2 in Py8119-OVA tumor cells were significantly elevated; moreover, MTDH KD Py8119-OVA tumor cells had higher Tap1/2 protein levels (FIGS. 20A, 20B). However, significant difference of B2m was not observed between MTDH-WT and KD Py8119-OVA tumor cells after co-culture (FIG. 20A). Of note, also observed was increased steady levels of Tap1/2 in MTDH KD Py8119-OVA tumor cells before the co-culture (FIG. 20A). Given the difference of RNA levels of Tap1/2, the stability of these two RNAs in the tumor cells was examined in co-culture. Py8119-OVA tumor cells with or without MTDH knockdown were co-cultured with OT-I splenocytes for 24 hours and then treated with 10 μg/ml of actinomycin D. The tumor cells were harvested at indicated time points after the treatment. Total RNA was extracted and qRT-PCR was performed to test Tap1/2 levels in the tumor cells. It was found that MTDH KD significantly attenuates the degradation of Tap1/2 (FIG. 20C). RNA-binding protein immunoprecipitation (RIP) assay further revealed that MTDH interacts with Tap1/2 (FIG. 20D), suggesting MTDH binds to Tap1/2 and promotes the degradation of these RNAs. Consistent with this molecular alteration, less OVA antigen and MIC-I complex presentation in MTDH-WT tumor cells was also observed compared to MTDH-KD cells before and after immune cell challenge (FIG. 20E and FIG. 29C).

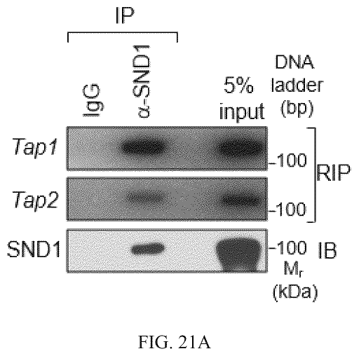

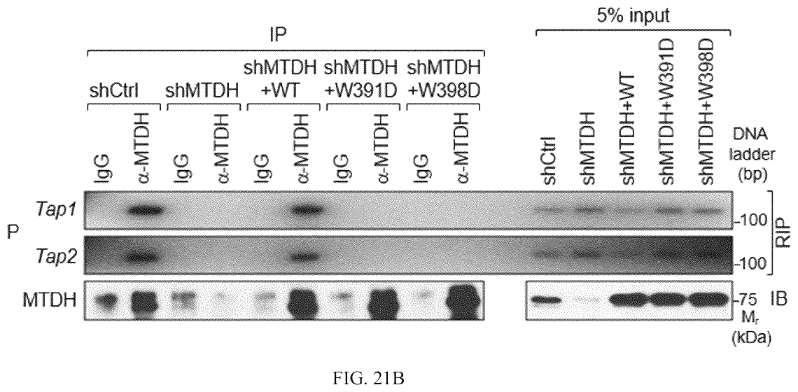

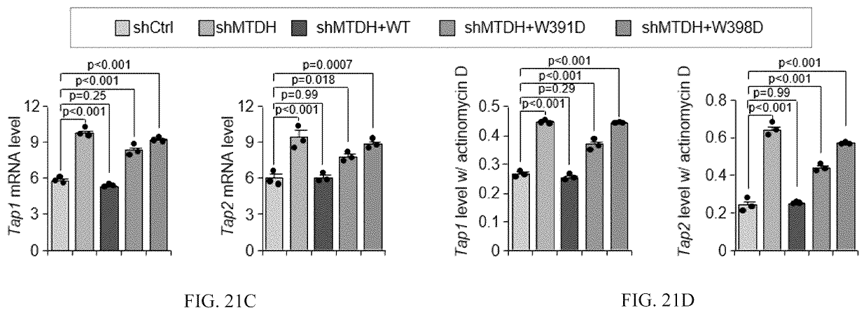

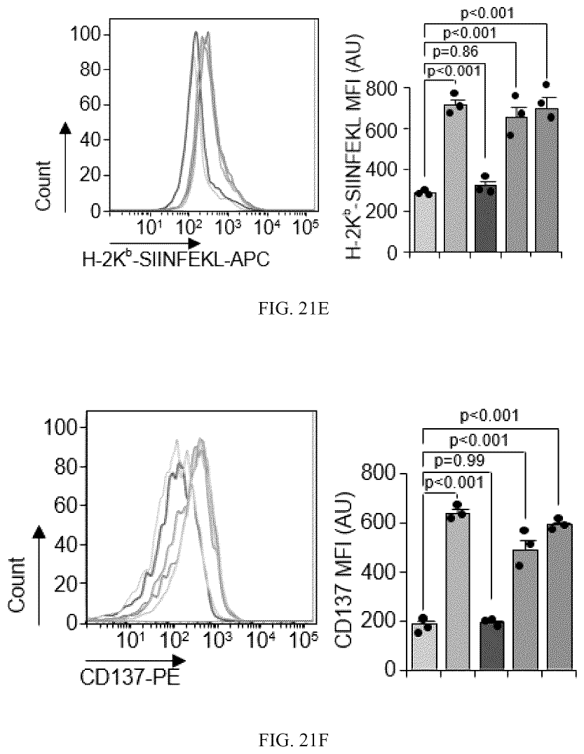

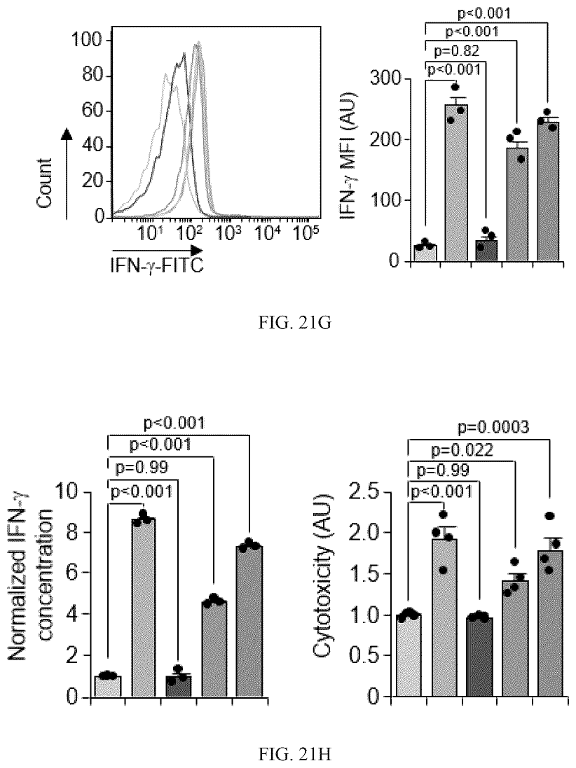

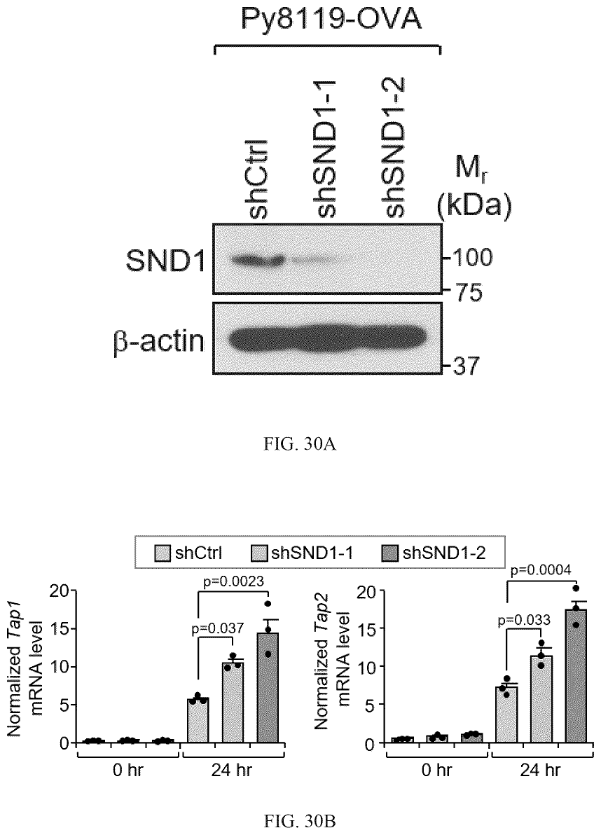

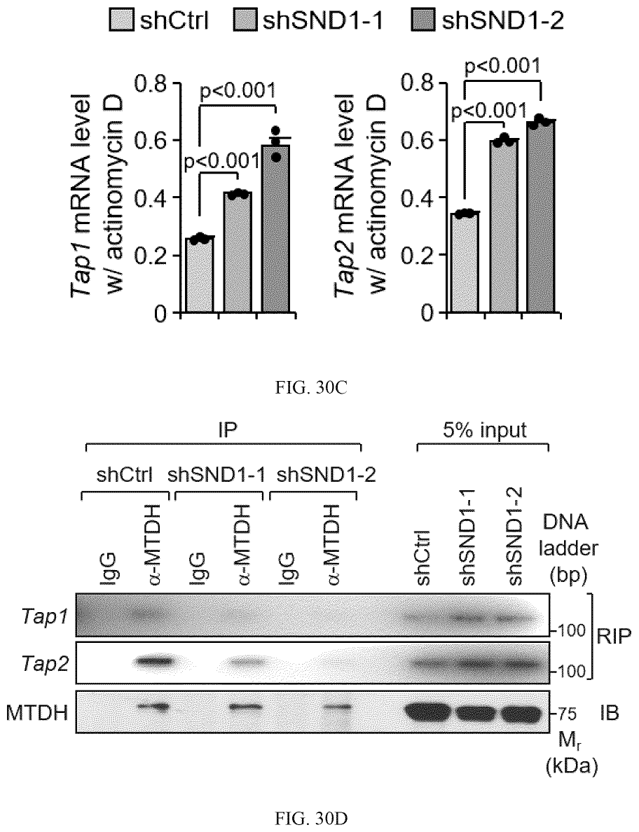

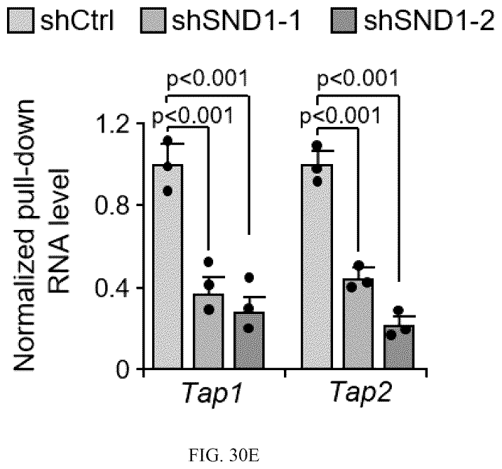

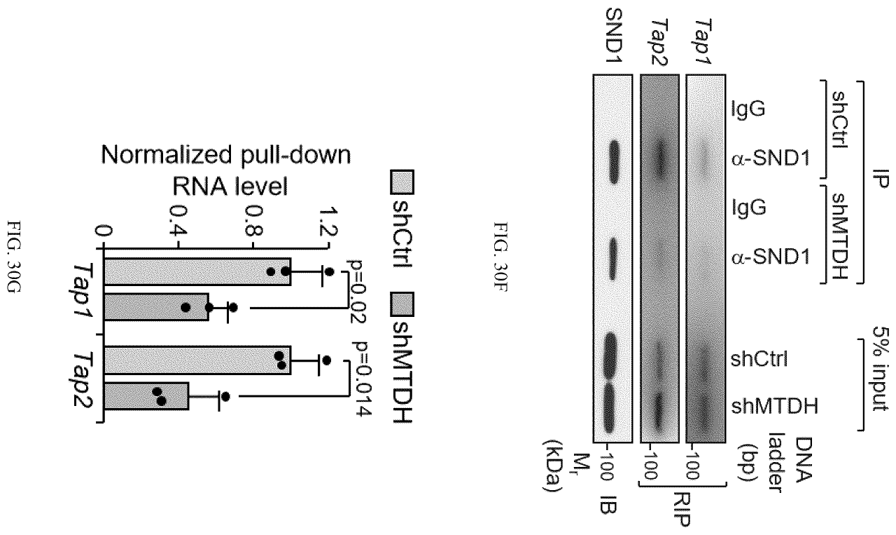

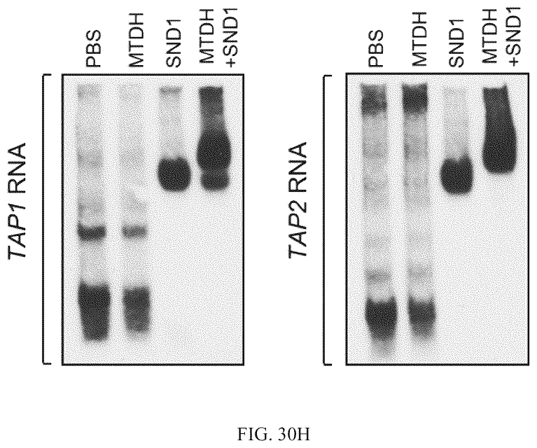

MTDH-SND1 interaction suppresses antigen presentation. Next, it was asked whether SND1 is also involved in MTDH-mediated suppression of antigen presentation. To address the question, Py8119-OVA SND1 KD cell lines were generated and were then subjected to OT-I splenocytes co-culture assay (FIG. 30A). Similar to MTDH-KD, SND1 KD significantly stabilized and increased the levels of Tap1/2 (FIGS. 30B, 30C). RIP assay also confirmed the interaction between SND1 and Tap1/2 (FIG. 21A). Interestingly, SND1 KD disrupted the interaction between MTDH and Tap1/2 (FIGS. 30D, 30E), indicating that SND1 is required for the binding of MTDH to these two RNAs. On the other hand, the binding between SND1 and Tap1/2 was also significantly reduced in the tumor cells with MTDH KD (FIGS. 30F, 30g), suggesting MTDH facilitates SND1's Tap1/2 binding. To investigate if MTDH or SND1 has direct interaction with Tap1/2, electrophoretic mobility gel shift assay was performed with in vitro transcribed human TAP1/2 mRNA and recombinant human MTDH and SND1 proteins. Consistent with in vivo findings, the interaction between MTDH and TAP1/2 mRNA was not detected in the absence of SND1, whereas SND1 alone could bind to TAP1/2 mRNA. Moreover, the addition of MTDH further enhanced the binding of the MTDH/SND1 complex to TAP1/2 RNA and induced a supershift (FIG. 30H). Taken together, the data suggest that MTDH and SND1 form a complex to bind Tap1/2 and promotes their degradation in vivo.

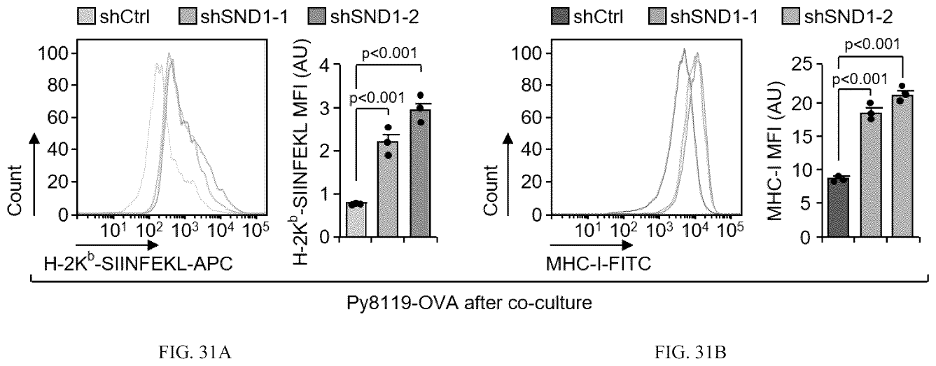

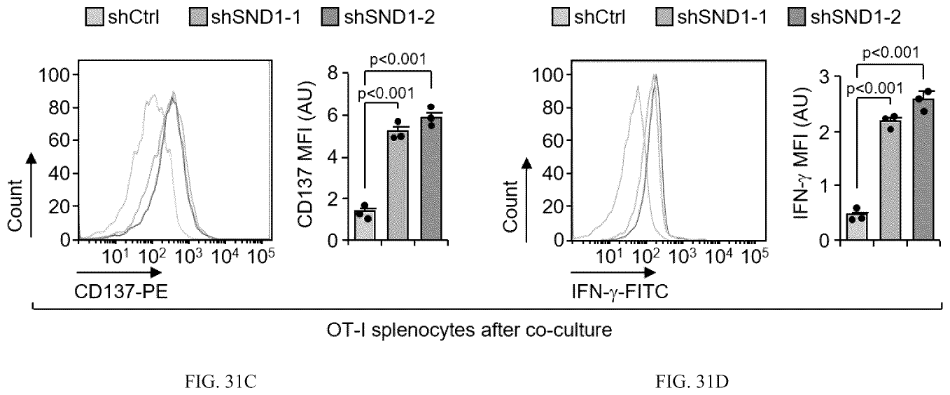

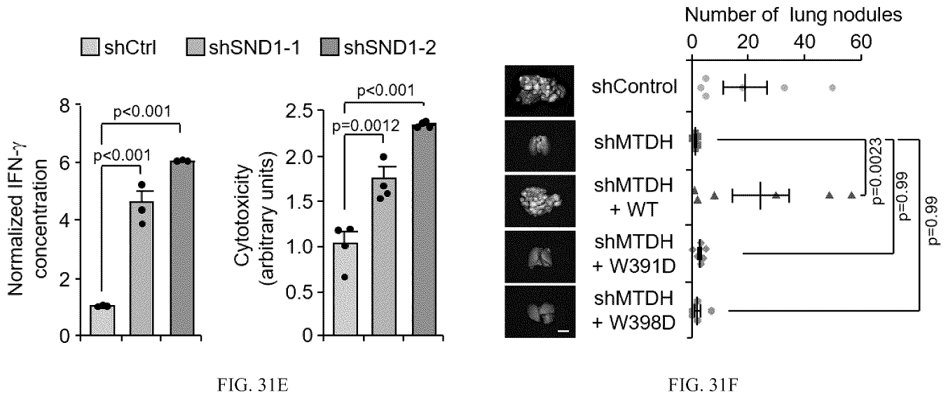

To further confirm the importance of the MTDH-SND1 binding in antigen presentation, MTDH KD Py8119-OVA tumor cells were rescued with wild type and SND1 interaction mutant forms of MTDH. Only WT, but not mutant MTDH (W391D and W398D), was found to bind and promote the degradation of Tap1/2 (FIGS. 21B-21D). Consistently, only the WT but not the mutant MTDH inhibits antigen presentation of tumor cells and activation of the T cells (FIGS. 21E-21H). Functionally, the immune cells have weaker killing effects on the tumor cells with wild type MTDH (FIG. 21H). Similar to MTDH KD, SND1 KD also dramatically enhanced the antigen presentation in tumor cells, and therefore, promoted the activation and killing effects of T cells (FIG. 31A-31E). The critical role of MTDH-SND1 complex in breast cancer metastasis has also been validated by in vivo lung metastasis assays using cell lines with or without MTDH-KD and subsequent rescue with WT or mutant forms of MTDH (FIG. 31F). In summary, these results indicate that the MTDH-SND1 complex promotes the immune evasion of metastatic breast cancer by down-regulating the tumor antigen presentation pathway.

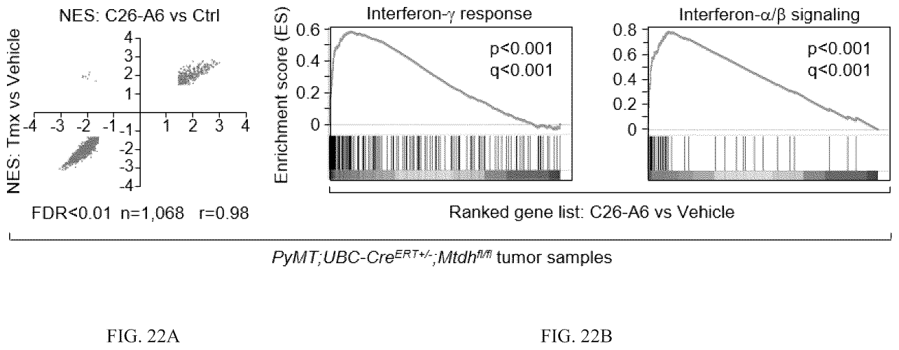

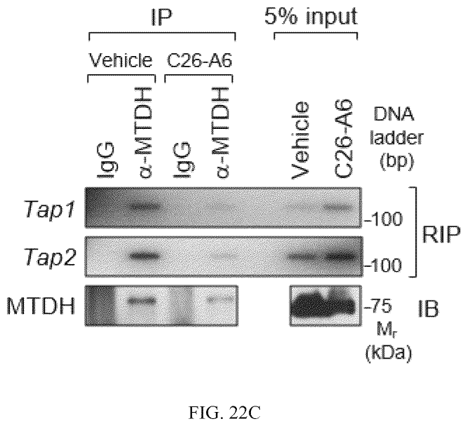

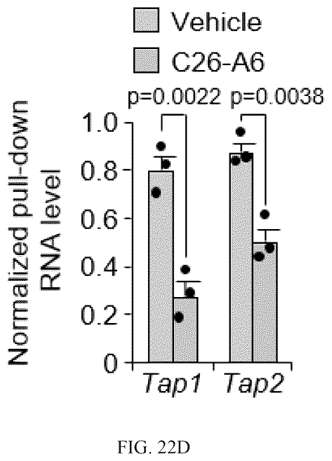

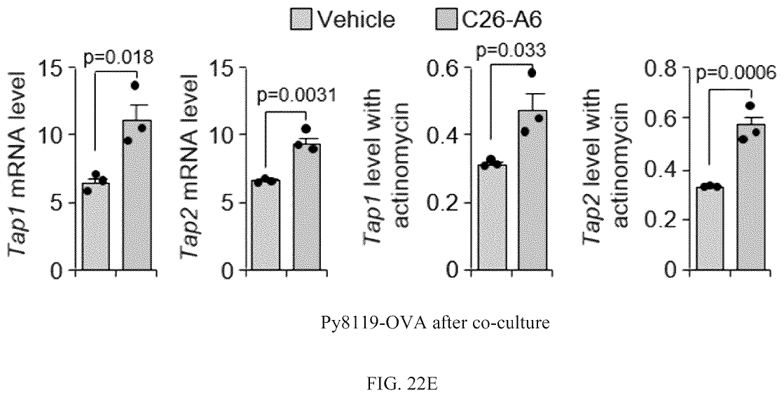

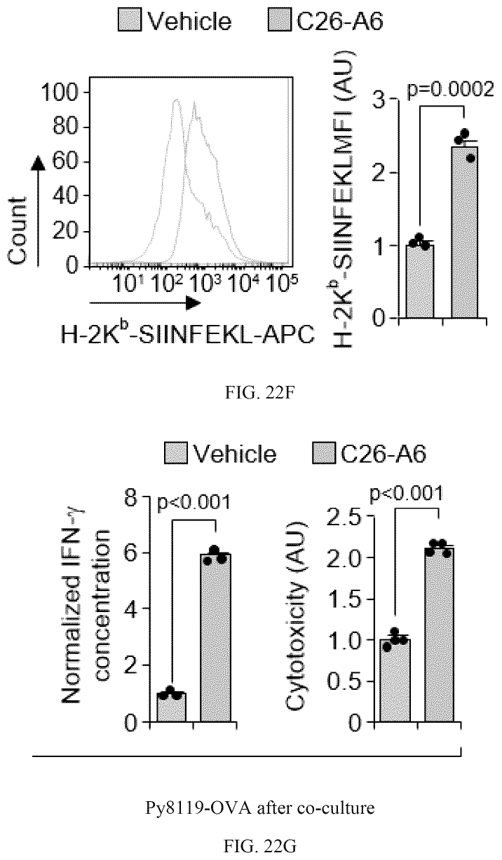

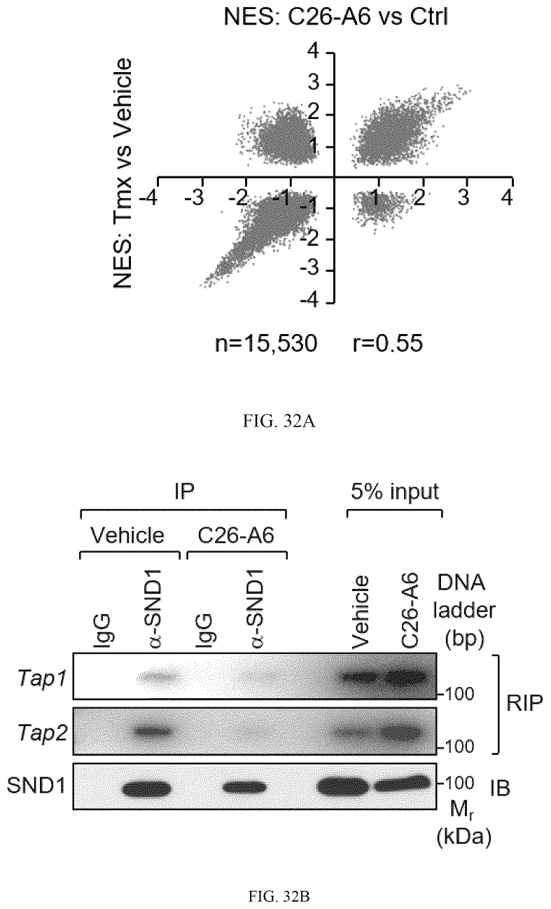

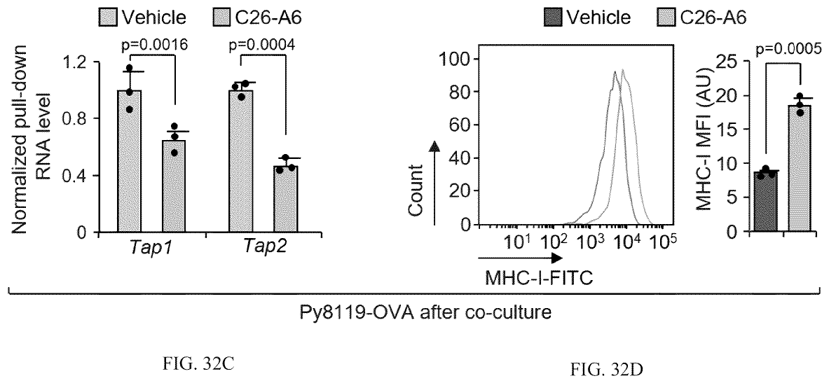

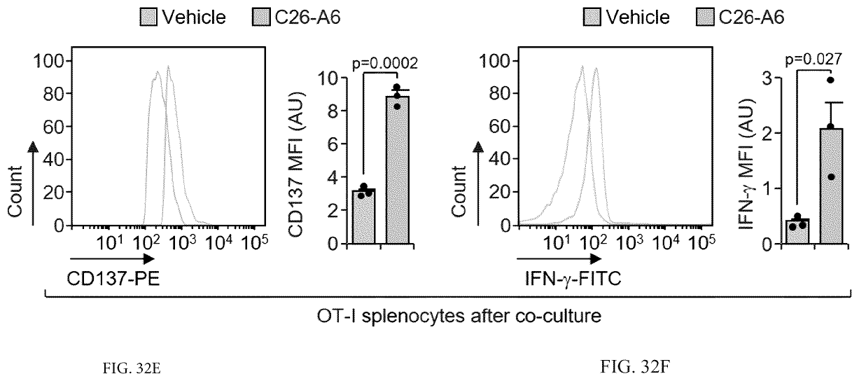

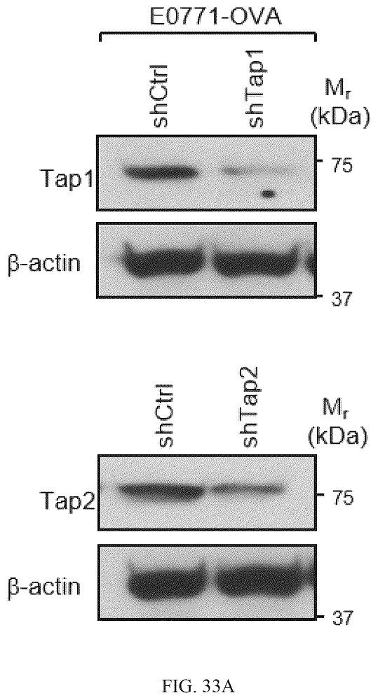

Inhibition of MTDH-SND1 enhances immune surveillance. Given the importance of the MTDH-SND1 complex in antigen presentation, whether compound C26-A6 could enhance immune surveillance in breast cancer was investigated. To this end, PyMT tumors treated with C26-A6 or control vehicle¹² were collected for RNA sequencing. Gene set enrichment analyses indicated a high degree of correlation between gene signatures that altered by acute genetic knockout of MTDH and by C26-A6 treatment (FIG. 32A), especially the ones that were most significantly inhibited or enhanced (FIG. 22A). Interferon signatures were dramatically enriched in C26-A6-treated tumors (FIG. 22B), which is consistent with the similar enrichment in MTDH-KO tumors (FIG. 18E), suggesting the enhanced immune surveillance upon C26-A6 treatment. Similarly, C26-A6 treatment decreased the Tap1/2 binding of MTDH and SND1 (FIGS. 22C, 22D and FIG. 32B, 32C), and increased the expression level of these mRNAs (FIG. 22E). These results further confirm the importance of MTDH-SND1 complex in promoting Tap1/2 degradation and suppressing antigen presentation. Consistent with these molecular alterations, MTDH-SND1 disruption by C26-A6 promotes OVA antigen and MHC-I complex presentation, T cell activation, and sensitizes the tumor cells to immune clearance (FIG. 22F, 22G and FIGS. 32D-32F).

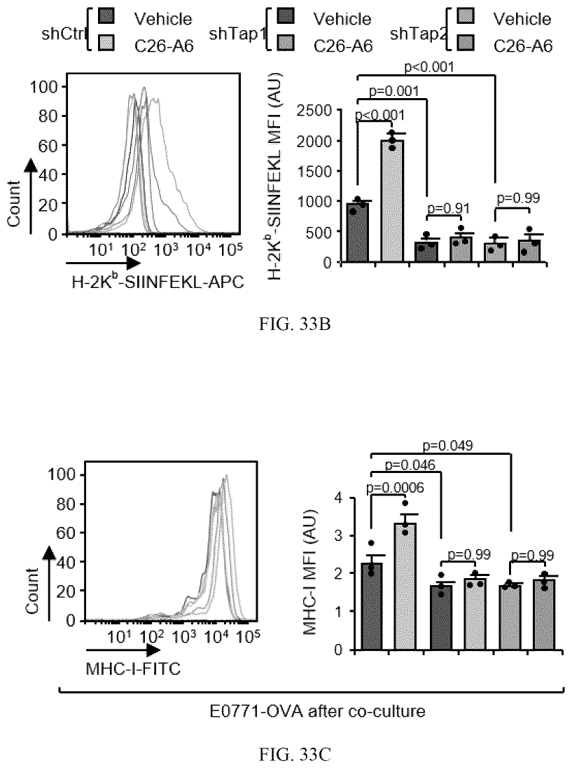

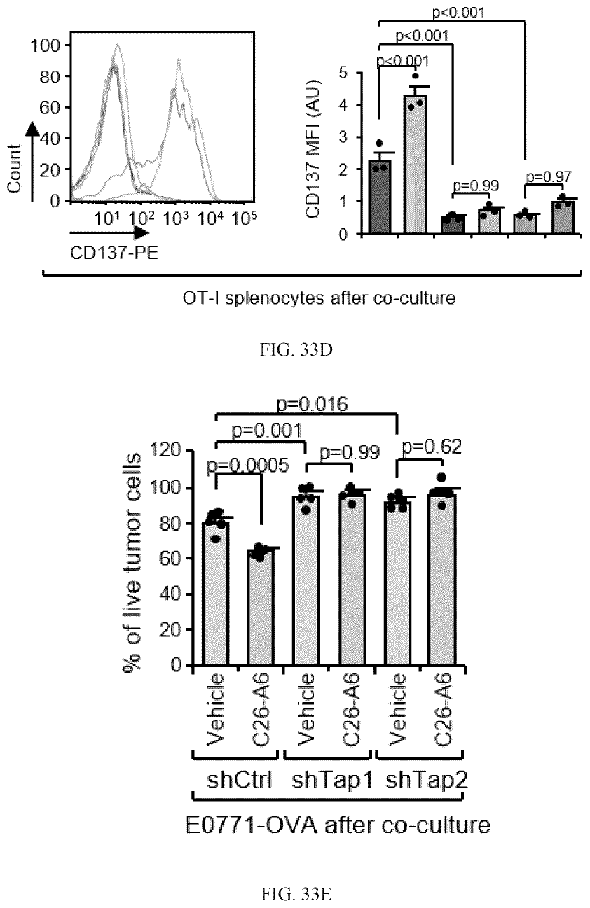

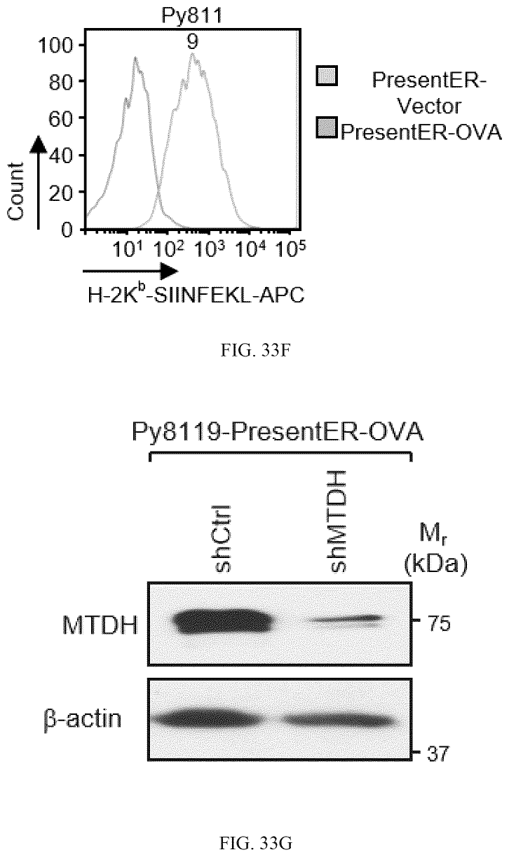

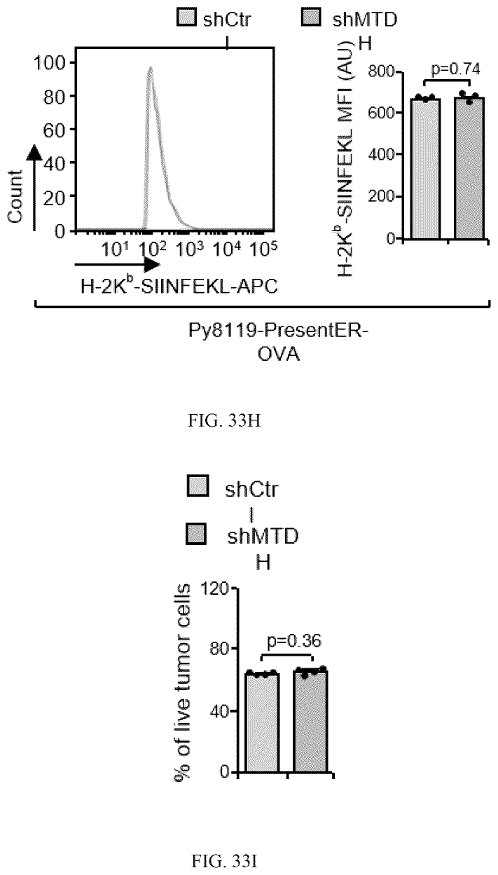

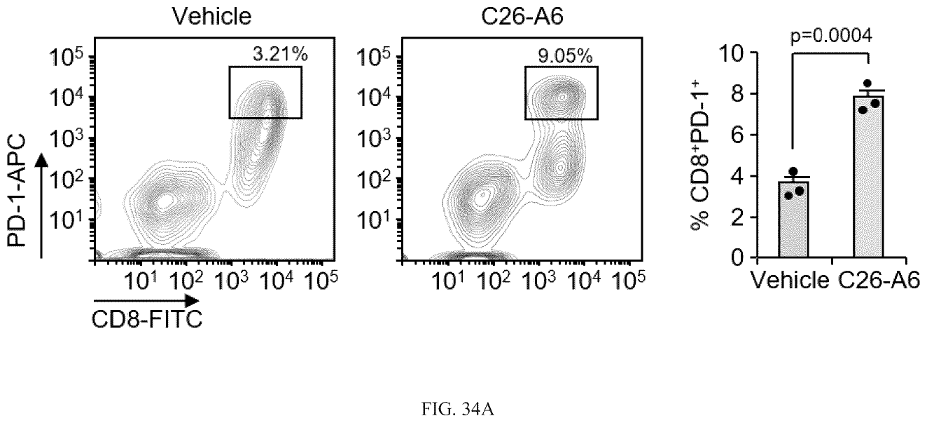

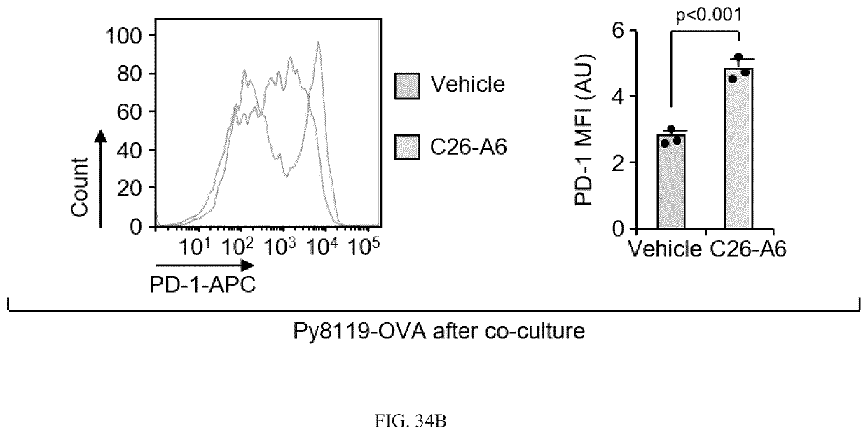

MTDH-SND1 suppresses antigen presentation by reducing Tap1/2. To confirm that MTDH-SND1 drives immune evasion through suppressing the Tap1/2 pathway, antigen presentation-deficient cells were generated by knocking down Tap1/2²⁰. E0771-OVA cells were transduced with lentiviruses expressing Tap1/2-targeting shRNAs. Tap1/2 KD was confirmed by western blot (FIG. 33A), and antigen presentation deficiency upon Tap1/2 KD was also validated (FIGS. 33B, 33C). Next, shCtrl (control shRNA) or Tap1/2 KD cells were co-cultured with OT-I splenocytes together with vehicle or C26-A6 treatment. Compared to a significant increase of antigen presentation and T cell activation in control cells after C26-A6 treatment, no such change occurred in Tap1/2 KD cells (FIGS. 33B-33D). Consistently, Tap1/2 KD significantly increased the survival of tumor cells in the co-culture with OT-I splenocytes, with no boosting of immune killing by C26-A6 treatment (FIG. 33E). These results indicate that MTDH-SND1 promotes immune evasion through suppressing Tap1/2.

To further confirm this finding, PresentER system-mediated OVA presentation was generated that bypass Tap1/2 (FIG. 33F)²¹. Py8119 cells with OVA presented by PresentER (Py8119-PresentER-OVA) were then transduced with lentiviral expressed MTDH-targeting shRNA. MTDH KD was confirmed by western blot (FIG. 33g). Py8119-PresentER-OVA cells with (shMTDH) or without (shCtrl) MTDH KD were next employed to co-culture with OT-I splenocytes. In such Tap1/2-bypassed antigen presentation system, MTDH KD did not enhance the OVA presentation, and moreover, MTDH KD did not sensitize tumor cells to the immune killing (FIGS. 33H, 33I). The results again validate that Tap1/2 is essential for the MTDH-SND1-mediated immune evasion.

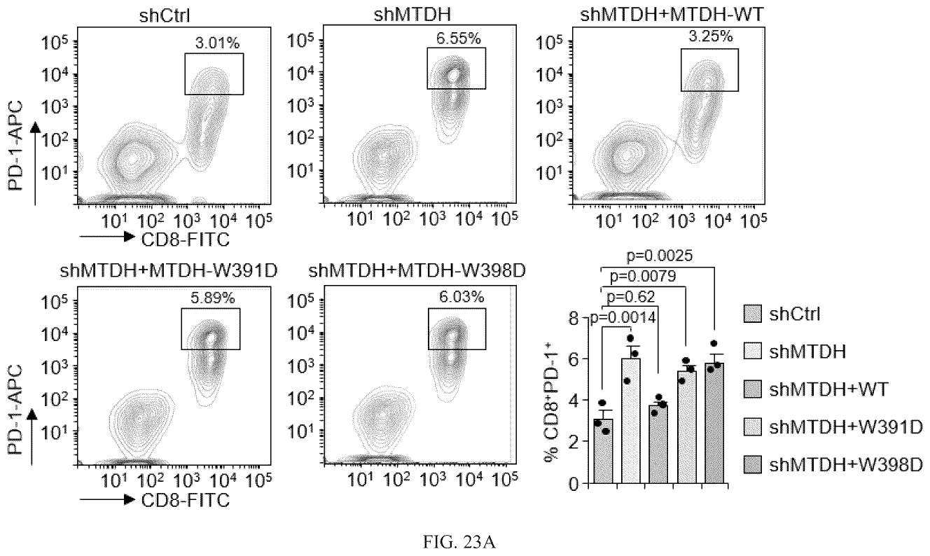

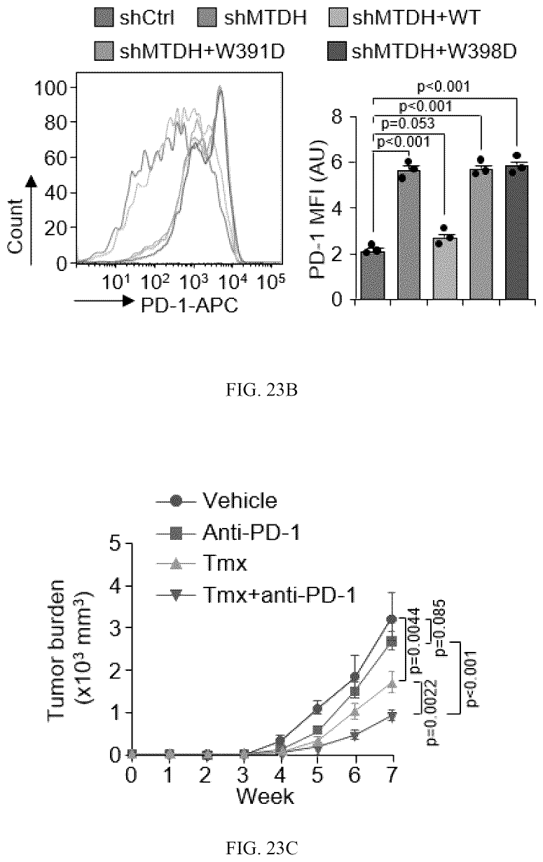

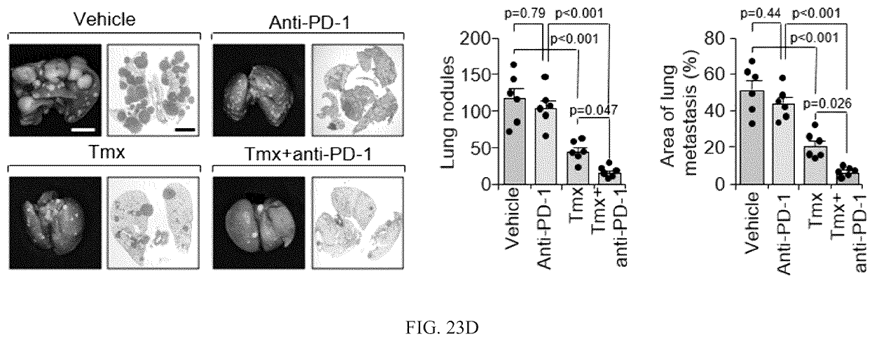

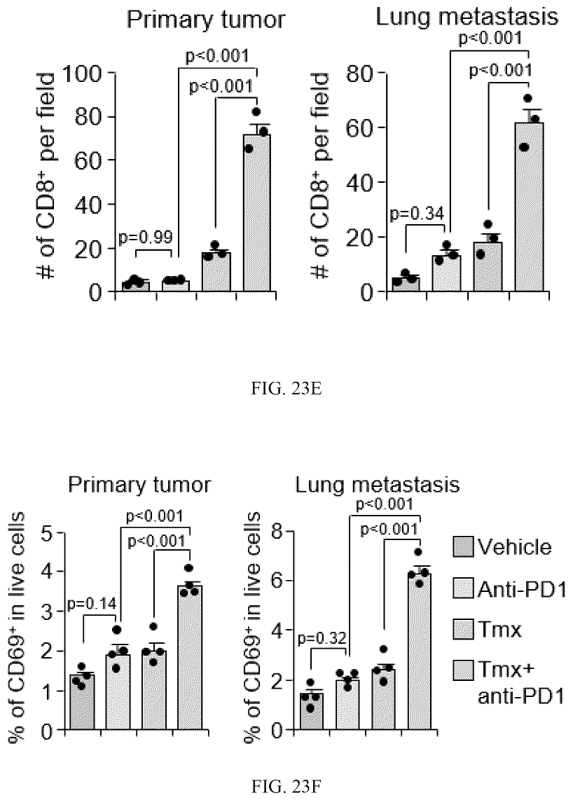

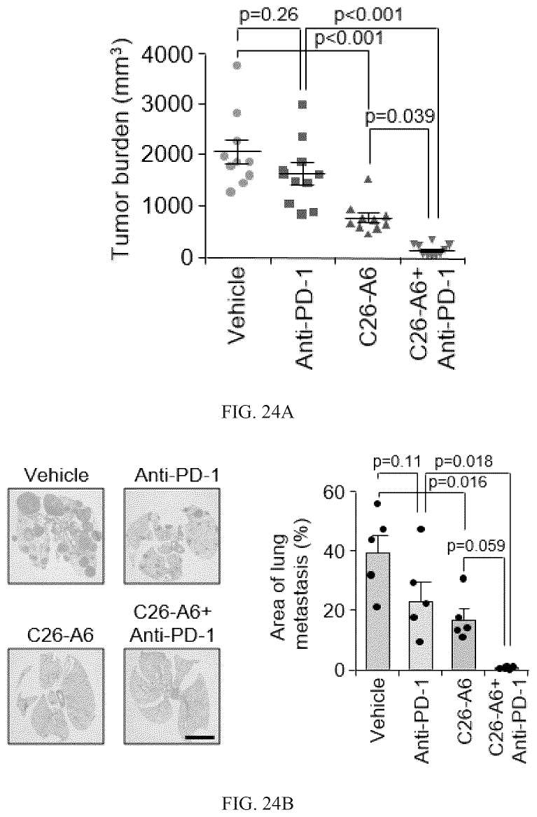

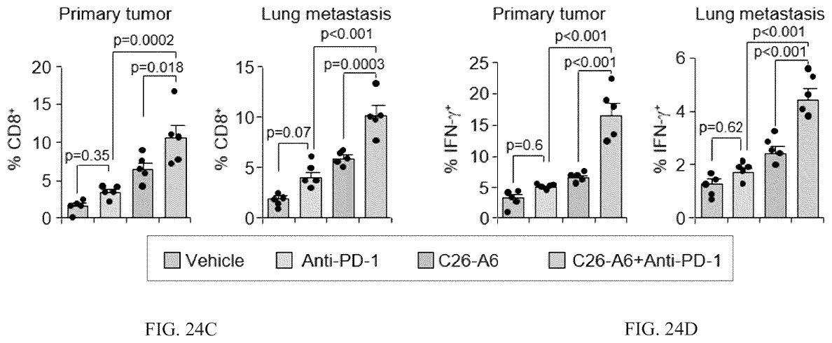

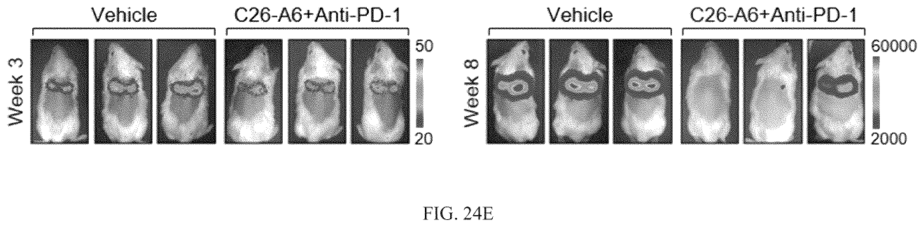

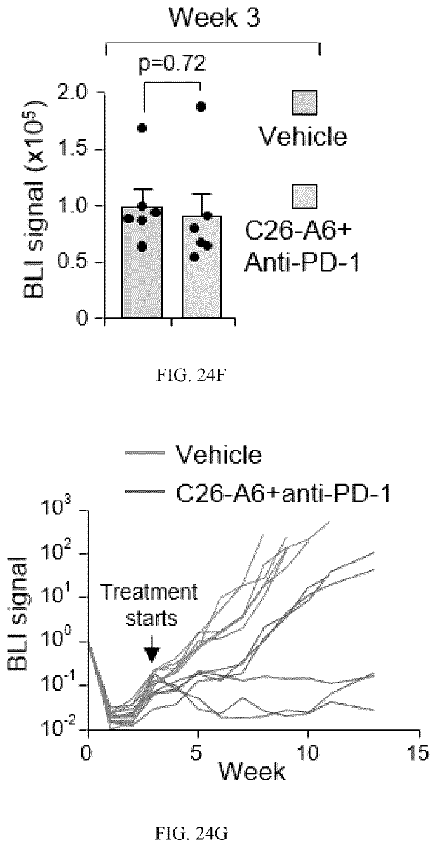

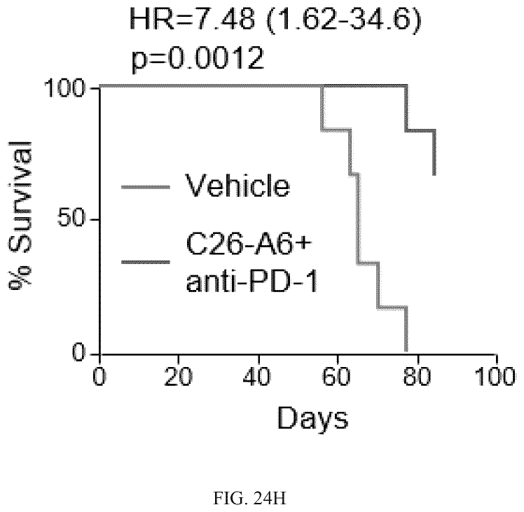

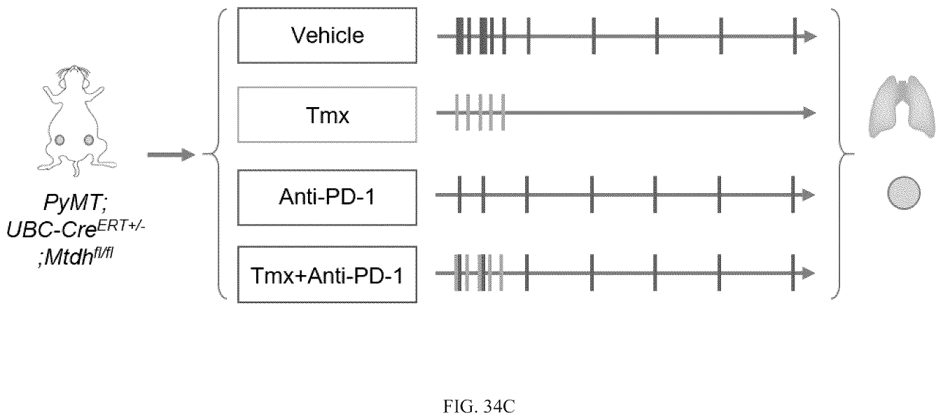

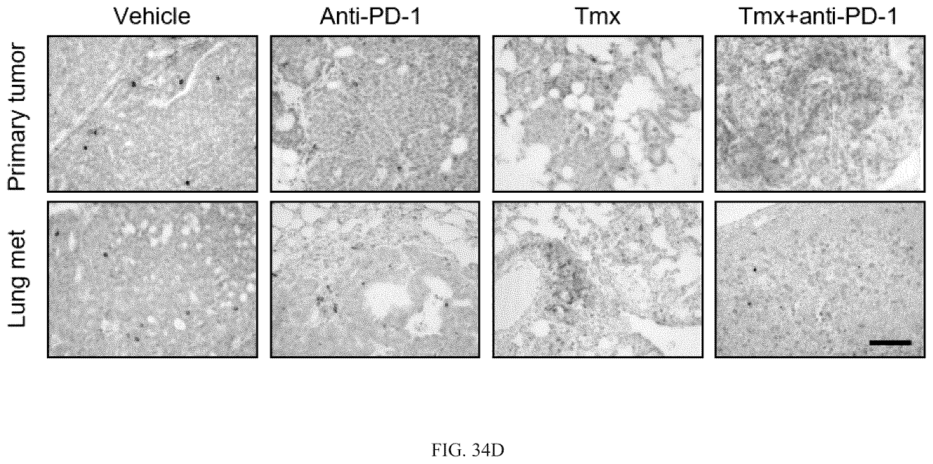

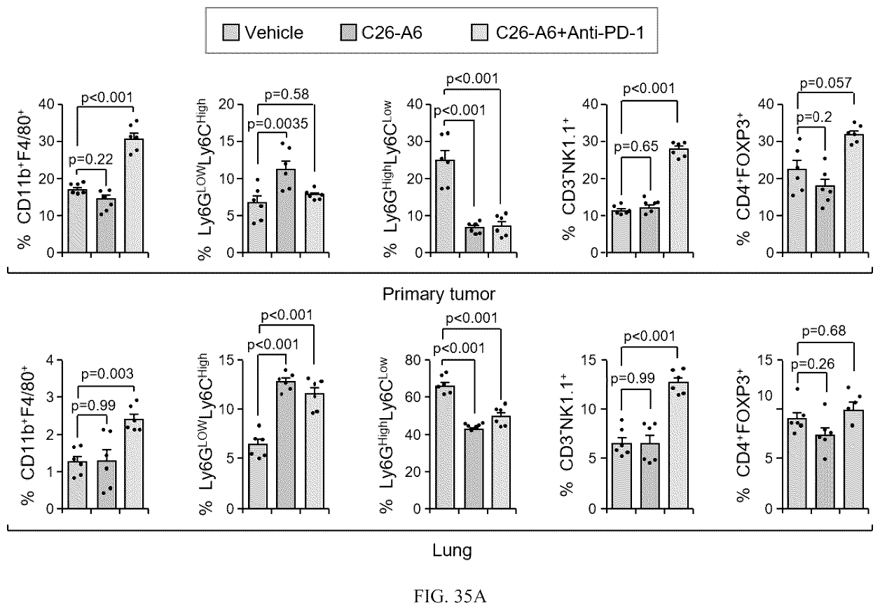

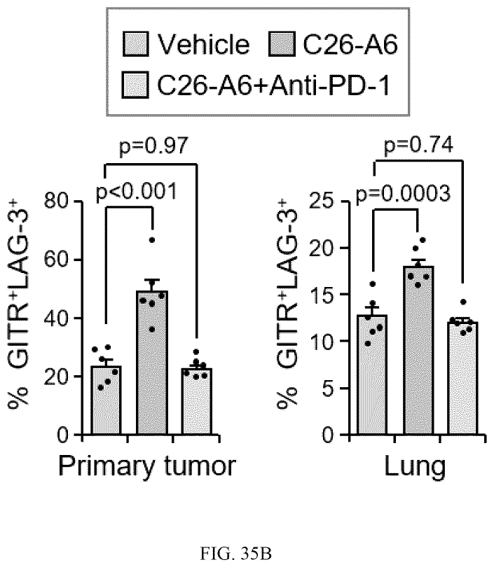

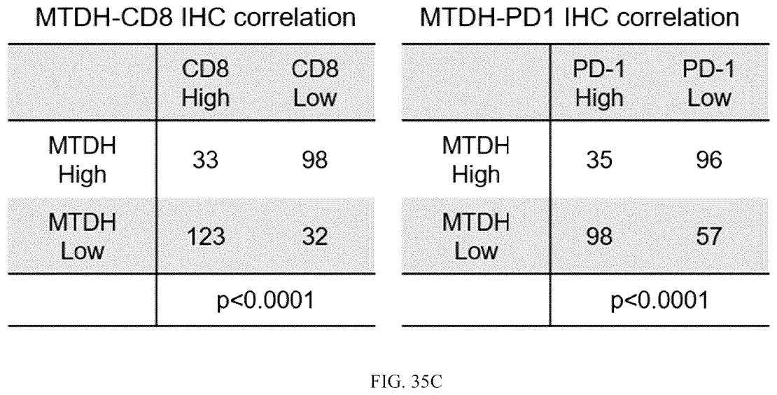

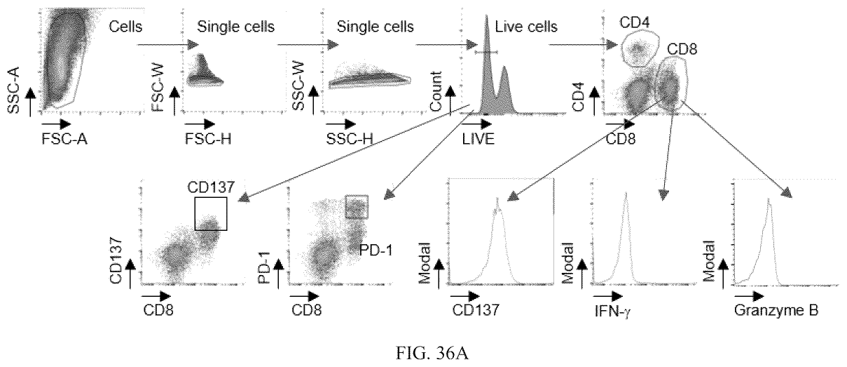

MTDH-SND1 disruption synergizes with anti-PD-1 therapy. Although disruption of MTDH-SND1 interaction activates CD8⁺ cytotoxic T cells (FIG. 20 to FIG. 22), increased T cell exhaustion was also observed, as indicated by PD-1 expression (FIGS. 23A, 23B and FIG. 34A, 34B). These observations suggested a potential synergistic anti-tumor effect of combining MTDH-SND1 blocking and anti-PD-1 therapy. To test this hypothesis, PyMT;UBC-Cre^{ERT+/-};Mtdh^{fl/fl} mice bearing developed PyMT tumors were treated with Tmx, anti-PD-1 alone or in combination (FIG. 23C; and FIG. 34C). Consistent with results from clinical trials²², anti-PD-1 alone did not significantly inhibit the progression of advanced breast cancer (FIGS. 23C, 23D). However, MTDH acute KO together with anti-PD-1 treatment demonstrated strong synergy in suppressing the growth and lung metastasis of MMTV-PyMT tumors (FIGS. 23C, 23D). Analysis of tumor samples indicated that MTDH-SND1 disruption and anti-PD-1 synergistically enhances T cell infiltration and activation (FIGS. 23E, 23F and FIG. 34E, 34F and FIG. 34D). Similarly, C26-A6 combined with anti-PD-1 markedly reduced primary tumor growth and lung metastasis, and such therapeutic response was significantly better than single treatments with C26-A6 or anti-PD-1 alone (FIGS. 24A, 24B). Consistent with this result, increased CD8⁺ T cell infiltration and activation were observed with the combination treatment (FIGS. 24C, 24d). In addition to examining the CD8⁺ T cells, a more detailed profiling of immune cell infiltration in mice was performed after such treatments. C26-A6 treatment alone significantly enhanced and reduced the infiltration of Ly6G^{low}Ly6C^{high} and Ly6G^{high}Ly6C^{low} sub-populations of MDSCs respectively, and also elevated CD8⁺ T cell exhaustion (FIGS. 35A, 35B). However, the populations of macrophages, NK cells and regulatory T cells were not significantly altered upon C26-A6 monotherapy (FIGS. 35A, 35B). Interestingly, combination with anti-PD-1 therapy increased the numbers of macrophage, NK cell and regulatory T cell in the microenvironment, and also reduced the exhausted CD8⁺ T cells (FIGS. 35A, 35B). These results collectively indicate that, in addition to affecting the CD8⁺ T cells, C26-A6 significantly reshaped the immune populations in the tumor microenvironment, especially in combination with anti-PD-1.

To more closely mimic the clinical treatment situation, it was tested whether C26-A6+anti-PD-1 treatment could control cancer progression in a model with established macro-metastases. To this end, FVB females with well-established lung macro-metastases 3 weeks after intravenous injection of PyMT tumor cells were generated (FIG. 24E). Six mice in each group were then randomized based on BLI signals (FIG. 24F), followed by treatment with vehicle or C26-A6+anti-PD-1. C26-A6+anti-PD1 treatment group in general had significantly slower metastasis progression pace compared to aggressive metastatic growth in the control group. Importantly, two mice in the combined treatment group had metastasis regression and the other one had stabilized disease (FIG. 24G). C26-A6+anti-PD1 treatment group also had significantly improved survival rate (FIG. 24H). The data suggest that C26-A6 combined with anti-PD1 therapy may have clinical benefits in metastatic breast cancer patients.

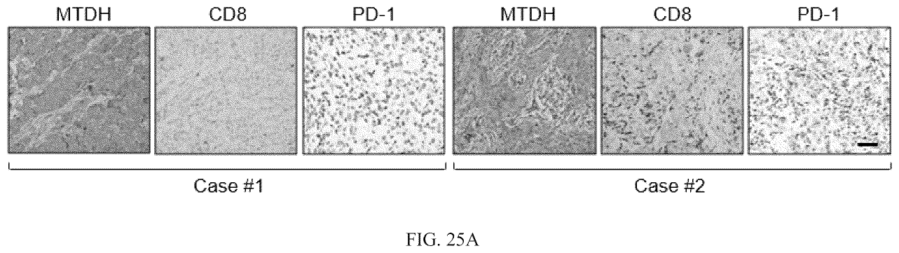

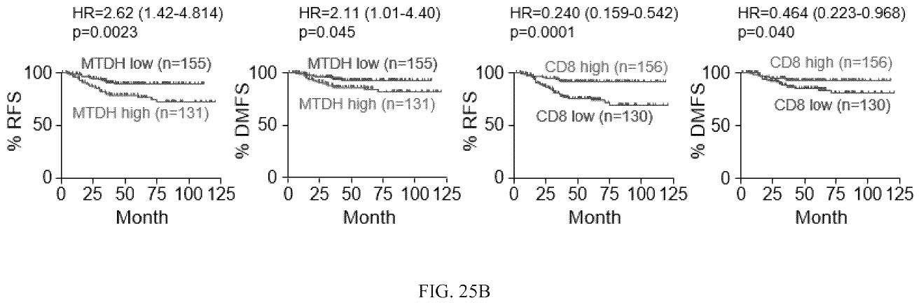

Consistent with these findings from mouse models, IHC staining of human triple-negative breast tumor samples showed that MTDH expression is negatively correlated with CD8⁺ cytotoxic T cell infiltration as well as PD-1 expression (FIG. 25B and FIG. 35C). Consistent with previous studies, MTDH-high patients have worse relapse-free and distant-metastasis free survival (FIG. 25B)^{7,10}. In contrast, CD8⁺ T cell infiltration was correlated with better survival (FIG. 25B). In summary, this study indicates MTDH-SND1 complex prevents tumor antigen presentation by degrading Tap1/2, and therefore, inhibits CD8⁺ T cell infiltration and activation to eventually promote immune evasion and metastasis. Importantly, MTDH-SND1 blocking improves response to anti-PD-1 therapy and synergistically suppresses metastatic breast cancer progression.

Discussion Small chemical inhibitors that disrupt MTDH-SND1 complex and show significant therapeutic potential in animal models¹² were developed as described in Example 1. However, the detailed molecular mechanism underlying this therapeutic effect was poorly understood. In this study, the PyMT mouse model in the FVB genetic background was used with constitutive or inducible KO of MTDH, which provided a clue that MTDH plays a role in regulating the anti-tumor immune response in immunocompetent mouse models. Moreover, Py8119 and E0771 models in C57BL/6 background were also employed, which enabled performance of tumor/immune cell co-culture to specifically investigate the mechanism underlying MTDH-SND1-mediated immune regulation. Combing these mouse models and human tumor sample studies, a new role of MTDH-SND1 in reducing the cell surface presentation of tumor antigen by destabilizing Tap1/2 mRNAs which encode key components of the antigen presentation machinery was discovered. As such, a high level of MTDH in tumors decreases the infiltration and activation of T cells, and facilitates immune evasion of metastatic breast cancer.

Immunotherapy has achieved exciting success in several cancers²⁴⁻²⁶, however, metastatic breast cancer patients did not respond well to this treatment in clinical trials^{27,28}. The resistance to immunotherapy, especially immune checkpoint blockade therapy, is partially due to the low immunogenicity of the disease^{29,30}, which resulted in limited immune cell infiltration. The presentation of tumor associated antigens attracts the CD8⁺ cytotoxic T cell infiltration, enhances the T cell activation, and consequently leads to tumor suppression^{31,32}. However, to escape such immunosurveillance, tumor cells may develop resistant mechanisms, including attenuating antigenicity. MTDH is a frequently overexpressed protein in breast cancer⁷, and in this study, was found to decrease the presenting of antigens on the surface of malignant cells (FIG. 20). In line with this finding, mice with MTDH-KO or breast cancer patients with lower MTDH had significantly elevated CD8⁺ cytotoxic T cell infiltration and activation (FIGS. 23 to 25).

In cancer context, tumor antigens are produced through proteasome-mediated degradation, endoplasmic reticulum loading (ER), and then cell surface presenting³³. At the molecular level, TAP1 and TAP2, which are members of the ATP-Binding Cassette (ABC) family³⁴, associate with other proteins to load the peptides to MHC-I-β2m complex, and then present antigens on the cell surface³⁵⁻³⁸. Given the critical roles of TAP1/2 in antigen processing and presentation, cells that lack TAP1 or TAP2 forms limited MHC-I-peptide complex^{39,40}, which in turn significantly affects the surface presenting of antigens. Consistent with this notion, breast cancer patients with lower TAP1/2 have significantly worse prognosis^{41,42}. Not only in breast cancer, reduced expression of TAP has also been observed in other tumor types, and it is thought to be one major mechanism of tumor immune evasion^{39,43}. In this study, MTDH was found to bind and destabilize Tap1/2, which resulted in less Tap1/2 in tumor cells (FIG. 3-2). This finding also explains reduced antigen presentation in the tumors with high MTDH expression and lower activation of the co-cultured immune cells.

MTDH and SND1 have both been reported as RNA binding proteins before⁴⁴⁻⁴⁹. The RNA binding of both MTDH and SND1 promotes cancer progression^{44,47-49}, suggesting the oncogenic role of these two proteins through the RNA regulation. SND1 is a member of RNA-induced silencing complex (RISC) that binds and degrades RNAs^{48,49}. In line with these previous findings, these data indicate SND1 interacts with and destabilizes Tap1/2 in association with MTDH (FIG. 4-2). Given the data that SND1 knockdown almost completely eliminates the binding between MTDH and Tap1/2 but not vice versa (FIG. 30), it is believed that instead of directly binding Tap1/2, MTDH assists Tap1/2 binding and degradation by SND1, and therefore inhibits the antigen presentation. Functionally, MTDH-SND1 disruption enhances immunosurveillance and inhibits metastatic breast cancer development.

Although MTDH-SND1 prevents the immune recognition of breast cancer cells, the tumor promoting function of MTDH-SND1 was also observed in immune deficient mouse models in Example 1. Based on the NGS analysis of gene expression changes in tumors upon acute MTDH depletion or C26-A6 treatment, two classes of functionally important genes were identified: genes that are up-regulated after MTDH depletion or C26-A6 treatment; and genes that are down-regulated. By analyzing MTDH depletion or C26-A6 treatment-downregulated genes, the negative enrichment of cell survival pathways and positive enrichment of necrosis and apoptotic pathways were found. These data suggested that MTDH-SND1 enhanced tumor-intrinsic proliferation and survival, which is also supported by Ki67 and cleaved caspase-3 IHC staining and the in vitro tumorsphere assay in Example 1. On the other hand, by analyzing MTDH depletion or C26-A6 treatment upregulated genes, the enrichment of immune related signatures was found (FIG. 18E and FIG. 26E); and further mechanistic studies indicated that MTDH-SND1 inhibits tumor antigen presentation and suppresses anti-tumor immune response in immunocompetent models. Collectively, MTDH-SND1 promoted tumor progression and metastasis by enhancing tumor-intrinsic proliferation and survival rates (see Example 1). Meanwhile, MTDH-SND1 also suppressed breast tumor progression by regulating stromal immune responses (current study). Given the data from CD8⁺ T cell depletion experiments (FIG. 18I, and FIGS. 27B-27D), it is believed that CD8⁺ T cells play critical roles in suppressing cancer progression after genetic disruption or therapeutic targeting of MTDH-SND1. In addition, NK cell has also been demonstrated to restrain breast cancer metastasis⁵⁰. However, as MTDH KO or C26-A6 treatment did not alter the NK cell population in the tumor microenvironment (FIGS. 26F, 26G and 35A), it is believed that the antitumor effects of NK cell is independent of MTDH. On the other hand, since C26-A6 also affected MDSC populations (FIG. 35A), the possibility that MDSC populations might also be involved in this effect cannot be excluded.

Taken together, the experimental results from the studies described in Examples 1 and 2 support the notion that both mechanisms contribute to the tumor promoting role of MTDH-SND1. Importantly, C26-A6 is able to block both MTDH-mediated mechanisms and achieve therapeutic benefits, making it an ideal candidate for further development as a novel therapeutic. Besides the increased CD8⁺ cytotoxic T infiltration and activation upon MTDH-SND1 disruption, elevated levels of PD-1 expression were also observed (FIG. 23A, 23B and FIG. 34A, 34B). Such a finding was also supported by patient samples (FIG. 25A), which supports the rationale for the combined therapy of immune checkpoint blockade and MTDH-SND1 disruption. Indeed, synergistic effects were obtained when metastatic breast cancer was treated together with C26-A6 and anti-PD-1 (FIG. 23 and FIG. 24). In summary, this study not only provides novel insights into the mechanism underlying MTDH-mediated immune evasion but also establishes a new strategy to enhance the immunotherapy response in metastatic breast cancer.

Methods: Animal models. All experimental protocols involving animals were conducted in compliance with the Institutional Animal Care and Use Committee (IACUC) of Princeton University. According to the approved IACUC protocol (1881-20), the primary humane endpoint of tumor burden for an individual mouse is 20 mm in any dimension or a total volume of 4000 mm³ for mice with multiple tumors. Mice were euthanized before exceeding the limit of tumor burden in this study. In the facility, mice were maintained at 20-22° C. with 14 h:10 h light:dark cycles at 40-70% relative humidity. MTDH knockout mice and backcrossed derivatives (MMTV-PyMT; Mtdh^{-/-10}, MMTV-PyMT;UBC-Cre^{ERT+/-};Mtdh^{fl/fl 12} on the FVB background were described previously, while the OT-I mice were obtained from Jackson Laboratory (Stock No: 003831). For spontaneous and experimental metastasis experiments, 8-10 weeks old female FVB, C57BL/6, or OT-I mice were injected with tumor cells by either tail vein or mammary fat pad injection as described. For spontaneous tumorigenesis studies, 4-6 weeks old mice were anaesthetized, and subsequently, a small incision was made to reveal the mammary gland. Single-cell suspensions of 10 μL PBS/Matrigel (1:1) were injected into the inguinal (#4) mammary fat pad following standard injection procedures⁵¹, and mice were examined weekly for mammary tumor development. Tumors were considered established when they became palpable for 2 consecutive weeks, and tumors were measured by calipers for calculation of tumor volumes (length×width²/2). For the cell lines that are stably labeled with a firefly luciferase expressing vector, lung metastases were monitored by bioluminescent imaging (BLI) and images were processed with Living Image 3D Analysis (version 1.0). Alternatively, lung nodules were counted directly after fixation in Bouin's solution or after sectioning and H&E staining of the lungs. 5-10 mice per experimental group were used as indicated in each experiment.

Generation of triple-negative breast cancer cohort. This study was approved by the independent ethics committee/institutional review board of FUSCC (Shanghai Cancer Center Ethics Committee). All patients gave their written informed consent before inclusion. The cohort was generated as described in detail previously^{52,53}. Detailed patient information has also been provided in Example 1.

Immunohistochemistry (IHC) staining and IHC-based classification. Immunohistochemistry (IHC) staining was performed on paraffin-embedded sections (4 μm thick) of tumor specimens to evaluate the expression of MTDH, CD8, and PD-1. IHC staining was performed using Ventana Benchmark ULTRA automated immunostainer (Ventana Medical Systems, Tucson, Arizona, USA). The following primary antibodies were used: anti-CD8 (SP57, Ventana, undiluted for patient samples; CST, #98941S, 1:100 dilution for mouse samples), anti-MTDH (Sigma, AMAB90762, 1:500 dilution), anti-PD-1 (CST, 43248S, 1:200 dilution), anti-CD3 (abcam, Ab16669, 1:200 dilution), anti-CD4 (CST, 25229S, 1:200 dilution), and anti-CD161 (CST, 391975, 1:200 dilution). Slides were then incubated with Goat Anti-Rabbit (Vector Laboratories, BA-1000, 1:300 dilution) IgG Antibody (H+L), Biotinylated. Images were taken with Carl Zeiss Zen (version 3.0) and processed with ImageJ (bundled with Java 1.8.0_172).

The IHC staining of MTDH was mainly found in the cytoplasm in tumor cells. The protein expression level of this marker was measured as the percentage of positive tumor cells (the number of positive tumor cells divided by the total number of tumor cells). By contrast, CD8 and PD-1 staining was primarily found in tumor-infiltrating lymphocytes (TILs), so the protein expression level of CD8 and PD-1 was measured as the percentage of positive cells (the number of positive cells divided by the total number of all types of cells). A cutoff of >7.5% positive tumor cells was employed to define MTDH high, cutoff of ≥10% positive cells to define CD8 high, and cutoff of >4% positive cells to define PD-1 high. All stained paraffin-embedded sections were independently evaluated by two experienced pathologists who were blinded to the patients' clinical information. Discrepancies between the two pathologists were resolved by discussion and consensus.

Cell lines. HEK293T (CRL-3216) were obtained from American Type Culture Collection (ATCC), PyMT cells (FVB background) were generated previously¹⁰, Py8119 (ATCC, CRL-3278) and E0771 (ATCC, CRL-3461) cells (C57BL/6 background) were obtained from Dr. Weizhou Zhang's lab. HEK293T, PyMT, and E0771 cells were cultured in DMEM media containing 10% FBS, 2 mM glutamine, and 100 U penicillin/0.1 mg/ml streptomycin, while Py8119 cells were cultured in DMEM/F12 (1:1) media containing 10% FBS, 20 ng/ml EGF, 5 μg/ml insulin, 2 μg/ml hydrocortisone and 100 U penicillin/0.1 mg/ml streptomycin. All cells were regularly checked for Mycoplasma and authenticated. Mouse splenocytes freshly isolated from OT-I mice were cultured in RPMI-1640 with 10% FBS, 1% HEPES, 1% sodium pyruvate, 0.05 mM β-mercaptoethanol, and 100 U penicillin/0.1 mg/ml streptomycin.

### a) NGS and GSEA Analysis

For Next-generation sequencing (NGS), PyMT;UBC-Cre^{ERT+/-};Mtdh^{fl/fl} tumorspheres were cultured as previously reported¹⁰. The tumorspheres were treated with vehicle or 0.02 μg/ml of 4-Hydroxytamoxifen (4-OHT) for 5 days. The 4-OHT containing media was changed with fresh media and the tumorspheres were cultured for another week. Total RNA samples were prepared from the tumorspheres using RNAeasy kit (Qiagen), and the following RNA preparation and sequencing procedures were performed as described in Example 1. Normalized gene expression data were pre-ranked based on the differences of expression (fold changes). The differential genes were identified as p<0.05 (moderated T-test) and fold change >2 folds. Gene Set Enrichment (GSEA) analysis was performed as described in Example 1.

### b) Viral Production and Infection

pLKO plasmids containing shRNA sequences that target murine Mtdh (shMTDH-1, TRCN0000125816; and shMTDH-2, TRCN0000313386), murine Snd1 (shSNDl-1, TRCN0000054742; and shSNDl-2, TRCN0000295753), murine Tap1 (TRCN0000066349), and murine Tap2 (TRCN0000066389) were purchased from Sigma-Aldrich (St Louis, MO, USA) and were cloned as described previously^{8,10}. For overexpression and rescue experiments, plasmids encoding wild-type Mtdh and mutant forms of Mtdh with SND1-interaction deficiency (W391D, W398D) were generated as previously described^{10,11}. To generate Py8119-OVA and E0771-OVA cell lines, Ovalbumin cDNA was obtained from pCl-neo-cOVA (Addgene, #25097). The Ova cDNA was cut out using NheI/SalI and ligated to IRES2-mCherry fragment from pIRES2-mCherry, and altogether cloned into the lentiviral vector pLEX replacing the IRES2-puro fragment. All plasmids were packaged into viruses using HEK293T cells as packaging cell lines along with helper plasmids, VSVG and dR8.9, following standard protocols. Viruses were collected 48-72 h after transfection. Target cells were infected with viral media in the presence of 5 μg/ml Polybrene. The infected cells were selected with puromycin (KD stable cell lines) or picking mCherry positive cells with flow (Py8119-OVA and E0771-OVA stable cell lines). E0771-OVA cell line was transduced with lentiviral expressing firefly luciferase with the same virus production and infection procedure as above to generated luciferase stably expressing E0771-OVA cell line.

Splenocyte isolation. OT-I mice were euthanized by cervical dislocation and spleens were collected into 50 ml conical tubes containing serum-free RPMI-1640 media. Spleens were smashed and passed through sterile mesh filters using 10 ml of media to wash the screen. Cells were spun down at 1200 rpm for 5 min, supernatant removed, and then resuspended in 10 ml ACK buffer (Fisher) for red blood cell lysis. Cells were briefly vortexed, allowed to sit at room temperature for 1 minute, and then quenched with 5 ml of culture media. Cells were then spun down and resuspended in culture media, counted, and plated in 6 well plates at 2×10⁶ cells/ml.

CD8⁺ T cell isolation. CD8⁺ T cells from OT-I splenocytes obtained above were further isolated with CD8a⁺ T Cell Isolation Kit (Miltenyi Biotec, #130-104-075). Briefly, Cells were resuspended in 40 μL of buffer (PBS with 0.5% BSA and 2 mM EDTA) per 107 total cells. 10 μL of Biotin-Antibody Cocktail was added followed by 5 min of incubation at 4° C. After adding another 30 μL of buffer, 20 μL of Anti-Biotin MicroBeads was added. The cells were incubated for 10 min at 4° C. and passed through LS column. The flow through CD8⁺ T cells were collected for future experiments.

Immune cell and tumor cell co-culture assay. Py8119-OVA tumor cells with various MTDH expression status were plated in 6-well plates for in vitro co-culture in DMEM/F12 media listed above. Once cells reached 50-75% confluency OT-I splenocytes or CD8⁺ T cells were added to the tumor cells at the 1:10 ratio (tumor cell. immune cell). After 24 hr of co-culture, the cells and culture media were collected for further experiments.

ELISA. Conditioned media was collected from OT-I and tumor cell co-cultures. IFN-γ Quantikine® ELISA kits (R&D systems) were utilized to measure the concentration of IFN-γ according to the manufacturer's instruction.

qRT-PCR analyses. Total RNA was isolated from tumor samples or cells using the Qiagen RNA extraction kit in accordance with the manufacturer's instructions and reverse transcript into cDNA with SuperScript™ IV kit. Real-time RT-PCR was performed on an ABI 7900 96 HT series PCR machine (Applied Biosystems) using SYBR Green Supermix (Bio-Rad Laboratories). The gene-specific primer sets were used at a final concentration of 0.2 μM and their sequences are listed in Table A. All qRT-PCR assays were performed in duplicate in at least three independent experiments using three different cell or tissue samples.

TABLE A
Primer Sets for qRT-PCR

| | | |
|---|---|---|
| SEQ ID NO: 7 | Tap1#1-F | CCAACGTGTGCGAGTCCATTA |
| SEQ ID NO: 8 | Tap1#1-R | AGAGTGAGGTACGGTGACCC |
| SEQ ID NO: 9 | Tap1#2-F | GGACTTGCCTTGTTCCGAGAG |
| SEQ ID NO: 10 | Tap1#2-R | GCTGCCACATAACTGATAGCGA |
| SEQ ID NO: 11 | Tap2#1-F | CCCTGTTTTTCTCGCTAAGAGC |
| SEQ ID NO: 12 | Tap2#1-R | CAACCGCTTCATCAGTGTTCT |
| SEQ ID NO: 13 | Tap2#2-F | CTGGCGGACATGGCTTTACTT |
| SEQ ID NO: 14 | Tap2#2-R | CTCCCACTTTTAGCAGTCCCC |
| SEQ ID NO: 15 | b2m#1-F | TTCTGGTGCTTGTCTCACTGA |
| SEQ ID NO: 16 | b2m#1-R | CAGTATGTTCGGCTTCCCATTC |
| SEQ ID NO: 17 | b2m#2-F | TTCTGGTGCTTGTCTCACTGA |
| SEQ ID NO: 18 | b2m#2-R | CAGTATGTTCGGCTTCCCATTC |
| SEQ ID NO: 19 | Gapdh-F | AAGATTGTCAGCAATGCATCC |
| SEQ ID NO: 20 | Gapdh-R | CTTCCACAATGCCAAAGTTGT |

RNA-Binding immunoprecipitation (RIP) and western blot (WB). Py8119-OVA tumor cells after co-culture for 24 hr were collected for RIP using RNA-Binding Protein Immunoprecipitation Kit (Millipore, #17-700). Briefly, 5×10⁷ tumor cells were resuspended with 200 μL of RIP lysis buffer and frozen at −80° C. for at least 1 hr to lyse the cells. The sample was then centrifuged at 14,000 rpm for 10 min at 4° C. 20 μL of supernatant was transferred to two new tubes with 10 μL each and frozen at −80° C. to serve as input of total. The rest of the supernatant was diluted with 1.8 ml of RIP immunoprecipitation buffer and split equally into two 1.5 ml EP tubes. 5 μg of MTDH (Invitrogen, catalogue #40-6500), SND1 (Santa Cruz, catalogue #sc-271590) or IgG (provided in the Kit) antibody was added into each tube and incubated at 4° C. overnight. The next day, 50 μL of magnetic beads was added and incubated for another 2 hr. The beads were then washed with 500 μL of RIP wash buffer on a magnetic separator for 5 times. During the final wash, 25 μL of the beads in the wash buffer was transferred into a new tube, stored at −80° C. to serve as input of IP. The rest of the beads together and 10 μL input of total were resuspended with 300 μL of proteinase K buffer respectively and incubated at 55° C. for 30 min to degrade proteins. RNA was extracted with phenol:chloroform:isoamyl alcohol and precipitated with 100 μL of Salt Solution I, 30 μL of Salt Solution II, and 10 μL of Precipitation Enhancer at −80° C. overnight. The samples were then spun at 14,000 rpm for 30 min and RNA was washed with 80% ethanol. Finally, RNA was resuspended with 10 μL of RNase-free water. qRT-PCR was performed to detect Tap1/2. The pulldown of MTDH and SND1 was confirmed with western blot.

For western bot, the input of total and IP described above, or proteins that extracted from Py8119 cells with/without co-culture in RIPA buffer were resolved with SDS-PAGE gel and immunoblotted with standard protocols. Antibodies against MTDH (invitrogen, catalogue #40-6500), SND1 (Santa cruz, catalogue #sc-271590), SND1 (Sigma, HPA002529), TAP1 (CST, #12341S), and TAP2 (Thermo Fisher Scientific, #PA5-37414) were diluted 1:1000, while antibody against β-actin control (Abcam, catalogue #ab6276) was diluted 1:5000.

Electrophoretic Mobility Gel Shift Assay (EMSA). TAP1 and TAP2 mRNA was prepared by in vitro transcription with Standard RNA Synthesis Kit (NEB, E2050). pcDNA3.1 with full-length TAP1 and TAP2 ORFs were used as template (GeneScript, OHu19274D and OHu24630D). The products were Biotin-labelled and purified with Pierce™ RNA 3′ End Biotinylation Kit (ThermoFisher, 20160). 100 nM of human recombinant MTDH protein (Abnova, H00092140-P01) or human recombinant SND1 protein (Abnova, H00027044-P01) alone or in combination were incubated with 10 nM of biotin-labelled TAP1 or TAP2 mRNA for 15 min in room temperature. For MTDH and SND1 in combination group, 100 nM of MTDH and SND1 proteins were preincubated on ice for 1 hr. After the incubation, EMSA assay was performed using LightShift™ Chemiluminescent EMSA Kit (ThermoFisher, 20148).

Cell viability and cytotoxicity assays. To assess tumor cell viability after CD8⁺ T cell co-culture, the LDH cytotoxicity assay (Promega, #G1780) was used to measure T-cell killing of Py8119-OVA tumor cells following the manufacturer's instructions. Briefly, after co-culture, 50 μL co-culture media was collected and added to 96-well plate. Same amount of CytoTox 96 reagent was added and incubated in room temperature for 30 min. Absorbance at 490 nm was measured to calculate cytotoxicity.

Antibody collection and T-cell depletion. TIB-207 (anti-CD4) and TIB-105 (anti-CD8) hybridoma cell lines were obtained from ATCC for the collection of T-cell depletion antibodies. CD hybridoma media (Gibco®) supplemented with 4% GlutaMAX™ was used for hybridoma cell culture. Conditioned media from >90% confluent 10 cm plates of hybridoma cells was collected, spun down to remove cell debris, and filtered through a 0.2 μm filter set before purification. The HiTrap Protein G HP column (GE Healthcare Life Sciences) was used to purify antibody using binding buffer (20 mM sodium phosphate pH 7.0) elution buffer (0.1 M glycine-HCl pH2.7) and neutralization buffer (1 M Tris-HCl pH 9.0) following the manufacturer's instructions. Purified antibodies were then concentrated using Amicon ultra-15 ml (3K) spin tubes (Millipore) at 4000 rpm for 45 min at 20° C. Concentrated antibody was dialyzed against PBS at 4° C. overnight and then antibody concentration was quantified by standard Bradford assay. For in vivo T-cell depletion experiments, animals were injected with either 125 μg/mouse of antibody or same amount of isotype control in PBS by i.p. injection three days prior to tumor cell injection and then every three days post tumor cell injection.

Flow cytometry. Dissociated tumors or lungs were dissected and prepared as previously described¹⁰. Specifically, samples were minced into small pieces and digested for 1 hr at 37° C. in culture medium (1:1 Dulbecco's modified Eagle's medium (DMEM): Ham's F-12 medium containing 5% FBS, 10 ng/ml epidermal growth factor [EGF], 500 ng/ml hydrocortisone, 5 μg/ml insulin, 20 ng/ml cholera toxin, and 1% Pen/Strep) supplemented with 300 U/ml type 1A collagenase (Thermofisher) and 100 U/ml hyaluronidase (Sigma) to prepare single cell suspensions as previously described⁵⁴. Organoids were sequentially suspended with 0.25% trypsin-EDTA for 1.5 min, 5 mg/ml Dispase (Invitrogen), and 0.1 mg/ml DNase (Sigma) for 5 min, and 0.64% ammonium chloride for 5 min at 37° C. before filtration through a 40 μm nylon cell strainer. Tumor or lung cell suspensions were incubated with an antibody cocktail in FACS buffer (PBS+3% BSA) for 30 minutes at 4° C., washed, and resuspended in FACS buffer for flow analysis. Isotype controls were used to assess specificity of antibody labeling. Antibodies (with 1:200 dilution) used for staining immune or tumor cells are as follows: DAPI (ThermalFisher, #62248, 1:1000) or Fixable Viability Dye eFluor™ 506 (ThermalFisher, #65-0866-14, 1:1000) served as live/dead indicator; PerCP-Cy5.5 anti-mouse CD45 (eBioscience, #45-0451-82); PE-Cy7 anti-mouse CD4 (BioLegend, #100422); APC anti-mouse CD25 (BioLegend, #102012); FITC anti-mouse CD8a (eBioscience, #11-0081-82), APC anti-mouse CD8a (BioLegend, #100712), APC-Cy7 anti-CD8a (BioLegend, #100714), APC anti-mouse PD-1 (BioLegend, #135210); APC-Cy7 anti-mouse CD3 (BioLegend, #100330); PE anti-mouse CD137 (BioLegend, #106106); FITC anti-mouse CD69 (BioLegend, #104506); PE anti-mouse Granzyme B (ThermoFisher, #12-8898-82); FITC anti-mouse IFN-γ (BioLegend, #505806); APC anti-mouse OVA256-264 peptide (SIINFEKL) bound to H-2Kb (BioLegend, #141605); FITC anti-mouse H-2K^{d}/H-2D^{d} (MHC-I) (BioLegend, #114606); PE-Cy7 anti-mouse CD11b (BioLegend, #101216), APC-Cy7 anti-mouse F4/80 (BioLegend, #123118), FITC anti-mouse Ly6G (BioLegend, #127606), PE anti-mouse Ly6C (BioLegend, #128008), PE anti-mouse NK1.1 (BioLegend, #108708), FITC anti-mouse FOXP3 (eBioscience, #11-5773-82), PE-Cy7 anti-mouse GITR (BioLegend, #126318), and PE anti-mouse LAG-3 (BioLegend, #125208). To test the expression of cytokines, such as IFN-γ and Granzyme B, cells were treated with 100 ng/ml of Phorbol 12-myristate 13-acetate (Sigma, #79364), Ionomycin (Sigma, #I0634), and protein transport inhibitor for 4 hr, before subjected to the antibody staining. Fixation/Permeabilization Solution Kit with BD GolgiStop™ Kit (BD, #554715) was employed for the cell staining. Samples were analyzed with BD FACSDiva v6 software and data was processed with FlowJo v10 software. Gating strategy is shown in Supplementary FIG. 1-2.

Tamoxifen (Tmx), C26-A6 and anti-PD-1 in vivo treatment. MMTV-PyMT or MMTV-PyMT;UBC-Cre^{ERT+/-};Mtdh^{fl/fl} females were divided into four groups when primary tumors have been established (tumors were considered established when they became palpable for 2 consecutive weeks). The mice were treated with C26-A6 and anti-PD-1 alone or in combination; or treated with Tmx and anti-PD-1 alone or in combination respectively.

Tmx (Sigma-Aldrich, #T5648) and C26-A6 was prepared and administrated as previously described. Briefly, for Tmx treatment, indicated mice were injection with 60 mg/kg of the solution via i.p. for 5 constitutive days. Such dosing regimen of tamoxifen was commonly used in conditional KO of gene of interest in mouse models of breast cancer including MMTV-PyMT and been shown to have no direct effect on tumor growth and metastasis^{14,15}. For C26-A6 treatment, the mice were injected via tail-vein (T.V.) 5 days per week at 15 mg/kg. For the mice that T.V. injection was failed due to the high frequency treatment at late timepoints, i.p. injection with 2× dose was performed instead.

PD-1 antibody (BioXcell, #BP0146) treatment was performed as previously reported⁵⁵. The mice were treated on days 0, 4, 7, and then once weekly at 200 μg/mouse via i.p. Rat IgG2a (BioXcell, #BP0089) was injected with the same scheme served as control.

Statistics and reproducibility. Animals were excluded only if they died or had to be euthanized according to the IACUC protocol. No statistical method was used to predetermine sample size. Data collection and analysis were not performed blinded to the conditions of the experiments. For in vivo experiments, animals were randomized and treated as indicated in each experiment. For in vitro experiments, all samples were analyzed equally with no sub-sampling; therefore, there was no requirement for randomization. The experiments in FIGS. 20B, 21A, 21B and in FIGS. 26A-26C; 28B, 28F, FIG. 30A, 30H; FIG. 33A, 33G; FIG. 34D have been repeated for at least 3 times with similar results. Statistical analyses were indicated in figure captions. Error bars indicate means±SEM. GraphPad Prism software (version 7) was used for statistical calculations.

Data availability. All RNA sequencing data generated in this study have been deposited as a superseries at the NCBI Gene Expression Omnibus with the accession code GSE174630.

### REFERENCES

1 Criscitiello, C. & Curigliano, G. Immunotherapeutics for breast cancer. Current opinion in oncology 25, 602-608 (2013).
2 Ernst, B. & Anderson, K. S. Immunotherapy for the treatment of breast cancer. Current oncology reports 17, 5 (2015).
3 Ravelli, A. et al. Immune-related strategies driving immunotherapy in breast cancer treatment: a real clinical opportunity. Expert review of anticancer therapy 15, 689-702 (2015).
4 Page, D. B., Naidoo, J. & McArthur, H. L. Emerging immunotherapy strategies in breast cancer. Immunotherapy 6, 195-209, doi:10.2217/imt.13.166 (2014).
5 Soliman, H. Immunotherapy strategies in the treatment of breast cancer. Cancer control: journal of the Moffitt Cancer Center 20, 17-21 (2013).
6 Emens, L. A. Breast cancer immunobiology driving immunotherapy: vaccines and immune checkpoint blockade. Expert review of anticancer therapy 12, 1597-1611, doi:10.1586/era.12.147 (2012).
7 Hu, G. et al. MTDH activation by 8q22 genomic gain promotes chemoresistance and metastasis of poor-prognosis breast cancer. Cancer cell 15, 9-20 (2009).
8 Blanco, M. A. et al. Identification of staphylococcal nuclease domain-containing 1 (SND1) as a Metadherin-interacting protein with metastasis-promoting functions. The Journal of biological chemistry 286, 19982-19992 (2011).
9 Qian, B. J. et al. MTDH/AEG-1-based DNA vaccine suppresses lung metastasis and enhances chemosensitivity to doxorubicin in breast cancer. Cancer Immunol Immunother 60, 883-893 (2011).
10 Wan, L. et al. MTDH-SND1 interaction is crucial for expansion and activity of tumor-initiating cells in diverse oncogene- and carcinogen-induced mammary tumors. Cancer cell 26, 92-105 (2014).
11 Guo, F. et al. Structural insights into the tumor-promoting function of the MTDH-SND1 complex. Cell reports 8, 1704-1713 (2014).
12 Shen, M. et al. Small Molecule Inhibitors that Disrupt the MTDH-SND1 Complex Suppress Breast Cancer Progression and Metastasis. Nature Cancer, Co-submitted companion manuscript (2021).
13 Ewens, A., Mihich, E. & Ehrke, M. J. Distant metastasis from subcutaneously grown E0771 medullary breast adenocarcinoma. Anticancer Res 25, 3905-3915 (2005).
14 Du, J. et al. PDK1 promotes tumor growth and metastasis in a spontaneous breast cancer model. Oncogene 35, 3314-3323, doi:10.1038/onc.2015.393 (2016).
15 Canovas, B. et al. Targeting p38alpha Increases DNA Damage, Chromosome Instability, and the Anti-tumoral Response to Taxanes in Breast Cancer Cells. Cancer cell 33, 1094-1110 e1098, doi:10.1016/j.ccell.2018.04.010 (2018).
16 Gibby, K. et al. Early vascular deficits are correlated with delayed mammary tumorigenesis in the MMTV-PyMT transgenic mouse following genetic ablation of the NG2 proteoglycan. Breast cancer research: BCR 14, R67, doi:10.1186/bcr3174 (2012).
17 Clarke, S. R. et al. Characterization of the ovalbumin-specific TCR transgenic line OT-I: MHC elements for positive and negative selection. Immunology and cell biology 78, 110-117 (2000).
18 Hogquist, K. A. et al. T cell receptor antagonist peptides induce positive selection. Cell 76, 17-27 (1994).
19 Leone, P. et al. MHC class I antigen processing and presenting machinery: organization, function, and defects in tumor cells. Journal of the National Cancer Institute 105, 1172-1187 (2013).
20 Das, K. et al. Generation of murine tumor cell lines deficient in MHC molecule surface expression using the CRISPR/Cas9 system. PLoS One 12, e0174077, doi:10.1371/journal.pone.0174077 (2017).
21 Gejman, R. S. et al. Rejection of immunogenic tumor clones is limited by clonal fraction. Elife 7, doi:10.7554/eLife.41090 (2018).
22 Solinas, C. et al. Targeting immune checkpoints in breast cancer: an update of early results. ESMO open 2, e000255 (2017).
23 Nguyen, D. X., Bos, P. D. & Massague, J. Metastasis: from dissemination to organ-specific colonization. Nature reviews 9, 274-284 (2009).
24 Mellman, I., Coukos, G. & Dranoff, G. Cancer immunotherapy comes of age. Nature 480, 480-489 (2011).
25 Park, T. S., Rosenberg, S. A. & Morgan, R. A. Treating cancer with genetically engineered T cells. Trends in biotechnology 29, 550-557 (2011).
26 Wolchok, J. D. & Chan, T. A. Cancer: Antitumour immunity gets a boost. Nature 515, 496-498 (2014).
27 Kroemer, G., Senovilla, L., Galluzzi, L., Andre, F. & Zitvogel, L. Natural and therapy-induced immunosurveillance in breast cancer. Nature medicine 21, 1128-1138 (2015).
28 Marme, F. Immunotherapy in Breast Cancer. Oncology research and treatment 39, 335-345 (2016).
29 Bates, J. P., Derakhshandeh, R., Jones, L. & Webb, T. J. Mechanisms of immune evasion in breast cancer. BMC cancer 18, 556 (2018).
30 Denkert, C. The immunogenicity of breast cancer—molecular subtypes matter. Ann Oncol 25, 1453-1455 (2014).
31 Lanitis, E., Dangaj, D., Irving, M. & Coukos, G. Mechanisms regulating T-cell infiltration and activity in solid tumors. Ann Oncol 28, xii18-xii32 (2017).
32 Wellenstein, M. D. & de Visser, K. E. Cancer-Cell-Intrinsic Mechanisms Shaping the Tumor Immune Landscape. Immunity 48, 399-416 (2018).
33 Johnsen, A. K., Templeton, D. J., Sy, M. & Harding, C. V. Deficiency of transporter for antigen presentation (TAP) in tumor cells allows evasion of immune surveillance and increases tumorigenesis. J Immunol 163, 4224-4231 (1999).
34 Oancea, G. et al. Structural arrangement of the transmission interface in the antigen ABC transport complex TAP. Proceedings of the National Academy of Sciences of the United States of America 106, 5551-5556 (2009).
35 Blum, J. S., Wearsch, P. A. & Cresswell, P. Pathways of antigen processing. Annual review of immunology 31, 443-473 (2013).
36 Leonhardt, R. M., Keusekotten, K., Bekpen, C. & Knittler, M. R. Critical role for the tapasin-docking site of TAP2 in the functional integrity of the MHC class I-peptide-loading complex. J Immunol 175, 5104-5114 (2005).
37 Panter, M. S., Jain, A., Leonhardt, R. M., Ha, T. & Cresswell, P. Dynamics of major histocompatibility complex class I association with the human peptide-loading complex. The Journal of biological chemistry 287, 31172-31184 (2012).
38 Sadasivan, B., Lehner, P. J., Ortmann, B., Spies, T. & Cresswell, P. Roles for calreticulin and a novel glycoprotein, tapasin, in the interaction of MHC class I molecules with TAP. Immunity 5, 103-114 (1996).
39 Gabathuler, R., Reid, G., Kolaitis, G., Driscoll, J. & Jefferies, W. A. Comparison of cell lines deficient in antigen presentation reveals a functional role for TAP-1 alone in antigen processing. The Journal of experimental medicine 180, 1415-1425 (1994).
40 Lampen, M. H. et al. CD8+ T cell responses against TAP-inhibited cells are readily detected in the human population. J Immunol 185, 6508-6517 (2010).
41 Henle, A. M., Nassar, A., Puglisi-Knutson, D., Youssef, B. & Knutson, K. L. Downregulation of TAP1 and TAP2 in early stage breast cancer. PloS one 12, e0187323 (2017).
42 Pedersen, M. H. et al. Downregulation of antigen presentation-associated pathway proteins is linked to poor outcome in triple-negative breast cancer patient tumors. Oncoimmunology 6, e1305531 (2017).
43 Qin, Z. et al. Increased tumorigenicity, but unchanged immunogenicity, of transporter for antigen presentation 1-deficient tumors. Cancer research 62, 2856-2860 (2002).
44 Meng, X. et al. Cytoplasmic Metadherin (MTDH) provides survival advantage under conditions of stress by acting as RNA-binding protein. The Journal of biological chemistry 287, 4485-4491 (2011).
Liu, R., Ma, Y. & Chen, X. Quantitative assessment of the association between TAP2 rs241447 polymorphism and cancer risk. Journal of cellular biochemistry 120, 15867-15873 (2019).
46 Chou, H. L., Tian, L., Kumamaru, T., Hamada, S. & Okita, T. W. Multifunctional RNA Binding Protein OsTudor-SN in Storage Protein mRNA Transport and Localization. Plant physiology 175, 1608-1623 (2017).
47 Hsu, J. C., Reid, D. W., Hoffman, A. M., Sarkar, D. & Nicchitta, C. V. Oncoprotein AEG-1 is an endoplasmic reticulum RNA-binding protein whose interactome is enriched in organelle resident protein-encoding mRNAs. RNA (New York, N.Y 24, 688-703 (2018).
48 Jariwala, N. et al. Role of the staphylococcal nuclease and tudor domain containing 1 in oncogenesis (review). International journal of oncology 46, 465-473 (2015).
49 Tsuchiya, N. et al. SND1, a component of RNA-induced silencing complex, is up-regulated in human colon cancers and implicated in early stage colon carcinogenesis. Cancer research 67, 9568-9576 (2007).
50 Ohs, I. et al. Restoration of Natural Killer Cell Antimetastatic Activity by IL12 and Checkpoint Blockade. Cancer research 77, 7059-7071, doi:10.1158/0008-5472.CAN-17-1032 (2017).
51 Shen, M. et al. Tinagl1 Suppresses Triple-Negative Breast Cancer Progression and Metastasis by Simultaneously Inhibiting Integrin/FAK and EGFR Signaling. Cancer cell 35, 64-80 e67 (2019).
52 Jiang, Y. Z. et al. Preoperative measurement of breast cancer overestimates tumor size compared to pathological measurement. PloS one 9, e86676, doi:10.1371/journal.pone.0086676 (2014).
53 Xiao, Y. et al. Multi-Omics Profiling Reveals Distinct Microenvironment Characterization and Suggests Immune Escape Mechanisms of Triple-Negative Breast Cancer. Clin Cancer Res 25, 5002-5014, doi:10.1158/1078-0432.CCR-18-3524 (2019).
54 Shackleton, M. et al. Generation of a functional mammary gland from a single stem cell. Nature 439, 84-88 (2006).
55 Twyman-Saint Victor, C. et al. Radiation and dual checkpoint blockade activate non-redundant immune mechanisms in cancer. Nature 520, 373-377 (2015).

The foregoing example has been published in Nat Cancer 3, 60-74 (2022), the entire content of which is incorporated herein by reference in its entirety.

### Example 3

### Synthetic Experimentals

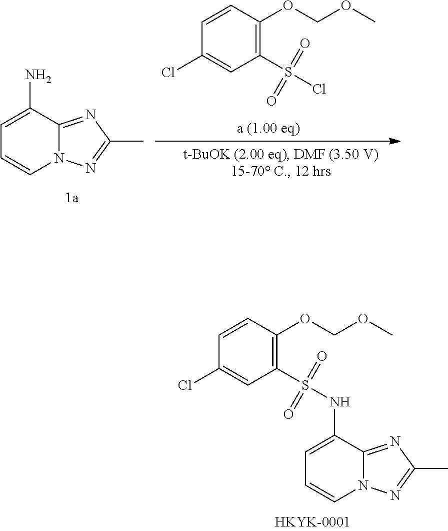 [SMILES: Cc1nc2c(N)cccn2n1 ; COCOc1ccc(Cl)cc1S(=O)(=O)Cl ; COCOc1ccc(Cl)cc1S(=O)(=O)Nc1cccn2nc(C)nc12]

Setup the reactor R-1 (50 mL). Charged compound 1a (20.0 mg, 135 μmol, 1.00 eq) and DMF (70.0 uL) into the R-1 at 15-20° C. Charged compound a (36.6 mg, 135 μmol, 1.00 eq) and t-BuOK (30.3 mg, 270 μmol, 2.00 eq) into the R-1 at 15-20° C. under N₂. The reaction mixture was stirred at 65-70° C. for 12 hrs. LCMS and HPLC showed the product was formed. The reaction mixture was concentration under vacuum. The crude product was purified by reversed-phase HPLC column: Phenomenex Luna C18 75*30 mm*3 μm; mobile phase: [water (TFA)-ACN]; B %: 25%-55%, 8 min. HKYK-0001 (10.0 mg, 16.8% yield, 86.9% purity) was obtained as brown solid. 1H NMR: (400 MHz, MeOD) δ 8.37 (d=6.8 MHz, 1H), 7.87 (d=2.8 MHz, 1H), 7.52 (t=8.8 MHz, 2H), 7.25 (d=8.8 MHz, 1H), 7.01 (t=8.8 MHz, 1H), 5.25 (s, 2H), 3.36 (s, 3H), 2.50 (s, 3H).

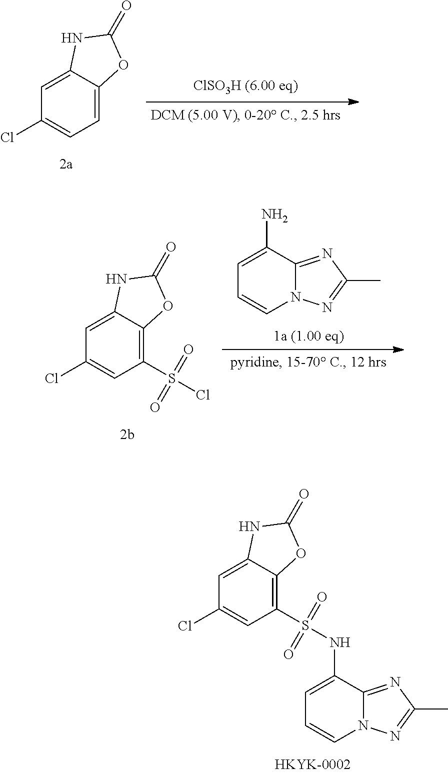 [SMILES: O=c1[nH]c2cc(Cl)ccc2o1 ; O=c1[nH]c2cc(Cl)cc(S(=O)(=O)Cl)c2o1 ; Cc1nc2c(N)cccn2n1 ; Cc1nc2c(NS(=O)(=O)c3cc(Cl)cc4[nH]c(=O)oc34)cccn2n1]

Set up the reactor R-1 (100 mL). Charged compound 2a (1.00 g, 5.90 mmol, 1.00 eq) and DCM (5.00 mL) at 15-20° C. Cooled to 0-5° C., dropwise sulfurochloridic acid (4.12 g, 35.4 mmol, 2.36 mL, 6.00 eq) in DCM (12.0 mL) to the R-1 at 0-5° C. The reaction mixture stirred at 15-20° C. for 2.5 hrs. LCMS showed the reaction product was formed. Added the reaction mixture into the H₂O (2.00 mL) at 0-5° C. Filtered and collected the filter cake and without purification. Compound 2b (1.00 g, 3.73 mmol, 31.6% yield) was obtained as off white solid. 1H NMR: (400 MHz, DMSO-d₆) δ 11.89 (s, 1H), 7.66 (s, 1H), 7.04 (s, 1H).

Setup the reactor R-1 (50 mL). Charged compound 1a (10.0 mg, 67.5 μmol, 1.00 eq) and pyridine (0.7 mL) into the R-1 at 15-20° C. Charged compound 2b (18.09 mg, 67.49 umol, 1 eq) into the R-1 at 15-20° C. under N₂. The reaction mixture was stirred at 65-70° C. for 12 hrs. HPLC and LCMS showed the product was formed. Four reactions were combined for workup. The reaction mixture was concentration vacuum. The crude product was purified by reversed-phase HPLC column: C18-1 150*30 mm*5 μm; mobile phase: [water (TFA)-ACN]; B %: 10%-55%, 8 min. HKYK-0002 (10.0 mg, 9.56% yield, 98.0% purity) was obtained as a white solid. ¹H NMR: (400 MHz, MeOD) δ 8.34 (d, J=6.8 Hz, 1H), 8.01 (s, 1H), 7.43 (d, J=6.8 Hz, 1H), 7.22 (s, 1H), 6.95 (t, J=7.2 Hz, 1H), 2.51 (s, 3H).

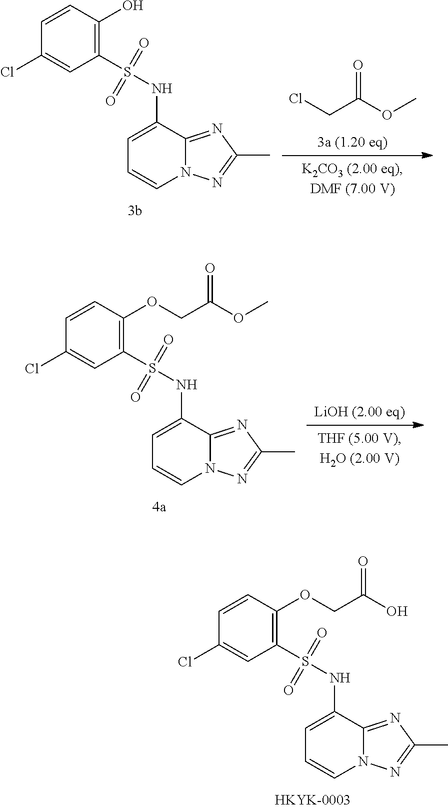

Set up the reactor R-1 (50 mL). Charged compound 3b (0.30 g, 885 μmol, 1.00 eq) and DMF (2.10 mL) into the R-1 at 15-20° C. Charged K₂CO₃ (244 mg, 1.77 mmol, 2.00 eq) into the R-1 at 15-20° C. under N₂. Warmed to 40° C. Charged methyl 2-chloroacetate (115 mg, 1.06 mmol, 1.20 eq) into the reaction mixture at 40° C. The reaction mixture was stirred at 40-50° C. for 1 hr. TLC (Petroleum ether:Ethyl acetate=2:1) showed the reaction was completed. The reaction mixture was concentration vacuum. The crude product was purified by prep-HPLC (column: Waters Xbridge BEH C18 100*30 mm*10 μm; mobile phase: [water(NH₄HCO₃)-ACN];B %: 10%-40%, 8 min) to get the product. 4a (0.1 g, 243 μmol, 27.5% yield) was obtained as white solid.

Set up the reactor R-1 (50 mL). Charged compound 4a (0.1 g, 243 μmol, 1.00 eq), THF (0.50 mL) and water (0.20 mL) into the R-1 at 15-20° C. Charged LiOH. H₂O (20.4 mg, 486 μmol, 2.00 eq) into the R-1 at 15-20° C. under N₂. Warmed to 40° C., and stirred for 1 hr. TLC (Petroleum ether:Ethyl acetate=1:1) showed the reaction was completed. The reaction mixture was concentration vacuum. The crude was purified by prep-TLC (Petroleum ether:Ethyl acetate=1:1) to get the product. HKYK-0003 (11.0 mg, 26.9 umol, 97.1% purity) was obtained as white solid. 1H NMR: (400 MHz, DMSO-d6) δ 8.58 (d, J=6.4 Hz, 1H), 7.54-7.61 (m, 2H), 7.12-7.28 (m, 1H), 6.90-7.03 (m, 1H), 4.72 (s, 2H), 2.42 (s, 3H).

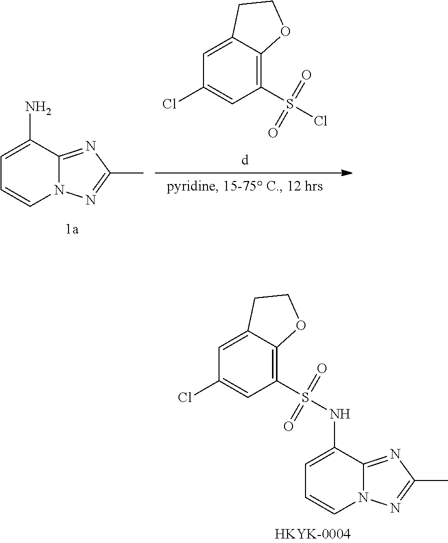 [SMILES: Cc1nc2c(N)cccn2n1 ; O=S(=O)(Cl)c1cc(Cl)cc2c1OCC2 ; Cc1nc2c(NS(=O)(=O)c3cc(Cl)cc4c3OCC4)cccn2n1]

Setup the reactor R-1 (50 mL). Charged compound 1a (10.0 mg, 67.5 μmol, 1.00 eq) and pyridine (0.70 mL) into the R-1 at 15-20° C. Charged compound d (17.1 mg, 67.5 μmol, 1.00 eq) into the R-1 at 15-20° C. under N₂. The reaction mixture was stirred at 70-75° C. for 12 hrs. LCMS showed the reaction was completely consumed, the product was formed. 43 reactions were combined for workup. The reaction mixture was concentrated under vacuum. The crude product was purified by reversed-phase HPLC column: Phenomenex Luna C18 75*30 mm*3 μm; mobile phase: [water (TFA)-ACN]; B %: 20%-50%, 7 min. HKYK-0004 (10.0 mg, 13.27% yield, 98.0% purity) was obtained as white solid which was checked by HNMR and LCMS. ¹H NMR: (400 MHz, CDCl₃) δ 8.25 (d, J=6.4 Hz, 1H), 7.77 (d, J=7.6 Hz, 1H), 7.60 (s, 1H), 7.27 (s, 1H), 7.04 (t, J=7.6 Hz, 1H), 4.70 (t, J=8.8 Hz, 2H), 3.18 (t, J=8.8 Hz, 2H), 2.61 (s, 3H).

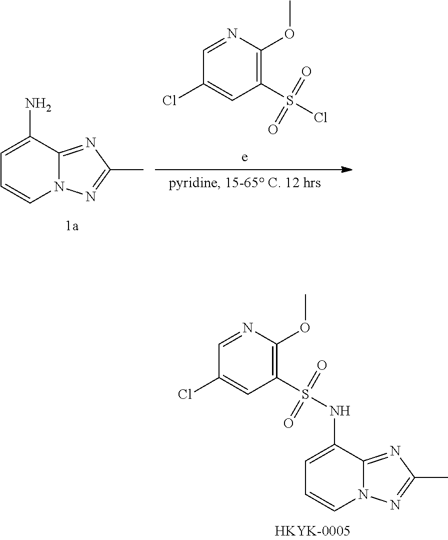 [SMILES: Cc1nc2c(N)cccn2n1 ; COc1ncc(Cl)cc1S(=O)(=O)Cl ; COc1ncc(Cl)cc1S(=O)(=O)Nc1cccn2nc(C)nc12]

Setup the reactor R-1 (50 ml). Charged compound 1a (10.0 mg, 67.5 μmol, 1.00 eq) and pyridine (70 μL) into the R-1 at 15-20° C. Charged compound e (16.3 mg, 67.5 μmol, 1.00 eq) into the R-1 at 15-20° C. under N₂. The reaction mixture was stirred at 60-65° C. for 12 hrs. LCMS showed the reaction product was formed. 4 reactions were combined for workup. The reaction mixture was concentrated under vacuum. The crude product was purified by reversed-phase HPLC: column: C18-1 150*30 mm*5 μm; mobile phase: [water (TFA)-ACN]; B %: 15%-60%, 8 min. HKYK-0005 (10.0 mg, 97.2% purity) was obtained as white solid which was checked by LCMS and ¹H NMR. 1H NMR: (400 MHz, DMSO-d₆) δ 8.66 (d, J=6.8 Hz, 1H), 8.44 (d, J=2.4 Hz, 1H), 8.16 (d, J=2.4 Hz, 1H), 7.44 (d, J=7.6 Hz, 1H), 7.07 (t, J=7.2 Hz, 1H), 3.75 (s, 3H), 2.39 (s, 3H).

Compounds HKYK-0007 and HKYK-0009-HKYK-0010 were synthesized according to the following synthetic route, wherein R corresponds to R⁴ in a compound of Structural Formula I.

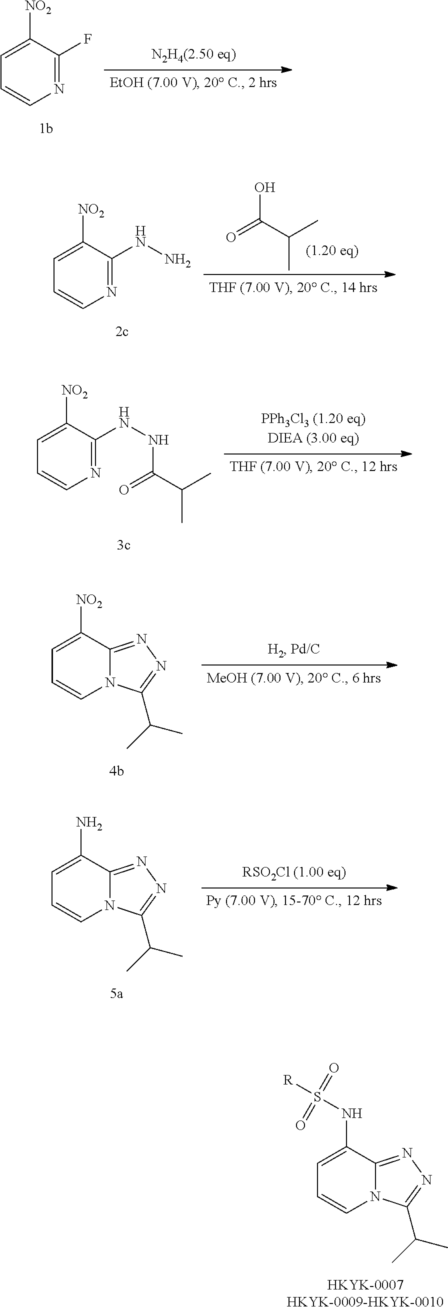

Set up a reactor R-1 with an agitator. (Note: the reactor R-1: 250 mL reactor). Charged EtOH (70.0 mL) into the reactor R-1 at 20-25° C. under nitrogen. Charged Compound 1b (10.0 g, 51.7 mmol, 1.00 eq) into the reactor R-1 at 20-25° C. under nitrogen. Cooled the mixture to 0-5° C. under nitrogen. Dropwise added N₂H₄ (6.41 g, 127 mmol, 6.20 mL, 2.50 eq) into the reactor R-1 at 0-5° C. under nitrogen. Stirred the mixture at 20-25° C. for 2 h under nitrogen. Took a sample (Start material RT=2.095 min, Product RT=0.129 min) that the reaction was finished completely. Filtered and rinsed the wet cake with ethyl alcohol (20 mL) twice. Collected the wet cake and concentrated at 40-45° C. to give the residual. Without purification and used to next step directly. Compound 2c (6.50 g, 44.9 mmol, 66.9% yield) as white solid.

Set up the reactor R-1. (Note: the reactor R-1: 10 mL). Charged compound 2c (5.00 g, 16.2 mmol, 1.00 eq) and THF (35.0 mL) into the reactor R-1 at 15-20° C. Charged isobutyric acid (1.74 g, 16.2 mmol, 1.20 eq) into the reaction mixture. Stirred the mixture at 20° C. for 14 hrs under N₂. TLC (Petroleum ether:Ethyl acetate=3:1) showed the starting material was consumed completely. Charged 20.0 mL ethyl acetate and 20.0 mL H₂O into the reactor R-1. Separated organic layers and collected. The aqueous layer was washed with ethyl acetate (10.0 mL). The combined organic layer was washed with brine water (10.0 mL). Compound 3c (2.10 g, crude) was obtained as yellow solid.

Set up the reactor R-1. (Note: the reactor R-1: 10 mL). Charged compound 3c (2.00 g, 8.92 mmol, 1.00 eq), THF (14.0 mL), PPh₃Cl₂ (3.57 g, 10.7 mmol, 1.20 eq) and DIEA (3.46 g, 26.8 mmol, 4.66 mL, 3.00 eq) into the reactor R-1 at 15-20° C. Cooled the mixture to 0-5° C. Stirred the mixture at 20° C. for 12 hrs under N₂. TLC (Petroleum ether:Ethyl acetate=3:1) showed the starting material was consumed completely. Charged 20.0 mL ethyl acetate and 20.0 mL H₂O into the reactor R-1. Separated organic layers and collected. The aqueous layer was washed with ethyl acetate (10.0 mL). The combined organic layer was washed with brine water (10.0 mL). Compound 4b (1.10 g, crude) was obtained as yellow solid.

Set up the reactor R-1. (Note: the reactor R-1: 10 mL). Charged compound 4b (1.40 g, 6.81 mmol, 1.00 eq), MeOH (7.00 mL) into the reactor R-1 at 15-20° C. Charged Pd/C (100 mg, 10%) into the reaction under N₂. Stirred the mixture at 20° C. for 6 hrs under H₂ (40 PSI). LCMS showed the reaction was completely consumed and the product was formed. The reaction mixture was filtered, and the filter was concentrated. Compound 5a (0.8 g, crude) was obtained as yellow solid.

To prepare HKYK-0007 from 5a: Set up the reactor R-1 (50 mL). Charged compound 5a (50.0 mg, 284 μmol, 1.00 eq) and pyridine (Py, 0.35 mL) into the R-1 at 15-20° C. Charged compound 7a (76.1 mg, 284 μmol, 1.00 eq) into the R-1 at 15-20° C. under N₂. The reaction mixture was stirred at 65-70° C. for 12 hrs. LCMS showed the product was formed. 2 reactions were worked up together. The reaction mixture was concentrated in vacuum. The crude product was purified by reversed-phase HPLC column: Phenomenex C18 80*30 mm*3 μm; mobile phase: [water (TFA)-ACN]; B %: 20%-50%, 8 min. HKYK-0007 (20.0 mg, 5.85% yield, 67.7% purity) was obtained as gray solid which was checked by ¹H NMR and LCMS. The crude product was purified by reversed-phase HPLC column: Waters Xbridge BEH C18 250*50 mm*10 μm; mobile phase: [water(NH₃H₂O+NH₄HCO₃)-ACN]; B %: 1%-40%, 8 min. HKYK-0007 (10 mg, 98.7% purity) was obtained as white solid which was checked by HNMR. 1H NMR: (400 MHz, MeOD) δ 7.92 (s, 1H), 7.16 (s, 1H), 7.08 (s, 1H), 6.84 (t, J=6.8 Hz, 1H), 3.49-3.52 (m, 1H), 1.47 (t, J=6.8 Hz, 1H).

To prepare HKYK-0009 from 5a: Set up the reactor R-1 (50 mL). Charged compound 5a (10.0 mg, 56.8 μmol, 1.00 eq) and Py (0.35 mL) into the R-1 at 15-20° C. Charged compound 9a (14.4 mg, 56.8 μmol, 1.00 eq) into the R-1 at 15-20° C. under N₂. The reaction mixture was stirred at 65-70° C. for 12 hrs. LCMS showed the product was formed. 4 reactions were worked up together. The reaction mixture was concentrated under vacuum. The crude product was purified by reversed-phase HPLC column column: Phenomenex C18 80*30 mm*3 μm; mobile phase: [water (TFA)-ACN]; B %: 20%-50%, 8 min. HKYK-0009 (10.0 mg, 10.98% yield, 97.9% purity) was obtained as an off white solid which was checked by ¹H NMR and LCMS. 1H NMR: (400 MHz, CDCl₃) δ 8.25 (d, J=6.4 Hz, 1H), 7.51 (s, 1H), 7.43 (s, 1H), 7.36 (d, J=7.2 Hz, 1H), 7.06 (t, J=7.2 Hz, 1H), 4.64 (t, J=8.8 Hz, 2H), 3.52-3.56 (m, 2H), 3.21 (t, J=8.8 Hz, 2H), 1.47 (d, J=6.8 Hz, 6H).

To prepare HKYK-0010 from 5a: Set up the reactor R-1 (50 mL). Charged compound 5a (10.0 mg, 56.7 μmol, 1.00 eq) and Py (0.35 mL) into the R-1 at 15-20° C. Charged compound 10a (13.7 mg, 56.7 μmol, 1.00 eq) into the R-1 at 15-20° C. under N₂. The reaction mixture was stirred at 65-70° C. for 12 hrs. LCMS showed the product was formed. 2 reactions were worked up together. The reaction mixture was concentrated under vacuum. The crude product was purified by reversed-phase HPLC column column column: Phenomenex C18 80*30 mm*3 μm; mobile phase: [water (TFA)-ACN]; B %:20%-50%, 8 min]. HKYK-0010 (10 mg, 23.4% yield, 97.6% purity) was obtained as an off white solid which was checked by ¹H NMR and LCMS.

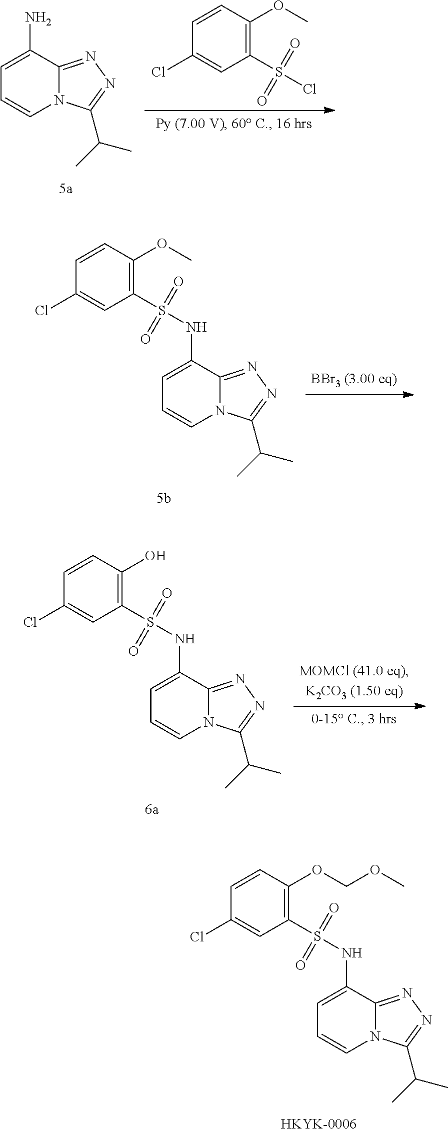 [SMILES: CC(C)c1nnc2c(N)cccn12 ; COc1ccc(Cl)cc1S(=O)(=O)Cl ; COc1ccc(Cl)cc1S(=O)(=O)Nc1cccn2c(C(C)C)nnc12 ; CC(C)c1nnc2c(NS(=O)(=O)c3cc(Cl)ccc3O)cccn12 ; COCOc1ccc(Cl)cc1S(=O)(=O)Nc1cccn2c(C(C)C)nnc12]

Set up the reactor R-1 (50 mL). Charged compound 5a (100 mg, 567.48 μmol, 1.00 eq) and Py (0.7 mL) into the R-1 at 15-20° C. Charged 5-chloro-2-methoxy-benzenesulfonyl chloride (136 mg, 567 μmol, 1.00) into the R-1 at 15-20° C. The reaction mixture was stirred at 60° C. for 16 hrs. LCMS showed the product was formed. The reaction mixture was concentrated under vacuum. The crude product was purified by reversed-phase HPLC column: Phenomenex C18, 80*30 mm*3 μm; mobile phase: [water(TFA)-ACN];B %: 25%-55%, 8 min. Compound 5b (50 mg, 124 μmol, 95% purity) was obtained as off white solid. 1H NMR: (400 MHz, MeOD) δ 8.32 (d, J=2.4 Hz, 1H), 8.24 (s, 1H), 8.22 (d, J=2.4 Hz, 1H), 7.39 (d, J=7.2 Hz, 1H), 7.03 (t, J=7.2 Hz, 1H), 3.92 (s, 3H), 3.50-3.57 (m, 1H), 1.47 (d, J=6.8 Hz, 6H).

Set up the reactor R-1 (50 mL). Charged compound 5b (50.0 mg, 131 μmol, 1.00 eq) and DCM (350 μL) into the R-1 at 15-20° C. Cooled to 0-5° C. Charged BBr₃ (98.7 mg, 394 μmol, 37.9 μL, 3.00 eq) into the R-1 at 0-5° C. under N₂. The reaction mixture was stirred at 15-20° C. for 2 hrs. LCMS showed the product was formed. The reaction mixture was quenched with MeOH (1.00 mL) and concentrated under vacuum. Without purification. Compound 6a (40 mg, 109.04 μmol, 41.53% yield) was obtained as yellow oil which was checked by ¹H NMR. 1H NMR: (400 MHz MeOD) δ 8.69 (d, J=7.2 Hz, 1H), 7.63 (s, 1H), 7.58 (d, J=6.8 Hz, 1H), 7.48 (d, J=7.2 Hz, 1H), 7.46 (t, J=6.8 Hz, 1H), 7.00 (d, J=9.2 Hz, 1H), 3.64-3.71 (m, 1H), 1.52 (d, J=6.8 Hz, 6H).

Set up the reactor R-1 (100 mL). Charged compound 6a (10.0 mg, 27.3 μmol, 1.00 eq) and ACN (70.0 μL) into the R-1 at 10-15° C. Charged K₂CO₃ (5.65 mg, 40.9 μmol, 1.50 eq) into the R-1 at 10-15° C. under N₂. Charged MOMCl (0.09 g, 1.12 mmol, 84.9 μL, 41.0 eq) into the R-1 at 0-5° C. under N₂. The reaction mixture was stirred at 10-15° C. for 3 hrs. LCMS showed the product was formed. The reaction mixture was quenched with H₂O (8.00 mL). Extracted with DCM (5.00 mL). The combined organic phase was concentrated under vacuum. The crude product was purified by reversed-phase HPLC column: Phenomenex Luna C1875*30 mm*3 μm; mobile phase: [water(TFA)-ACN];B %: 25%-55%, 8 min. Compound HKYK-0006 (6 mg, 12.85 μmol, 47.14% yield, 88% purity) was obtained as white solid which was checked by ¹H NMR and LCMS. 1H NMR: (400 MHz, CDCl₃) δ 8.04 (d, J=6.8 Hz, 1H), 7.52 (s, 1H), 7.45 (d, J=7.6 Hz, 1H), 7.25-7.29 (m, 1H), 6.86 (d, J=8.8 Hz, 1H), 6.35 (s, 1H), 3.50-3.58 (m, 1H), 3.49 (s, 3H), 1.48 (d, J=7.6 Hz, 6H).

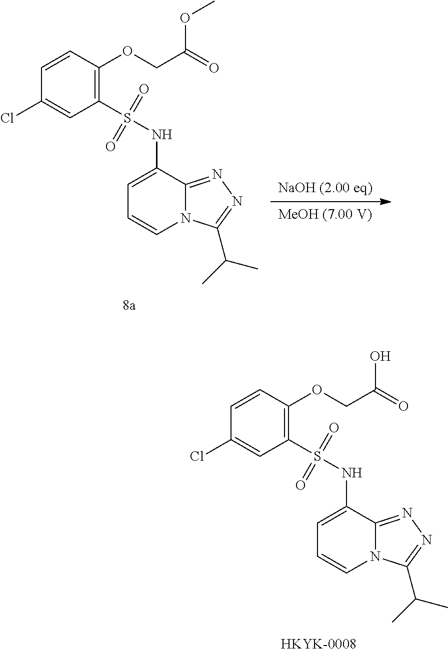 [SMILES: COC(=O)COc1ccc(Cl)cc1S(=O)(=O)Nc1cccn2c(C(C)C)nnc12 ; CC(C)c1nnc2c(NS(=O)(=O)c3cc(Cl)ccc3OCC(=O)O)cccn12]

Set up the reactor R-1 (50 ml). Charged 8a (20 mg, 45.57 μmol, 1.00 eq) and MeOH (0.14 mL) into the R-1 at 15-20° C. Charged NaOH (7 M, 13.02 μL, 2 eq) into the R-1 at 15-20° C. under N₂. The reaction mixture was stirred at 15-20° C. for 2 hrs. LCMS showed the reaction was completely consumed. The reaction mixture was concentrated under vacuum at 40-45° C. The crude product was purified by reversed-phase HPLC column: Phenomenex Luna C18 75*30 mm*3 μm; mobile phase: [water (TFA)-ACN]; B %: 20%-50%, 8 min. HKYK-0008 (10 mg, 23.54 μmol, 51.6% yield, 100% purity) was obtained as white solid, which was checked by ¹H NMR and LCMS. 1H NMR: (400 MHz, MeOD) δ 8.04 (d, J=7.2 Hz, 1H), 7.77 (d, J=2.8 Hz, 1H), 7.51 (d, J=2.4 Hz, 1H), 7.48 (d, J=2.4 Hz, 1H), 7.04 (d, J=8.8 Hz, 1H), 6.89 (t, J=7.2 Hz, 1H), 3.42-3.48 (m, 1H), 3.49 (s, 3H), 1.42 (d, J=6.8 Hz, 6H).

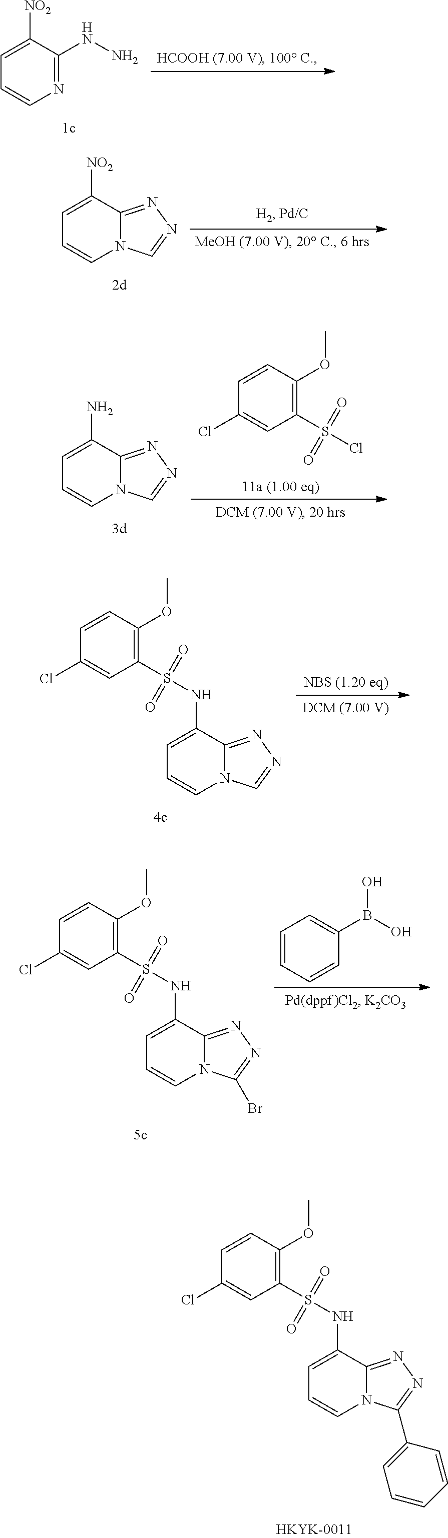

Set up the reactor R-1 (50 ml). Charged compound 1c (2.00 g, 1.00 eq) and HCOOH (14.0 mL) into the R-1 at 15-20° C. The reaction mixture was stirred at 100° C. for 12 hrs. LCMS showed the reaction was completely consumed. The reaction mixture was concentrated under vacuum at 40-45° C. The crude was purified by silica gel chromatography (Petroleum ether:Ethyl acetate=1:0˜0:1) to get the product. 2d (1.10 g, 51.6% yield) was obtained as white solid.

Set up the reactor R-1 (35 mL). To a solution of compound 2d (1.00 g, 6.09 mmol, 1.0 eq) MeOH (7.00 mL) was added Pd/C (10.0 mg, 10% purity) under N₂. The suspension was degassed under vacuum and purged with H₂ three times. The mixture was stirred under H₂ (6.09 mmol) (40 psi) at 20° C. for 6 hrs. TLC (Petroleum ether:Ethyl acetate=2:1) showed the reaction was completely consumed and the product was formed. The reaction mixture was filtered, and the filter was concentrated. Without purification. Compound 3d (1.20 g, 8.95 mmol, 73.4% yield) was obtained as gray solid.

Set up the reactor R-1 (50 mL). Charged compound 3d (200 mg, 1.49 mmol, 1.00 eq) and Py (1.40 mL) into the R-1 at 15-20° C. Charged compound 11a (359 mg, 1.49 mmol, 1.00 eq) into the R-1 at 15-20° C. under N₂. The reaction mixture was stirred at 70-75° C. for 12 hrs. TLC (PE:EA=1:1) showed the reaction was consumed completely. The reaction mixture was concentrated under vacuum. The crude product was triturated with MeOH at 15° C. for 1 hr. Compound 4c (80.00 mg, 236 μmol, 15.8% yield) was obtained as off white solid.

Set up the reactor R-1 (50 mL). Charged compound 4c (50.0 mg, 147 μmol, 1.00 eq) and DCM (0.35 mL) into the R-1 at 0-5° C. Charged NBS (31.52 mg, 177.11 μmol, 1.20 eq) into the R-1 at 0-5° C. under N₂. The reaction mixture was stirred at 0-5° C. for 2 hrs. TLC (Petroleum ether:Ethyl acetate=4:1) showed the reaction was consumed completely. The reaction mixture was quenched with NaHCO₃ (3.00 mL) and extracted with DCM (3.00 mL). The combined organic phase was concentrated under vacuum. Without purification. Compound 5c (50 mg, 81.11% yield) was obtained as off white solid.

Set up the reactor R-1 (50 mL). Charged compound 5c (50.0 mg, 120 μmol, 1.00 eq) and 1.4-dioxane (0.35 mL) into the R-1 at 15-20° C. Charged phenylboronic acid (21.9 mg, 180 μmol, 1.50 eq) and Pd(dppf)Cl₂. CH2Cl₂ (9.78 mg, 12.0 μmol, 0.1 eq) and KOAc (47.0 mg, 479 μmol, 4.00 eq) into the R-1 at 10-15° C. under N₂. The reaction mixture was stirred at 110-120° C. for 2 hrs. LCMS showed the product was formed. The reaction mixture was concentrated under vacuum. The crude product was purified by reversed-phase HPLC column: Phenomenex Luna C18 75*30 mm*3 μm; mobile phase: [water(TFA)-ACN];B %: 45%-75%, 8 min. HKYK-0011 (10.0 mg, 19.43% yield, 96.5% purity) was obtained as white solid which was checked by ¹H NMR and LCMS. 1H NMR: (400 MHz, DMSO-d6). δ 8.49 (s 1H), 7.95 (d, J=2.0 Hz, 2H), 7.78 (d, J=2.8 Hz, 1H), 7.56 (d, J=8.8 Hz, 1H), 7.50-7.54 (m, 5H), 7.34 (d, J=8.0 Hz, 1H), 7.18 (d, J=8.8 Hz, 1H), 3.63 (s, 3H).

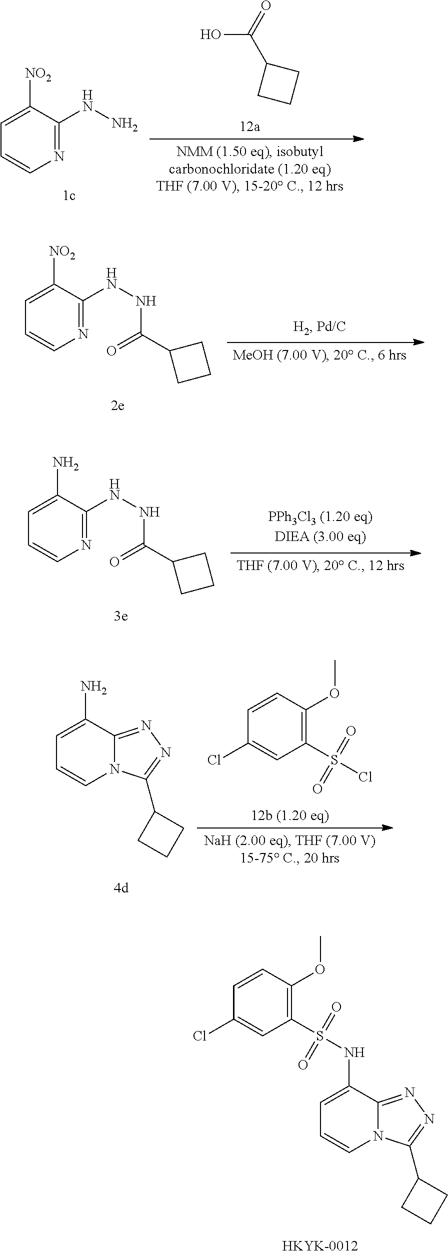

Set up the reactor R-1. (Note: the reactor R-1: 100 mL). Charged compound 12a (3.90 g, 38.9 mmol, 3.71 mL, 1.20 eq), NMM (4.92 g, 48.7 mmol, 5.35 mL, 1.50 eq), isobutyl carbonochloridate (5.32 g, 38.9 mmol, 5.11 mL, 1.20 eq), THF (35.0 mL) into the reactor R-1 at 15-20° C. Charged compound 1c (5.00 g, 32.4 mmol, 1.00 eq) into the reactor R-1 at 15-20° C. Stirred the mixture at 15-20° C. for 12 hrs under N₂. TLC (Petroleum ether:Ethyl acetate=2:1) showed the starting material was consumed completely. Charged 20.0 mL ethyl acetate and 20.0 mL H₂O into the reactor R-1. Separated organic layers and collected. The aqueous layer was washed with ethyl acetate (10.0 mL). The combined organic layer was washed with brine water (10 mL). Without purification. Compound 2e (6.00 g, crude) was obtained as a brown solid.

Set up the reactor R-1 (35 mL). To a solution of compound 2e (5.00 g, 6.09 mmol, 1.0 eq) MeOH (35.0 mL) was added and Pd/C (500 mg, 10% purity) under N₂. The suspension was degassed under vacuum and purged with H₂ three times. The mixture was stirred under H₂ (40 psi) at 20° C. for 6 hrs. TLC (Petroleum ether:Ethyl acetate=2:1) showed the reaction was completely consumed and the product was formed. The reaction mixture was filtered, and the filter was concentrated. Without purification. Compound 3e (3.00 g, 73.4% yield) was obtained as gray solid.

Set up the reactor R-1. (Note: the reactor R-1: 10 mL). Charged compound 3e (2.00 g, 8.47 mmol, 1.00 eq), THF (1.40 mL), 1,1,1,2,2,2-hexachloroethane (4.01 g, 16.93 mmol, 1.92 mL, 2 eq) and DIEA (3.28 g, 25.40 mmol, 4.42 mL, 3 eq) into the reactor R-1 at 15-20° C. Cooled the mixture to 0-5° C. Charged PPh₃Cl₂ (3.39 g, 10.2 mmol, 1.20 eq) into the reactor R-1 at 0-5° C. Stirred the mixture at 15° C. for 12 hrs under N₂. TLC (Petroleum ether:Ethyl acetate=1:1) showed the starting material was consumed completely. Charged 20.0 mL ethyl acetate and 20.0 mL H₂O into the reactor R-1. Separated organic layers and collected. The aqueous layer was washed with ethyl acetate (10.0 mL). The combined organic layer was washed with brine water (10.0 mL). Without purification. Compound 4d (1.10 g, crude) was obtained as yellow solid.

Set up the reactor R-1 (50 mL). Charged compound 4d (50.0 mg, 266 μmol, 1.00 eq) and THF (0.35 mL) into the R-1 at 15-20° C. Charged compound 12b (64.0 mg, 266 μmol, 1.00 eq) and NaH (213 mg, 53 μmol, 60% purity, 2.00 eq) into the R-1 at 15-20° C. under N₂. The reaction mixture was stirred at 70-75° C. for 20 hrs. LCMS showed the product was formed. The reaction mixture was concentrated under vacuum. The crude product was purified by reversed-phase HPLC column: Phenomenex Luna C18 75*30 mm*3 μm; mobile phase: [water(TFA)-ACN]; B %: 25%-55%, 8 min. HKYK-0012 (10.0 mg, 25.4 μmol, 9.58% yield) was obtained as white solid which was checked by ¹H NMR. ¹H NMR: (400 MHz, MeOD) δ 8.05 (d, J=6.8 Hz, 1H), 7.81 (s, 2H), 7.34 (d, J=6.8 Hz, 1H), 7.14 (d, J=8.8 Hz, 1H), 7.01 (t, J=6.8 Hz, 1H), 4.09 (q, J=5.2 Hz, 1H), 3.88 (s, 3H), 2.51-2.62 (m, 4H), 2.19-2.21 (m, 1H), 2.01-2.09 (m, 1H).

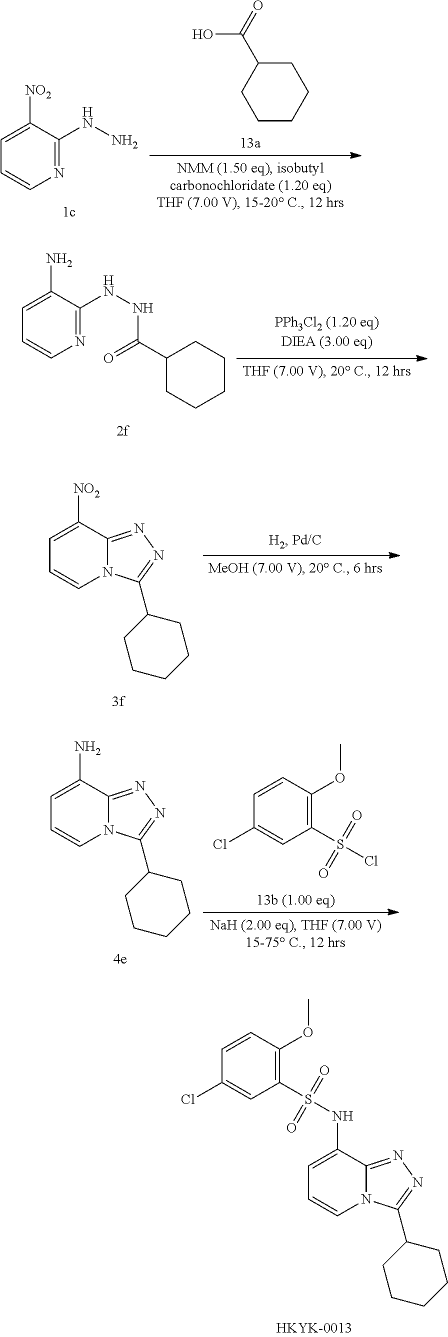

Set up the reactor R-1. (Note: the reactor R-1: 100 mL). Charged compound 13a (4.99 g, 38.9 mmol, 4.84 mL, 1.20 eq), NMM (4.92 g, 48.7 mmol, 5.35 mL, 1.50 eq), isobutyl carbonochloridate (5.32 g, 38.9 mmol, 5.11 mL, 1.20 eq) THF (35.0 mL) into the reactor R-1 at 15-20° C. Charged compound 1c (5.00 g, 32.4 mmol, 1.00 eq) into the reactor R-1 at 15-20° C. Stirred the mixture at 15-20° C. for 12 hrs under N₂. TLC (Petroleum ether:Ethyl acetate=2:1) showed the starting material was consumed completely. Charged 10.0 mL ethyl acetate and 10.0 mL H₂O into the reactor R-1. The aqueous layer was washed with ethyl acetate (10.0 mL). The combined organic layer was washed with brine water (10.0 mL). Without purification. Compound 2f (6.00 g, 22.7 mmol, 70.0% yield) was obtained as brown solid.

Set up the reactor R-1. (Note: the reactor R-1: 10 mL). Charged compound 2f (2.00 g, 7.57 mmol, 1.00 eq), THF (1.40 mL), 1,1,1,2,2,2-hexachloroethane (3.58 g, 15.1 mmol, 1.71 mL, 2.00 eq) and DIEA (2.93 g, 22.7 mmol, 3.95 mL, 3.00 eq) into the reactor R-1 at 15-20° C. Cooled the mixture to 0-5° C. Charged PPh₃Cl₂ (3.03 g, 9.08 mmol, 1.20 eq) into the reactor R-1 at 0-5° C. Stirred the mixture at 20° C. for 12 hrs under N₂. TLC (Petroleum ether:Ethyl acetate=1:1) showed the starting material was consumed completely. Charged 20.0 mL ethyl acetate and 20.0 mL H₂O into the reactor R-1. Separated organic layers and collected. The aqueous layer was washed with ethyl acetate (10.0 mL). The combined organic layer was washed with brine water (10.0 mL). Without purification. Compound 3f (1.10 g, crude) was obtained as yellow solid.

Set up the reactor R-1. (Note: the reactor R-1:35 mL). Charged Pd/C (100 mg, 10% purity), compound 3f (1.00 g, 4.06 mmol, 1.00 eq) and MeOH (7.00 mL) into the reactor R-1 at 15° C. under Ar₂. Heated the mixture to 40° C. Stirred the reactor mixture at 40° C. for 6 hrs under H₂ (45 psi). TLC (Petroleum ether:Ethyl acetate=1:1) showed the starting material was consumed completely. The mixture was filtered under reduced pressure to filtrate. Concentrated it in vacuum at 45° C. to remove MeOH. Without purification. Compound 4e (550 mg, crude) was obtained as yellow solid.

Set up the reactor R-1 (50 mL). Charged compound 4e (50.0 mg, 231 μmol, 1.00 eq) and THF (0.35 mL) into the R-1 at 15-20° C. Charged compound 13b (55.7 mg, 231 μmol, 1.00 eq) and NaH (18.5 mg, 462 μmol, 60% purity, 2.00 eq) into the R-1 at 15-20° C. under N₂. The reaction mixture was stirred at 70-75° C. for 12 hrs. LCMS showed the product was formed. The reaction was concentrated under vacuum. The crude product was purified by reversed-phase HPLC (column: Phenomenex Luna C18 75*30 mm*3 μm; mobile phase: [water(TFA)-ACN]; B %: 30%-60%, 8 min. HKYK-0013 (10.0 mg, 10.28% yield) was obtained as white solid which was checked by ¹H NMR. 1H NMR: (400 MHz, MeOD) δ 8.21 (d, J=7.2 Hz, 1H), 7.83 (s, 1H), 7.55 (d, J=8.8 Hz, 1H), 7.33 (d, J=7.2 Hz, 1H), 7.13 (d, J=7.2 Hz, 1H), 7.00 (t, J=7.2 Hz, 1H), 3.87 (s, 3H), 3.21-3.27 (m, 1H), 2.10 (d, J=11.2 Hz, 2H), 1.92 (d, J=11.2 Hz, 2H), 1.75-7.78 (m, 1H), 1.61-1.72 (m, 2H), 1.49-1.57 (m, 2H), 1.48-1.49 (m, 1H).

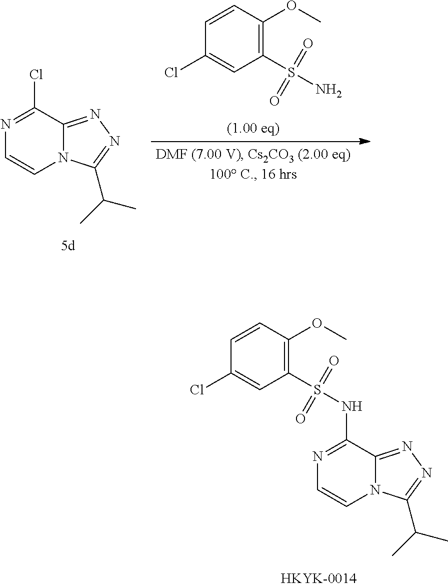 [SMILES: CC(C)c1nnc2c(Cl)nccn12 ; COc1ccc(Cl)cc1S(N)(=O)=O ; COc1ccc(Cl)cc1S(=O)(=O)Nc1nccn2c(C(C)C)nnc12]

Set up the reactor R-1 (50 mL). Charged compound 5d (10.0 mg, 50.9 μmol, 1.00 eq) and DMF (0.70 μL) into the R-1 at 15-20° C. Charged 5-chloro-2-methoxy-benzenesulfonamide (11.3 mg, 50.9 umol, 1.00 eq) and Cs₂CO₃ (33.1 mg, 102 μmol, 2.00 eq) into the R-1 at 15-20° C. under N₂. The reaction mixture was stirred at 100° C. for 16 hrs. LCMS showed the product was formed. The reaction mixture was concentrated under vacuum. The crude product was purified by reversed-phase HPLC column: Phenomenex Luna C1875*30 mm*3 μm; mobile phase: [water(TFA)-ACN];B %: 15%-45%, 7 min. HKYK-0014 (10 mg, 50.5% yield, 98.0% purity) was obtained as white solid which was checked by ¹H NMR and LCMS. ¹H NMR: (400 MHz, CDCl₃) δ 7.94 (s, 1H), 7.73 (d, J=5.6 Hz, 1H), 7.56 (dd, J=8.8 Hz, 1H), 7.3-7.17 (m, 2H), 3.77 (s, 3H), 3.43-3.50 (m, 1H), 1.46 (d, J=6.8 Hz, 6H).

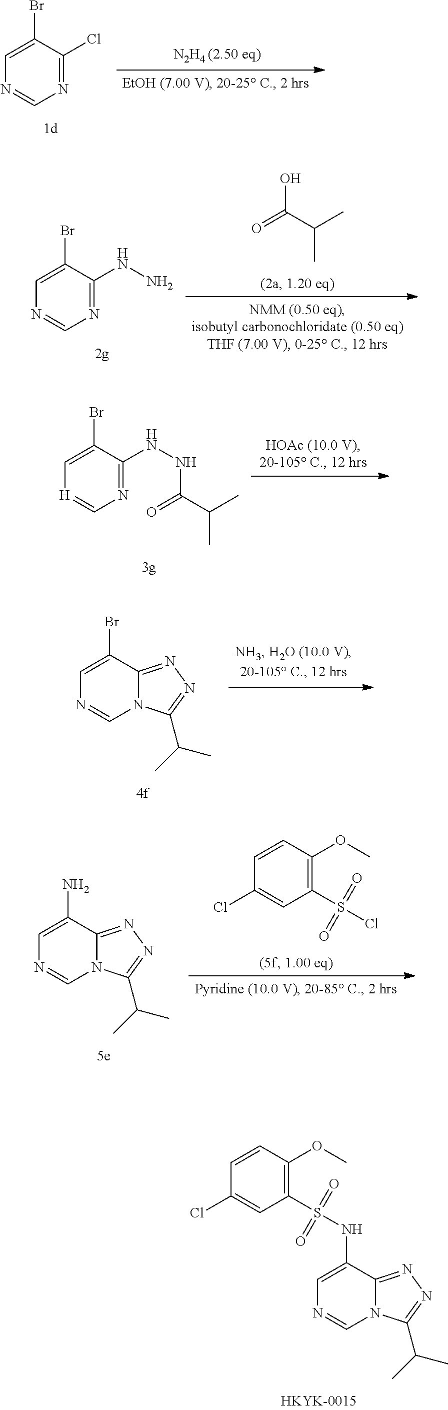

Set up a reactor R-1 with an agitator. (Note: the reactor R-1: 250 mL reactor). Charged EtOH (70.0 mL) into the reactor R-1 at 20-25° C. under nitrogen. Charged Compound 1d (10.0 g, 51.7 mmol, 1.00 eq) into the reactor R-1 at 20-25° C. under nitrogen. Cooled the mixture to 0-5° C. under nitrogen. Dropwise added N₂H₄ (6.41 g, 127 mmol, 6.20 mL, 2.50 eq) into the reactor R-1 at 0-5° C. under nitrogen. Stirred the mixture at 20-25° C. for 2 h under nitrogen. Took a sample (Starting material RT=2.095 min, Product RT=0.129 min) that the reaction was finished completely. Filtered and rinsed the wet cake with ethyl alcohol (20 mL) twice. Collected the wet cake and concentrated at 40-45° C. to give the residual. Without purification and used in next step directly. Gave Compound 2g (8.50 g, 44.9 mmol, 86.9% yield) as white solid, which was confirmed by ¹H NMR. 1H NMR: (400 MHz, DMSO-d6) δ ppm 8.53 (br s, 1H), 8.45 (s, 1H), 8.26 (s, 1H), 4.51 (br s, 2H).

Set up a reactor R-1 with an agitator. (Note: the reactor R-1: 250 mL Reactor). Charged Compound 2g (7.50 g, 39.6 mmol, 1.00 eq), Compound 2a (4.20 g, 47.6 mmol, 4.42 mL, 1.20 eq) and THF (52.5 mL) into the reactor R-1 at 20-25° C. Cooled the mixture to 0-5° C. under nitrogen. Added NMM (2.01 g, 19.8 mmol, 2.18 mL, 0.50 eq) and isobutyl carbonochloridate (2.71 g, 19.8 mmol, 2.61 mL, 0.50 eq) into the reactor R-1 at 0-5° C. under nitrogen. Stirred the mixture at 20-25° C. for 12 hrs under nitrogen. Took a sample (Product RT=0.371 min, Compound 2g RT=0.129 min) indicated the reaction was finished completely. Added H₂O (21.0 mL) into the mixture and extracted with ethyl acetate (21.0 mL×2). Washed the organic phase with brine (21.0 mL) and dried with Na₂SO₄. Concentrated the organic phase at 40-45° C. under reduced pressure to give a residue. The residue was purified by column chromatography (SiO₂, Petroleum ether/Ethyl acetate=10/1 to 0/1). Gave Compound 3g (7.00 g, 27.0 mmol, 68.0% yield) as white solid.

Set up a reactor R-1 with an agitator. (Note: the reactor R-1: 100 mL reactor). Charged Compound 3g (4.00 g, 15.4 mmol, 1.00 eq) and HOAc (40.0 mL) into the reactor R-1 at 20-25° C. under nitrogen. Adjusted the temperature to 100-105° C. under nitrogen. Stirred the mixture at 100-105° C. for 12 hrs under nitrogen. Took a sample (Compound 3f RT=0.382 min, Product RT=0.609 min) indicated the reaction was finished completely. Concentrated the mixture at 40-45° C. under reduced pressure to give a residue. The residue was purified by column chromatography (SiO₂, Petroleum ether/Ethyl acetate=10/1 to 0/1). To give Compound 4f (1.00 g, 4.15 mmol, 26.87% yield) as white solid, which was confirmed by ¹H NMR. ¹H NMR: (400 MHz, DMSO-d6) δ ppm 9.74 (s, 1H), 8.52 (s, 1H), 3.23 (dt, J=13.9, 6.92 Hz, 1H), 1.37 (d, J=6.97 Hz, 6H).

Set up a reactor R-1 with an agitator. (Note: the reactor R-1: 35.0 mL reactor). Charged Compound 4f (0.50 g, 2.07 mmol, 1.00 eq) and NH₃·H₂O (5.00 mL, 28.0% purity) into the reactor R-1 at 20-25° C. under nitrogen. Adjusted the temperature to 100-105° C. Took a sample (Compound 4e RT=0.609 min, Product RT=0.860 min) indicated the reaction was finished completely. Concentrated the mixture at 40-45° C. under reduced pressure to give a residue. The crude product was purified by reversed-phase HPLC. To give Compound 5e (0.10 g, 564 μmol, 27.2% yield) as white solid, which was confirmed by ¹H NMR. 1H NMR: (400 MHz, DMSO-d6) δ ppm 7.80 (br s, 1H), 7.65 (br s, 1H), 2.88-3.14 (m, 1H), 1.19-1.34 (m, 6H).

Set up a reactor R-1 with an agitator. (Note: the reactor R-1: 10.0 mL reactor). Charged Compound 5e (25.0 mg, 141 μmol, 1.00 eq) and pyridine (0.25 mL) into the reactor R-1 at 20-25° C. under nitrogen. Charged Compound 5f (34.0 mg, 141 μmol, 1.00 eq) into the reactor R-1 at 20˜25° C. under nitrogen. Adjusted the temperature to 80˜85° C. Stirred the mixture at 80-85° C. for 2 hrs under nitrogen. Took a sample (Compound 5e RT=0.860 min, Product RT=1.85 min) indicated the reaction was finished completely. Concentrated the mixture at 40-45° C. under reduced pressure to give a residue. The crude product was purified by reversed-phase HPLC to give HKYK-0015 (4.62 mg, 12.1 μmol, 8.58% yield, 95.1% purity) as white solid, which was confirmed by ¹H NMR. 1H NMR: (400 MHz, CDCl₃) δ ppm 8.20 (s, 1H), 8.08 (d, J=2.63 Hz, 1H), 7.96-8.03 (m, 1H), 7.61 (dd, J=8.91, 2.64 Hz, 1H), 6.96 (d, J=9.03 Hz, 1H), 3.91 (s, 3H), 3.17 (dt, J=13.96, 7.14 Hz, 1H), 1.40 (d, J=6.90 Hz, 6H). LCMS (Product RT=2.287 min).

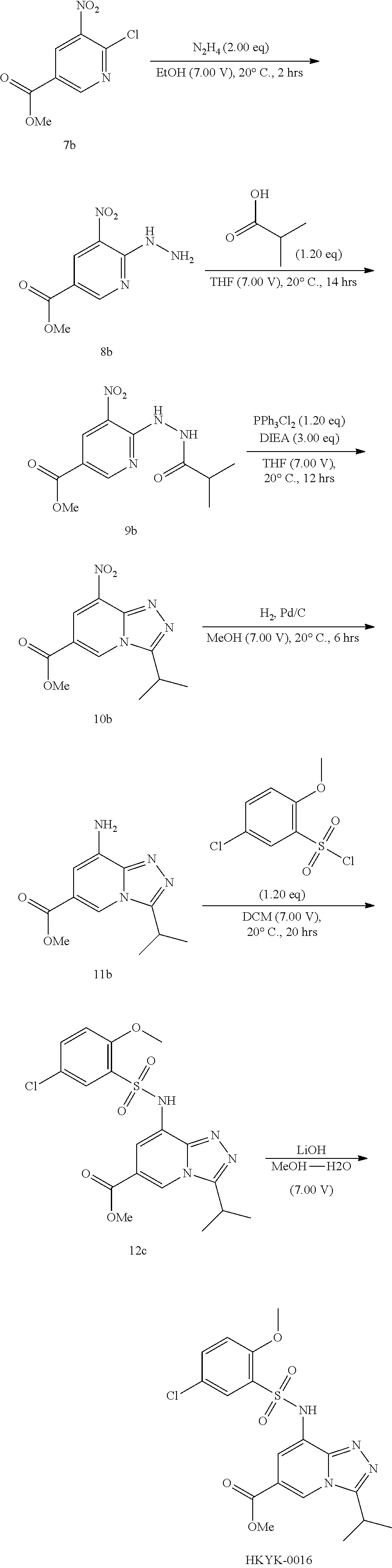

Set up a reactor R-1 with an agitator. (Note: the reactor R-1: 100 mL reactor). Charged EtOH (21.0 mL) into the reactor R-1 at 20-25° C. under nitrogen. Charged Compound 7b (3.00 g, 1.00 eq) into the reactor R-1 at 20-25° C. under nitrogen. Cooled the mixture to 0-5° C. under nitrogen. Dropwise added N₂H₄ (2.20 g, 2.00 eq) into the reactor R-1 at 0-5° C. under nitrogen. Stirred the mixture at 20-25° C. for 2 h under nitrogen. Took a sample that indicated the reaction was finished completely. Filtered and rinsed the wet cake with ethyl alcohol (20 mL) twice. Collected the wet cake and concentrated at 40-45° C. to give the residual. Without purification and used in next step directly. To give Compound 8b (1.50 g) as white solid.

Set up the reactor R-1. (Note: the reactor R-1: 10 mL). Charged 2-methylpropanoic acid (1.46 g, 16.6 mmol, 1.54 mL, 1.20 eq), NMM (2.10 g, 20.8 mmol, 2.28 mL, 1.50 eq), isobutyl carbonochloridate (2.27 g, 16.6 mmol, 2.18 mL, 1.20 eq) and THF (21.0 mL) into the reactor R-1 at 15-20° C. Charged compound 8b (3.00 g, 13.9 mmol, 1.00 eq) into the reactor R-1 at 15-20° C. Stirred the mixture at 15-20° C. for 14 hrs under N₂. TLC (Petroleum ether:Ethyl acetate=2:1) showed the starting material was consumed completely. Charged 2.00 mL ethyl acetate and 2.00 mL H₂O into the reactor R-1. The aqueous layer was washed with ethyl acetate (1.00 mL). The combined organic layer was washed with brine water (1.00 mL). Without purification. Compound 9b (1.80 g, crude) was obtained as brown solid.

Set up the reactor R-1 (Note: the reactor R-1: 10 mL). Charged compound 9b (1.84 g, 5.53 mmol, 1.20 eq) and THF (9.00 mL) into the reactor R-1 at 15-20° C. Cooled the mixture to 0-5° C. Charged DIEA (1.79 g, 13.8 mmol, 2.41 mL, 3.00 eq) and 1,1,1,2,2,2-hexachloroethane (2.18 g, 9.21 mmol, 1.04 mL, 2.00 eq) into the reactor R-1 at 0-5° C. Stirred the mixture at 20° C. for 12 hrs under N₂. TLC (Petroleum ether:Ethyl acetate=1:1) showed the starting material was consumed completely. Charged 7.00 mL ethyl acetate and 7.00 mL H₂O into the reactor R-1. The aqueous layer was washed with ethyl acetate (7.00 mL). The combined organic layer was washed with brine water (7.00 mL). Without purification. Compound 10b (1.20 g, crude) was obtained as brown solid.

Set up the reactor R-1 (Note: the reactor R-1:35 mL). Charged Pd/C (100 mg, 10% purity), compound 10b (1.00 g, 3.78 mmol, 1.00 eq) and MeOH (7.00 mL) into the reactor R-1 at 15° C. under Ar₂. Heated the mixture to 40° C. Stirred the reactor mixture at 40° C. for 6 hrs under H₂. TLC (Petroleum ether:Ethyl acetate=1:1) showed the starting material was consumed completely. The mixture was fitered under reduced pressure to filtrate. Concentrated under vacuum at 45° C. to remove MeOH. Without purification. Compound 11b (800 mg, crude) was obtained as yellow solid.

Set up the reactor R-1 (50 mL). Charged compound 11b (500 mg, 2.68 mmol, 1.00 eq) and Py (3.50 mL) into the R-1 at 15-20° C. Charged 5-chloro-2-methoxy-benzenesulfonyl chloride (775 mg, 3.22 mmol, 1.20 eq) into the R-1 at 15-20° C. under N₂. The reaction mixture was stirred at 70-75° C. for 12 hrs. TLC (Petroleum ether:Ethyl acetate=1:1) showed the product was formed. The reaction mixture was concentrated under vacuum. The crude product was purified by reversed-phase HPLC column: Phenomenex luna C18100*40 mm*3 μm; mobile phase: [water(TFA)-ACN];B %: 35%-80%, 8 min. Compound 12c (100 mg, 227.85 μmol, 8.50% yield) was obtained as off white solid.

Set up the reactor R-1 (50 mL). Charged compound 12c (150 mg, 342 μmol, 1.00 eq) and THF (0.30 mL) and MeOH (0.20 mL) and H₂O (0.10 mL) into the R-1 at 15-20° C. Charged LiOH H₂O (28.7 mg, 683 μmol, 2.00 eq) into the R-1 at 15-20° C. under N₂. The reaction mixture was stirred at 15-20° C. for 2 hrs. LCMS showed the product was formed. The reaction mixture was concentrated under vacuum. The crude product was purified by reversed-phase HPLC column: Phenomenex C18 80*30 mm*3 μm; mobile phase: [water(TFA)-ACN];B %: 35%-65%, 8 min. HKYK-0016 (20.0 mg, 13.6% yield, 99.0% purity) was obtained as white solid which was checked by ¹H NMR. 1H NMR: (400 MHz, CDCl₃) δ 8.97 (s, 1H), 8.00 (s, 1H), 7.87 (d, J=2.4 Hz, 1H), 7.53 (dd, J=8.8 Hz, 1H), 7.09 (d, J=8.8 Hz, 1H), 3.84 (s, 3H), 3.19-3.22 (m, 1H), 1.40 (d, J=7.2 Hz, 6H).

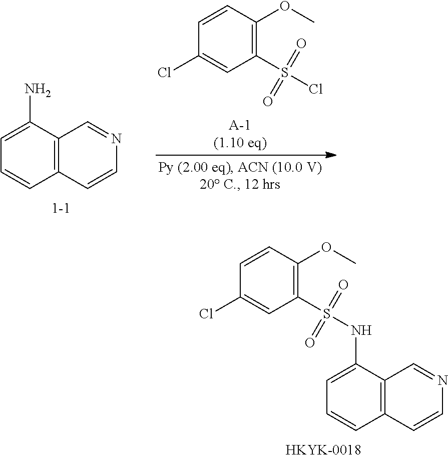 [SMILES: Nc1cccc2ccncc12 ; COc1ccc(Cl)cc1S(=O)(=O)Cl ; COc1ccc(Cl)cc1S(=O)(=O)Nc1cccc2ccncc12]

Set up a reactor R-1. Charged compound 1-1 (100 mg, 1.00 eq), Py (386 mg, 2.00 eq) in ACN (1.00 mL) into reactor R-1 at 20° C. under N₂. Charged compound A-1 (184 mg, 1.10 eq) into reactor R-1 at 0° C. under N₂. Stirred the mixture at 20° C. for 12 hrs. TLC (petroleum ether/ethyl acetate=0/1, product: R_{f}=0.52) shows the starting material was consumed. Charged HCl (2.00 mL) into the reaction mixture. Extracted the reaction mixture with DCM (2.00 mL). Washed the combined organic layer with brine (1.00 mL). Dried the organic layer with Na₂SO₄. Concentrated the organic layer to obtain the residue. The crude product was triturated with EtOAc (2.00 mL) and MTBE (2.00 mL) at 20° C. for 2 hrs. Filtered the mixture and dried the filter cake to obtain the desired product. HKYK-0018 (23.1 mg, 98.7% purity, 43.6% yield) was obtained as a light yellow solid. 1H NMR: (400 MHz DMSO-d₆) δ 10.6 (s, 1H), 9.48 (s, 1H), 4.60 (s, 1H), 8.48 (t, J=5.6 Hz, 1H), 7.79 (t, J=2.8 Hz, 2H), 7.79-7.69 (m, 1H), 7.58 (t, J=20 Hz, 1H), 7.58 (s, 1H), 7.42 (t, J=7.6 Hz, 1H), 7.22 (t, J=9.2 Hz, 1H), 3.78 (s, 3H).

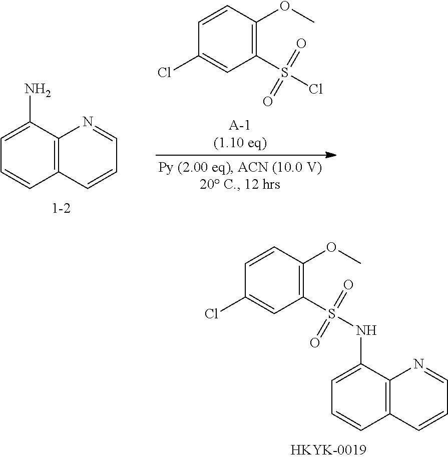 [SMILES: Nc1cccc2cccnc12 ; COc1ccc(Cl)cc1S(=O)(=O)Cl ; COc1ccc(Cl)cc1S(=O)(=O)Nc1cccc2cccnc12]

Set up a reactor R-1. (Note: R-1 is a 100 mL three-neck bottle). Charged compound 1-2 (100 mg, 1.00 eq), Py (386 mg, 2.00 eq) in ACN (1.00 mL) into reactor R-1 at 20° C. under N₂. Charged compound A-1 (184 mg, 1.10 eq) intoto reactor R-1 at 0° C. under N₂. Stirred the mixture at 20° C. for 12 hrs. TLC (petroleum ether/ethyl acetate=0/1, product: R_{f}=0.52) showed the starting material was consumed. Charged HCl (2.00 mL, 20.0× by volume, 1 mol/mL) into the reaction mixture. Extracted the reaction mixture with DCM (2.00 mL, 20.0× by volume) twice. Washed the combined organic layer with brine (1.00 mL, 10.0× by volume). Dried the organic layer with Na₂SO₄. Concentrated the organic layer to obtain the residue. The crude product was triturated with EtOAc (2.00 mL, 20.0× by volume) and MTBE (2.00 mL, 20.0× by volume) at 20° C. for 2 hrs. Filtered the mixture and dried the filter cake to obtain the desired product. HKYK-0019 (27.2 mg, 95.8% purity, 43.6% yield) was obtained as light yellow solid. 1H NMR: (400 MHz DMSO-d₆) δ 9.77 (s, 1H), 8.95 (t, J=4.4 Hz, 1H), 8.39 (t, J=8.0 Hz, 1H), 7.79 (s, 1H), 7.79-7.68 (m, 3H), 7.66-7.52 (m, 2H), 7.11 (t, J=9.2 Hz, 1H), 3.75 (t, J=8.0 Hz, 3H).

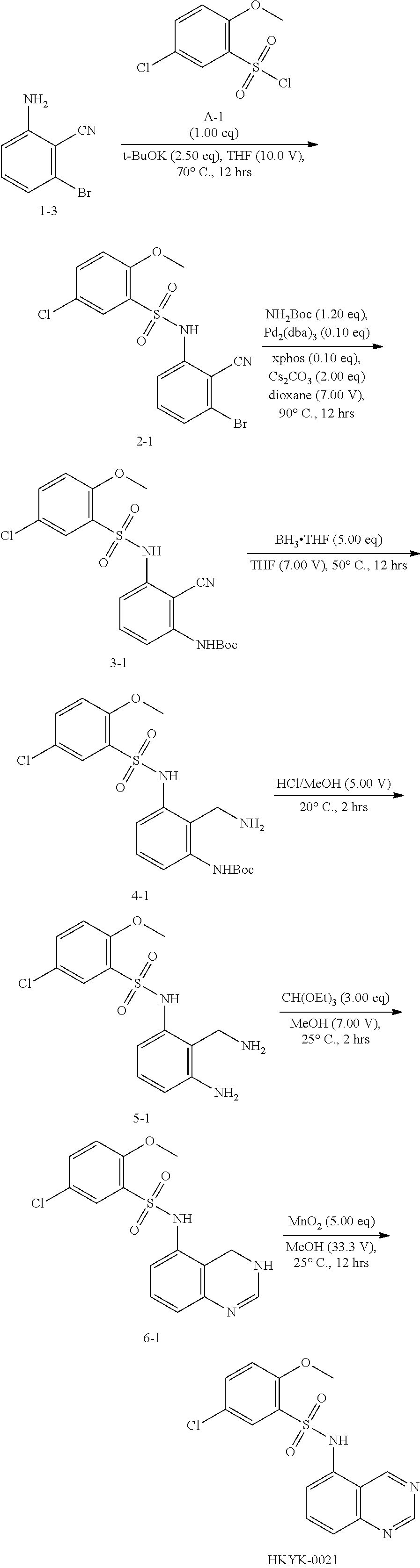

Set up the reactor R-1 with an agitator. Charged compound 1-3 (5.00 g, 25.4 mmol, 1.00 eq) into reactor R-1. Charged compound A-1 (6.10 g, 25.4 mmol, 1.00 eq) into reactor R-1. Charged THF (50.0 mL) into reactor R-1. Charged t-BuOK (1.00 M, 63.5 mL, 2.50 eq) into reactor R-1. Stirred the reaction mixture at 70° C. for 12 hrs. TLC (petroleum ether/ethyl acetate=2/1, product R_{f}=0.42) showed the reactant was consumed, desired spot was observed. Diluted with H₂O (10.0 mL). Extracted with ethyl acetate (10.0 mL). Concentrated to give crude product. Compound 2-1 (5.00 g, crude) was obtained as yellow solid.

Set up the reactor R-1 with an agitator. (Note: the reactor R-1: 100 mL bottle). Charged compound 2-1 (2.00 g, 4.97 mmol, 1.00 eq) into reactor R-1. Charged NH₂Boc (700 mg, 5.96 mmol, 1.20 eq) into reactor R-1. Charged dioxane (14.0 mL) into reactor R-1. Charged Cs₂CO₃ (3.24 g, 9.94 mmol, 2.00 eq) into reactor R-1. Charged XPhos (236 mg, 497 μmol, 0.10 eq) into reactor R-1. Charged Pd₂(dba)₃ (455 mg, 497 μmol, 0.10 eq) into reactor R-1. Stirred the reaction mixture at 90° C. for 12 hrs. TLC (petroleum ether/ethyl acetate=2/1, product R_{f}=0.40) showed the reactant was consumed, desired spot was observed. Diluted with ethyl acetate (20.0 mL). Washed with H₂O (20.0 mL). Concentrated to give crude product. Purified the crude product by silica gel column (eluting with 5% to 50% ethyl acetate in petroleum ether). Compound 3-1 (1.10 g, 2.51 mmol, 56.0% yield) was obtained as a yellow solid. 1H NMR: (400 MHz, CDCl₃) δ 7.91-7.84 (m, 2H), 7.48-7.42 (m, 3H), 6.99-6.95 (m, 2H), 6.87 (s, 1H), 4.04 (s, 3H), 1.50 (s, 9H).

Set up the reactor R-1 with an agitator. (Note: the reactor R-1: 100 mL bottle). Charged compound 3-1 (1.20 g, 2.74 mmol, 1.00 eq) into reactor R-1. Charged THF (8.40 mL) into reactor R-1. Charged BH₃·THF (1 M, 13.7 mL, 5.00 eq) into reactor R-1 at 0° C. Stirred the reaction mixture at 50° C. for 12 hrs. LCMS (product: RT=0.675 min) showed the reactant was consumed, desired product was observed. Charged MeOH (20.0 mL) into the reactor to quench the reaction. Concentrated to give crude product. Compound 4-1 (0.80 g, crude) was obtained as a brown solid.

Set up the reactor R-1 with an agitator. Charged compound 4-1 (0.80 g, 1.81 mmol, 1.00 eq) into reactor R-1. Charged HCl/MeOH (4.00 mL) into reactor R-1. Stirred the reaction mixture at 20° C. for 2 hrs. TLC (petroleum ether/ethyl acetate=2/1, product: R_{f}=0.00) showed the reactant was completed. Concentrated to give crude product. Compound 5-1 (0.60 g, crude, HCl) was obtained as a brown solid.

Set up the reactor R-1 with an agitator. (Note: the reactor R-1: 100 mL bottle). Charged compound 5-1 (0.45 g, 1.31 mmol, 1.00 eq) into reactor R-1. Charged MeOH (5.00 mL) into reactor R-1. Charged CH(OEt)₃ (585 mg, 3.93 mmol, 3.00 eq) into reactor R-1. Stirred the reaction mixture at 25° C. for 2 hrs. LCMS (product RT=0.595 min) showed the reactant was consumed, desired product was observed. Concentrated to give crude product. Treated the crude product with ethyl acetate (5.00 mL). Filtered to get the solid. Compound 6-1 (0.20 g, 568 μmol, 38.8% yield) was obtained as a brown solid. 1H NMR: (400 MHz, DMSO-d6) δ 7.57-7.50 (m, 3H), 7.15 (d, J=4.4 Hz, 1H), 6.85 (t, J=8.0 Hz, 1H), 6.65 (d, J=8.0 Hz, 1H), 6.33 (s, 1H), 4.40 (s, 2H), 3.81 (s, 3H).

Set up the reactor R-1 with an agitator. (Note: the reactor R-1: 100 mL bottle). Charged compound 6-1 (150 mg, 0.43 mmol, 1.00 eq) into reactor R-1. Charged MeOH (5.00 mL) into reactor R-1. Charged MnO₂ (185 mg, 2.13 mmol, 5.00 eq) into reactor R-1. Stirred the reaction mixture at 25° C. for 12 hrs. LCMS (product RT=0.722 min) showed the reactant was consumed, desired product was observed. Filtered to get the filtrate. Concentrated to give crude product. Purified the crude product by prep-HPLC (column: Phenomenex C18 80×40 mm×3 μm; mobile phase: [water (NH₄HCO₃)-ACN]; B %: 5%-35%, 8 min). HKYK-0021 (73.4 mg, 100% purity) was obtained as a yellow solid. 1H NMR: (400 MHz, DMSO-d6) δ 9.72 (s, 1H), 9.12 (s, 1H), 7.71-7.65 (m, 2H), 7.49-7.47 (m, 1H), 7.42-7.28 (m, 2H), 7.10-7.08 (m, 2H), 3.69 (s, 3H).

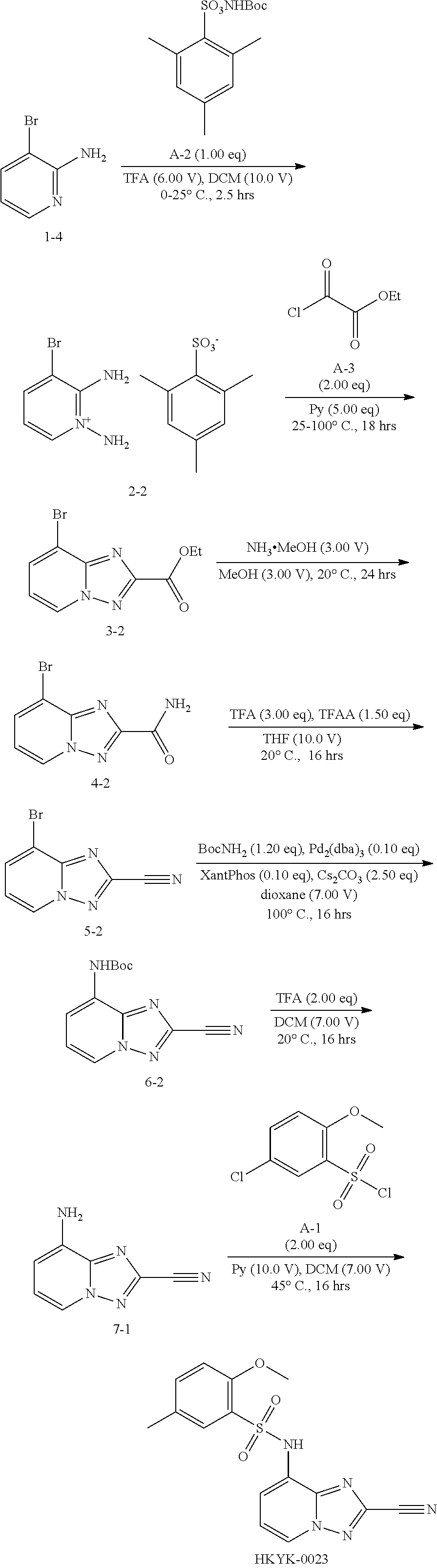

Set up a reactor R-1 with an overhead agitator. Charged compound 1-4 (10.0 g, 1.00 eq), TFA (92.4 g, 810 mmol) into the reactor R-1 at 20˜30° C. Charged compound A-2 (18.2 g, 57.8 mmol) into the reactor R-1 at 0° C. After addition, stirred the mixture at 0° C. for 2 hrs. The reaction mixture was added with ice-water and the precipitate was recovered by filtration. The wet cake was washed with water and dissolved in DCM (50.0 mL) and dried over sodium sulfate. The resulting solution was added dropwise to a stirred solution of compound 1-4 (10.0 g, 57.8 mmol) in DCM (20.0 mL) at 0° C. Stirred 1 hr at 0° C. After addition, stirred the mixture at 20° C. for 12 hrs. TLC (petroleum ether/ethyl acetate=1/1, R_{f}=0.01) indicated reactant was consumed completely. The precipitate was filtered and dried under vacuum. Compound 2-2 (18.0 g, crude) was obtained as a white solid.

Set up a reactor R-1 with an overhead agitator. Charged compound 2-2 (15.0 g, 38.7 mmol) into the reactor R-1 at 25° C. Charged Py (75.0 mL) into the reactor R-1 at 25° C. Charged compound A-3 (10.5 g, 77.2 mmol, 8.65 mL) into the reactor R-1 at 25° C. After addition, stirred the mixture at 100° C. for 12 hrs. TLC (Petroleum ether/Ethyl acetate=1/1, R_{f}=0.43) indicated reactant was consumed completely. Pyridine was then removed under vacuum and saturated bicarb solution (50.0 mL) was added slowly to quench the residual acid at 0° C. The aqueous mixture was then extracted with DCM (50.0 mL×2), dried over MgSO₄ and concentrated under vacuum. The residue was purified by column chromatography (SiO₂, Petroleum ether/Ethyl acetate=50/1 to 1/1). Compound 3-2 (9.00 g) was obtained as a light yellow solid. 1H NMR: (400 MHz, CDCl₃) δ 8.65 (m, J=6.8, 0.8 Hz, 1H), 7.86 (m, J=7.6, 0.8 Hz, 1H), 7.07 (t, J=7.2 Hz, 1H), 4.58 (q, J=7.2 Hz, 2H), 1.49 (t, J=7.2 Hz, 3H).

Set up a reactor R-1 with an overhead agitator. Charged compound 3-2 (1.00 g, 3.70 mmol) into the reactor R-1. Charged MeOH (3.00 mL) into the reactor R-1. Charged NH₃/MeOH (7 M, 3.00 mL) into the reactor R-1. After addition, stirred the mixture at 20° C. for 24 hrs. TLC (petroleum ether/ethyl acetate=3/1, R_{f}=0.22) indicated reactant was consumed completely. The reaction mixture was concentrated under reduced pressure to give a residue. Compound 4-2 (0.80 g, 96.0% purity) was obtained as a white solid. 1H NMR: (400 MHz, DMSO-d₆) δ 9.02 (d, J=6.8 Hz, 1H), 8.09 (d, J=7.6 Hz, 2H), 7.87 (s, 1H), 7.24 (t, J=7.2 Hz, 1H).

Set up a reactor R-1 with an overhead agitator. Charged compound 4-2 (0.80 g, 3.33 mmol) into the reactor R-1 at 20° C. Charged THF (8.00 mL) into the reactor R-1 at 20° C. Charged TEA (1.00 g, 9.94 mmol, 0.83 mL) into the reactor R-1 at 20° C. Charged TFAA (0.63 g, 2.97 mmol, 0.41 mL) into the reactor R-1 at 20° C. After addition, stirred the mixture at 20° C. for 12 hrs. TLC (petroleum ether/ethyl acetate=1/1, R_{f}=0.42) indicated reactant was consumed completely. The reaction mixture was diluted with H₂O (100 mL) and extracted with ethyl acetate (100 mL×3), dried over Na₂SO₄, filtered and concentrated under reduced pressure to give a residue. Compound 5-2 (0.70 g) was obtained as a white solid. 1H NMR: (400 MHz, CDCl₃) δ 8.24 (dd, J=6.88, 0.8 Hz, 1H), 7.55, (d, J=7.50, 0.8 Hz, 1H), 7.17 (t, J=7.19 Hz, 1H).

Set up a reactor R-1 with an overhead agitator. Charged compound 5-2 (0.70 g, 3.14 mmol) into the reactor R-1. Charged dioxane (4.90 mL) into the reactor R-1. Charged Cs₂CO₃ (2.55 g, 7.84 mmol) into the reactor R-1. Charged BocNH₂ (0.44 g, 3.78 mmol) into the reactor R-1. Charged Xantphos (182 mg, 314 μmol) and Pd₂(dba)₃ (600 mg, 314 μmol) into the reactor R-1 at 20˜30° C. After addition, stirred the mixture at 100° C. for 16 hrs. TLC (Petroleum ether/Ethyl acetate=3/1, R_{f}=0.43) indicated reactant was consumed completely. The reaction mixture was diluted with H₂O (200 mL) and extracted with ethyl acetate (100 mL×3), dried over Na₂SO₄, filtered and concentrated under reduced pressure to give a residue. The residue was purified by column chromatography (SiO₂, petroleum ether/ethyl acetate=60/1 to 3/1). Compound 6-2 (0.50 g, 95.3% purity) was obtained as a white solid. 1H NMR: (400 MHz, CDCl₃) δ 8.32-8.21 (m, 2H), 7.65 (s, 1H), 7.24-7.15 (m, 1H), 1.56 (s, 9H).

Set up a reactor R-1 with an overhead agitator. Charged compound 6-2 (0.50 g, 1.93 mmol) into the reactor R-1 at 20˜30° C. Charged DCM (3.50 mL) into the reactor R-1 at 20˜30° C. Charged TFA (0.44 g, 3.86 mmol, 2.00 eq) into the reactor R-1 at 20˜30° C. After addition, stirred the mixture at 20˜30° C. for 5 hrs. TLC (petroleum ether/ethyl acetate=3/1, R_{f}=0.01) indicated reactant was consumed completely. The reaction mixture was concentrated under reduced pressure to give a residue. Compound 7-1 (0.30 g) was obtained as a white solid. 1H NMR: (400 MHz, MeOD) δ 8.37 (m, J=6.8, 1.0 Hz, 1H), 7.21 (m, J=7.8, 6.8 Hz, 1H), 7.16-7.08 (m, 1H).

Set up a reactor R-1 with an overhead agitator. Charged compound 7-1 (0.30 g, 1.53 mmol, HCl) into the reactor R-1 at 45° C. Charged compound A-1 (739 mg, 3.06 mmol) in DCM (2.10 mL) dropwise at 45° C. over 5 mins. After addition, stirred the mixture at 45° C. for 12 hrs. TLC (Petroleum ether/Ethyl acetate=3/1, R_{f}=0.49) indicated reactant was consumed completely. The reaction mixture was diluted with H₂O (20.0 mL) and MeOH (20.0 mL), filtered. The filter cake was concentrated under vacuum. HKYK-0023 (27.8 mg, 97.1% purity) was obtained as a white solid.

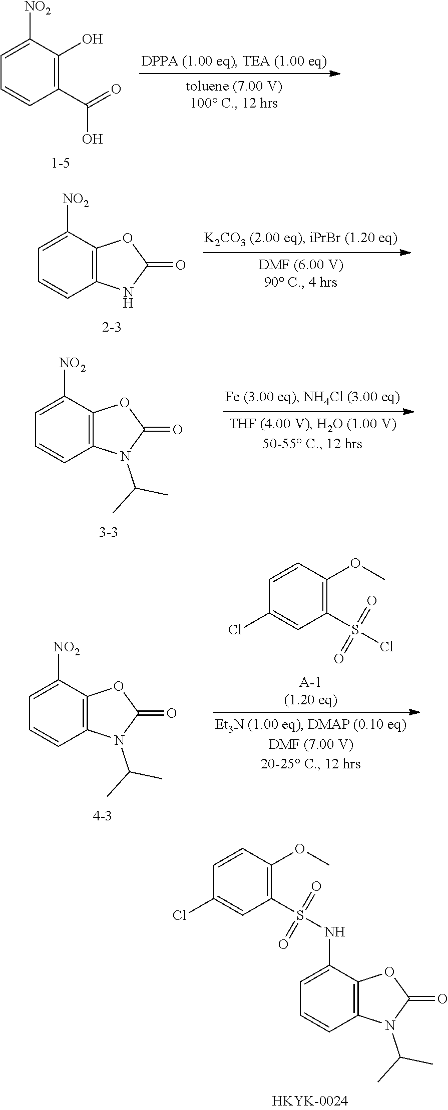 [SMILES: O=C(O)c1cccc([N+](=O)[O-])c1O ; O=c1[nH]c2cccc([N+](=O)[O-])c2o1 ; CC(C)n1c(=O)oc2c([N+](=O)[O-])cccc21 ; CC(C)n1c(=O)oc2c([N+](=O)[O-])cccc21 ; COc1ccc(Cl)cc1S(=O)(=O)Cl ; COc1ccc(Cl)cc1S(=O)(=O)Nc1cccc2c1oc(=O)n2C(C)C]

Set up a reactor R-1. Charged compound 1-5 (1.00 g, 5.45 mmol, 1.00 eq) and toluene (7.00 mL, 7.00 V) into R-1. Charged DPPA (1.50 g, 5.45 mmol, 1.00 eq) and TEA (0.55 g, 5.45 mmol, 1.00 eq) into R-1. Stirred the mixture at 100° C. for 12 hrs. HPLC (product: RT=0.96 min) showed the reaction was completed. Concentrated the mixture in vacuum. The residue was purified by column chromatography (SiO₂, petroleum ether/ethyl acetate=5/1 to 0/1). Compound 2-3 (0.50 g) was obtained as a yellow solid.

Set up a reactor R-1. Charged compound 2-3 (0.50 g, 2.78 mmol, 1.00 eq) and DMSO (3.50 mL, 7.00 V) into R-1. Charged K₂CO₃ (767 mg, 5.55 mmol, 2.00 eq) and iPrBr (377 mg, 3.33 mmol, 1.20 eq) into R-1. Stirred the mixture at 90° C. for 4 hrs. TLC (petroleum ether/ethyl acetate=0/1, R_{f} (product)=0.8) showed the reaction was completed. Charged water (10.0 mL) into the mixture and stirred for 10 mins. Filtered the mixture and washed the filter cake with water (5.00 mL) to give the product. Compound 3-3 (0.30 g, 48.6% yield) was obtained as yellow solid. 1H NMR: (400 MHz, DMSO-d₆) δ 7.88-7.83 (m, J=8.8 Hz, 2H), 7.41 (t, J=8.8 Hz, 1H), 4.55 (t, J=6.8 Hz, 1H), 1.47 (d, J=6.8 Hz, 6H)

Set up a reactor R-1. Charged compound 3-3 (0.30 g, 1.35 mmol, 1.00 eq) and THF (1.20 mL, 4.00 V) and H₂O (0.30 mL, 4.00 V) into R-1. Charged NH₄Cl (216 mg, 4.05 mmol, 3.00 eq) and Fe (226 mg, 4.05 mmol, 3.00 eq) into R-1 at 20-25° C. Stirred the mixture at 50-55° C. for 12 hrs. LCMS (product: RT=0.63 min) showed the reaction was completed. Filtered the mixture and concentrated the filtrate under vacuum. Compound 4-3 (0.20 g, 77.0% yield) was obtained as a yellow oil.

Set up a reactor R-1. Charged compound 4-3 (0.20 g, 1.04 mmol, 1.00 eq) and DMF (1.40 mL, 7.00 V) into R-1. Charged Et₃N (105 mg, 1.04 mmol, 1.00 eq) and DMAP (12.7 mg, 0.10 mmol, 0.10 eq) into R-1 at 20-25° C. Charged Compound A-1 (300 mg, 1.24 mmol, 1.20 eq) into R-1 at 0° C. Stirred the mixture at 20-25° C. for 12 hrs. LCMS (product: RT=1.92 min) showed the reaction was completed. The residue was purified by prep-HPLC (column: Phenomenex luna C18 100*40 mm*3 μm; mobile phase: [water(TFA)-ACN];B %: 30%-75%, 8 min). HKYK-0024 (23.9 mg, 98.3% purity) was obtained as a light yellow solid. 1H NMR: (400 MHz, DMSO-d₆) δ 10.24 (s, 1H), 7.66-7.58 (m, 2H), 7.26-7.20 (m, 2H), 7.11-7.09 (m, 2H), 6.88-6.86 (m, 1H), 4.44-4.40 (m, 1H), 3.85 (s, 3H), 1.41 (d, J=6.8 Hz, 6H).

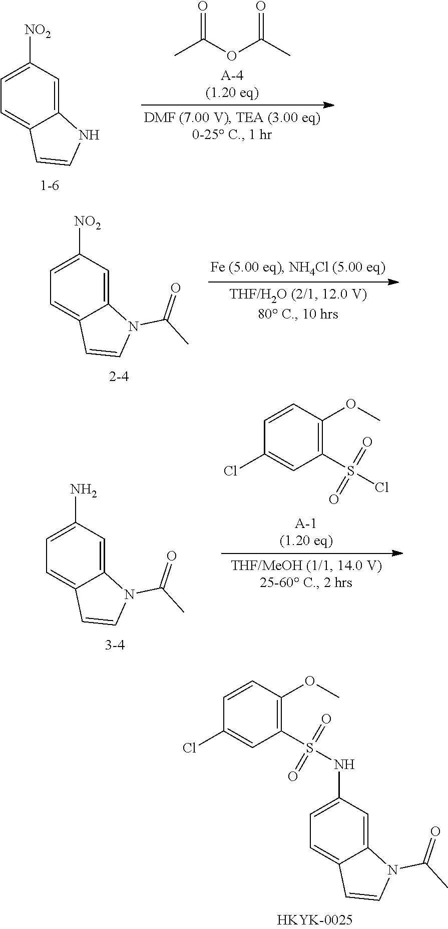

Set up a reactor R-1. (Note: R-1 is a 100 mL three-neck bottle). Charged compound 1-6 (1.00 g, 1.00 eq), compound A-4 (0.76 g, 1.20 eq), TEA (1.87 g, 3.00 eq), in DMF (7.00 mL) into reactor R-1 at 0˜25° C. under N₂. Stirred the mixture at 0˜25° C. for 1 hr. LCMS (product: RT=1.66 min) showed the starting material was consumed. Charged H₂O (3.00 mL, 3.00× by volume) into the reaction mixture. Extracted the reaction mixture with EtOAc (3.00 mL×2). Washed the combined organic layer with brine (3.00 mL). Dried the organic layer with Na₂SO₄. Concentrated the organic layer to obtain the residue. Compound 2-4 (1.00 g, 99.2% yield) was obtained as a yellow solid. ¹H NMR: (400 MHz DMSO-d₆) δ 9.08 (s, 1H), 8.21 (t, J=3.6 Hz, 1H), 8.11-8.09 (m, 1H), 7.80 (t, J=8.8 Hz, 1H), 6.90 (t, J=3.6 Hz, 1H), 2.70 (s, 3H).

Set up a reactor R-1. (Note: R-1 is a 100 mL three-neck bottle). Charged Fe (685 mg, 5.00 eq), compound 2-4 (500 mg, 1.00 eq) in THF (4.00 mL) into reactor R-1 at 20° C. under N₂. Charged NH₄Cl (655 mg, 5.00 eq) and H₂O (2.00 mL) into reactor R-1 at 80° C. under N₂. Stirred the mixture at 80° C. for 10 hrs. TLC (petroleum ether/ethyl acetate=2/1, R_{f}=0.50) showed that the starting material was consumed, and a new point formed. Charged H₂O (3.00 mL) into the reaction mixture. Extracted the reaction mixture with EtOAc (3.00 mL) twice. Washed the combined organic layer with brine (3.00 mL). Dried the organic layer with Na₂SO₄. Concentrated the organic layer to obtain the residue. Compound 3-4 (500 mg) was obtained as a brown solid.

Set up a reactor R-1. Charged compound 3-4 (400 mg, 1.00 eq), compound A-1 (664 mg, 1.20 eq) in THF (2.80 mL) and MeOH (2.80 mL) into reactor R-1 at 25° C. under N₂. Stir the mixture at 60° C. for 2 hrs. HPLC (product: RT=4.52 min) showed the starting material was consumed. Charged H₂O (2.80 mL) into the reaction mixture. Extracted the reaction mixture with EtOAc (2.80 mL) twice. Washed the combined organic layer with brine (2.80 mL). Dried the organic layer with Na₂SO₄. Concentrated the organic layer to obtain the residue. HKYK-0025 (21.0 mg, 99.2% purity, 2.40% yield) was obtained as a light yellow solid. 1H NMR: (400 MHz DMSO-d₆) δ 10.1 (s, 1H), 8.17 (s, 1H), 7.73 (t, J=4.0 Hz, 1H), 7.61-7.56 (m, 2H), 7.44 (t, J=8.4 Hz, 1H), 7.20 (t, J=8.8 Hz, 1H), 7.06 (t, J=0.8 Hz, 3H), 3.93 (s, 1H), 2.59 (s, 3H).

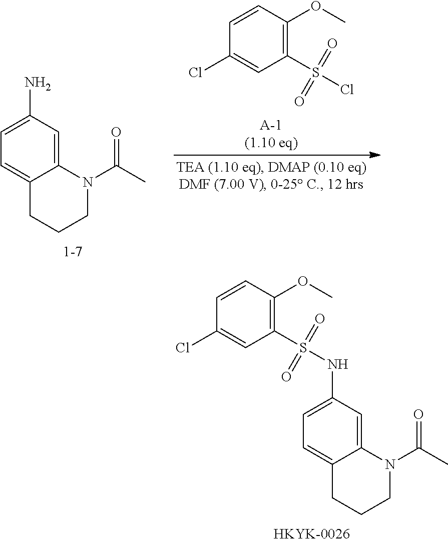 [SMILES: CC(=O)N1CCCc2ccc(N)cc21 ; COc1ccc(Cl)cc1S(=O)(=O)Cl ; COc1ccc(Cl)cc1S(=O)(=O)Nc1ccc2c(c1)N(C(C)=O)CCC2]

Set up the reactor R-1 with an agitator. Charged compound 1-7 (100 mg, 0.52 mmol, 1.00 eq), TEA (58.5 mg, 0.58 mmol, 1.10 eq), DMAP (6.42 mg, 0.05 mmol, 0.10 eq), DMF (0.70 mL) into reactor R-1. Charged compound A-1 (139 mg, 0.58 mmol, 1.10 eq) into reactor R-1 at 0° C. under N2. Stirred the mixture at 0-25° C. for 1 hr. TLC (petroleum ether/ethyl acetate=0/1, product R_{f}=0.25) showed the reactant was consumed, desired spot was observed. Filtered and concentrated the reaction mixture to give the residue. The crude product was triturated with MTBE at 25° C. for 2 hrs. HKYK-0026 (20.7 mg, 96.3% purity) was obtained as a white solid. ¹HNMR: (400 MHz, DMSO-d₆) δ 10.3 (s, 1H), 7.62-7.65 (m, 2H), 7.20 (d, J=9.2 Hz, 2H), 7.00 (d, J=8.4 Hz, 1H), 6.82 (t, J=2.0 Hz, 1H), 4.02 (s, 3H), 3.60-3.90 (m, 2H), 2.55-2.58 (m, 2H), 1.80 (s, 3H), 1.73-1.79 (m, 2H).

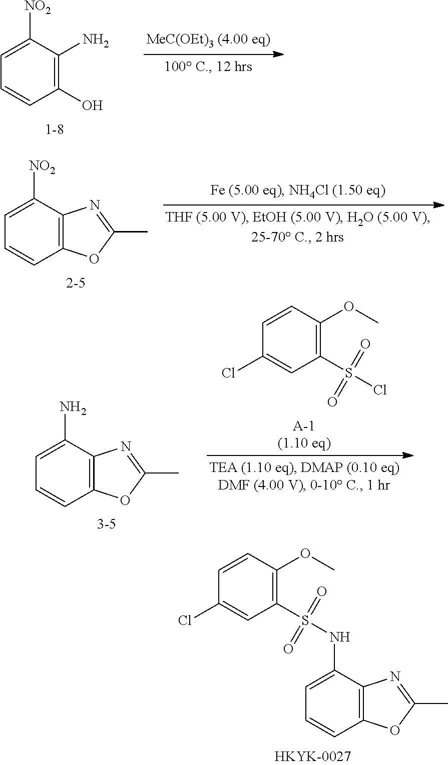 [SMILES: Nc1c(O)cccc1[N+](=O)[O-] ; Cc1nc2c([N+](=O)[O-])cccc2o1 ; COc1ccc(Cl)cc1S(=O)(=O)Cl ; Cc1nc2c(N)cccc2o1 ; COc1ccc(Cl)cc1S(=O)(=O)Nc1cccc2oc(C)nc12]

Set up a reactor R-1. To a solution of compound 1-8 (5.00 g, 32.4 mmol) was added MeC(OEt)₃ (21.0 g, 129 mmol) and the mixture was stirred at 100° C. for 12 hrs to give a dark red solution. TLC (petroleum ether/ethyl acetate=3/1, the starting material R_{f}=0.47) indicated compound 1-8 was consumed completely and one new spot formed. The crude product was triturated with hexane (10.0 mL) at 25° C. for 30 mins. Compound 2-5 (4.00 g, 22.45 mmol, 69.2% yield) was obtained as a brown solid. ¹HNMR: (400 MHz, CDCl₃) δ 2.78 (s, 3H), 7.41-7.49 (m, 1H) 7.82 (d, J=8.00 Hz, 1H) 8.17 (d, J=8.38 Hz, 1H).

To a solution of Compound 2-5 (4.00 g, 22.4 mmol) in THF (20.0 mL), EtOH (20.0 mL), and H₂O (20.0 mL) and Fe (5.02 g, 89.8 mmol) and NH₄Cl (1.80 g, 33.6 mmol) were added to the solution at 25° C. The mixture was stirred at 70° C. for 2 hrs. TLC (petroleum ether/ethyl acetate=2/1, the starting material R_{f}=0.49) indicated Reactant 2-5 was consumed completely and one new spot formed. The mixture was cooled to 25° C., and filtered the filter cake was washed with EtOAc (100 mL.) The residue layer was extracted with ethyl acetate (20.0 ml), washed with saturated saline, dried over anhydrous Na₂SO₄, and concentrated under reduced pressure. Compound 3-5 (2.00 g, 13.50 mmol, 6.1% yield) was obtained as a brown solid

Set up a reactor R-1. To a mixture of compound 3-5 (0.50 g, 3.37 mmol) in DMF (4.00 mL). TEA (375 mg, 3.71 mmol) and DMAP (41.2 mg, 337 umol) were added to the above solution at 0° C., under the nitrogen atmosphere. Then compound A-1 (895 mg, 3.71 mmol) was dropwise added to the reaction solution while the reaction temperature was maintained at 0° C. After completion of the dropwise addition, the temperature was gradually raised and the reaction is continued for 1 hr at 10° C. TLC (petroleum ether/ethyl acetate=1/1, the starting material R_{f}=0.51) indicated Reactant 3-5 was consumed completely and one new spot formed. Charge water (20.0 mL) into the mixture and filtered to give the product. HKYK-0027 (9.70 mg) was obtained as a white solid. 1H NMR: (400 MHz, CDCl₃) δ 7.86-7.96 (m, 2H) 7.36-7.45 (m, 2H) 7.12-7.22 (m, 2H) 6.85 (d, J=8.80 Hz, 1H) 3.98 (s, 3H) 2.59 (s, 3H).

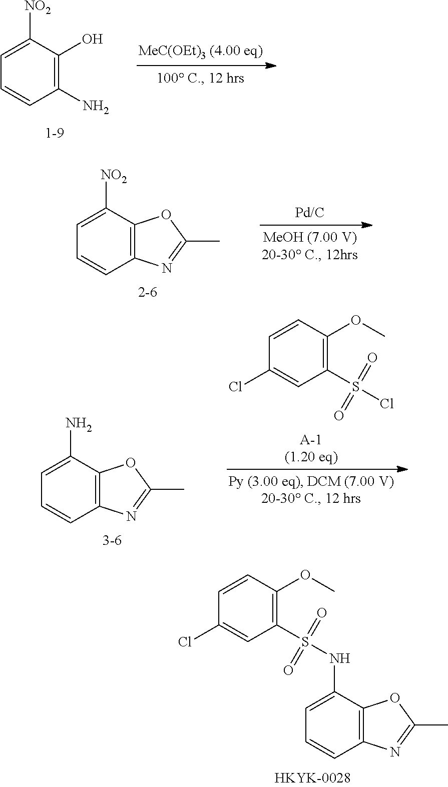 [SMILES: Nc1cccc([N+](=O)[O-])c1O ; Cc1nc2cccc([N+](=O)[O-])c2o1 ; COc1ccc(Cl)cc1S(=O)(=O)Cl ; Cc1nc2cccc(N)c2o1 ; COc1ccc(Cl)cc1S(=O)(=O)Nc1cccc2nc(C)oc12]

Charged compound 1-9 (0.70 g, 4.50 mmol, 1.00 eq) into R-1. Charged MeC(OEt)₃ (2.94 g, 18.0 mmol, 4.00 eq) into the mixture. Stirred the mixture for 12 hrs at 100° C. LCMS (RT=0.66 min) indicated that the reaction was complete. The reaction mixture was concentrated under reduced pressure to give a residue. The residue was purified by column chromatography (SiO₂, petroleum ether/ethyl acetate=1/0 to 10:1). Compound 2-6 (0.50 g) was obtained as a white solid.

Charged compound 2-6 (0.50 g, 2.80 mmol, 1.00 eq) and MeOH (3.50 mL) into R-1. Charged Pd/C (0.05 g, 10.0% purity) into the mixture. Stirred the mixture under H₂ (40 Psi) atmosphere for 12 hrs at 20-30° C. LCMS (RT=0.487 min) indicated that the reaction was completed. The reaction mixture was concentrated under reduced pressure to give a residue. Compound 3-6 (0.20 g, crude) was obtained as a white solid.

Charged compound 3-6 (0.20 g, 1.35 mmol, 1.00 eq), DCM (1.40 mL) and Py (320 mg, 4.05 mmol, 326 μL, 3.00 eq) into R-1. Charged compound A-1 (423 mg, 1.75 mmol, 1.20 eq) into the mixture. Stirred the mixture for 12 hrs at 20-30° C. LCMS (RT=0.73) indicated that the reaction was completed. The reaction mixture was concentrated under reduced pressure to give a residue. The crude product was purified by prep-HPLC (column: Phenomenex C18 75*30 mm*3 μm; mobile phase: [water (NH₄HCO₃)-ACN]; B %: 25%-65%, 8 min). Compound HKYK-0028 (0.026 g, 56.4 μmol, 4.18% yield, 99.5% purity) was obtained as a brown solid. 1H NMR: (400 MHz, MeOD) δ=7.70 (d, J=2.6 Hz, 1H), 7.53 (dd, J=2.8, 8.9 Hz, 1H), 7.37 (dd, J=2.1, 6.9 Hz, 1H), 7.28-7.12 (m, 3H), 3.95 (s, 3H), 2.60 (s, 3H).

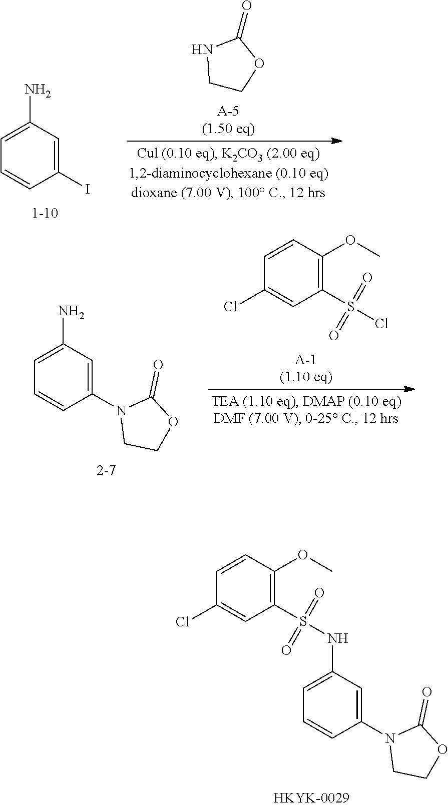 [SMILES: Nc1cccc(I)c1 ; O=C1NCCO1 ; Nc1cccc(N2CCOC2=O)c1 ; COc1ccc(Cl)cc1S(=O)(=O)Cl ; COc1ccc(Cl)cc1S(=O)(=O)Nc1cccc(N2CCOC2=O)c1]

Set up the reactor R-1 with an agitator. Charged compound 1-10 (1.00 g, 4.57 mmol, 1.00 eq) and dioxane (7.00 mL) into reactor R-1. Charged compound A-5 (0.59 g, 6.86 mmol, 1.50 eq), K₂CO₃ (1.26 g, 9.14 mmol, 2.00 eq), cyclohexane-1,2-diamine (52.1 mg, 0.45 mmol, 0.10 eq) to reactor R-1 under N₂. Charged CuI (87.0 mg, 0.45 mol, 0.10 eq) into reactor R-1 under N₂. Stirred the mixture at 100° C. for 12 hrs. TLC (petroleum ether/ethyl acetate=3/1, product R_{f}=0.25) showed the reactant was consumed, desired spot was observed. Filtered and concentrated the reaction mixture to give the residue. Concentrated to give crude product. The residue was purified by column chromatography (SiO₂, petroleum ether/ethyl acetate=50/1 to 10/1) to obtain the desired product. Compound 2-7 (0.50 g, crude) was obtained as a light-yellow solid.

Set up the reactor R-1 with an agitator. Charged compound 2-7 (100 mg, 0.56 mmol, 1.00 eq), TEA (62.5 mg, 0.62 mmol, 1.10 eq), DMAP (6.86 mg, 0.05 mmol, 0.10 eq), DMF (0.70 mL) into reactor R-1. Charged compound A-1 (148 mg, 0.68 mmol, 1.10 eq) into reactor R-1 at 0° C. under N₂. Stirred the mixture at 25° C. for 12 hrs. Filtered and concentrated the reaction mixture to give the residue. The residue was purified by prep-HPLC (TFA condition). HKYK-0029 (30.0 mg, 99.3% purity) was obtained as a brown solid. 1H NMR: (400 MHz, DMSO-d₆) δ 10.2 (s, 1H), 7.60-7.68 (m, 2H), 7.49 (s, 1H), 7.19-7.21 (m, 2H), 7.06-7.09 (m, 1H), 6.90-6.86 (m, 1H), 4.37-4.41 (m, 2H), 3.38-3.96 (m, 5H).

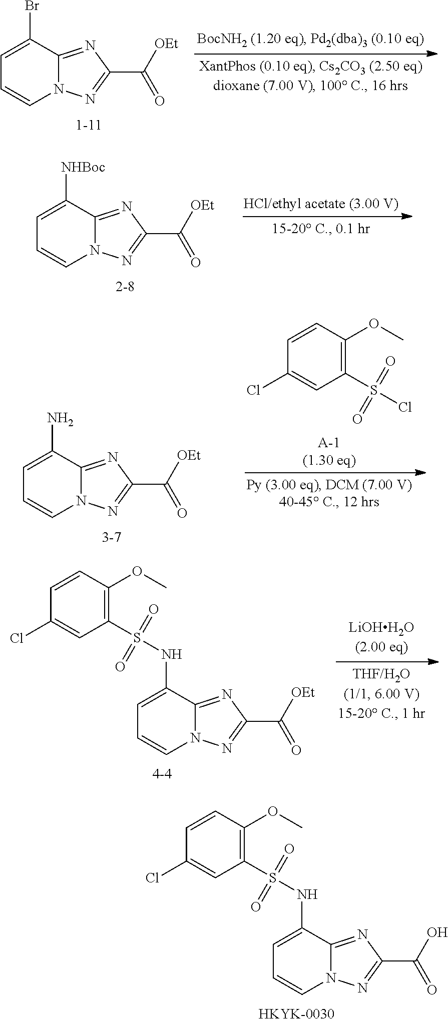 [SMILES: CCOC(=O)c1nc2c(Br)cccn2n1 ; CCOC(=O)c1nc2c(NC(=O)OC(C)(C)C)cccn2n1 ; CCOC(=O)c1nc2c(N)cccn2n1 ; COc1ccc(Cl)cc1S(=O)(=O)Cl ; CCOC(=O)c1nc2c(NS(=O)(=O)c3cc(Cl)ccc3OC)cccn2n1 ; COc1ccc(Cl)cc1S(=O)(=O)Nc1cccn2nc(C(=O)O)nc12]

Charged compound 1-11 (3.00 g, 1.00 eq) and dioxane (21.0 mL) into R-1. Charged NH₂Boc (1.56 g, 1.20 eq), Cs₂CO₃ (9.05 g, 2.50 eq), Pd₂(dba)₃ (1.02 g, 0.10 eq) and Xantphos (642 mg, 0.10 eq) into the mixture. Stirred the mixture for 16 hrs at 100° C. LCMS (RT=1.79 min) indicated that the reaction was complete. The reaction mixture was quenched by addition of brine (20 mL) at 10-20° C., and then extracted with ethyl acetate (20 mL). The combined organic layers were washed with brine (20 mL), dried over Na₂SO₄, filtered and concentrated under reduced pressure to give a residue. The residue was purified by column chromatography (SiO₂, Petroleum ether/Ethyl acetate=1/0 to 0/1). Compound 2-8 (2.00 g, crude) was obtained as a yellow solid.

Charged compound 2-8 (2.00 g, 1.00 eq) and HCl/EtOAc (6.00 mL) into R-1. Stirred the mixture for 0.1 hr at 15-20° C. LCMS (RT=0.192 min) indicated that the reaction was complete. The reaction mixture was filtered and concentrated under reduced pressure to give a residue. Compound 3-7 (0.40 g, crude) was obtained as a white solid. 1H NMR: (400 MHz, DMSO) δ 8.21 (dd, J=0.8, 6.6 Hz, 1H), 7.47-7.19 (m, 2H), 7.11-7.01 (m, 2H), 6.70-6.63 (m, 1H), 4.46-4.36 (m, 2H), 1.39 (br s, 3H).

Charged compound 3-7 (0.40 g, 1.94 mmol, 1.00 eq), DCM (2.80 mL) and Py (460 mg, 5.82 mmol, 469 μL, 3.00 eq) into R-1. Charged compound A-1 (607 mg, 2.52 mmol, 1.30 eq) into the mixture. Stirred the mixture for 12 hrs at 40-45° C. LCMS (RT=0.735 min) indicated that the reaction was complete. The reaction mixture was filtered and concentrated under reduced pressure to give a residue. The residue was purified by column chromatography (SiO₂, petroleum ether/ethyl acetate=1/0 to 10:1). Compound 4-4 (0.20 g, 25.1% yield) was obtained as a yellow solid. 1H NMR: (400 MHz, CDCl₃) δ 8.18 (d, J=6.1 Hz, 1H), 7.84 (d, J=2.4 Hz, 1H), 7.48-7.38 (m, 1H), 7.35-7.23 (m, 2H), 6.96 (t, J=7.2 Hz, 1H), 6.78 (d, J=8.8 Hz, 1H), 4.49 (q, J=7.0 Hz, 2H), 3.92 (s, 3H), 3.48 (s, 3H), 1.43 (t, J=7.1 Hz, 3H).

Charged compound 4-4 (0.20 g, 486 μmol, 1.00 eq), THF (0.60 mL) and H₂O (0.60 mL) into R-1. Charged LiOH H₂O (40.8 mg, 973 μmol, 2.00 eq) into the mixture. Stirred the mixture for 1 hr at 15-20° C. LCMS (RT=0.66 min) indicated that the reaction was completed. The pH was adjusted to around 6 by progressively adding solid HCl. The reaction mixture was concentrated under reduced pressure to give a residue. The crude product was purified by prep-HPLC (column: Phenomenex Luna C18 75*30 mm*3 μm; mobile phase: [water (HCl)-ACN]; B %: 20%-50%, 8 min). Compound HKYK-0030 (20.9 mg, 100% purity) was obtained as a white solid. 1H NMR: (400 MHz, DMSO) δ 8.79 (d, J=6.8 Hz, 1H), 7.71 (d, J=2.6 Hz, 1H), 7.62 (dd, J=2.7, 8.9 Hz, 1H), 7.54 (d, J=7.2 Hz, 1H), 7.31-7.24 (m, 1H), 7.16 (d, J=8.9 Hz, 1H), 3.67 (s, 3H).

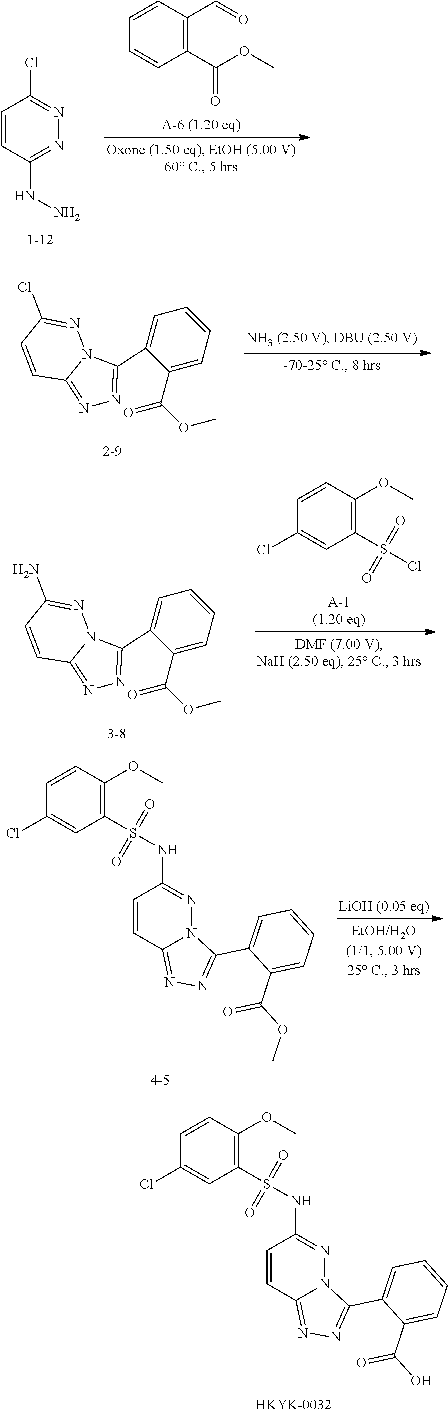

Set up a reactor R-1. (Note: R-1 is a 100 mL three-neck bottle). Charged compound 1-12 (1.00 g, 1.00 eq), compound A-6 (1.25 g, 1.20 eq), Oxone (1.74 g, 1.50 eq) and EtOH (5.00 mL, 5.00× by volume) into reactor R-1 under N₂. After addition, the reaction mixture was stirred at 60° C. for 5 hrs. LCMS (product: RT=1.37 min) showed the starting material was consumed. Charged H₂O (14.0 mL) into the reaction mixture. Extracted the reaction mixture with ethyl acetate (14.0 mL) three times. The combined organic phase was washed with brine (14.0 mL), dried with anhydrous Na₂SO₄, filtered and concentrated under vacuum. The residue was purified by column chromatography (SiO₂, petroleum ether/ethyl acetate=50/1 to 1/1) to obtain the desired product. Compound 2-9 (1.00 g, 71.5% yield) was obtained as a white solid. 1H NMR: (400 MHz CDCl₃) δ 8.32 (d, J=46.8 Hz, 1H), 8.12 (s, 1H), 8.10-8.05 (m, 2H), 7.21 (d, J=48.0 Hz, 1H), 7.06 (d, J=58.8 Hz, 1H), 3.64 (s, 3H).

Set up a reactor R-1. (Note: R-1 is a 100 mL Stuffy tank). Charged compound 2-9 (1.00 g, 1.00 eq) into reactor R-1 at 25° C. Charged DBU (2.50 mL) and NH₃ (2.50 mL) into reactor R-1 at −70° C. After addition, the reaction mixture was stirred at 25° C. for 8 hrs. LCMS (product: RT=0.48 min) showed the starting material was consumed. Combined 2 batches together to work-up. Concentrated the reaction mixture to give the residue. The residue was purified by column chromatography (SiO₂, petroleum ether/ethyl acetate=20/1 to 5/1) to obtain the desired product. Compound 3-8 (0.70 g) was obtained as an off-white solid.

Set up a reactor R-1. Charged compound 3-8 (0.50 g, 1.00 eq) and compound A-1 (0.53 g, 1.20 eq) in DMF (3.50 mL) into reactor R-1 at 25° C. Charged NaH (0.13 g, 2.50 eq) into reactor R-1 at 25° C. After addition, the reaction mixture was stirred at 25° C. for 3 hrs. TLC (ethyl acetate/methanol=5/1, product: R_{f}=0.31) showed the starting material was consumed. Charged H₂O (2.10 mL, 7.00× by volume) into reactor R-1 at 0° C. for 10 mins. Charged ethyl acetate (2.10 mL, 7.00× by volume) into the reaction mixture. Filtered and concentrated the reaction mixture to give the residue. The crude product was purified by reversed-phase HPLC [water (TFA)-ACN]; B %: 5%-35%, 20 min) to obtain the desired product. Compound 4-5 (0.20 g, 37.8% yield) was obtained as a brown solid.

Set up a reactor R-1. (Note: R-1 is a 100 mL three-neck bottle). Charged compound 4-5 (0.20 g, 1.00× by weight), LiOH (0.05 g, 0.25× by weight), EtOH (0.50 mL, 2.50× by volume) and EtOH (0.50 mL, 2.50× by volume) into reactor R-1 at 25° C. After addition, the reaction mixture was stirred at 25° C. for 3 hrs. TLC (ethyl acetate/methanol=3/1, product: R_{f}=0.41) showed the starting material was consumed. Filtered and concentrated the reaction mixture to give the residue. HKYK-0032 (100 mg) was obtained as a brown solid. 1H NMR: (400 MHz DMSO-d₆) δ 7.87 (s, 1H), 7.76 (d, J=10.0 Hz, 1H), 7.53 (s, 1H), 7.53 (d, J=3.20 Hz, 2H), 7.34 (s, 1H), 7.32 (d, J=4.40 Hz, 1H), 7.02 (d, J=10.0 Hz, 1H), 6.96 (d, J=8.80 Hz, 1H), 3.61 (s, 3H).

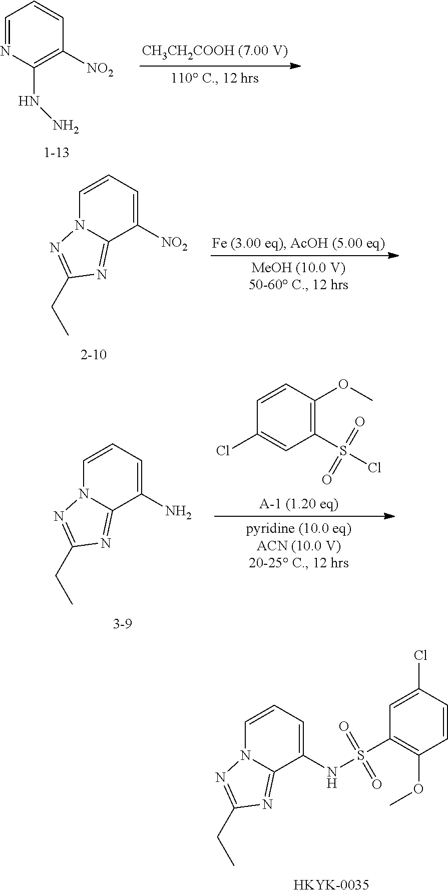 [SMILES: NNc1ncccc1[N+](=O)[O-] ; CCc1nc2c([N+](=O)[O-])cccn2n1 ; CCc1nc2c(N)cccn2n1 ; COc1ccc(Cl)cc1S(=O)(=O)Cl ; CCc1nc2c(NS(=O)(=O)c3cc(Cl)ccc3OC)cccn2n1]

Set up a reactor R-1. Charged compound 1-13 (1.00 g, 6.49 mmol, 1.00 eq) and CH₃CH₂COOH (7.00 mL, 7.00 V) into R-1 at 25° C. Stirred the mixture at 110° C. for 12 hrs. TLC (petroleum ether/ethyl acetate=1/1, R_{f}=0.60) showed the reaction was completed. Charged water (10.0 mL) into the mixture at 0° C. Extracted the mixture with ethyl acetate (10.0 mL) and washed with Na₂CO₃ (10.0 mL×2). Concentrated the organic phase under vacuum. The residue was purified by column chromatography (SiO₂, petroleum ether/ethyl acetate=5/1 to 1/1). Compound 2-10 (0.40 g, 32.1% yield) was obtained as a yellow solid. 1H NMR: (400 MHz, CDCl₃) δ 8.85-8.83 (d, J=6.8 Hz, 1H), 8.52-8.50 (d, J=8.0 Hz, 1H), 7.17-7.13 (t, J=7.6 Hz, 1H), 3.11-3.06 (m, 2H), 1.48-1.45 (t, J=7.6 Hz, 3H).

Set up a reactor R-1. Charged Compound 2-10 (0.30 g, 1.56 mmol, 1.00 eq) and MeOH (3.00 mL, 10.0 V) into R-1. Charged Fe (261 mg, 4.68 mmol, 3.00 eq) into R-1 at 25° C. Charged AcOH (468 mg, 7.81 mmol, 5.00 eq) into R-1 at 50-60° C. Stirred the mixture at 50-60° C. for 12 hrs. TLC (petroleum ether/ethyl acetate=1/1, Rf=0.4) showed the reaction was completed. Filtered the mixture and concentrated the filtrate under vacuum. The residue was purified by column chromatography (SiO₂, petroleum ether/ethyl acetate=3/1 to 1/1). Compound 3-9 (0.20 g, 78.9% yield) was obtained as a yellow oil. 1H NMR: (400 MHz, CDCl3) δ 7.96-7.92 (m, 1H), 6.78-6.71 (m, 1H), 6.61-6.55 (m, 1H), 1.43-1.39 (t, J=7.6 Hz, 2H).

Set up a reactor R-1. Charged compound 3-9 (0.20 g, 1.23 mmol, 1.00 eq) and ACN (2.00 mL, 10.0 V) into R-1. Charged pyridine (975 mg, 12.3 mmol, 10.0 eq) into R-1 at 20-25° C. Charged compound A-1 (357 mg, 1.48 mmol, 1.20 eq) into R-1 at 20-25° C. Stirred the mixture at 20-25° C. for 12 hrs. LCMS (product: RT=0.708 min) showed the reaction was completed. Extracted the mixture with water (5.00 mL) and ethyl acetate (5.00 mL×2). Dried over with Na₂SO₄ and concentrated under vacuum. The residue was purified by column chromatography (SiO₂, petroleum ether/ethyl acetate=5/1 to 1/1). HKYK-0035 (20.5 mg, 4.32% yield) was obtained as a white solid. 1H NMR: (400 MHz, CDCl₃) δ 8.17-8.16 (d, J=6.0 Hz, 1H), 7.89-7.88 (d, J=2.8 Hz, 1H), 7.54-7.52 (d, J=7.6 Hz, 1H), 7.43-7.40 (d, J=11.6 Hz, 1H), 6.87-6.82 (m, 2H), 3.97 (s, 3H), 2.92-2.86 (m, 2H), 1.40-1.37 (t, J=7.6 Hz, 3H).

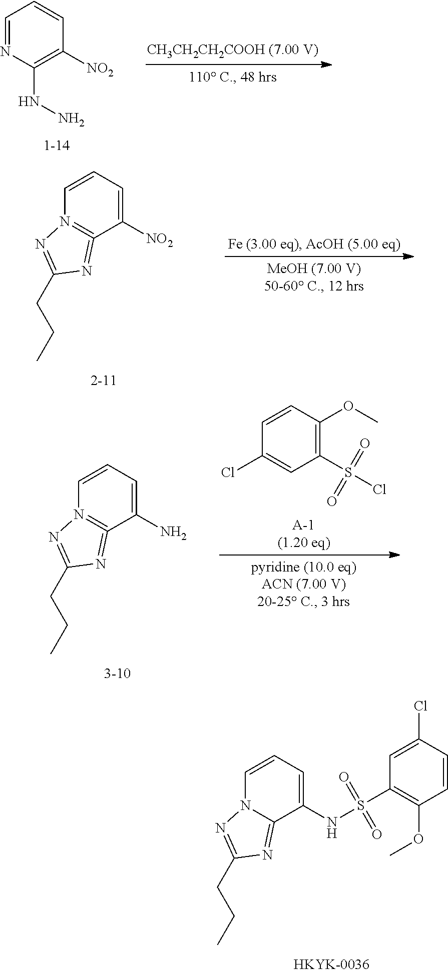

Set up a reactor R-1. (Note: R-1 is a 100 mL three-neck bottle). Charged CH₃CH₂CH₂COOH (7.00 mL, 7.00× by volume) into reactor R-1 at 25° C. Charged compound 1-14 (1.00 g, 1.00× by weight) into reactor R-1 at 25° C. The reaction mixture was degassed and purged with N₂ 3 times. Stirred the mixture at 110° C. for 48 hrs. TLC (petroleum ether/ethyl acetate=1/1, R_{f}=0.35) analysis showed the starting material was consumed. Charged H₂O (3.50 mL, 3.50× by volume) into the reaction mixture. Extracted the reaction mixture with ethyl acetate (3.50 mL, 3.50× by volume) three times. Combined the organic phase. The combined organic phase was washed with brine (3.50 mL, 3.50× by volume), dried with anhydrous Na₂SO₄, filtered, and concentrated under vacuum to obtain the crude product. The crude product was purified by column chromatography (SiO₂, petroleum ether/ethyl acetate=20/1 to 1/1) to obtain the desired product. Compound 2-11 (1.00 g, 72.7% yield) was obtained as a white solid

Set up a reactor R-1. (Note: R-1 is a 100 mL three-neck bottle). Charged compound 2-11 (1.00 g, 1× by weight), AcOH (1.46 g, 1.46× by weight), Fe (0.81 g, 0.81× by weight) in MeOH (7.00 mL, 7.00× by volume) into reactor R-1 at 25° C. The reaction mixture was degassed and purged with N₂ 3 times. After addition, the reaction mixture was stirred at 50-60° C. for 12 hrs. TLC (Petroleum ether/Ethyl acetate=1:1, R_{f}=0.35) showed the starting material was consumed. Charged H₂O (3.50 mL, 3.50× by volume) into the reaction mixture. Extracted the reaction mixture with ethyl acetate (3.50 mL, 3.50× by volume) three times. Combined the organic phase. The combined organic phase was washed with brine (3.50 mL, 3.50× by volume), dried with anhydrous Na₂SO₄, filtered and concentrated under vacuum to obtain the crude product. The crude product was purified by column chromatography (SiO₂, petroleum ether/ethyl acetate=20/1 to 1/1) to obtain the desired product. Compound 3-10 (0.50 g) was obtained as a light yellow solid.

Set up a reactor R-1. Charged compound 3-10 (0.50 g, 1.00× by weight), compound A-1 (0.821 g, 1.64× by weight), Pyridine (2.24 g, 4.48× by weight) in ACN (2.50 mL, 5.00× by volume) into reactor R-1 at 25° C. The reaction mixture was degassed and purged with N₂ for 3 times. Stirred the mixture at 25° C. for 3 hrs. TLC (Petroleum ether/Ethyl acetate=1/1, R_{f}=0.35) showed the starting material was consumed. Charged H₂O (1.20 mL, 3.50× by volume) into the reaction mixture. Extracted the reaction mixture with ethyl acetate (1.20 mL, 3.50× by volume) three times. Combined the organic phase. The combined organic phase was washed with brine (1.20 mL, 3.50× by volume), dried with anhydrous Na₂SO₄, filtered, and concentrated under vacuum to obtain the crude product. The crude product was purified by column chromatography (SiO₂, petroleum ether/ethyl acetate=20/1 to 1/1) to obtain the desired product. HKYK-0036 (1.20 g, 79.3% yield, 99.8% purity) was obtained as a white solid. 1H NMR: (400 MHz DMSO-d₆) δ 8.19-8.14 (m, 1H), 7.91-7.86 (m, 1H), 7.55-7.50 (m, 1H), 7.45-7.38 (m, 1H), 6.90-6.75 (m, 2H), 4.00-3.90 (s, 3H), 2.87-2.76 (m, 2H), 1.91-1.76 (m, 2H), 1.00 (t, J=7.40 Hz, 1H).

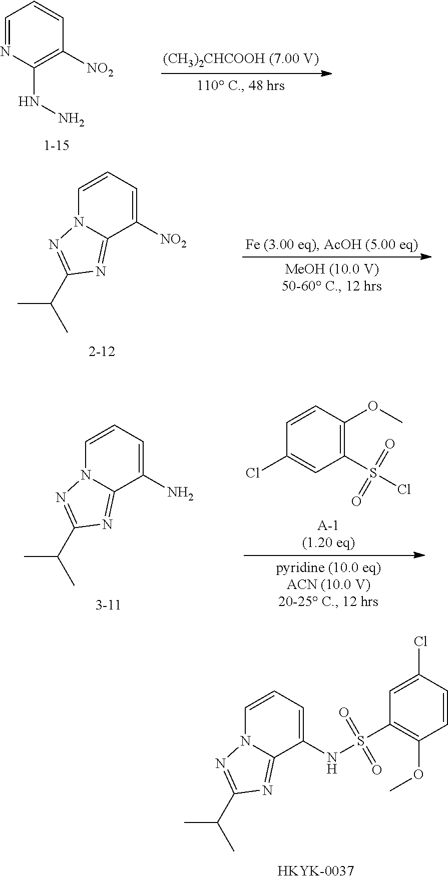 [SMILES: NNc1ncccc1[N+](=O)[O-] ; CC(C)c1nc2c([N+](=O)[O-])cccn2n1 ; CC(C)c1nc2c(N)cccn2n1 ; COc1ccc(Cl)cc1S(=O)(=O)Cl ; COc1ccc(Cl)cc1S(=O)(=O)Nc1cccn2nc(C(C)C)nc12]

Set up a reactor R-1. Charged compound 1-15 (1.00 g, 6.49 mmol, 1.00 eq) and (CH₃)₂CHCOOH (7.00 mL, 7.00 V) into R-1 at 25° C. Stirred the mixture at 110° C. for 48 hrs. TLC (petroleum ether/ethyl acetate=1/1, R_{f} (product)=0.70) showed the reaction was completed. Charged water (10.0 mL) into the mixture at 0° C. Extracted the mixture with ethyl acetate (10.0 mL) and washed with Na₂CO₃ (10.0 mL×2). Concentrated the organic phase under vacuum. The residue was purified by column chromatography (SiO₂, petroleum ether/ethyl acetate=5/1 to 1/1). Compound 2-12 (0.70 g, 52.0% yield) was obtained as a yellow solid.

Set up a reactor R-1. Charged compound 2-12 (0.70 g, 3.39 mmol, 1.00 eq) and MeOH (7.00 mL, 10.0 V) into R-1. Charged Fe (568 mg, 10.1 mmol, 3.00 eq) into R-1 at 25° C. Charged AcOH (1.02 g, 16.9 mmol, 5.00 eq) into R-1 at 50-60° C. Stirred the mixture at 50-60° C. for 12 hrs. TLC (petroleum ether/thyl acetate=1/1, R_{f}=0.5) showed the reaction was completed. Filtered the mixture and concentrated the filtrate under vacuum. The residue was purified by column chromatography (SiO₂, petroleum ether/ethyl acetate=5/1 to 1/1). Compound 3-11 (0.60 g, 85.7% yield) was obtained as a yellow oil.

Set up a reactor R-1. Charged compound 3-11 (0.50 g, 2.84 mmol, 1.00 eq) and ACN (5.00 mL, 10.0 V) into R-1. Charged pyridine (2.24 g, 28.3 mmol, 10.0 eq) into R-1 at 20-25° C. Charged compound A-1 (820 mg, 3.40 mmol, 1.20 eq) into R-1 at 20-25° C. Stirred the mixture at 20-25° C. for 12 hrs. LCMS (product: RT=1.91 min) showed the reaction was completed. Extracted the mixture with water (5.00 mL) and ethyl acetate (5.00 mL×2). Dried over Na₂SO₄ and concentrated under vacuum. The residue was purified by column chromatography (SiO₂, petroleum ether/ethyl acetate=10/1 to 8/1). HKYK-0037 (80.1 mg, 16.02% yield) was obtained as a white solid. 1H NMR: (400 MHz, CDCl₃) δ1.32 (d, J=6.8 Hz, 6H), 3.13 (dt, J=14.0, 6.97 Hz, 1H), 3.90 (s, 3H), 6.70-6.83 (m, 2H), 7.34 (dd, J=8.86, 2.63 Hz, 1H), 7.46 (d, J=7.70 Hz, 1H) 7.81 (d, J=2.4 Hz, 1H) 8.10 (d, J=6.8 Hz, 1H).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the claims.

What is claimed is:

1. A method of treating cancer in a subject in need thereof, comprising administering to the subject an effective amount of a compound of the following structural formula: 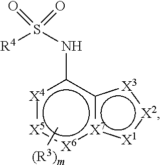
or a pharmaceutically acceptable salt thereof, wherein:
X¹—X²—X³ is N—C(R²⁰)—N, C(R¹⁰)—N—N, C(R¹⁰)—C(R²⁰)—N, N—C(R²⁰)—O, O—C(R²⁰)—N, C(R¹⁰)—N—O, N—C(R²⁰)—S, S—C(R²⁰)—N, C(R¹⁰)—C(R²⁰)—O, N(R¹¹)—C(R²⁰)—N or N(H)—C(O)—O;
R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹¹ is H, (C₁-C₆)alkyl, (C₁-C₆)haloalkyl, (C₃-C₁₀)cycloalkyl, (C₃-C₁₀)heterocyclyl, (C₆-C₁₀)aryl or (C₅-C₁₀)heteroaryl;
R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
X⁴, X⁵ and X⁶ are each independently C(H) or N;
X⁷ is C or N;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
each R³⁰ is independently hydroxy, (C₁-C₆)alkoxy, amino, (C₁-C₆)alkylamino or (C₁-C₆)dialkylamino;
R⁴ is (C₃-C₁₀)cycloalkyl, (C₃-C₁₀)heterocyclyl, (C₆-C₁₀)aryl or (C₅-C₁₀)heteroaryl optionally substituted with one or more R⁴⁰;
R⁴⁰, for each occurrence, is independently halo, cyano, (C₁-C₆)alkyl, (C₁-C₆)haloalkyl, (C₁-C₆)alkoxy(C₁-C₆)alkoxy, carboxy(C₁-C₆)alkoxy, HO—N(H)C(O)—(C₁-C₆)alkoxy, HOS(O)₂—(C₁-C₆)alkoxy, H₂NS(O)₂—(C₁-C₆)alkoxy, P(O)(OH)₂—(C₁-C₆)alkoxy, P(O)(OH)(H)—(C₁-C₆)alkoxy, (HO)₂B—(C₁-C₆)alkoxy, tetrazole-(C₁-C₆)alkoxy, thiazolidinedione-(C₁-C₆)alkoxy, oxazolidinedione-(C₁-C₆)alkoxy, isothiazole-(C₁-C₆)alkoxy, isoxazole-(C₁-C₆)alkoxy, oxooxadiazole-(C₁-C₆)alkoxy, oxothiadiazole-(C₁-C₆)alkoxy, thioxooxadiazole-(C₁-C₆)alkoxy, (C₁-C₆)alkoxy, (C₁-C₆)haloalkoxy, amino, (C₁-C₆)alkylamino, (C₁-C₆)dialkylamino, —C(O)R⁴¹, (C₃-C₁₀)cycloalkyl, (C₃-C₁₀)heterocyclyl, (C₆-C₁₀)aryl or (C₅-C₁₀)heteroaryl; or
two R⁴⁰ on adjacent atoms of R⁴, taken together with the atoms to which they are attached, form a 5- or 6-membered cycle optionally substituted with one or more R⁴²;
R⁴¹ is (C₁-C₆)alkoxy, amino, (C₁-C₆)alkylamino or (C₁-C₆)dialkylamino;
R⁴², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

2. A method of inhibiting metastasis of a cancer in a subject having the cancer; sensitizing a cancer in a subject in need thereof to treatment with a radiation therapy, chemotherapy or immune therapy; or promoting T-cell activation or infiltration or both in response to a cancer in a subject in need thereof, comprising administering to the subject an effective amount of a compound of the following structural formula: 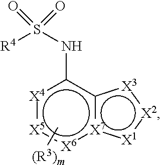
or a pharmaceutically acceptable salt thereof, wherein:
X¹—X²—X³ is N—C(R²⁰)—N, C(R¹⁰)—N—N, C(R¹⁰)—C(R²⁰)—N, N—C(R²⁰)—O, O—C(R²⁰)—N, C(R¹⁰)—N—O, N—C(R²⁰)—S, S—C(R²⁰)—N, C(R¹⁰)—C(R²⁰)—O, N(R¹¹)—C(R²⁰)—N or N(H)—C(O)—O;
R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹¹ is H, (C₁-C₆)alkyl, (C₁-C₆)haloalkyl, (C₃-C₁₀)cycloalkyl, (C₃-C₁₀)heterocyclyl, (C₆-C₁₀)aryl or (C₅-C₁₀)heteroaryl;
R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
X⁴, X⁵ and X⁶ are each independently C(H) or N;
X⁷ is C or N;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
each R³⁰ is independently hydroxy, (C₁-C₆)alkoxy, amino, (C₁-C₆)alkylamino or (C₁-C₆)dialkylamino;
R⁴ is (C₃-C₁₀)cycloalkyl, (C₃-C₁₀)heterocyclyl, (C₆-C₁₀)aryl or (C₅-C₁₀)heteroaryl optionally substituted with one or more R⁴⁰;
R⁴⁰, for each occurrence, is independently halo, cyano, (C₁-C₆)alkyl, (C₁-C₆)haloalkyl, (C₁-C₆)alkoxy(C₁-C₆)alkoxy, carboxy(C₁-C₆)alkoxy, HO—N(H)C(O)—(C₁-C₆)alkoxy, HOS(O)₂—(C₁-C₆)alkoxy, H₂NS(O)₂—(C₁-C₆)alkoxy, P(O)(OH)₂—(C₁-C₆)alkoxy, P(O)(OH)(H)—(C₁-C₆)alkoxy, (HO)₂B—(C₁-C₆)alkoxy, tetrazole-(C₁-C₆)alkoxy, thiazolidinedione-(C₁-C₆)alkoxy, oxazolidinedione-(C₁-C₆)alkoxy, isothiazole-(C₁-C₆)alkoxy, isoxazole-(C₁-C₆)alkoxy, oxooxadiazole-(C₁-C₆)alkoxy, oxothiadiazole-(C₁-C₆)alkoxy, thioxooxadiazole-(C₁-C₆)alkoxy, (C₁-C₆)alkoxy, (C₁-C₆)haloalkoxy, amino, (C₁-C₆)alkylamino, (C₁-C₆)dialkylamino, —C(O)R⁴¹, (C₃-C₁₀)cycloalkyl, (C₃-C₁₀)heterocyclyl, (C₆-C₁₀)aryl or (C₅-C₁₀)heteroaryl; or
two R⁴⁰ on adjacent atoms of R⁴, taken together with the atoms to which they are attached, form a 5- or 6-membered cycle optionally substituted with one or more R⁴²;
R⁴¹ is (C₁-C₆)alkoxy, amino, (C₁-C₆)alkylamino or (C₁-C₆)dialkylamino;
R⁴², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

3. The method of claim 1 or 2, wherein the cancer is chemoresistant.

4. The method of any one of claims 1-3, wherein the cancer is metastatic.

5. The method of any one of claims 1-4, wherein the cancer is a hematologic cancer.

6. The method of any one of claims 1-4, wherein the cancer is a solid tumor cancer.

7. The method of claim 6, wherein the cancer is breast cancer, liver cancer, lung cancer, colorectal cancer, glioblastoma, prostate cancer, melanoma, bladder cancer, pancreatic cancer, kidney cancer or gastric cancer.

8. The method of any one of claims 1-7, further comprising administering to the subject an additional therapy.

9. The method of claim 8, wherein the additional therapy comprises radiation therapy.

10. The method of claim 8 or 9, wherein the additional therapy comprises a chemotherapy.

11. The method of claim 10, wherein the chemotherapy comprises, consists essentially of or consists of a taxoid.

12. The method of any one of claims 8-11, wherein the additional therapy comprises an immune therapy.

13. The method of claim 12, wherein the immune therapy comprises, consists essentially of or consists of a PD-1 inhibitor.

14. A method of inhibiting an interaction between metadherin (MTDH) and Staphylococcal nuclease domain containing 1 (SND1) in a cell expressing MTDH and SND1; or (i) stabilizing or increasing the level or expression of transporter associated with antigen processing (TAP), (ii) inhibiting degradation of Tap, or (iii) promoting tumor antigen presentation in a cell, the method comprising contacting the cell with a compound of the following structural formula: 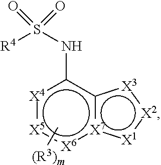
or a pharmaceutically acceptable salt thereof, wherein:
X¹—X²—X³ is N—C(R²⁰)—N, C(R¹⁰)—N—N, C(R¹⁰)—C(R²⁰)—N, N—C(R²⁰)—O, O—C(R²⁰)—N, C(R¹⁰)—N—O, N—C(R²⁰)—S, S—C(R²⁰)—N, C(R¹⁰)—C(R²⁰)—O, N(R¹¹)—C(R²⁰)—N or N(H)—C(O)—O;
R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹¹ is H, (C₁-C₆)alkyl, (C₁-C₆)haloalkyl, (C₃-C₁₀)cycloalkyl, (C₃-C₁₀)heterocyclyl, (C₆-C₁₀)aryl or (C₅-C₁₀)heteroaryl;
R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
X⁴, X⁵ and X⁶ are each independently C(H) or N;
X⁷ is C or N;
each R³ is independently hydroxy, halo, cyano, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, —C(O)R³⁰, (C₃-C₁₀)cycloalkyl, (C₃-C₁₀)heterocyclyl, (C₆-C₁₀)aryl or (C₅-C₁₀)heteroaryl;
each R³⁰ is independently hydroxy, (C₁-C₆)alkoxy, amino, (C₁-C₆)alkylamino or (C₁-C₆)dialkylamino;
R⁴ is (C₃-C₁₀)cycloalkyl, (C₃-C₁₀)heterocyclyl, (C₆-C₁₀)aryl or (C₅-C₁₀)heteroaryl optionally substituted with one or more R⁴⁰;
R⁴⁰, for each occurrence, is independently halo, cyano, (C₁-C₆)alkyl, (C₁-C₆)haloalkyl, (C₁-C₆)alkoxy(C₁-C₆)alkoxy, carboxy(C₁-C₆)alkoxy, HO—N(H)C(O)—(C₁-C₆)alkoxy, HOS(O)₂—(C₁-C₆)alkoxy, H₂NS(O)₂—(C₁-C₆)alkoxy, P(O)(OH)₂—(C₁-C₆)alkoxy, P(O)(OH)(H)—(C₁-C₆)alkoxy, (HO)₂B—(C₁-C₆)alkoxy, tetrazole-(C₁-C₆)alkoxy, thiazolidinedione-(C₁-C₆)alkoxy, oxazolidinedione-(C₁-C₆)alkoxy, isothiazole-(C₁-C₆)alkoxy, isoxazole-(C₁-C₆)alkoxy, oxooxadiazole-(C₁-C₆)alkoxy, oxothiadiazole-(C₁-C₆)alkoxy, thioxooxadiazole-(C₁-C₆)alkoxy, (C₁-C₆)alkoxy, (C₁-C₆)haloalkoxy, amino, (C₁-C₆)alkylamino, (C₁-C₆)dialkylamino, —C(O)R⁴¹, (C₃-C₁₀)cycloalkyl, (C₃-C₁₀)heterocyclyl, (C₆-C₁₀)aryl or (C₅-C₁₀)heteroaryl; or
two R⁴⁰ on adjacent atoms of R⁴, taken together with the atoms to which they are attached, form a 5- or 6-membered cycle optionally substituted with one or more R⁴²;
R⁴¹ is (C₁-C₆)alkoxy, amino, (C₁-C₆)alkylamino or (C₁-C₆)dialkylamino;
R⁴², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

15. The method of any one of claims 1-14, wherein X¹—X²—X³ is N—C(R²⁰)—N or C(R¹⁰)—N—N.

16. The method of any one of claims 1-15, wherein R¹⁰ is H, (C₁-C₆)alkyl or (C₃-C₁₀)cycloalkyl.

17. The method of claim 16, wherein R¹⁰ is H, (C₁-C₃)alkyl or (C₃-C₆)cycloalkyl.

18. The method of any one of claims 1-15, wherein R¹⁰ is H, methyl, ethyl, n-propyl, iso-propyl, cyclopropyl, cyclobutyl, cyclopentyl, cyclohexyl, phenyl, carboxyphenyl, cyano, or carboxy.

19. The method of any one of claims 1-18, wherein R²⁰ is H, (C₁-C₆)alkyl or (C₃-C₁₀)cycloalkyl.

20. The method of claim 19, wherein R²⁰ is H, (C₁-C₃)alkyl or (C₃-C₆)cycloalkyl.

21. The method of any one of claims 1-18, wherein R²⁰ is H, methyl, ethyl, n-propyl, iso-propyl, cyclopropyl, cyclobutyl, cyclopentyl, cyclohexyl, phenyl, carboxyphenyl, cyano, or carboxy.

22. The method of any one of claims 1-21, wherein X⁴, X⁵ and X⁶ are each C(H); and X⁷ is N.

23. The method of any one of claims 1-22, wherein each R³ is independently halo.

24. The method of any one of claims 1-23, wherein R⁴ is optionally substituted (C₆-C₁₀)aryl or (C₅-C₁₀)heteroaryl.

25. The method of claim 24, wherein R⁴ is optionally substituted phenyl or pyridinyl.

26. The method of any one of claims 1-25, wherein R⁴⁰, for each occurrence, is independently halo, (C₁-C₆)alkyl, (C₁-C₆)alkoxy, (C₁-C₆)alkoxy(C₁-C₆)alkoxy or carboxy(C₁-C₆)alkoxy, or two R⁴⁰ on adjacent atoms of R⁴, taken together with the atoms to which they are attached, form a 5- or 6-membered cycle optionally substituted with one or more R⁴².

27. The method of claim 26, wherein R⁴⁰, for each occurrence, is independently fluoro, chloro, methyl, trifluoromethyl, difluoromethyl, fluoromethyl, methoxy, methoxymethoxy or —OCH₂CO₂H, or two R⁴⁰ on adjacent atoms of R⁴ are —N(H)C(O)O— or —CH₂CH₂O—.

28. The method of any one of claims 1-27, wherein m is 0.

29. The method of any one of claims 1-14, 16-21 and 28, wherein the compound is: 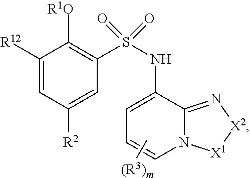
or a pharmaceutically acceptable salt thereof, wherein:
X¹ is N and X² is C(R²⁰), or X₁ is C(R¹⁰) and X² is N;
R¹⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹ is (C₁-C₆)alkyl, (C₁-C₆)alkoxy(C₁-C₆)alkyl, carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl;
R² is halo, (C₁-C₆)alkyl, (C₁-C₆)haloalkyl, (C₁-C₆)alkoxy or (C₁-C₆)haloalkoxy;
each R³ is independently halo;
R¹² is hydrogen or R¹ and R¹², taken together with their intervening atoms, form a 5- or 6-membered cycle optionally substituted with one or more R²²;
R²², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3.

30. The method of claim 29, wherein X¹ is N and X² is C(R²⁰).

31. The method of claim 29, wherein X₁ is C(R¹⁰) and X² is N.

32. The method of any one of claims 29-31, wherein R¹ is (C₁-C₆)alkyl or (C₁-C₆)alkoxy(C₁-C₆)alkyl and R¹² is hydrogen, or R¹ and R¹², taken together with their intervening atoms, form a 5- or 6-membered cycle optionally substituted with one or more R²².

33. The method of any one of claims 29-31, wherein R¹ is carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl.

34. The method of any one of claims 29-31, wherein R¹ is methyl, methoxymethyl or —CH₂CO₂H and R¹² is hydrogen, or R¹ and R¹², taken together, are —N(H)C(O)— or —CH₂CH₂—.

35. The method of any one of claims 29-34, wherein R² is chloro, fluoro, methyl, trifluoromethyl, difluoromethyl or fluoromethyl.

36. The method of claim 35, wherein R² is chloro.

37. The method of any one of claims 29-36, wherein R¹² is hydrogen.

38. A compound represented by the following structural formula: 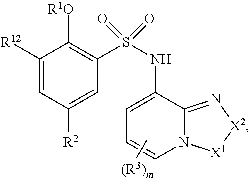
or a pharmaceutically acceptable salt thereof, wherein:
X¹ is N and X² is C(R²⁰);
R²⁰ is H, OH, halo, cyano, carboxy, (C₁-C₆)alkyl, (C₁-C₆)haloalkyl, (C₁-C₆)alkoxy, (C₁-C₆)haloalkoxy, amino, (C₁-C₆)alkylamino, (C₁-C₆)dialkylamino, (C₃-C₁₀)cycloalkyl, (C₃-C₁₀)heterocyclyl, (C₆-C₁₀)aryl, carboxy(C₆-C₁₀)aryl or (C₅-C₁₀)heteroaryl;
R¹ is (C₁-C₆)alkyl, (C₁-C₆)alkoxy(C₁-C₆)alkyl, carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl;
R² is halo, (C₁-C₆)alkyl, (C₁-C₆)haloalkyl, (C₁-C₆)alkoxy or (C₁-C₆)haloalkoxy;
each R³ is independently halo;
R¹² is hydrogen or R¹ and R¹², taken together with their intervening atoms, form a 5- or 6-membered cycle optionally substituted with one or more R²²;
R²², for each occurrence, is independently oxo or halo, cyano, (C₁-C₆)alkyl, (C₁-C₆)haloalkyl, (C₁-C₆)alkoxy or (C₁-C₆)haloalkoxy; and
m is 0, 1, 2 or 3, provided (i) R¹ is not methyl; or (ii) the compound is not 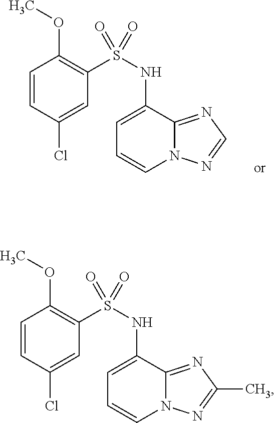 [SMILES: COc1ccc(Cl)cc1S(=O)(=O)Nc1cccn2ncnc12 ; COc1ccc(Cl)cc1S(=O)(=O)Nc1cccn2nc(C)nc12]
or a pharmaceutically acceptable salt thereof.

39. The compound of claim 38, wherein R²⁰ is H, (C₁-C₆)alkyl or (C₃-C₁₀)cycloalkyl.

40. The compound of claim 39, wherein R²⁰ is H, (C₁-C₃)alkyl or (C₃-C₆)cycloalkyl.

41. The compound of claim 38, wherein R²⁰ is H, methyl, ethyl, n-propyl, iso-propyl, cyclopropyl, cyclobutyl, cyclopentyl, cyclohexyl, phenyl, carboxyphenyl, cyano, or carboxy.

42. The compound of any one of claims 38-41, wherein R¹ is (C₁-C₆)alkyl or (C₁-C₆)alkoxy(C₁-C₆)alkyl and R¹² is hydrogen, or R¹ and R¹², taken together with their intervening atoms, form a 5- or 6-membered cycle optionally substituted with one or more R²².

43. The compound of any one of claims 38-41, wherein R¹ is carboxy(C₁-C₆)alkyl, HO—N(H)C(O)—(C₁-C₆)alkyl, HOS(O)₂—(C₁-C₆)alkyl, H₂NS(O)₂—(C₁-C₆)alkyl, P(O)(OH)₂—(C₁-C₆)alkyl, P(O)(OH)(H)—(C₁-C₆)alkyl, (HO)₂B—(C₁-C₆)alkyl, tetrazole-(C₁-C₆)alkyl, thiazolidinedione-(C₁-C₆)alkyl, oxazolidinedione-(C₁-C₆)alkyl, isothiazole-(C₁-C₆)alkyl, isoxazole-(C₁-C₆)alkyl, oxooxadiazole-(C₁-C₆)alkyl, oxothiadiazole-(C₁-C₆)alkyl or thioxooxadiazole-(C₁-C₆)alkyl.

44. The compound of any one of claims 38-41, wherein R¹ is methyl, methoxymethyl or —CH₂CO₂H and R¹² is hydrogen, or R¹ and R¹², taken together, are —N(H)C(O)— or —CH₂CH₂—.

45. The compound of any one of claims 38-44, wherein R² is chloro, fluoro, methyl, trifluoromethyl, difluoromethyl or fluoromethyl.

46. The compound of claim 45, wherein R² is chloro.

47. The compound of any one of claims 38-46, wherein R¹² is hydrogen.

48. The compound of any one of claims 38-47, wherein m is 0.

49. A pharmaceutical composition comprising a compound of any one of claims 38-48, or a pharmaceutically acceptable salt thereof, and one or more pharmaceutically acceptable carriers.

50. A nucleic acid molecule comprising the nucleotide sequence of SEQ ID NO:1, or a nucleotide sequence having at least 75%, at least 85%, at least 90% or at least 95% identity to the nucleotide sequence of SEQ ID NO:1.

51. A protein comprising the amino acid sequence of SEQ ID NO:2, or an amino acid sequence having at least 75%, at least 85%, at least 90% or at least 95% identity to the amino acid sequence of SEQ ID NO:2.

52. A nucleic acid molecule comprising the nucleotide sequence of SEQ ID NO:3, or a nucleotide sequence having at least 75%, at least 85%, at least 90% or at least 95% identity to the nucleic acid sequence of SEQ ID NO:3.

53. A protein comprising the amino acid sequence of SEQ ID NO:4, or an amino acid sequence having at least 75%, at least 85%, at least 90% or at least 95% identity to the amino acid sequence of SEQ ID NO:4.

54. A kit comprising a nucleic acid molecule of claim 50 and a nucleic acid molecule of claim 52.

55. A kit comprising a protein of claim 51 and a protein of claim 53.


SEQUENCE LISTING (22 sequences)

SEQ ID NO: 1 - DNA, 2283 bp, synthetic construct
  misc_feature 1..2283: note = SND1-NLuc DNA Sequence
  source 1..2283: mol_type = other DNA
gagcagaaactcatctctgaagaggatctggtccccaccgtgcagcggggcatcatcaagatggtcctctcagggtgcgccatcattgtccgaggtcagcctcgtggtgggcctcctcctgagcggcagatcaacctcagcaacattcgtgctggaaatcttgctcgccgggcagccgccacacaacctgatgcaaaggatacccctgatgagccctgggcatttccagctcgagagttccttcgaaagaagctgattgggaaggaagtctgtttcacgatagaaaacaagactccccaggggcgagagtatggcatgatctaccttggaaaagataccaatggggaaaacattgcagaatcactggttgcagagggcttagccacccggagagaaggcatgagagctaataatcctgagcagaaccggctttcagaatgtgaagaacaagcaaaggcagccaagaaagggatgtggagtgaggggaacggttcacatactatccgggatctcaagtataccattgaaaacccaaggcactttgtggactcacaccaccagaagcctgttaatgctatcatcgagcatgtgcgggacggcagtgtggtcagggccctgctcctcccagattactacctggttacagtcatgctgtcaggcatcaagtgcccaacttttcgacgggaagcagatggcagtgaaactccagagccttttgctgcagaagccaaatttttcactgagtcgcgactgcttcagagagatgttcagatcattctggagagctgccacaaccagaacattctgggtaccatccttcatccaaatggcaacatcacagagctcctcctgaaggaaggtttcgcacgctgtgtggactggtcgattgcagtttacacccggggcgcagaaaagctgagggcggcagagaggtttgccaaagagcgcaggctgagaatatggagagactatgtggctcccacagctaatttggaccaaaagggaggtggttcaggaggaggtagtggtggaggaagtgaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatgga

SEQ ID NO: 2 - protein, 761 aa, synthetic construct
  REGION 1..761: note = SND1-NLuc Protein Sequence
EQKLISEEDLVPTVQRGIIKMVLSGCAIIVRGQPRGGPPPERQINLSNIRAGNLARRAAATQPDAKDTPDEPWAFPAREFLRKKLIGKEVCFTIENKTPQGREYGMIYLGKDTNGENIAESLVAEGLATRREGMRANNPEQNRLSECEEQAKAAKKGMWSEGNGSHTIRDLKYTIENPRHFVDSHHQKPVNAIIEHVRDGSVVRALLLPDYYLVTVMLSGIKCPTFRREADGSETPEPFAAEAKFFTESRLLQRDVQIILESCHNQNILGTILHPNGNITELLLKEGFARCVDWSIAVYTRGAEKLRAAERFAKERRLRIWRDYVAPTANLDQKGGGSGGGSGGGSEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDG

SEQ ID NO: 3 - DNA, 576 bp, synthetic construct
  misc_feature 1..576: note = CLuc-MTDH DNA Sequence
  source 1..576: mol_type = other DNA
atgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgggaggtggttcaggaggaggtagttcttctgctgatcccaactctgattggaatgcaccagcagaagagtggggcaattgggtagacgaatacccatacgatgttccagattacgct

SEQ ID NO: 4 - protein, 192 aa, synthetic construct
  REGION 1..192: note = CLuc-MTDH Protein Sequence
MSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLGGGSGGGSSSADPNSDWNAPAEEWGNWVDEYPYDVPDYA

SEQ ID NO: 5 - DNA, 1797 bp, synthetic construct
  misc_feature 1..1797: note = Linked-luciferase DNA Sequence
  source 1..1797: mol_type = other DNA
gagcagaaactcatctctgaagaggatctggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggaggaggtggttcaggaggaggtagtggtggaggaagtatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtacccatacgatgttccagattacgct

SEQ ID NO: 6 - protein, 599 aa, synthetic construct
  REGION 1..599: note = Linked Luciferase Protein Sequence
EQKLISEEDLEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGGGGSGGGSGGGSMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKLYPYDVPDYA

SEQ ID NO: 7 - DNA, 21 bp, synthetic construct
  misc_feature 1..21: note = Tap1#1-F
  source 1..21: mol_type = other DNA
ccaacgtgtgcgagtccatta

SEQ ID NO: 8 - DNA, 20 bp, synthetic construct
  misc_feature 1..20: note = Tap1#1-R
  source 1..20: mol_type = other DNA
agagtgaggtacggtgaccc

SEQ ID NO: 9 - DNA, 21 bp, synthetic construct
  misc_feature 1..21: note = Tap1#2-F
  source 1..21: mol_type = other DNA
ggacttgccttgttccgagag

SEQ ID NO: 10 - DNA, 22 bp, synthetic construct
  misc_feature 1..22: note = Tap1#2-R
  source 1..22: mol_type = other DNA
gctgccacataactgatagcga

SEQ ID NO: 11 - DNA, 22 bp, synthetic construct
  misc_feature 1..22: note = Tap2#1-F
  source 1..22: mol_type = other DNA
ccctgtttttctcgctaagagc

SEQ ID NO: 12 - DNA, 21 bp, synthetic construct
  misc_feature 1..21: note = Tap2#1-R
  source 1..21: mol_type = other DNA
caaccgcttcatcagtgttct

SEQ ID NO: 13 - DNA, 21 bp, synthetic construct
  misc_feature 1..21: note = Tap2#2-F
  source 1..21: mol_type = other DNA
ctggcggacatggctttactt

SEQ ID NO: 14 - DNA, 21 bp, synthetic construct
  misc_feature 1..21: note = Tap2#2-R
  source 1..21: mol_type = other DNA
ctcccacttttagcagtcccc

SEQ ID NO: 15 - DNA, 21 bp, synthetic construct
  misc_feature 1..21: note = b2m#1-F
  source 1..21: mol_type = other DNA
ttctggtgcttgtctcactga

SEQ ID NO: 16 - DNA, 22 bp, synthetic construct
  misc_feature 1..22: note = b2m#1-R
  source 1..22: mol_type = other DNA
cagtatgttcggcttcccattc

SEQ ID NO: 17 - DNA, 21 bp, synthetic construct
  misc_feature 1..21: note = b2m#2-F
  source 1..21: mol_type = other DNA
ttctggtgcttgtctcactga

SEQ ID NO: 18 - DNA, 22 bp, synthetic construct
  misc_feature 1..22: note = b2m#2-R
  source 1..22: mol_type = other DNA
cagtatgttcggcttcccattc

SEQ ID NO: 19 - DNA, 21 bp, synthetic construct
  misc_feature 1..21: note = Gapdh-F
  source 1..21: mol_type = other DNA
aagattgtcagcaatgcatcc

SEQ ID NO: 20 - DNA, 21 bp, synthetic construct
  misc_feature 1..21: note = Gapdh-R
  source 1..21: mol_type = other DNA
cttccacaatgccaaagttgt

SEQ ID NO: 21 - DNA, 23 bp, synthetic construct
  misc_feature 1..23: note = Forward primer
  source 1..23: mol_type = other DNA
cccaccccgctttgaccaaatac

SEQ ID NO: 22 - DNA, 21 bp, synthetic construct
  misc_feature 1..21: note = Reverse primer
  source 1..21: mol_type = other DNA
gtgccaccactgcccagcttc